SEQUENCE LISTING (38 sequences)

SEQ ID NO: 1 - DNA, 540 bp, Homo sapiens
  source 1..540: mol_type = genomic DNA
atgcaggccgagggcagaggaacaggcggcagcacaggcgacgcagatggaccaggcggccctggaatccctgatggcccaggcggcaatgctgggggaccaggagaagctggcgccacaggcgggagaggacctagaggagctggagccgctagagcttctggacctgggggaggcgcccctagaggaccacatggaggcgctgccagcggcctgaatggctgctgcagatgcggcgccagaggccctgagagccggctgctggaattctacctggccatgcccttcgccacccccatggaagccgagctggccagaagatccctggctcaggacgctcctcctctgcctgtgcccggcgtgctgctgaaagaattcaccgtgtccggcaacatcctgaccatcagactgacagccgccgatcacagacagctccagctgagcatcagctcttgcctgcagcagctgagcctgctgatgtggatcacccagtgctttctgcccgtgttcctggcccagccacccagcggacagagaagg

SEQ ID NO: 2 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 3 - DNA, 942 bp, Homo sapiens
  source 1..942: mol_type = genomic DNA
atgccccttgaacagcgctcacagcactgcaaacctgaggagggccttgaagcaaggggcgaagctctggggttggtcggtgcacaagcacccgccactgaggaacaggaagccgcgtctagctcatcaaccctggttgaagtgacactgggcgaagtgcctgctgcggagagtccagaccctccccagtcccctcaaggcgcttctagcctgcctaccacgatgaactacccactgtggtcacagagctatgaggacagttccaatcaagaagaagaaggcccgtctaccttccccgatcttgagtccgagtttcaggccgctctgtcccggaaggtggcagagctcgtgcactttctcctgttgaagtatcgagcccgggagcctgtcactaaggccgaaatgctgggctctgtagtggggaattggcagtatttcttccccgtgatcttcagcaaagcctccagcagcctgcaattggtgttcggtattgaactgatggaagtagatccgattgggcatctgtacatctttgcgacatgtctgggactgtcctatgacggactgctcggggataaccagattatgccgaaagccggtctgctgatcatagttctcgccatcattgccagagagggagattgtgctccagaggagaagatctgggaggaattgtctgtgctggaggtctttgagggtagggaggacagcattctcggcgatcccaagaaactcctgacccagcactttgtccaggagaactacctcgaatacagacaggttccaggcagtgaccctgcttgctacgagttcctttggggaccccgtgcattggtagagacaagctatgtcaaagtgctgcaccatatggtgaagatatctggaggaccacacatcagttacccaccccttcatgagtgggttctgcgcgaaggggaggag

SEQ ID NO: 4 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 5 - DNA, 1431 bp, Homo sapiens
  source 1..1431: mol_type = genomic DNA
atgctgctggccgtgctgtactgcctgctgtggagctttcagaccagcgccggacacttccctagagcctgcgtgagcagcaagaacctgatggaaaaagagtgctgccccccttggagcggcgatagaagcccctgtggccagctgagcggcagaggctcctgccagaacatcctgctgagcaacgcccctctgggcccccagttcccctttaccggcgtggacgacagagaaagctggcccagcgtgttctacaaccggacctgccagtgcagcggcaacttcatgggcttcaactgcggcaactgcaagttcggcttctggggacccaactgcaccgagagaaggctgctggtgcggagaaacatcttcgacctgagcgcccctgagaaggacaagttcttcgcctacctgaccctggccaagcacaccatcagcagcgactacgtgatccccatcggcacctacggccagatgaagaacggcagcacccccatgttcaacgacatcaacatctacgatctgttcgtgtggatgcactactacgtgtccatggacgccctgctgggcggcagcgagatctggagagacatcgactttgcccacgaggcccctgcctttctgccctggcaccggctgtttctgctgagatgggagcaggaaatccagaagctgaccggcgacgagaacttcaccatcccctactgggactggcgggacgccgagaagtgcgacatctgcaccgacgagtacatgggcggccagcaccccaccaaccccaatctgctgagccccgccagcttcttcagcagctggcagatcgtgtgctcccggctggaggagtacaacagccaccagagcctgtgcaatggcacccccgagggccctctgagaagaaaccccggcaaccacgacaagagccggacccccagactgcctagcagcgccgacgtggagttctgcctgagcctgacccagtacgagagcggcagcatggacaaggccgccaacttcagcttccggaacaccctggaaggcttcgccagccctctgaccggcattgccgacgccagccagagcagcatgcacaacgccctgcacatctacatgaatggaaccatgagccaggtgcagggcagcgccaacgaccccatcttcctgctgcaccacgccttcgtggacagcatcttcgagcagtggctgcggagacacagacccctgcaggaagtgtaccccgaggccaacgcccctatcggccacaaccgggagagctacatggtgcccttcatccccctgtaccggaacggcgacttcttcatcagctccaaggacctgggctacgactacagctacctgcaggacagcgaccccgacagcttccaggactacatcaagagctacctggaacaggccagcagaatctggtcctgg

SEQ ID NO: 6 - protein, 477 aa, Homo sapiens
MLLAVLYCLLWSFQTSAGHFPRACVSSKNLMEKECCPPWSGDRSPCGQLSGRGSCQNILLSNAPLGPQFPFTGVDDRESWPSVFYNRTCQCSGNFMGFNCGNCKFGFWGPNCTERRLLVRRNIFDLSAPEKDKFFAYLTLAKHTISSDYVIPIGTYGQMKNGSTPMFNDINIYDLFVWMHYYVSMDALLGGSEIWRDIDFAHEAPAFLPWHRLFLLRWEQEIQKLTGDENFTIPYWDWRDAEKCDICTDEYMGGQHPTNPNLLSPASFFSSWQIVCSRLEEYNSHQSLCNGTPEGPLRRNPGNHDKSRTPRLPSSADVEFCLSLTQYESGSMDKAANFSFRNTLEGFASPLTGIADASQSSMHNALHIYMNGTMSQVQGSANDPIFLLHHAFVDSIFEQWLRRHRPLQEVYPEANAPIGHNRESYMVPFIPLYRNGDFFISSKDLGYDYSYLQDSDPDSFQDYIKSYLEQASRIWSW

SEQ ID NO: 7 - DNA, 1653 bp, Homo sapiens
  source 1..1653: mol_type = genomic DNA
atgaacgagagccccgaccctacagatctggccggcgtgatcatcgagctgggacccaacgatagccctcagaccagcgagttcaagggggccacagaggaagcccctgccaaagagagcccccacacctccgagtttaagggcgctgctcgggtgtcccctatcagcgagagcgtgctggcccggctgagcaagttcgaggtggaggacgccgagaacgtggccagctacgacagcaagatcaagaaaatcgtgcacagcatcgtgtccagcttcgccttcggcctgttcggcgtgttcctggtgctgctggacgtgacactgatcctggccgacctgatcttcaccgacagcaagctgtacatccccctggaataccggtccatcagcctggccattgccctgttctttctgatggacgtgctgctgcgggtgttcgtggagcggcggcagcagtacttcagcgacctgttcaacatcctggacaccgccatcatcgtgattctgctgctggtggatgtggtgtacatcttcttcgacatcaagctgctgagaaacatcccccggtggacccatctgctgcggctgctgagactgatcatcctgctgcggatcttccacctgttccaccagaagcggcagctggaaaagctgatcagacggcgggtgtccgagaacaagcggcggtacaccagggacggcttcgacctggacctgacctacgtgaccgagcggatcattgccatgagcttccccagcagcggcagacagagcttctaccggaaccccatcaaagaagtggtgcggttcctggacaagaagcaccggaaccactaccgggtgtacaacctgtgcagcgagcgggcctacgaccccaagcacttccacaaccgggtggtgcggatcatgatcgacgaccacaacgtgcccaccctgcaccagatggtggtgttcaccaaagaagtgaacgagtggatggcccaggacctggaaaacatcgtggccatccactgcaagggcggcaccgacagaaccggcaccatggtgtgcgcctttctgatcgccagcgagatctgtagcaccgccaaagagtccctgtactacttcggcgagcggagaaccgacaagacccacagcgagaagttccagggcgtggagacacccagccagaaaagatatgtggcttacttcgcccaggtgaagcacctgtacaactggaacctgccccccagacggattctgttcatcaagcacttcatcatctacagcatccccagatacgtgcgggacctgaagatccagatcgagatggaaaagaaagtggtgttcagcaccatctccctgggcaagtgcagcgtgctggacaacatcaccaccgacaagatcctgatcgacgtgttcgacggcctgcccctgtacgacgacgtgaaggtgcagttcttctacagcaacctgcccacctactacgacaattgcagcttctacttctggctgcacaccagcttcatcgagaacaacaggctgtacctgcccaagaacgagctggacaacctgcacaagcagaaggccagaagaatctaccccagcgacttcgccgtggagatcctgtttggcgagaagatgaccagcagcgacgtggtggccggcagcgac

SEQ ID NO: 8 - protein, 551 aa, Homo sapiens
MNESPDPTDLAGVIIELGPNDSPQTSEFKGATEEAPAKESPHTSEFKGAARVSPISESVLARLSKFEVEDAENVASYDSKIKKIVHSIVSSFAFGLFGVFLVLLDVTLILADLIFTDSKLYIPLEYRSISLAIALFFLMDVLLRVFVERRQQYFSDLFNILDTAIIVILLLVDVVYIFFDIKLLRNIPRWTHLLRLLRLIILLRIFHLFHQKRQLEKLIRRRVSENKRRYTRDGFDLDLTYVTERIIAMSFPSSGRQSFYRNPIKEVVRFLDKKHRNHYRVYNLCSERAYDPKHFHNRVVRIMIDDHNVPTLHQMVVFTKEVNEWMAQDLENIVAIHCKGGTDRTGTMVCAFLIASEICSTAKESLYYFGERRTDKTHSEKFQGVETPSQKRYVAYFAQVKHLYNWNLPPRRILFIKHFIIYSIPRYVRDLKIQIEMEKKVVFSTISLGKCSVLDNITTDKILIDVFDGLPLYDDVKVQFFYSNLPTYYDNCSFYFWLHTSFIENNRLYLPKNELDNLHKQKARRIYPSDFAVEILFGEKMTSSDVVAGSD

SEQ ID NO: 9 - DNA, 1528 bp, synthetic construct
  source 1..1528: mol_type = other DNA
gggcgaactagtattcttctggtccccacagactcagagagaacccgccaccatgagagtgaccgcccccagaaccctgatcctgctgctgtctggcgccctggccctgacagagacatgggccggaagcggaggatctatgcaggccgagggcagaggaacaggcggcagcacaggcgacgcagatggaccaggcggccctggaatccctgatggcccaggcggcaatgctgggggaccaggagaagctggcgccacaggcgggagaggacctagaggagctggagccgctagagcttctggacctgggggaggcgcccctagaggaccacatggaggcgctgccagcggcctgaatggctgctgcagatgcggcgccagaggccctgagagccggctgctggaattctacctggccatgcccttcgccacccccatggaagccgagctggccagaagatccctggctcaggacgctcctcctctgcctgtgcccggcgtgctgctgaaagaattcaccgtgtccggcaacatcctgaccatcagactgacagccgccgatcacagacagctccagctgagcatcagctcttgcctgcagcagctgagcctgctgatgtggatcacccagtgctttctgcccgtgttcctggcccagccacccagcggacagagaaggggcggatccctgggaggcgggggaagcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcgggggatctagcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgactcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - DNA, 1565 bp, synthetic construct
  source 1..1565: mol_type = other DNA
gggcgaactagtattcttctggtccccacagactcagagagaacccgccaccatgagagtgatggcccccagaaccctgatcctgctgctgtctggcgccctggccctgacagagacatgggccggaagcggcggctctggaggaggcggctccggaggcatgcaggccgagggcagaggaacaggcggcagcacaggcgacgcagatggaccaggcggccctggaatccctgatggcccaggcggcaatgctgggggaccaggagaagctggcgccacaggcgggagaggacctagaggagctggagccgctagagcttctggacctgggggaggcgcccctagaggaccacatggaggcgctgccagcggcctgaatggctgctgcagatgcggcgccagaggccctgagagccggctgctggaattctacctggccatgcccttcgccacccccatggaagccgagctggccagaagatccctggctcaggacgctcctcctctgcctgtgcccggcgtgctgctgaaagaattcaccgtgtccggcaacatcctgaccatcagactgacagccgccgatcacagacagctccagctgagcatcagctcttgcctgcagcagctgagcctgctgatgtggatcacccagtgctttctgcccgtgttcctggcccagccacccagcggacagagaaggggaggatccggtggtggcggcagcggcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcgggggatctagcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 11 - protein, 353 aa, synthetic construct
MRVTAPRTLILLLSGALALTETWAGSGGSMQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRRGGSLGGGGSGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 12 - protein, 360 aa, synthetic construct
MRVMAPRTLILLLSGALALTETWAGSGGSGGGGSGGMQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRRGGSGGGGSGGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 13 - DNA, 2329 bp, synthetic construct
  source 1..2329: mol_type = other DNA
gggcgaactagtattcttctggtccccacagactcagagagaacccgccaccatgcttctggctgtgctctattgcttgctgtggtccttccagacttctgccggacactttcccagagcatgtgtgtcatccaaaaacctcatggagaaagaatgctgtcctccttggagtggtgatagatccccatgtggacagctctcaggcagaggatcttgccagaacattctgctgagcaatgcacctcttggcccacagtttccctttaccggagtagatgacagagaaagttggccctccgtgttctacaacagaacatgccaatgcagcggcaattttatgggatttaactgcggaaattgcaaatttggattttggggcccaaactgtacagaaagaagactgctcgttagaagaaacatctttgatctgagcgcacccgaaaaagacaaattcttcgcttatctgactctcgccaaacacacaataagcagtgattatgtcattcccattggcacttatggacagatgaaaaatggctcaactcccatgttcaacgacatcaacatatacgatctgtttgtgtggatgcattactacgtgagtatggatgctctgctgggtggctccgaaatatggagggatatagattttgcacacgaagctcctgccttccttccatggcatagactgttcctgctgagatgggaacaagaaatccagaaacttacaggcgatgaaaacttcactatcccttattgggattggagagatgctgagaaatgcgatatctgtaccgatgaatacatgggtggtcaacacccaaccaaccccaatctcctgagccctgcctctttcttcagttcttggcagattgtctgttcaagattggaagaatacaattcccatcagtccctgtgtaacggaacaccagaaggacctctgagaagaaaccctggcaatcatgacaaaagcaggacacctagacttccctcctctgccgacgttgaattttgcctctctctgacccagtacgaaagcggtagcatggacaaagccgccaatttcagctttagaaataccttggaaggatttgcctcacctctgacaggaattgctgatgctagccagtcatccatgcacaacgctttgcacatctatatgaatggaaccatgagtcaggttcagggatctgccaatgatcctattttcctgctgcaccatgcattcgtggactctatctttgagcagtggcttagaagacacagaccattgcaggaagtctatccagaggccaacgcaccaatcggccataatagagaaagctacatggtacccttcattcctctgtacagaaatggagatttcttcatcagctccaaagacctgggctacgattactcatatctgcaagacagtgatcccgatagcttccaagactacattaagtcttatctcgaacaggcgagcagaatctggtcctggggatccctgggaggcgggggaagcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcggagccgtggtggctaccgtgatgtgcagacggaagtccagcggaggcaagggcggcagctaagccgtggtggctaccgtgatgtgcagacggaagtccagcggaggcaagggcggcagctacagccaggccgccagctctgatagcgcccagggcagcgacgtgtcactgacagcctgactcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - DNA, 2348 bp, synthetic construct
  source 1..2348: mol_type = other DNA
gggcgaactagtattcttctggtccccacagactcagagagaacccgccaccatgcttctggctgtgctctattgcttgctgtggtccttccagacttctgccggacactttcccagagcatgtgtgtcatccaaaaacctcatggagaaagaatgctgtcctccttggagtggtgatagatccccatgtggacagctctcaggcagaggatcttgccagaacattctgctgagcaatgcacctcttggcccacagtttccctttaccggagtagatgacagagaaagttggccctccgtgttctacaacagaacatgccaatgcagcggcaattttatgggatttaactgcggaaattgcaaatttggattttggggcccaaactgtacagaaagaagactgctcgttagaagaaacatctttgatctgagcgcacccgaaaaagacaaattcttcgcttatctgactctcgccaaacacacaataagcagtgattatgtcattcccattggcacttatggacagatgaaaaatggctcaactcccatgttcaacgacatcaacatatacgatctgtttgtgtggatgcattactacgtgagtatggatgctctgctgggtggctccgaaatatggagggatatagattttgcacacgaagctcctgccttccttccatggcatagactgttcctgctgagatgggaacaagaaatccagaaacttacaggcgatgaaaacttcactatcccttattgggattggagagatgctgagaaatgcgatatctgtaccgatgaatacatgggtggtcaacacccaaccaaccccaatctcctgagccctgcctctttcttcagttcttggcagattgtctgttcaagattggaagaatacaattcccatcagtccctgtgtaacggaacaccagaaggacctctgagaagaaaccctggcaatcatgacaaaagcaggacacctagacttccctcctctgccgacgttgaattttgcctctctctgacccagtacgaaagcggtagcatggacaaagccgccaatttcagctttagaaataccttggaaggatttgcctcacctctgacaggaattgctgatgctagccagtcatccatgcacaacgctttgcacatctatatgaatggaaccatgagtcaggttcagggatctgccaatgatcctattttcctgctgcaccatgcattcgtggactctatctttgagcagtggcttagaagacacagaccattgcaggaagtctatccagaggccaacgcaccaatcggccataatagagaaagctacatggtacccttcattcctctgtacagaaatggagatttcttcatcagctccaaagacctgggctacgattactcatatctgcaagacagtgatcccgatagcttccaagactacattaagtcttatctcgaacaggcgagcagaatctggtcctggggaggatccggtggtggcggcagcggcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcggagccgtggtggctaccgtgatgtgcagacggaagtccagcggaggcaagggcggcagctaagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 15 - protein, 620 aa, synthetic construct
MLLAVLYCLLWSFQTSAGHFPRACVSSKNLMEKECCPPWSGDRSPCGQLSGRGSCQNILLSNAPLGPQFPFTGVDDRESWPSVFYNRTCQCSGNFMGFNCGNCKFGFWGPNCTERRLLVRRNIFDLSAPEKDKFFAYLTLAKHTISSDYVIPIGTYGQMKNGSTPMFNDINIYDLFVWMHYYVSMDALLGGSEIWRDIDFAHEAPAFLPWHRLFLLRWEQEIQKLTGDENFTIPYWDWRDAEKCDICTDEYMGGQHPTNPNLLSPASFFSSWQIVCSRLEEYNSHQSLCNGTPEGPLRRNPGNHDKSRTPRLPSSADVEFCLSLTQYESGSMDKAANFSFRNTLEGFASPLTGIADASQSSMHNALHIYMNGTMSQVQGSANDPIFLLHHAFVDSIFEQWLRRHRPLQEVYPEANAPIGHNRESYMVPFIPLYRNGDFFISSKDLGYDYSYLQDSDPDSFQDYIKSYLEQASRIWSWGSLGGGGSGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 16 - protein, 621 aa, synthetic construct
MLLAVLYCLLWSFQTSAGHFPRACVSSKNLMEKECCPPWSGDRSPCGQLSGRGSCQNILLSNAPLGPQFPFTGVDDRESWPSVFYNRTCQCSGNFMGFNCGNCKFGFWGPNCTERRLLVRRNIFDLSAPEKDKFFAYLTLAKHTISSDYVIPIGTYGQMKNGSTPMFNDINIYDLFVWMHYYVSMDALLGGSEIWRDIDFAHEAPAFLPWHRLFLLRWEQEIQKLTGDENFTIPYWDWRDAEKCDICTDEYMGGQHPTNPNLLSPASFFSSWQIVCSRLEEYNSHQSLCNGTPEGPLRRNPGNHDKSRTPRLPSSADVEFCLSLTQYESGSMDKAANFSFRNTLEGFASPLTGIADASQSSMHNALHIYMNGTMSQVQGSANDPIFLLHHAFVDSIFEQWLRRHRPLQEVYPEANAPIGHNRESYMVPFIPLYRNGDFFISSKDLGYDYSYLQDSDPDSFQDYIKSYLEQASRIWSWGGSGGGGSGGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 17 - DNA, 1936 bp, synthetic construct
  source 1..1936: mol_type = other DNA
gggcgaactagtattcttctggtccccacagactcagagagaacccgccaccatgagagtgaccgcccccagaaccctgatcctgctgctgtctggcgccctggccctgacagagacatgggccggaagcctgcagggaggatctatgccccttgaacagcgctcacagcactgcaaacctgaggagggccttgaagcaaggggcgaagctctggggttggtcggtgcacaagcacccgccactgaggaacaggaagccgcgtctagctcatcaaccctggttgaagtgacactgggcgaagtgcctgctgcggagagtccagaccctccccagtcccctcaaggcgcttctagcctgcctaccacgatgaactacccactgtggtcacagagctatgaggacagttccaatcaagaagaagaaggcccgtctaccttccccgatcttgagtccgagtttcaggccgctctgtcccggaaggtggcagagctcgtgcactttctcctgttgaagtatcgagcccgggagcctgtcactaaggccgaaatgctgggctctgtagtggggaattggcagtatttcttccccgtgatcttcagcaaagcctccagcagcctgcaattggtgttcggtattgaactgatggaagtagatccgattgggcatctgtacatctttgcgacatgtctgggactgtcctatgacggactgctcggggataaccagattatgccgaaagccggtctgctgatcatagttctcgccatcattgccagagagggagattgtgctccagaggagaagatctgggaggaattgtctgtgctggaggtctttgagggtagggaggacagcattctcggcgatcccaagaaactcctgacccagcactttgtccaggagaactacctcgaatacagacaggttccaggcagtgaccctgcttgctacgagttcctttggggaccccgtgcattggtagagacaagctatgtcaaagtgctgcaccatatggtgaagatatctggaggaccacacatcagttacccaccccttcatgagtgggttctgcgcgaaggggaggagggaggatccctgggaggcgggggaagcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcgggggatctagcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgactcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - DNA, 1967 bp, synthetic construct
  source 1..1967: mol_type = other DNA
gggcgaactagtattcttctggtccccacagactcagagagaacccgccaccatgagagtgatggcccccagaaccctgatcctgctgctgtctggcgccctggccctgacagagacatgggccggaagcggcggctctggaggaggcggctccggaggcatgccccttgaacagcgctcacagcactgcaaacctgaggagggccttgaagcaaggggcgaagctctggggttggtcggtgcacaagcacccgccactgaggaacaggaagccgcgtctagctcatcaaccctggttgaagtgacactgggcgaagtgcctgctgcggagagtccagaccctccccagtcccctcaaggcgcttctagcctgcctaccacgatgaactacccactgtggtcacagagctatgaggacagttccaatcaagaagaagaaggcccgtctaccttccccgatcttgagtccgagtttcaggccgctctgtcccggaaggtggcagagctcgtgcactttctcctgttgaagtatcgagcccgggagcctgtcactaaggccgaaatgctgggctctgtagtggggaattggcagtatttcttccccgtgatcttcagcaaagcctccagcagcctgcaattggtgttcggtattgaactgatggaagtagatccgattgggcatctgtacatctttgcgacatgtctgggactgtcctatgacggactgctcggggataaccagattatgccgaaagccggtctgctgatcatagttctcgccatcattgccagagagggagattgtgctccagaggagaagatctgggaggaattgtctgtgctggaggtctttgagggtagggaggacagcattctcggcgatcccaagaaactcctgacccagcactttgtccaggagaactacctcgaatacagacaggttccaggcagtgaccctgcttgctacgagttcctttggggaccccgtgcattggtagagacaagctatgtcaaagtgctgcaccatatggtgaagatatctggaggaccacacatcagttacccaccccttcatgagtgggttctgcgcgaaggggaggagggaggatccggtggtggcggcagcggcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcgggggatctagcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 19 - protein, 489 aa, synthetic construct
MRVTAPRTLILLLSGALALTETWAGSLQGGSMPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEGGSLGGGGSGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 20 - protein, 494 aa, synthetic construct
MRVMAPRTLILLLSGALALTETWAGSGGSGGGGSGGMPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEGGSGGGGSGGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 21 - DNA, 2635 bp, synthetic construct
  source 1..2635: mol_type = other DNA
gggcgaactagtattcttctggtccccacagactcagagagaacccgccaccatgagagtgaccgcccccagaaccctgatcctgctgctgtctggcgccctggccctgacagagacatgggccggaagcctgcagatgaacgagagccccgaccctacagatctggccggcgtgatcatcgagctgggacccaacgatagccctcagaccagcgagttcaagggggccacagaggaagcccctgccaaagagagcccccacacctccgagtttaagggcgctgctcgggtgtcccctatcagcgagagcgtgctggcccggctgagcaagttcgaggtggaggacgccgagaacgtggccagctacgacagcaagatcaagaaaatcgtgcacagcatcgtgtccagcttcgccttcggcctgttcggcgtgttcctggtgctgctggacgtgacactgatcctggccgacctgatcttcaccgacagcaagctgtacatccccctggaataccggtccatcagcctggccattgccctgttctttctgatggacgtgctgctgcgggtgttcgtggagcggcggcagcagtacttcagcgacctgttcaacatcctggacaccgccatcatcgtgattctgctgctggtggatgtggtgtacatcttcttcgacatcaagctgctgagaaacatcccccggtggacccatctgctgcggctgctgagactgatcatcctgctgcggatcttccacctgttccaccagaagcggcagctggaaaagctgatcagacggcgggtgtccgagaacaagcggcggtacaccagggacggcttcgacctggacctgacctacgtgaccgagcggatcattgccatgagcttccccagcagcggcagacagagcttctaccggaaccccatcaaagaagtggtgcggttcctggacaagaagcaccggaaccactaccgggtgtacaacctgtgcagcgagcgggcctacgaccccaagcacttccacaaccgggtggtgcggatcatgatcgacgaccacaacgtgcccaccctgcaccagatggtggtgttcaccaaagaagtgaacgagtggatggcccaggacctggaaaacatcgtggccatccactgcaagggcggcaccgacagaaccggcaccatggtgtgcgcctttctgatcgccagcgagatctgtagcaccgccaaagagtccctgtactacttcggcgagcggagaaccgacaagacccacagcgagaagttccagggcgtggagacacccagccagaaaagatatgtggcttacttcgcccaggtgaagcacctgtacaactggaacctgccccccagacggattctgttcatcaagcacttcatcatctacagcatccccagatacgtgcgggacctgaagatccagatcgagatggaaaagaaagtggtgttcagcaccatctccctgggcaagtgcagcgtgctggacaacatcaccaccgacaagatcctgatcgacgtgttcgacggcctgcccctgtacgacgacgtgaaggtgcagttcttctacagcaacctgcccacctactacgacaattgcagcttctacttctggctgcacaccagcttcatcgagaacaacaggctgtacctgcccaagaacgagctggacaacctgcacaagcagaaggccagaagaatctaccccagcgacttcgccgtggagatcctgtttggcgagaagatgaccagcagcgacgtggtggccggcagcgacggatccctgggaggcgggggaagcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcgggggatctagcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctgactcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 22 - DNA, 2675 bp, synthetic construct
  source 1..2675: mol_type = other DNA
gggcgaactagtattcttctggtccccacagactcagagagaacccgccaccatgagagtgatggcccccagaaccctgatcctgctgctgtctggcgccctggccctgacagagacatgggccggaagcggcggctctggaggaggcggctccggaggcatgaacgagagccccgaccctacagatctggccggcgtgatcatcgagctgggacccaacgatagccctcagaccagcgagttcaagggggccacagaggaagcccctgccaaagagagcccccacacctccgagtttaagggcgctgctcgggtgtcccctatcagcgagagcgtgctggcccggctgagcaagttcgaggtggaggacgccgagaacgtggccagctacgacagcaagatcaagaaaatcgtgcacagcatcgtgtccagcttcgccttcggcctgttcggcgtgttcctggtgctgctggacgtgacactgatcctggccgacctgatcttcaccgacagcaagctgtacatccccctggaataccggtccatcagcctggccattgccctgttctttctgatggacgtgctgctgcgggtgttcgtggagcggcggcagcagtacttcagcgacctgttcaacatcctggacaccgccatcatcgtgattctgctgctggtggatgtggtgtacatcttcttcgacatcaagctgctgagaaacatcccccggtggacccatctgctgcggctgctgagactgatcatcctgctgcggatcttccacctgttccaccagaagcggcagctggaaaagctgatcagacggcgggtgtccgagaacaagcggcggtacaccagggacggcttcgacctggacctgacctacgtgaccgagcggatcattgccatgagcttccccagcagcggcagacagagcttctaccggaaccccatcaaagaagtggtgcggttcctggacaagaagcaccggaaccactaccgggtgtacaacctgtgcagcgagcgggcctacgaccccaagcacttccacaaccgggtggtgcggatcatgatcgacgaccacaacgtgcccaccctgcaccagatggtggtgttcaccaaagaagtgaacgagtggatggcccaggacctggaaaacatcgtggccatccactgcaagggcggcaccgacagaaccggcaccatggtgtgcgcctttctgatcgccagcgagatctgtagcaccgccaaagagtccctgtactacttcggcgagcggagaaccgacaagacccacagcgagaagttccagggcgtggagacacccagccagaaaagatatgtggcttacttcgcccaggtgaagcacctgtacaactggaacctgccccccagacggattctgttcatcaagcacttcatcatctacagcatccccagatacgtgcgggacctgaagatccagatcgagatggaaaagaaagtggtgttcagcaccatctccctgggcaagtgcagcgtgctggacaacatcaccaccgacaagatcctgatcgacgtgttcgacggcctgcccctgtacgacgacgtgaaggtgcagttcttctacagcaacctgcccacctactacgacaattgcagcttctacttctggctgcacaccagcttcatcgagaacaacaggctgtacctgcccaagaacgagctggacaacctgcacaagcagaaggccagaagaatctaccccagcgacttcgccgtggagatcctgtttggcgagaagatgaccagcagcgacgtggtggccggcagcggaggatccggtggtggcggcagcggcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcgggggatctagcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 23 - protein, 722 aa, synthetic construct
MRVTAPRTLILLLSGALALTETWAGSLQMNESPDPTDLAGVIIELGPNDSPQTSEFKGATEEAPAKESPHTSEFKGAARVSPISESVLARLSKFEVEDAENVASYDSKIKKIVHSIVSSFAFGLFGVFLVLLDVTLILADLIFTDSKLYIPLEYRSISLAIALFFLMDVLLRVFVERRQQYFSDLFNILDTAIIVILLLVDVVYIFFDIKLLRNIPRWTHLLRLLRLIILLRIFHLFHQKRQLEKLIRRRVSENKRRYTRDGFDLDLTYVTERIIAMSFPSSGRQSFYRNPIKEVVRFLDKKHRNHYRVYNLCSERAYDPKHFHNRVVRIMIDDHNVPTLHQMVVFTKEVNEWMAQDLENIVAIHCKGGTDRTGTMVCAFLIASEICSTAKESLYYFGERRTDKTHSEKFQGVETPSQKRYVAYFAQVKHLYNWNLPPRRILFIKHFIIYSIPRYVRDLKIQIEMEKKVVFSTISLGKCSVLDNITTDKILIDVFDGLPLYDDVKVQFFYSNLPTYYDNCSFYFWLHTSFIENNRLYLPKNELDNLHKQKARRIYPSDFAVEILFGEKMTSSDVVAGSDGSLGGGGSGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 24 - protein, 730 aa, synthetic construct
MRVMAPRTLILLLSGALALTETWAGSGGSGGGGSGGMNESPDPTDLAGVIIELGPNDSPQTSEFKGATEEAPAKESPHTSEFKGAARVSPISESVLARLSKFEVEDAENVASYDSKIKKIVHSIVSSFAFGLFGVFLVLLDVTLILADLIFTDSKLYIPLEYRSISLAIALFFLMDVLLRVFVERRQQYFSDLFNILDTAIIVILLLVDVVYIFFDIKLLRNIPRWTHLLRLLRLIILLRIFHLFHQKRQLEKLIRRRVSENKRRYTRDGFDLDLTYVTERIIAMSFPSSGRQSFYRNPIKEVVRFLDKKHRNHYRVYNLCSERAYDPKHFHNRVVRIMIDDHNVPTLHQMVVFTKEVNEWMAQDLENIVAIHCKGGTDRTGTMVCAFLIASEICSTAKESLYYFGERRTDKTHSEKFQGVETPSQKRYVAYFAQVKHLYNWNLPPRRILFIKHFIIYSIPRYVRDLKIQIEMEKKVVFSTISLGKCSVLDNITTDKILIDVFDGLPLYDDVKVQFFYSNLPTYYDNCSFYFWLHTSFIENNRLYLPKNELDNLHKQKARRIYPSDFAVEILFGEKMTSSDVVAGSGGSGGGGSGGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 25 - RNA, 30 nt, synthetic construct
  source 1..30: mol_type = other RNA
aaaaaaaaaaaaaaaaaaaaaaaaaaaaaa

SEQ ID NO: 26 - RNA, 70 nt, synthetic construct
  source 1..70: mol_type = other RNA
aaaaaaaaaaaaaaaaaaaaaaaaaaaaaaaaaaaaaaaaaaaaaaaaaaaaaaaaaaaaaaaaaaaaaa

SEQ ID NO: 27 - DNA, 31 bp, synthetic construct
  source 1..31: mol_type = other DNA
  modified_base 1: mod_base = OTHER; note = Phosphate-Guanine
gccaccatgatcctaaacaaagctctgmtgc

SEQ ID NO: 28 - DNA, 33 bp, synthetic construct
  source 1..33: mol_type = other DNA
tatgcgatcgctcacaakggcccytggtgtctg

SEQ ID NO: 29 - DNA, 23 bp, synthetic construct
  source 1..23: mol_type = other DNA
catcacaggaactttctgggctg

SEQ ID NO: 30 - DNA, 24 bp, synthetic construct
  source 1..24: mol_type = other DNA
gctggtaggacaccgaggtaaagc

SEQ ID NO: 31 - DNA, 24 bp, synthetic construct
  source 1..24: mol_type = other DNA
gctggtaagactcggaggtgaagc

SEQ ID NO: 32 - DNA, 20 bp, synthetic construct
  source 1..20: mol_type = other DNA
cacgtggtcggggwagaagc

SEQ ID NO: 33 - DNA, 53 bp, synthetic construct
  source 1..53: mol_type = other DNA
  misc_difference 26: note = a, c, t, g, unknown, or other
  misc_difference 27: note = a, c, t, g, unknown, or other
  misc_difference 28: note = a, c, t, g, unknown, or other
  misc_difference 29: note = a, c, t, g, unknown, or other
  misc_difference 30: note = a, c, t, g, unknown, or other
cgatccagactagacgctcaggaagnnnnnaagcagtggtatcaacgcagagt

SEQ ID NO: 34 - DNA, 53 bp, synthetic construct
  source 1..53: mol_type = other DNA
caatatgtgaccgccgagtcccaggttagagtctctcagctggtacacggcag

SEQ ID NO: 35 - DNA, 50 bp, synthetic construct
  source 1..50: mol_type = other DNA
caatatgtgaccgccgagtcccaggggctcaaacacagcgacctcgggtg

SEQ ID NO: 36 - DNA, 30 bp, synthetic construct
  source 1..30: mol_type = other DNA
  misc_difference 1: note = a, c, t, g, unknown, or other
  misc_difference 1: note = This position may be deleted
  misc_difference 2: note = a, c, t, g, unknown, or other
  misc_difference 3: note = a, c, t, g, unknown, or other
  misc_difference 4: note = a, c, t, g, unknown, or other
  misc_difference 5: note = a, c, t, g, unknown, or other
nnnnncgatccagactagacgctcaggaag

SEQ ID NO: 37 - DNA, 30 bp, synthetic construct
  source 1..30: mol_type = other DNA
  misc_difference 1: note = a, c, t, g, unknown, or other
  misc_difference 2: note = a, c, t, g, unknown, or other
  misc_difference 3: note = a, c, t, g, unknown, or other
  misc_difference 4: note = a, c, t, g, unknown, or other
  misc_difference 5: note = a, c, t, g, unknown, or other
nnnnncaatatgtgaccgccgagtcccagg

SEQ ID NO: 38 - RNA, 120 nt, synthetic construct
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2025/0345417 A1
(43) Pub. Date: Nov. 13, 2025

(54) COMPOSITIONS AND METHODS FOR TREATMENT OF MELANOMA

(71) Applicant: BioNTech SE, Mainz (DE)

(72) Inventors: Ugur Sahin, Mainz (DE); Robert A. Jabulowsky, Offenbach (DE); Doreen Schwarck-Kokarakis, Bingen (DE); Özlem Türeci, Mainz (DE)

(21) Appl. No.: 18/292,232

(22) PCT Filed: Jul. 28, 2022
(86) PCT No.: PCT/EP2022/071276

§ 371 Date: Jan. 25, 2024

Related U.S. Application Data
(60) Provisional application No. 63/227,323, filed on Jul. 29, 2021.
(60) Provisional application No. 63/256,377, filed on Oct. 15, 2021.

(51) Int. Cl. A61K 39/395; A61K 9/14; A61K 31/7105; A61K 39/00; A61P 35/00; C07K 16/28

(52) U.S. Cl. CPC A61K 39/3955; A61K 9/14; A61K 31/7105; A61K 39/0011; A61P 35/00; C07K 16/2896; A61K 2039/57; A61K 2039/876

(57) ABSTRACT

The present disclosure provides compositions and methods for treatment of melanoma.

### CROSS-REFERENCE TO RELATED APPLICATIONS

This application is the U.S. national stage of PCT/EP22/71276, filed Jul. 28, 2022, which claims priority to U.S. Appl. No. 63/227,323, filed Jul. 29, 2021, and U.S. Appl. No. 63/256,377, filed Oct. 15, 2021, the entire contents of each of which are hereby incorporated by reference.

### SEQUENCE LISTING

The present application contains a Sequence Listing which has been submitted in XML format via EFS-Web and is hereby incorporated by reference in its entirety. The XML copy, created on Aug. 24, 2022, is named “2013237_generic_SL.xml” and is 68,559 bytes in size.

### BACKGROUND

Cancer is the second leading cause of death globally. Conventional therapies such as chemotherapy, radiotherapy, surgery, and targeted therapies (e.g., including recent advances in immunotherapies) have improved outcomes in patients with advanced solid tumors. In the last few years, the Food and Drug Administration (FDA) and European Medicines Agency (EMA) have approved checkpoint inhibitors (targeting the CTLA-4 pathway, ipilimumab, and targeting programmed death receptor/ligand [PD/PD-L1], including atezolizumab, avelumab, durvalumab, nivolumab, cemiplimab and pembrolizumab), for the treatment of patients with multiple cancer types, mainly solid tumors, including melanoma. However, success with these therapies has not been shown in advanced stage patients with treatment refractory tumors. Similarly, clinical efforts to treat cancer using vaccines that stimulate a targeted immune response against the tumors have also been unsuccessful in such advanced stage patients.

### SUMMARY

The poor prognosis of certain cancers such as, e.g., melanoma, highlights the need for additional treatment approaches. The present disclosure, among other things, provides an insight that a pharmaceutical composition (e.g., an immunogenic composition such as, e.g., in some embodiments a vaccine) that delivers RNA molecules encoding melanoma tumor-associated antigens (TAA) (e.g., melanoma TAAs) represents a particularly efficacious treatment option for patients suffering from melanoma. Such RNA molecules can, e.g., target dendritic cells in lymphoid tissues. The present disclosure, among other things, also provides an insight that pharmaceutical compositions described herein are particularly useful and/or effective when administered to patients with advanced-stage melanoma (e.g., stage III or stage IV melanoma). Advanced stage cancer, e.g., advanced stage melanoma is also referred to as “late stage” cancer. Moreover, the present disclosure provides a particular insight that patients with no evidence of disease at time of first administration of pharmaceutical compositions described herein (e.g., in some embodiments patients whose melanoma have been fully resected) can still benefit from anti-tumor immunity induced by such pharmaceutical compositions.

Without wishing to be bound by any particular theory, as TAA are typically non-mutated self-antigens, central T-cell tolerance may contribute to largely weak, clinically ineffective T-cell responses observed in certain clinical trials for cancer vaccine. The present disclosure, among other things, provides an insight that a combination of tumor associated antigens including a New York oesophageal squamous cell carcinoma (NY-ESO-1) antigen, a melanoma-associated antigen A3 (MAGE-A3) antigen, a tyrosinase antigen, and a transmembrane phosphatase with tensin homology (TPTE) antigen represents a particular useful set of tumor-associated antigens for targeted immunotherapies. Without wishing to be bound by any particular theory, the present disclosure notes that the restricted normal tissue expression of such a combination of tumor associated antigens and its high prevalence in melanoma (e.g., over 90% of melanoma patients expressing at least one of the tumor associated antigens NY-ESO-1 antigen, MAGE-A3 antigen, tyrosinase antigen and TPTE antigen) may contribute to its usefulness in treatment of melanoma.

The present disclosure further provides the insight that compositions disclosed herein can induce de novo antigen-specific anti-tumor immune responses and enhances pre-existing immune responses against the vaccine antigens.

Still further, the present disclosure provides a particular insight that delivery of tumor associated antigens (NY-ESO-1 antigen, MAGE-A3 antigen, tyrosinase antigen and TPTE antigen) by RNA via lipid particles (e.g., lipoplexes or lipid nanoparticles) that target dendritic cells (e.g., immature dendritic cells) where the RNA is translated for antigen presentation (e.g., augmented presentation) on HLA class I and II molecules, may be a particularly beneficial strategy for cancer vaccine. Without wishing to be bound by a particular theory, in some embodiments, RNA compositions described herein can align vaccine antigen delivery temporospatially with co-stimulation through toll-like receptor (TLR)-mediated, type-I-interferon driven antiviral immune mechanisms, and results in profound expansion of antigen specific T-cells. The present disclosure, among other things, also provides an insight that RNA compositions described herein are not only effective as monotherapy for treatment of melanoma, but can also synergize with an immune checkpoint inhibitor (e.g., an anti-PD1 therapy) in melanoma patients, who in some embodiments may have been previously treated with an immune checkpoint inhibitor. To date, no therapy comprising a cancer vaccine comprising ribonucleic acid encoding tumor associated antigen(s) and lipid particles (e.g., lipoplexes or lipid nanoparticles) has been approved for treatment of cancer (e.g., melanoma). Those skilled in the art will be aware of the burgeoning field of nucleic acid therapeutics, and moreover of RNA (e.g., mRNA) therapeutics (see, for example, mRNA-encoding proteins and/or cytokines). Various embodiments of technologies provided herein may utilize particular features of developed RNA (e.g., mRNA) therapeutic technologies and/or delivery systems. For example, in some embodiments, an administered RNA (e.g., mRNA) may comprise non-nucleoside modified nucleotides. In some embodiments, an administered RNA (e.g., mRNA) may comprise one or more modified nucleotides (e.g., but not limited to pseudouridine), nucleosides, and/or linkages. Alternatively or additionally, in some embodiments, an administered RNA (e.g., mRNA) may comprise a modified polyA sequence (e.g., a disrupted polyA sequence) that enhances stability and/or translation efficiency. Alternatively or additionally, in some embodiments, an administered RNA (e.g., mRNA) may comprise a specific combination of at least two 3′UTR sequences (e.g., a combination of a sequence element of an amino terminal enhancer of split RNA and a sequence derived from a mitochondrially encoded 12S RNA). Alternatively or additionally, in some embodiments, an administered RNA (e.g., mRNA) may comprise a ′5 UTR sequence that is derived from human α-globin mRNA. Alternatively or additionally, in some embodiments, an administered RNA (e.g., mRNA) may comprise a 5′ cap analog, e.g., for co-transcriptionally capping. Alternatively or additionally, in some embodiments, an administered RNA (e.g., mRNA) may comprise a secretion signal-coding region with reduced immunogenicity (e.g., a human secretion signal-coding sequence). In some embodiments, an administered RNA (e.g., mRNA) may comprise a MHC trafficking domain. In some embodiments, an administered RNA may be formulated in or with one or more delivery vehicles (e.g., lipid particles, e.g., lipoplexes or lipid nanoparticles).

In one aspect, the present disclosure, among other things, provides a method comprising: administering at least one dose of a pharmaceutical composition to a patient suffering from cancer, wherein the pharmaceutical composition comprises: (a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and (b) lipid particles.

In some embodiments, a patient amenable to technologies described herein (including, e.g., methods and/or pharmaceutical compositions, etc.) is classified as having evidence of disease at the time of administration.

In some embodiments, a patient amenable to technologies described herein (including, e.g., methods and/or pharmaceutical compositions, etc.) is classified as having no evidence of disease at the time of administration.

Accordingly, certain aspects of the present disclosure provide a method comprising: administering to a patient at least one dose of a pharmaceutical composition comprising: (a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and (b) lipid particles; wherein the patient was diagnosed with cancer prior to the time of administration, but the patient is classified as having no evidence of disease at the time of administration.

In some embodiments, evidence of disease or no evidence of disease is or was determined by applying an immune-related Response Evaluation Criteria In Solid Tumors (irRECIST) standard or RECIST 1.1 standard.

In some embodiments, technologies described herein involve a pharmaceutical composition that comprises one or more RNA molecules comprising: (i) a first RNA molecule encoding the NY-ESO-1 antigen, (ii) a second RNA molecule encoding a MAGE-A3 antigen, (iii) a third RNA molecule encoding a tyrosinase antigen, and (iv) a fourth RNA molecule encoding a TPTE antigen. In some embodiments, a single RNA molecule of the one or more RNA molecules encodes at least two of a NY-ESO-1 antigen, a MAGE-A3 antigen, a tyrosinase antigen, and a TPTE antigen.

In some embodiments, technologies described herein involve a pharmaceutical composition that comprises a single RNA molecule encoding a polyepitopic polypeptide, wherein the polyepitopic polypeptide comprises at least two of a NY-ESO-1 antigen, a MAGE-A3 antigen, a tyrosinase antigen, and a TPTE antigen.

In some embodiments, one or more RNA molecules present in a pharmaceutical composition described herein can further comprise at least one sequence that encodes a CD4+ epitope. For example, in some embodiments, a CD4+ epitope is delivered by the same RNA molecule that encodes at least one of a NY-ESO-1 antigen, a MAGE-A3 antigen, a tyrosinase antigen, and a TPTE antigen.

In some embodiments, one or more RNA molecules present in a pharmaceutical composition described herein can further comprise at least one sequence that encodes tetanus toxoid P2, a sequence that encodes tetanus toxoid P16, or both. In some embodiments, inclusion of P2 and/or P16 in an RNA molecule can improve immune stimulation as compared to a comparable RNA molecule without P2 or P16. Without wishing to be bound to any particular theory, P2 and/or P16 can provide CD4⁺ mediated T cell help during priming. Demotz et al. 1989; Dredge et al. 2002; Livingston et al. 2013, each of which is incorporated herein by reference in its entirety.

In some embodiments, one or more RNA molecules present in a pharmaceutical composition described herein can comprise at least one of the following: a sequence encoding an MHC class I trafficking domain; a 5′ cap or 5′ cap analogue; a sequence encoding a signal peptide; at least one non-coding regulatory element; at least one a poly-adenine tail; at least one 5′ untranslated region (UTR) and/or at least one 3′ UTR; and combinations thereof. In some embodiments, a poly-adenine tail to be included in one or more RNA molecules is or comprises a modified adenine sequence.

In some embodiments, one or more RNA molecules present in a pharmaceutical composition described herein can comprise in 5′ to 3′ order: (i) a 5′ cap or 5′ cap analogue; (ii) at least one 5′ UTR; (iii) a signal peptide; (iv) a coding region that encodes at least one of a NY-ESO-1 antigen, a MAGE-A3 antigen, a tyrosinase antigen, and a TPTE antigen; (v) at least one sequence that encodes tetanus toxoid P2, tetanus toxoid P16, or both; (vi) a sequence encoding an MHC class I trafficking domain; (vii) at least one 3′UTR; and (viii) a poly-adenine tail.

In some embodiments, one or more RNA molecules present in a pharmaceutical composition described herein comprise natural ribonucleotides. In some embodiments, one or more RNA molecules present in a pharmaceutical composition described herein comprise modified or synthetic ribonucleotides.

In some embodiments, at least one of tumor associated antigens (e.g., ones described herein) encoded by one or more RNA molecules is a full-length antigen. In some embodiments, at least one of tumor associated antigens (e.g., ones described herein) encoded by one or more RNA molecules is a truncated antigen. In some embodiments, at least one of tumor associated antigens (e.g., ones described herein) encoded by one or more RNA molecules is a non-mutated antigen. For example, in some embodiments, at least one of a NY-ESO-1 antigen, a MAGE-A3 antigen, a tyrosinase antigen, and a TPTE antigen is full-length, non-mutated antigen. In some embodiments, a NY-ESO-1 antigen is a full-length antigen (e.g., in some embodiments, a full-length, non-mutated antigen). In some embodiments, a MAGE-A3 antigen is a full-length antigen (e.g., in some embodiments, a full-length, non-mutated antigen). In some embodiments, a tyrosinase antigen is a truncated antigen (e.g., in some embodiments a truncated, non-mutated antigen). In some embodiments, a TPTE antigen is a truncated antigen (e.g., in some embodiments a truncated, non-mutated antigen).

In some embodiments, at least one of a NY-ESO-1 antigen, a MAGE-A3 antigen, a tyrosinase antigen, and a TPTE antigen are expressed from dendritic cells in lymphoid tissues of the patient. In some embodiments, at least one of a NY-ESO-1 antigen, a MAGE-A3 antigen, a tyrosinase antigen, and a TPTE antigen are present in cancer.

In some embodiments, lipid particles of a pharmaceutical composition described herein comprise liposomes. In some embodiments, lipid particles of a pharmaceutical composition described herein comprise cationic liposomes. In some embodiments, lipid particles of a pharmaceutical composition described herein comprise lipid nanoparticles.

In some embodiments, lipid particles of a pharmaceutical composition described herein comprise N,N,N trimethyl-2-3-dioleyloxy-1-propanaminium chloride (DOTMA), 1,2-dioleoyl-sn-glycero-3-phosphoethanolamine phospholipid (DOPE), or both.

In some embodiments, lipid particles of a pharmaceutical composition described herein comprise at least one ionizable aminolipid. In some embodiments, lipid particles of a pharmaceutical composition described herein comprise at least one ionizable aminolipid and a helper lipid. In some embodiments, an exemplary helper lipid is or comprises a phospholipid. In some embodiments, an exemplary helper lipid is or comprises a sterol. In some embodiments, lipid particles of a pharmaceutical composition described herein comprises at least one polymer-conjugated lipid (e.g., in some embodiments, a PEG-conjugated lipid).

In some embodiments, technologies provided herein are useful for a human patient. In some embodiments, technologies provided herein are useful for treating cancer and/or prolonging time to relapse. In some embodiments, a cancer is an epithelial cancer. In some embodiments, a cancer is a melanoma. In some embodiments, a cancer is advanced stage. In some embodiments, a cancer is Stage II, Stage III or Stage IV. In some embodiments, a cancer is Stage IIIB, Stage IIIC, or Stage IV melanoma. In some embodiments, a cancer is fully resected, there is no evidence of disease, or both.

In some embodiments, methods described herein comprise administering a second dose of a provided pharmaceutical composition (e.g., ones described herein) to a patient (e.g., in some embodiments a patient suffering from melanoma or a patient having no evidence of disease). In some embodiments, methods described herein comprise administering at least two doses of a pharmaceutical composition to a patient (e.g., in some embodiments a patient suffering from melanoma or a patient having no evidence of disease). In some embodiments, methods described herein comprise administering at least three doses of a pharmaceutical composition to a patient (e.g., in some embodiments a patient suffering from melanoma or a patient having no evidence of disease).

In some embodiments, the present disclosure provides dosing schedules that are particularly useful for the purposes described herein. For example, in some embodiments, at least one dose of the at least three doses is administered to a patient (e.g., in some embodiments a patient suffering from melanoma or a patient having no evidence of disease) within 8 days of the patient having received another dose of the at least three doses. In some embodiments, at least one dose of the at least three doses is administered to a patient (e.g., in some embodiments a patient suffering from melanoma or a patient having no evidence of disease) within 15 days of the patient having received another dose of the at least three doses. In some embodiments, a dosing schedule in accordance with the present disclosure comprises administering at least 8 doses of a pharmaceutical composition described herein to a patient (e.g., in some embodiments a patient suffering from melanoma or a patient having no evidence of disease) within 10 weeks. In some embodiments, a dosing schedule in accordance with the present disclosure comprises administering a dose of a pharmaceutical composition described herein to a patient (e.g., in some embodiments a patient suffering from melanoma or a patient having no evidence of disease) weekly for a period of 6 weeks, and then administering a dose of a pharmaceutical composition described herein every two weeks for a period of 4 weeks. In some embodiments, a dosing schedule in accordance with the present disclosure comprises administering a dose of a pharmaceutical composition described herein to a patient (e.g., in some embodiments a patient suffering from melanoma or a patient having no evidence of disease) monthly following an initial dosing regimen (e.g., an initial dosing regimen comprising at least 8 doses). In some embodiments, a dosing schedule comprises administering a dose of a pharmaceutical composition described herein to a patient (e.g., in some embodiments a patient suffering from melanoma or a patient having no evidence of disease) on a weekly basis for a period of 7 weeks. In some embodiments, a dosing schedule comprises administering a dose of a pharmaceutical composition described herein to a patient (e.g., in some embodiments a patient suffering from melanoma or a patient having no evidence of disease) every three weeks.

In some embodiments, an administered dose (e.g., a first dose and/or a second dose) is 5 μg to 500 μg total RNA. In some embodiments, an administered dose (e.g., a first dose and/or a second dose) is 7.2 μg to 400 μg total RNA. In some embodiments, an administered dose (e.g., a first dose and/or a second dose) is 10 μg to 20 μg total RNA. In some embodiments, an administered dose (e.g., a first dose and/or a second dose) is about 14.4 μg total RNA. In some embodiments, an administered dose (e.g., a first dose and/or a second dose) is about 25 μg total RNA. In some embodiments, an administered dose (e.g., a first dose and/or a second dose) is about 50 μg total RNA. In some embodiments, an administered dose (e.g., a first dose and/or a second dose) is about 100 μg total RNA. In some embodiments, administration can be performed systemically. In some embodiments, administration can be performed intravenously. In some embodiments, administration can be performed intramuscularly. In some embodiments, administration can be performed subcutaneously.

In some embodiments, pharmaceutical compositions described herein can be administered as monotherapy. In some embodiments, pharmaceutical compositions described herein can be administered as part of combination therapy. In some embodiments, a combination therapy can comprise a provided pharmaceutical composition and an immune checkpoint inhibitor. In some embodiments, technologies described herein can be useful for patients who have previously received an immune checkpoint inhibitor. In some embodiments, technologies described herein can further comprise administering to a patient an immune checkpoint inhibitor. Examples of immune checkpoint inhibitors include but are not limited to a PD-1 inhibitor, a PDL-1 inhibitor, a CTLA4 inhibitor, a Lag-3 inhibitor, or a combination thereof. In some embodiments, an immune checkpoint inhibitor is or comprises an antibody. In some embodiments, an immune checkpoint inhibitor is or comprises an inhibitor listed in Table 4 or in Example 8 herein. In some embodiments, an immune checkpoint inhibitor is or comprises ipilimumab, nivolumab pembrolizumab, avelumab, cemiplimab, atezolizumab, duralumab, or a combination thereof. In some embodiments, an immune checkpoint inhibitor that may be particularly useful in accordance with the present disclosure is or comprises ipilimumab. In some embodiments, an immune checkpoint inhibitor that may be particularly useful in accordance with the present disclosure is or comprises ipilimumab and nivolumab. In some embodiments, an immune checkpoint inhibitor that may be particularly useful in accordance with the present disclosure is or comprises cemiplimab.

In some embodiments, technologies described herein are useful for inducing an immune response in a patient receiving a pharmaceutical composition described herein. In some embodiments, a pharmaceutical composition described herein can induce an immune response in the patient.

In some embodiments, methods described herein can further comprise determining a level of an immune response in a patient. In some embodiments, such methods described herein can further comprise comparing a level of the immune response in the patient with a level of the immune response in a second patient to which a pharmaceutical composition has been administered, wherein the second patient was diagnosed with cancer prior to the time of administration and is classified as having evidence of disease at the time of administration. In some such embodiments, an administered pharmaceutical composition induces a level of the immune response in the patient that is comparable to a level of the immune response in a second patient to which the pharmaceutical composition has been administered, has previously been diagnosed with cancer, and is classified as having evidence of disease at the time of administration. In some embodiments, a level of the immune response is a de novo immune response induced by a pharmaceutical composition described herein.

In some embodiments, methods described herein further comprise determining a level of the immune response in a patient before and after administration of a pharmaceutical composition described herein. In some such embodiments, methods further comprise comparing the level of the immune response in the patient after administration of the pharmaceutical composition with the level of the immune response in the patient before administration of the pharmaceutical composition. In some embodiments, the level of the immune response in the patient after administration of the pharmaceutical composition is increased compared with the level of the immune response in the patient before administration of the pharmaceutical composition. In some embodiments, the level of the immune response in the patient after administration of the pharmaceutical composition is maintained compared with the level of the immune response in the patient before administration of the pharmaceutical composition.

In some embodiments, technologies described herein can induce an adaptive response in patients receiving pharmaceutical compositions described herein. In some embodiments, technologies described herein can induce a T-cell response in patients receiving pharmaceutical compositions described herein. In some embodiments, a T-cell response is or comprises a CD4+ response. In some embodiments, a T-cell response is or comprises a CD8+ response. Methods of determining a level of immune response are known in the art. In some embodiments, a level of the immune response in a patient can be determined using an interferon-γ enzyme-linked immune absorbent spot (ELISpot) assay.

In some embodiments, methods described herein further comprise measuring a level of one or more of a NY-ESO-1 antigen, a MAGE-A3 antigen, a tyrosinase antigen, and a TPTE antigen in lymphoid tissue of a patient. In some embodiments, methods described herein further comprise measuring a level of one or more of a NY-ESO-1 antigen, a MAGE-A3 antigen, a tyrosinase antigen, and a TPTE antigen in the cancer.

In some embodiments, methods described herein further comprise measuring a level of metabolic activity in a patient's spleen. In some embodiments, methods described herein further comprise measuring a level of metabolic activity in a patient's spleen before and after administration of a pharmaceutical composition described herein. A level of metabolic activity in a patient's spleen can be measured by using a suitable method known in the art, for example, in some embodiments, using positron emission tomography (PET), computerized tomography (CT) scans, magnetic resonance imaging (MRI), or a combination thereof.

In some embodiments, methods described herein further comprise measuring an amount of one or more cytokines in a patient's plasma. In some embodiments, methods described herein further comprise measuring an amount of one or more cytokines in a patient's plasma before and after administration of a pharmaceutical composition described herein. Non-limiting examples of one or more cytokines to be measured include interferon (IFN)-α, IFN-γ, interleukin (IL)-6, IFN-inducible protein (IP)-10, IL-12 p70 subunit, or a combination thereof.

In some embodiments, methods described herein further comprise measuring a number of cancer lesions in a patient. In some embodiments, methods described herein further comprise measuring a number of cancer lesions in a patient before and after administration of a pharmaceutical composition described herein. In some such embodiments, fewer cancer lesions are detected in the patient after administration of the pharmaceutical composition than before administration of the pharmaceutical composition.

In some embodiments, methods described herein further comprise measuring a number of T cells induced by a pharmaceutical composition described herein in a patient. In some embodiments, methods described herein further comprise measuring a number of T cells induced by a pharmaceutical composition described herein in a patient at a plurality of time points following administration of the pharmaceutical composition. In some embodiments, methods described herein further comprise measuring a number of T cells induced by a pharmaceutical composition in a patient following administration of a first dose of the pharmaceutical composition and following administration of a second dose of the pharmaceutical composition. In some such embodiments, the number of T cells induced by an administered pharmaceutical composition in a patient is greater following administration of a second dose of the pharmaceutical composition than following administration of a first dose of the pharmaceutical composition.

In some embodiments, methods described herein further comprise determining a phenotype of T cells induced by a pharmaceutical composition in a patient following administration of the pharmaceutical composition. In some embodiments, at least a subset of T cells induced by an administered pharmaceutical composition in a patient have a T-helper-1 phenotype. In some embodiments, T cells induced by an administered pharmaceutical composition in a patient comprise T cells having a PD1+ effector memory phenotype.

In some embodiments, technologies described herein are useful for administration to a patient who is classified as having evidence of disease. In some such embodiments, methods described herein for a patient classified as having evidence of disease further comprise measuring a size of one or more cancer lesions. In some embodiments, methods described herein further comprise measuring a size of one or more cancer lesions in a patient before and after administration of a pharmaceutical composition described herein. In some embodiments, methods described herein further comprise comparing the size of one or more cancer lesions in the patient before and after administration of the pharmaceutical composition. In some such embodiments, the size of at least one cancer lesion in the patient after administration of the pharmaceutical composition is equal to or smaller than the size of the at least one cancer lesion before administration of the pharmaceutical composition.

In some embodiments, methods described herein for a patient classified as having evidence of disease further comprise monitoring a duration of progression-free survival. In some such embodiments, methods described herein comprise comparing the duration of progression-free survival of a patient with a reference duration of progression-free survival. In some embodiments, an exemplary reference duration of progression-free survival is an average duration of progression-free survival of a plurality of comparable patients who have not received a pharmaceutical composition described herein. In some embodiments, duration of progression-free survival of a patient administered with a pharmaceutical composition described herein is longer in time than a reference duration of progression-free survival.

In some embodiments, methods described herein for a patient classified as having evidence of disease further comprise measuring a duration of disease stabilization. In some embodiments, disease stabilization can be determined by applying an irRECIST or RECIST 1.1 standard. In some embodiments, methods described herein further comprise comparing duration of disease stabilization of a patient to a reference duration of disease stabilization. In some embodiments, such a reference duration of disease stabilization is an average duration of disease stabilization of a plurality of comparable patients who have not received a pharmaceutical composition described herein. In some embodiments, a patient administered with a pharmaceutical composition described herein exhibits an increased duration of disease stabilization compared to a reference duration of disease stabilization.

In some embodiments, methods described herein for a patient classified as having evidence of disease further comprise measuring a duration of tumor responsiveness. In some embodiments, tumor responsiveness is determined by applying an irRECIST or RECIST 1.1 standard. In some embodiments, methods described herein further comprise comparing duration of tumor responsiveness of a patient administered with a pharmaceutical composition described herein to a reference duration of tumor responsiveness. In some embodiments, such a reference duration of tumor responsiveness is an average duration of tumor responsiveness of a plurality of comparable patients who have not received a pharmaceutical composition described herein. In some embodiments, a patient administered with a pharmaceutical composition described herein exhibits an increased duration of tumor responsiveness compared to a reference duration of tumor responsiveness.

In some embodiments, technologies described herein are useful for administration to a patient who is classified as having no evidence of disease. In some such embodiments, methods described herein further comprise monitoring a duration of disease-free survival. In some embodiments, methods described herein further comprise comparing a duration disease-free survival of a patient to a reference duration of disease-free survival. In some embodiments, such a reference duration of disease-free survival is an average duration of disease-free survival of a plurality of comparable patients who have not received a pharmaceutical composition described herein. In some embodiments, a patient administered with a pharmaceutical composition described herein exhibits an increased duration of disease-free survival compared to a reference duration of disease-free survival.

In some embodiments, methods described herein for a patient classified as having no evidence of disease can further comprise measuring a duration to disease relapse. In some embodiments, disease relapse is determined by applying an irRECIST or RECIST 1.1 standard. In some embodiments, methods described herein further comprise comparing the duration to disease relapse of a patient administered with a pharmaceutical composition described herein to a reference duration to disease relapse. In some embodiments, such a reference duration to disease relapse is an average duration to disease relapse of a plurality of comparable patients who have not received a pharmaceutical composition described herein. In some embodiments, a patient administered with a pharmaceutical composition described herein exhibits an increased duration to disease relapse compared to a reference duration to disease relapse.

In some embodiments, technologies described herein are useful to prolong overall survival of patients. In some embodiments, patients are classified as having evidence of disease. In some embodiments, patients are classified as having no evidence of disease.

In some aspects, pharmaceutical compositions for use in inducing an immune response against cancer in a patient are also provided herein. In some embodiments, such a patient is classified as having no evidence of disease, but has previously been diagnosed with cancer. In some embodiments, a pharmaceutical composition comprises: (a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and (b) lipid particles.

In some aspects, pharmaceutical compositions for use in treating cancer are also provided herein. In some embodiments, such a patient is classified as having no evidence of disease, but has previously been diagnosed with cancer. In some embodiments, a pharmaceutical composition comprises: (a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and (b) lipid particles. In some embodiments, pharmaceutical compositions described herein are particularly useful for administering to patients with melanoma.

Use of pharmaceutical compositions described herein are also within the scope of the present disclosure. In some embodiments, pharmaceutical compositions described herein are useful for inducing an immune response against cancer in patients, for example, in some embodiments patients who are classified as having no evidence of disease, but have previously been diagnosed with cancer. In some embodiments, pharmaceutical composition described herein are useful for treating cancer in patients, for example in some embodiments patients who are classified as having no evidence of disease, but have previously been diagnosed with cancer. In some embodiments, a cancer is melanoma. In some embodiments, a pharmaceutical composition comprises: (a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and (b) lipid particles.

### BRIEF DESCRIPTION OF THE DRAWINGS

FIGS. 1a-1d depict exemplary TAA constructs, trial design, and vaccine-mediated immune activation. FIG. 1a, Structure of the TAA RNAs. The 5′-cap analogue, 5′- and 3′-untranslated regions (UTRs) and poly(A) tail were optimized for stability and translational efficiency. In addition the TAA-encoding sequence was tagged with a signal peptide (SP), tetanus toxoid CD4+ epitopes P2 and P16, and the MHC class I trafficking domain (MITD) for enhanced HLA presentation and immunogenicity. FIG. 1b, Clinical trial design. FIG. 1c, Metabolic activity in the spleen, measured by trans-axial [18F] FDG-PET/CT at baseline (pre) and 4 h after the sixth vaccine injection (post). FIG. 1d, Plasma levels of cytokines (before and 2 h, 6 h and 24 h (and, in some cases, 48 h) after each vaccine injection) and body temperature of a patient (from cohort V) injected weekly with six escalated doses. Dotted horizontal lines indicate the upper limit of normal.

FIGS. 2a-2k depict T-cell immunity and clinical activity of FixVac. FIG. 2a, 2c, Proportion of patients with vaccine-induced T-cell responses (de novo or amplified) as analyzed by IFN-γ ELISpot pre- and post-vaccination measured ex vivo (a; n=50) or after IVS (c; n=20). PBLs, peripheral blood lymphocytes. FIG. 2b, Ex vivo CD8+ T-cell responses for patient A2-09, measured using TAA PepMix-pulsed CD4-depleted PBMCs. Control, PBMCs with medium. FIG. 2d, Post-IVS CD4+ T-cell responses of patient 42-06, measured using autologous dendritic cells loaded with TAA PepMixes as targets. Control, luciferase-transfected dendritic cells. FIG. 2e, Ex vivo frequency of HLA multimer stained NY-ESO-1-specific T cells from patient 12-01 (cohort 1, six vaccine doses). Dashed lines indicate vaccinations. FIG. 2f-2i, De novo induced HLA-B*3503-restricted NY-ESO-1-specific T cells from patient A2-09 (cohort A, continued vaccination). Dashed lines indicate vaccinations. FIG. 2f, Phenotype of NY-ESO-1/HLA-B*3501 multimer stained PBMCs. Multimer-positive CD8+ T cells are shown in red. BV421 and BV650 are immunofluorescent labels. FIG. 2g, Left, multimer analysis and right, ICS of T cells stimulated with single peptides or PepMix. FIG. 2h, ICS of ex vivo NY-ESO-1 peptide stimulated CD8+ T cells. FIG. 2i, Specific lysis of melanoma cell lines by healthy donor CD8+ T cells transfected with HLA-B*3503-restricted NY-ESO-1-specific TCRs cloned from patient A2-09 (effector:target (E:T) ratio=20:1). SK-MEL-37 and SK-MEL-28 are melanoma cell lines. PD-Cy7 is an immunofluorescent label. FIG. 2j, FIG. 2k, Clinical activity, assessed as the effect of FixVac without/with anti-PD1 antibody on target lesions (n=38; 4 patients had no target lesions at baseline). FIG. 2j, Asterisks indicate combination with anti-PD1 antibody. PD, progressive disease; PR, partial response.

FIGS. 3a-3g depict T-cell immunity in patient 53-02 treated with FixVac monotherapy. FIG. 3a, Top, NY-ESO-196-104-specific Cw*0304-restricted CD8+ T cells analyzed by HLA multimer staining. Control, cytomegalovirus (CMV)-pp65 multimer. Bottom, exemplary flow cytometry. FIG. 3b, Melanoma lesions as assessed by CT scan. Lesions smaller than a quantifiable size are plotted as having a diameter of 0.1 mm. NT, non-target lesions; T, target lesions; according to the immune-related response evaluation criteria in solid tumours (irRECIST) version 1.1. FIG. 3c, Top, ex vivo frequency of NY-ESO-196-104-specific, cytokine-secreting CD8+ T cells analyzed by ICS. Bottom, exemplary flow cytometry. FIG. 3d, Top, killing of melanoma cell lines by CD8+ T cells from IVS cultures (E:T=20:1). Bottom, frequencies of NY-ESO-196-104 multimer specific CD8+ T cells after IVS of PBMCs from different treatment time points (−1, baseline; day 22, after 3 vaccinations; day 64, after 7 vaccinations). FIG. 3e, Cytotoxicity of two HLA-B*4001-restricted NY-ESO-1-specific TCRs cloned from post-vaccination samples and transfected into healthy donor CD8+ T cells against melanoma cell lines (E:T=20:1). FIG. 3f, Frequency of the TCRs from e in peripheral blood, measured by ex vivo TCR repertoire analysis. TRB, T-cell receptor-β. FIG. 3g, Top, kinetics of the ex vivo frequency of MAGE-A3167-176-specific, cytokine-secreting CD8+ T cells. Bottom, exemplary flow cytometry.

FIGS. 4a-4g depict T-cell immunity in partial-response patients treated with the FixVac/anti-PD1 combination. FIG. 4a-4c, Patient C2-28. a, Size of the target lesions; FIG. 4b, de novo MAGE-A3-specific CD8+ T cells analyzed by HLA multimer staining (top), with exemplary flow cytometry (bottom). FIG. 4c, Recognition of melanoma cells by MAGE-A3168-176-specific TCRs. FIG. 4d, CT scans of lung lesions in patient C2-31. FIGS. 4e-4f, Patient C1-40. FIG. 4e, MAGE-A3168-176-specific HLA-A*0101-restricted T cells analyzed by HLA-multimer staining. FIG. 4f, Top, lysis of melanoma cell lines by CD8+ T cells from IVS cultures of PBMCs collected before and under treatment (E:T=8.5:1). Bottom, MAGE-A3168-176-specific CD8+ T cells after IVS. FIG. 4g, Correlation of FixVac TAA transcript expression and number of non-synonymous single-nucleotide variants (snSNVs) in melanomas from three independent cohorts (n=50). RPKM, reads per kilobase per million mapped reads.

FIG. 5 depict patient subsets. Patients had advanced melanoma either with radiographically measurable disease or with non-measurable disease at baseline. Immune monitoring was performed for 49 patients across all subgroups. Clinical antitumour activity was assessed in those 42 (1 unresected stage III C, 41 stage IV) of a total of 56 patients with measurable disease at baseline for whom follow-up imaging data were available at data cut-off (25 treated with FixVac monotherapy, 17 FixVac in combination with anti-PD1 therapy). The remaining 14 patients (5 receiving FixVac in monotherapy and 9 in combination with anti-PD1 therapy) were not included in the efficacy analyses for the reasons noted in the previous sentence. PD, progressive disease; PR, partial response; SD, stable disease (best objective overall responses as per irRECIST1.1). CR* refers to metabolic complete response of a patient with SD as best response, according to irRECIST1.1. Thirty-three patients with radiographically non-measurable disease at baseline were not subject to exploratory analysis for objective best overall response and are in follow-up for recurrence-free survival.

FIGS. 6a-6c depict characterization of cytokine secretion. FIG. 6a, 6b, Peak plasma cytokine levels (6 h after vaccine injection) and body temperature (4 h after vaccine injection) for: FIG. 6a, all available patients; and FIG. 6b, patients treated with RNA-lipoplex (LPX) target doses of 50 μg or 100 μg either alone (‘Mono’) or in combination with anti-PD1 therapy (‘aPD1’). Boxes show 25th to 75th quantiles with lines representing medians; whiskers show minimum to maximum values; grey dots show individual values per dose level; dashed lines indicate upper limits of normal. Sample numbers (n) are indicated in the figure. FIG. 6c, Correlation of plasma cytokine levels (y-axis) with plasma IFN-α concentration 6 h after RNA-LPX administration (n=147 for IFN-γ, IL-12 p70 and IL-6; n=147 for IP-10).

FIGS. 7a-7f depict T-cell immunity induced by FixVac. FIG. 7a, Phenotype (left) and quality (middle and right) of TAA-specific T cells measured by IFN-γ ELISpot post-IVS (left and middle) or ex vivo (right). Only positive responses are shown. FIG. 7b, Example flow cytometry of PBMCs from patient 12-01 stained with NY-ESO-192-100/Cw*0304 multimer. FIG. 7c, Flow cytometry gating strategy for phenotypic characterization of multimer+ T cells. Upper row, from left to right: starting with events acquired with a constant flow stream and fluorescence intensity, we identified single events (singlets). Dump-negative events (viable, CD4−, CD14−, CD16−, CD19−) and lymphocytes were identified and gated. Within lymphocytes, CD8+ HLA multimer positive T cells were gated for further analysis. Lower row, left plot: different subsets of CD8+ T cells (indicated in black) and NY-ESO-1 multimer positive CD8+ T cells (red) were gated on the basis of CD45RA and CCR7 expression into four subsets, analyzed for CD27 and CD28 expression in the right-hand plots—central memory (CCR7+ CD45RA−), naive (CCR7+ CD45RA+) effector memory (CCR7− CD45RA−) and effector memory re-expressing RA (CCR7− CD45RA+). The expression of PD1 and OX40 was analyzed for multimer-positive (red) and multimer-negative (black) CD8+ T cells. FIG. 7d, Detection of CD8+ T cells of patient A2-09 secreting IFN-γ and TNF after stimulation with MAGE-A3212-220 peptide. FIG. 7e, Comparison of fold induction of ex vivo spot counts after vaccination, between patients with measurable (n=27) or non-measurable (n=30) disease (left), patients treated with different vaccine doses (14.4 μg (n=17), 50 μg (n=10), 100 μg (n=24); middle), and patients treated with FixVac alone (Mono (n=44)) or in combination with anti-PD1 therapy (aPD1 (n=12); right). Only positive responses at the post-vaccination visit are shown. A fold change of more than 2 compared with baseline was considered as a response to vaccine. If both CD4 and CD8 results were positive at post-treatment, only the ratio of the higher spot count is shown. FIG. 7f, Proportion of patients with vaccine-induced T-cell responses (de novo or amplified) determined by IFN-γ ELISpot pre- and post-vaccination, measured ex vivo from patients treated with FixVac alone (n=14) or in combination with anti-PD1 therapy (n=12). Data from patients with measurable disease only are shown.

FIGS. 8a-8d depict disease responses and treatment schedules for patients evaluated for clinical activity. FIG. 8a, 8b, Swimmer plots for patients evaluable for efficacy assessments from the start of treatment to disease progression or continued treatment. FIG. 8a, Patients treated with melanoma FixVac in monotherapy. The numbers on the y axis represent individual patients. CR=Complete response; PR=Partial Response; SD=Stable Disease; and PD=Progressive Disease. The grey line indicates the time when the initial treatment phase was finished and when the continued treatment started. FIG. 8a includes data obtained from patients with evidence of disease (ED patients) who received BNT111 as monotherapy. FIG. 8b, Patients treated with FixVac and anti-PD1 therapy. Dark green triangles indicate treatment start and completion. Dark green arrows show patients who are still receiving treatment. Red crosses mark disease progression; patients are sorted by best overall response and progression-free survival time (CR, PD, PR, SD). Light green stars indicate first documented objective responses and light green arrows indicate ongoing disease control. The black vertical lines mark the day planned for the eighth vaccination (study day 64). Single asterisks indicate patients for whom the clinical course and treatment schedules are shown in d. CR**, metabolic complete response of a patient with stable disease as the best response according to irRECIST1.1. Patients with radiologically non-measurable disease at baseline are in follow-up for recurrence-free survival and were not subject to clinical efficacy assessment. FIG. 8c, Tumour burden at baseline in relation to the clinical response upon FixVac treatment. PD, progressive disease; PR, partial response; SD, stable disease. FIG. 8d, Clinical course and treatment schedules of patients Pt 53-02, A2-09, C2-28, A2-10, C2-31 and C1-40. FD, first diagnosis of melanoma at any stage. FD stage IV, first diagnosis of melanoma at stage IV. *New bone lesion diagnosed and treated with radiotherapy.

FIGS. 9a-9j depict T-cell immunity in patient 53-02 with partial response under FixVac monotherapy. FIG. 9a, CT scans of the lower and middle lobes of the right lung before (pre) and after starting (post) melanoma FixVac treatment. FIG. 9b, Kinetics of a NY-ESO-196-104-specific, HLA-Cw*0304-restricted CD8+ T-cell response (see also FIG. 3a). FIGS. 9c-f, Discovery and characterization of a NY-ESO-196-104-specific HLA-Cw*0304-restricted TCR. FIG. 9c, Sorting gate of multimer-positive CD8+ T cells (gated within the single, live, CD3+ lymphocyte population) for TCR cloning. Control, fluorescence minus one (FMO) sample. FIG. 9d, Recognition of peptide-pulsed HLA-Cw*0304-transfected K562 cells by NY-ESO-1-TCR-transfected CD8+ T cells in IFN-γ ELISpot. Control, HIV-gag PepMix; NY-ESO-1, NY-ESO-1 PepMix. FIG. 9e, Cytotoxicity of NY-ESO-1-TCR transfected healthy donor CD8+ T cells after 24 h of co-culture with HLA-transfected melanoma cell lines (SK-MEL-37 and SK-MEL-28; E:T=50:1). FIG. 9f, Kinetics of NY-ESO-1-specific TCR clonotype frequency in TCR repertoire data obtained from pre- and post-vaccination PBMCs. FIGS. 9g-9j, Discovery and characterization of two NY-ESO-1124-133-specific HLA-B*4001-restricted TCRs. FIG. 9g, PBMCs were stimulated with NY-ESO-1 PepMix, and single IFN-γ positive CD8+ T cells were sorted via flow cytometry for TCR cloning (control, HIV-gag PepMix). FIGS. 9h, 9i, HLA restriction and epitope specificity of NY-ESO-1-TCRs analyzed after co-culture of TCR-transfected CD8+ T cells with peptide-pulsed HLA-transfected K562 cells using IFN-γ ELISpot. NY-ESO-1, NY-ESO-1 PepMix. FIG. 9j, Cytotoxicity of NY-ESO-1-specific TCRs identified in post-vaccination samples of the patient. TCR-transfected healthy donor CD8+ T cells were stimulated with HLA-transfected melanoma cell lines (SK-MEL-37, SK-MEL-28) for 12 h at an effector to target ratio of 20:1.

FIGS. 10a-10i depict T-cell immunity in patients A2-10, C2-31 and C1-40. FIG. 10a-10f, Patient A2-10, with CPI-refractory melanoma, developed a partial response under FixVac monotherapy. FIG. 10a, CT scans of an inguinal lymph node metastasis obtained before and after the start of vaccination. FIG. 10b, Post-IVS CD4+ T-cell responses pre-vaccination and after eight vaccinations, restimulated in an IFN-γ ELISpot assay with autologous dendritic cells transfected with RNA (encoding one of the TAAs or luciferase as control), or pulsed with TAA-encoding PepMix versus unpulsed dendritic cells (no peptide). FIG. 10c, Cytokine-secreting CD8+ and CD4+ T cells after intradermal challenge with NY-ESO-1 RNA. Skin-infiltrating lymphocytes were recovered from a punch biopsy 15 days after 8 weekly vaccinations and stimulated with PepMix encoding NY-ESO-1 or tyrosinase. FIGS. 10d-10f, Discovery and characterization of HLA II restricted TAA-specific TCRs. d, CD4+ T cells from IVS cultures were restimulated with PepMix-pulsed dendritic cells and sorted via flow cytometry for TCR cloning (control, HIV-gag PepMix). APC and PE are fluorochrome labels. FIG. 10e, Determination of HLA restriction and epitope specificity using TCR-transfected healthy donor CD4+ T cells and RNA-transfected or peptide-pulsed HLA-transfected K562 cells by IFN-γ ELISpot. DRA, DRB, DQA and DQB numbers refer to specific HLA alleles. Control, K562 cells without peptide (−). FIG. 10f, Kinetics of TCR clonotype frequencies in peripheral blood by ex vivo TCR repertoire analysis. FIG. 10g, TAA-specific CD8+ and CD4+ T-cell responses of patient C2-31 by IFN-γ ELISpot on peptide-loaded autologous dendritic cells after IVS with TAA PepMix. Control, dendritic cells loaded with irrelevant peptide. FIGS. 10h, 10i, Clinical and immune responses of patient C1-40, with CPI-refractory melanoma, who developed a partial response under melanoma FixVac combined with nivolumab. FIG. 10h, CT scans of the right middle and left lower lung lobes before and after the start of melanoma FixVac treatment. FIG. 10i, Ex vivo frequencies of MAGE-A3168-176-specific A*0101-restricted (left panel) and NY-ESO-192-100-specific HLA_Cw*0304-restricted (right panel) CD8+ T cells analyzed by HLA multimer staining.

FIG. 11 depicts gating strategy for flow cytometry analysis of data shown in FIG. 2e (Pt 12-01 up to day 50). Flow cytometry gating strategy for identification of vaccine-induced T cells. (Upper row left to right) Starting with events acquired with a constant flow stream and fluorescence intensity the single events were identified. Viable cells and lymphocytes were identified and gated. Within lymphocytes Dump-negative events (CD4−, CD14−, CD16−, CD19− negative) were gated to exclude them for further analysis. Within Dump-negative events, CD8+ HLA-multimer-positive T cells were gated for further analysis (bottom row).

FIG. 12 depicts gating strategy for flow cytometry analysis of data shown in FIG. 2f, FIG. 2g (Pt A2-09), FIG. 2e (Pt 12-01 after day 50), FIG. 3a (Pt 53-02) and FIG. 7c (Pt A2-09), FIG. 9b (Pt 53-02). Flow cytometry gating strategy for phenotype characterization of vaccine-induced T cells. (Upper row left to right) Starting with events acquired with a constant flow stream and fluorescence intensity the single events were identified. Dump-negative events (viable, CD4-negative, CD14-negative, CD16-negative, CD19-negative) and lymphocytes were identified and gated. Within lymphocytes, CD8+ HLAmultimer− positive T cells were gated for further analysis. (Middle row left plot). The expression of PD1 and OX40 were analyzed for multimer-positive (red) and multimer-negative (black) CD8+ T cells (Middle row middle and right plot). Different subsets of CD8+ T cells (indicated in black) and multimer-positive CD8+ T cells (highlighted in red) were gated based on CD45RA and CCR7 into four subsets: central memory (CD45RA− CCR7+), naive (CD45RA+ CCR7+), effector memory (CD45RA− CCR7−) and effector memory re-expressing RA (CD45RA+ CCR7−). The expression of CD27 and CD28 was analyzed in each subset.

FIG. 13 depicts gating strategy for flow cytometry analysis of data shown in FIG. 2h, FIG. 2g (Pt A2-09) and FIG. 7d (Pt A2-09). Flow cytometry gating strategy for identification of cytokine responses in vaccine-induced T cells. (Upper row left to right) Starting with events acquired with a constant flow stream and fluorescence intensity the single events were identified. Dump-negative events (viable, CD14−, CD16−, CD19-negative) and lymphocytes were identified and gated. Within lymphocytes, CD8+ and CD4+ T cells were gated for further analysis (bottom row left plot). Production of the effector cytokines TNF and IFNγ in CD8+ (bottom row middle plot) and CD4+ T cells (bottom row right plot) were gated and analyzed.

FIG. 14 depicts gating strategy for flow cytometry analysis of data shown in FIG. 3c and FIG. 4g (Pt 53-02). Flow cytometry gating strategy for identification of cytokine responses in vaccine-induced T cells. (Upper row left to right) Starting with events acquired with a constant flow stream and fluorescence intensity the single events were identified. Lymphocytes were identified and gated in the next step. Within lymphocytes, CD8+ and CD4+ T cells were gated for further analysis (bottom row left plot). Production of the effector cytokines TNF and IFNγ in CD8+ (bottom row middle plot) and CD4+ T cells (bottom row right plot) were gated and analyzed.

FIG. 15 depicts dating strategy for flow cytometry based detection of multimer positive T cells of patient 53-02 after IVS shown in FIG. 3d. For the detection of NY-ESO-196-104 multimer-specific T cells first single events and lymphocytes were identified. Within single lymphocytes CD3+ viable cells were gated. Within the viable CD3+ cells CD8+/multimer+ were identified. The gating strategy for sample day 64 is shown as an example for multimer analysis depicted in FIG. 3d.

FIG. 16 depicts flow cytometry gating strategy for single cell sorting of TAA-specific T cells for TCR cloning shown in FIG. 9c, 9g and FIG. 10d. For detection of TAA-specific T cells based on (a) multimer staining or (b, c) IFNγ secretion first single events and lymphocytes were identified. Within single lymphocytes CD3+ viable cells were gated. Within the viable CD3+ cells either (a) CD8+/multimer+, (b) CD8+/IFNy+ or (c) CD4+/IFNy+ T cells were gated. The sorting gates are highlighted in red. The gating strategies for NY-ESO-1-specific T cells of patient 53-02 after (a) multimer staining or (b) IFNγ secretion assay are shown corresponding to data depicted in FIG. 9c, 9g as well as for MAGE-A3-specific T cells of patient A2-10 shown in FIG. 10d.

FIG. 17 depicts gating strategy for flow cytometry analysis of data shown in FIG. 10c (Pt A2-10). Flow cytometry gating strategy for identification of cytokine responses in vaccine-induced T cells. (Upper row left to right) Starting with events acquired with a constant flow stream and fluorescence intensity the single events were identified. Dump-negative events (viable cells) and lymphocytes were identified and gated. Within lymphocytes, CD8+ and CD4+ T cells were gated for further analysis (bottom row left plot). Production of the effector cytokines TNF and IFNγ in CD8+ (bottom row middle plot) and CD4+ T cells (bottom row right plot) were gated and analyzed.

FIG. 18 depicts gating strategy for flow cytometry analysis of data shown in FIG. 4b (Pt C2-028), FIG. 4e (Pt C1-040) and FIG. 10i (Pt C1-40). Flow cytometry gating strategy for identification of vaccine-induced T cells. (Upper row left to right) Starting with events acquired with a constant flow stream and fluorescence intensity the single events were identified. Dump-negative events (viable, CD4−, CD14−, CD16−, CD19-negative) and lymphocytes were identified and gated. Within lymphocytes, CD8+ HLA multimer-positive T cells were gated for further analysis (bottom row).

FIG. 19 depicts flow cytometry gating strategy for detection of multimer positive T cells of patient C1-40 after IVS shown in FIG. 4f. For the detection of MAGE-A3168-176 multimer-specific T cells first single events and lymphocytes were identified. Within single lymphocytes CD3+ viable cells were gated. Within the viable CD3+ cells CD8+/multimer+ were identified. The gating strategy for sample day 129 is shown as an example for multimer analysis depicted in FIG. 4f.

FIGS. 20a-20c depict ex-vivo ELISPOT CD4+ or CD8+ (FIG. 20a), CD8+ (FIG. 20b) or CD4+ (FIG. 20c) responses. Frequency of patients with vaccine-induced (amplified or de novo) response. Numbers in bar segments represent number of evaluated patients per segment. Only patients treated in monotherapy are included.

FIG. 21 depicts ex-vivo ELISPOT response by cell type. Numbers and percentage of evaluable ELISPOT responses. Only non-bulk measurements with evaluable results for both CD4 and CD8 from patients treated in monotherapy are included.

FIG. 22 depicts vaccine-induced ex-vivo ELISPOT CD4+ or CD8+ responses to any cell type. Fraction of de novo and amplified responses. Only patients treated in monotherapy are included.

FIGS. 23a-23c depict ex-vivo ELISPOT CD4+ or CD8+ (FIG. 23a), CD8+ (FIG. 23b) or CD4+ (FIG. 23c) responses. Frequency of patients with vaccine-induced (amplified or de novo) response. Numbers in bar segments represent number of evaluated patients per segment. Only patients treated in monotherapy are included.

FIG. 24 depicts ex-vivo ELISPOT CD4+ or CD8+ response by clinical best response for non-evaluable disease patients. Numbers in bar segments represent number of patients with evaluated ex-vivo ELISPOT measurements per segment. Only patients treated in monotherapy are included. Patients without evaluable ELISPOT results or recorded clinical best are excluded.

FIG. 25 depicts ex-vivo ELISPOT CD4+ or CD8+ response by clinical best response for evaluable disease patients. Numbers in bar segments represent number of patients with evaluated ex-vivo ELISPOT measurements per segment. Only patients treated in monotherapy are included. Patients without evaluable ELISPOT results or recorded clinical best are excluded.

FIGS. 26a-26b depict summary of disease free survival data for NED patients, and Kaplan-Meier summary of disease free survival data for NED patients.

FIGS. 27a-27f depict summary of overall survival data for ED patients (FIG. 27a), NED patients (FIG. 27b), and combined NED and ED patients (FIG. 27c); and Kaplan-Meier summary of overall survival data for ED patients (FIG. 27d), NED patients (FIG. 27e), and combined ED and NED patients (FIG. 27f).

FIGS. 28a-28c depict summary of adverse events for ED patients (FIG. 28a), NED patients (FIG. 28b) and combined ED and NED patients (FIG. 28c).

FIG. 29 depicts patient disposition data. Among the total number of 89 patients, 3 patients who enrolled twice were counted only once, on their first enrolment (2 patients were treated in cohort CI and later enrolled in cohort CIII, and 1 patient from cohort CII later enrolled in expanded cohort Exp. A). Starting doses are in blue and target doses in orange. In cohorts CII to CVII and Exp. A, B and C, patients received 8 doses of melanoma FixVac (on days 1, 8, 15, 22, 29, 36, 50 and 64). Patients in cohort CI received 6 doses only (on days 1, 8, 15, 22, 29 and 43). Patients with measurable disease at baseline were allowed optional continued treatment (Q4W) until disease progression or drug-related toxicity. When FixVac was to be combined with anti-PD1 therapy, this happened from the first dose, except in the case of one patient (asterisk) for whom anti-PD1 therapy was added during treatment.

FIG. 30 depicts characteristics and prior treatments of patients in the clinical analysis set.

FIG. 31 depicts data for spleen FDR upstate as measured by PET/CT imaging. FDG uptake in the spleen was assessed by PET/CT imaging and quantified in selected patients at baseline and at different time points after the fourth (71-27), fifth (C1-45) or sixth (C1-44) vaccination cycles. Total and relative FDG uptake in spleen are displayed. SUV, standardized uptake value.

FIG. 32 depicts data for related adverse events that emerge after treatment in more than 5% of patients.

FIG. 33 depicts data for antigen specific α/β TCRs isolated from single T cells of melanoma patients.

FIG. 34 includes a schematic showing an exemplary mRNA molecule as described herein and the modes-of action of the mRNA complexed in a lipoplex.

FIG. 35 includes a table providing various characteristics associated with patients who participated in a study of the safety and efficacy of an exemplary composition described herein (BNT111).

FIG. 36 includes a table providing various characteristics associated with patients who participated in a study of the safety and efficacy of an exemplary composition described herein (BNT111).

FIG. 37 includes a swimmer plot sorted by best clinical response and duration of disease free survival. Bar length indicates duration of disease control. Dashed line indicates approximate day of the last BNT111 administration during initial trial treatment according to the clinical trial protocol. DFS=disease-free survival; LTFU=long-term follow-up; PD refers to progressive disease. Data in this ploy was obtained from patients treated with BNT111 monotherapy.

FIG. 38 includes bar graphs showing ex vivo responses from patients determined by ELISpot. Ex vivo responses were detected in 14/22 (64%) and 19/28 (68%) ED and NED patients, respectively.

FIG. 39 includes bar graphs showing post-in vitro stimulation responses from patients determined by ELISpot. Post-in vitro stimulation (IVS) ELISpot was carried out in 9 ED patients and 6 NED patients (smaller sample size due to limited sample availability). In all 15 patients, a T-cell response against at least one TAA was observed.

FIG. 40 includes a bar graph showing treatment-emergent serious adverse events having ≥10% incidence in any subgroup of patients following treatment with an exemplary composition described herein (BNT111).

FIG. 41 includes a bar graph showing related treatment-emergent serious adverse events with a common terminology criteria for adverse events grade of greater than or equal to 3 following treatment with an exemplary composition described herein (BNT111).

FIG. 42 includes a table providing an overview of preliminary efficacy in patients with evaluable disease according to irRECIST.

FIG. 43 includes a waterfall plot of the best change from baseline observed in target lesions according to irRECIST in patients with measurable disease at baseline treated with an exemplary monotherapy (BNT111) or combination with a PD-1 Inhibitor or BRAF/MEK inhibition.

### CERTAIN DEFINITIONS

About or approximately: As used herein, the term “approximately” or “about,” as applied to one or more values of interest, refers to a value that is similar to a stated reference value. In general, those skilled in the art, familiar within the context, will appreciate the relevant degree of variance encompassed by “about” or “approximately” in that context. For example, in some embodiments, the term “approximately” or “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administering: As used herein, the term “administering” or “administration” typically refers to the administration of a composition to a subject to achieve delivery of an agent that is, or is included in, a composition to a target site or a site to be treated. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may be parenteral. In some embodiments, administration may be oral. In some particular embodiments, administration may be intravenous. In some particular embodiments, administration may be subcutaneous. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration may comprise a prime-and-boost protocol. A prime-and-boost protocol can include administration of a first dose of a pharmaceutical composition (e.g., an immunogenic composition, e.g., a vaccine) followed by, after an interval of time, administration of a second dose of a pharmaceutical composition (e.g., an immunogenic composition, e.g., a vaccine). In the case of an immunogenic composition, a prime-and-boost protocol can result in an increased immune response in a patient.

Antibody: As used herein, the term “antibody agent” refers to an agent that specifically binds to a particular antigen. In some embodiments, the term encompasses any polypeptide or polypeptide complex that includes immunoglobulin structural elements sufficient to confer specific binding. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc., as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present disclosure is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated complementarity determining regions (CDRs) or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.].

Associated with: Two events or entities are “associated” with one another, as that term is used herein, if the presence, level and/or form of one is correlated with that of the other. For example, a particular biological phenomenon is considered to be associated with a particular disease, disorder, or condition (e.g., cancer), if its presence correlates with incidence of and/or susceptibility of the disease, disorder, or condition (e.g., across a relevant population), or likelihood of responsiveness to a treatment.

Blood-derived sample: The term “blood-derived sample,” as used herein, refers to a sample derived from a blood sample (i.e., a whole blood sample) of a subject in need thereof. Examples of blood-derived samples include, but are not limited to, blood plasma (including, e.g., fresh frozen plasma), blood serum, blood fractions, plasma fractions, serum fractions, blood fractions comprising red blood cells (RBC), platelets, leukocytes, etc., and cell lysates including fractions thereof (for example, cells, such as red blood cells, white blood cells, etc., may be harvested and lysed to obtain a cell lysate). In some embodiments, a blood-derived sample that is used for characterization described herein is a plasma sample.

Cancer: The term “cancer” is used herein to generally refer to a disease or condition in which cells of a tissue of interest exhibit relatively abnormal, uncontrolled, and/or autonomous growth, so that they exhibit an aberrant growth phenotype characterized by a significant loss of control of cell proliferation. In some embodiments, cancer may comprise cells that are precancerous (e.g., benign), malignant, pre-metastatic, metastatic, and/or non-metastatic. In some embodiments, cancer may be characterized by a solid tumor. In some embodiments, cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, ovarian cancer, breast cancer, glioblastomas, colorectal cancer, gastro-intestinal cancers and nervous system cancers, benign lesions such as papillomas, and the like. In particular embodiments, a cancer can be a melanoma.

Cap: As used herein, the term “cap” refers to a structure comprising or essentially consisting of a nucleoside-5′-triphosphate that is typically joined to a 5′-end of an uncapped RNA (e.g., an uncapped RNA having a 5′-diphosphate). In some embodiments, a cap is or comprises a guanine nucleotide. In some embodiments, a cap is or comprises a naturally-occurring RNA 5′ cap, including, e.g., but not limited to a 7-methylguanosine cap, which has a structure designated as “m7G.” In some embodiments, a cap is or comprises a synthetic cap analog that resembles an RNA cap structure and possesses the ability to stabilize RNA if attached thereto, including, e.g., but not limited to anti-reverse cap analogs (ARCAs) known in the art). Those skilled in the art will appreciate that methods for joining a cap to a 5′ end of an RNA are known in the art. For example, in some embodiments, a capped RNA may be obtained by in vitro capping of RNA that has a 5′ triphosphate group or RNA that has a 5′ diphosphate group with a capping enzyme system (including, e.g., but not limited to vaccinia capping enzyme system or Saccharomyces cerevisiae capping enzyme system). Alternatively, a capped RNA can be obtained by in vitro transcription (IVT) of a single-stranded DNA template, wherein, in addition to the GTP, an IVT system also contains a dinucleotide cap analog (including, e.g., a m7GpppG cap analog or an N7-methyl, 2′-O-methyl-GpppG ARCA cap analog or an N7-methyl, 3′-O-methyl-GpppG ARCA cap analog) using methods known in the art.

Co-administration: As used herein, the term “co-administration” refers to use of a pharmaceutical composition described herein and an additional therapeutic agent (e.g., a chemotherapeutic agent described herein). The combined use of a pharmaceutical composition described herein and an additional therapeutic agent (e.g., a chemotherapeutic agent described herein) may be performed concurrently or separately (e.g., sequentially in any order). In some embodiments of a pharmaceutical composition described herein, a pharmaceutical composition described herein and an additional therapeutic agent (e.g., a chemotherapeutic agent described herein) be combined in one pharmaceutically-acceptable carrier, or they may be placed in separate carriers and delivered to a target cell or administered to a subject at different times. Each of these situations is contemplated as falling within the meaning of “co-administration” or “combination,” provided that a pharmaceutical composition described herein and an additional therapeutic agent (e.g., a chemotherapeutic agent described herein) are delivered or administered sufficiently close in time that there is at least some temporal overlap in biological effect(s) generated by each on a target cell or a subject being treated.

Combination therapy: As used herein, the term “combination therapy” refers to those situations in which a subject is simultaneously exposed to two or more therapeutic regimens (e.g., two or more therapeutic agents). In some embodiments, two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Complementary: As used herein, the term “complementary” is used in reference to oligonucleotide hybridization related by base-pairing rules. For example, the sequence “C-A-G-T” is complementary to the sequence “G-T-C-A.” Complementarity can be partial or total. Thus, any degree of partial complementarity is intended to be included within the scope of the term “complementary” provided that the partial complementarity permits oligonucleotide hybridization. Partial complementarity is where one or more nucleic acid bases is not matched according to the base pairing rules. Total or complete complementarity between nucleic acids is where each and every nucleic acid base is matched with another base under the base pairing rules.

Contacting: As used interchangeably herein, the term “delivery,” “delivering,” or “contacting” refers to introduction of ssRNA(s) or a composition comprising the same into a target cell (e.g., cytosol of a target cell). A target cell can be cultured in vitro or ex vivo or be present in a subject (in vivo). Methods of introducing ssRNA(s) or a composition comprising the same into a target cell can vary with in vitro, ex vivo, or in vivo applications. In some embodiments, ssRNA(s) or a composition comprising the same can be introduced into a target cell in a cell culture by in vitro transfection. In some embodiments, ssRNA(s) or a composition comprising the same can be introduced into a target cell via delivery vehicles (e.g., lipid nanoparticles described herein). In some embodiments, ssRNA(s) or a composition comprising the same can be introduced into a target cell in a subject by administering a pharmaceutical composition described herein to a subject.

Detecting: The term “detecting” is used broadly herein to include appropriate means of determining the presence or absence of an entity of interest or any form of measurement of an entity of interest in a sample. Thus, “detecting” may include determining, measuring, assessing, or assaying the presence or absence, level, amount, and/or location of an entity of interest. Quantitative and qualitative determinations, measurements or assessments are included, including semi-quantitative. Such determinations, measurements or assessments may be relative, for example when an entity of interest is being detected relative to a control reference, or absolute. As such, the term “quantifying” when used in the context of quantifying an entity of interest can refer to absolute or to relative quantification. Absolute quantification may be accomplished by correlating a detected level of an entity of interest to known control standards (e.g., through generation of a standard curve). Alternatively, relative quantification can be accomplished by comparison of detected levels or amounts between two or more different entities of interest to provide a relative quantification of each of the two or more different entities of interest, i.e., relative to each other.

Disease: As used herein, the term “disease” refers to a disorder or condition that typically impairs normal functioning of a tissue or system in a subject (e.g., a human subject) and is typically manifested by characteristic signs and/or symptoms. In some embodiments, an exemplary disease is cancer.

Encode: As used herein, the term “encode” or “encoding” refers to sequence information of a first molecule that guides production of a second molecule having a defined sequence of nucleotides (e.g., mRNA) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or an ssRNA (e.g., an mRNA) encodes a polypeptide if transcription and translation of mRNA corresponding to that gene produces the polypeptide in a cell or other biological system. In some embodiments, a coding region of an ssRNA encoding a tumor-associated antigen (TAA) refers to a coding strand, the nucleotide sequence of which is identical to the mRNA sequence of such a tumor-associated antigen. In some embodiments, a coding region of an ssRNA encoding a TAA refers to a non-coding strand of such a TAA, which may be used as a template for transcription of a gene or cDNA.

Epitope: As used herein, the term “epitope” includes any moiety that is specifically recognized by an immune system of a patient. For example, an epitope may be any moiety that is specifically recognized by a T cell, a B cell, an immunoglobulin (e.g., antibody or receptor), immunoglobulin (e.g., antibody or receptor), binding component or an aptamer. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Five prime untranslated region: As used herein, the terms “five prime untranslated region” or “5′ UTR” refer to a sequence of an mRNA molecule between a transcription start site and a start codon of a coding region of an RNA. In some embodiments, “5′ UTR” refers to a sequence of an mRNA molecule that begins at a transcription start site and ends one nucleotide (nt) before a start codon (usually AUG) of a coding region of an RNA, e.g., in its natural context.

Homology: As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, polynucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Identity: As used herein, the term “identity” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or between polypeptide molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

RECIST Standard: As used herein, the term “RECIST” or “RECIST standard” refers to Response Evaluation criteria for In Solid Tumors. For example, RECSIT standards are as described in Eisenhauer et al. (European J. Cancer 45: 228-247 (2009)), which is herein incorporated by reference in its entirety). In some embodiments, a RECIST standard is RECIST 1.1. In some embodiments, a RECIST standard is iRECIST. For example, iRECIST standards are as described in Seymour, L. et al. (Lancet Oncol. 18:3 e143-e152 (2017)), which is herein incorporated by reference in its entirety). In some embodiments, a RECIST standard is an “irRECIST standard,” which is an immune-related Response Evaluation Criteria for In Solid Tumors. For example, irRECIST standards are as described in Nishino et al. (Clin Cancer Res 19:3936-43 (2013)), which is herein incorporated by reference in its entirety). In some embodiments, an irRECIST standard is irRECIST 1.1. In some embodiments, a RECIST standard is an “imRECIST standard,” which is an immune-modified Response Evaluation Criteria for In Solid Tumors. For example, irRECIST standards are as described in Hodi et al. (J Clin Oncol 36:850-8 (2018)), which is herein incorporated by reference in its entirety).

Locally advanced tumor: As used herein, the term “locally advanced tumor” or “locally advanced cancer” refers to its art-recognized meaning, which may vary with different types of cancer. For example, in some embodiments, a locally advanced tumor refers to a tumor that is large but has not yet spread to another body part. In some embodiments, a locally advanced tumor is used to describe cancer that has grown outside the tissue or organ it started but has not yet spread to distant sites in the body of a subject. By way of example only, in some embodiments, locally advanced pancreatic cancer typically refers to stage III disease with tumor extension to adjacent organs (e.g., lymph nodes, liver, duodenum, superior mesenteric artery, and/or celiac trunk) but no signs of metastatic disease; yet complete surgical excision with negative pathologic margins is not possible.

Nucleic acid/Polynucleotide: As used herein, the term “nucleic acid” refers to a polymer of at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleic acid particle: A “nucleic acid particle” can be used to deliver nucleic acid to a target site of interest (e.g., cell, tissue, organ, and the like). A nucleic acid particle may be formed from at least one cationic or cationically ionizable lipid or lipid-like material, at least one cationic polymer such as protamine, or a mixture thereof and nucleic acid. Nucleic acid particles include lipid nanoparticle (LNP)-based and lipoplex (LPX)-based formulations.

Nucleotide: As used herein, the term “nucleotide” refers to its art-recognized meaning. When a number of nucleotides is used as an indication of size, e.g., of a polynucleotide, a certain number of nucleotides refers to the number of nucleotides on a single strand, e.g., of a polynucleotide.

Patient: As used herein, the term “patient” refers to any organism who is suffering or at risk of a disease or disorder or condition. Typical patients include animals (e.g., mammals such as mice, rats, rabbits, non-human primates, and/or humans). In some embodiments, a patient is a human. In some embodiments, a patient is suffering from or susceptible to one or more diseases or disorders or conditions. In some embodiments, a patient displays one or more symptoms of a disease or disorder or condition. In some embodiments, a patient has been diagnosed with one or more diseases or disorders or conditions. In some embodiments, a disease or disorder or condition that is amenable to provided technologies is or includes cancer, or presence of one or more tumors. In some embodiments, a patient is receiving or has received certain therapy to diagnose and/or to treat a disease, disorder, or condition. In some embodiments, a patient is a cancer patient.

Polypeptide: The term “polypeptide”, as used herein, typically has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. In some embodiments, polypeptides may contain L-amino acids, D-amino acids, or both and/or may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof (e.g., may be or comprise peptidomimetics).

Reference/Reference standard: As used herein, “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. In some embodiments, a reference or control is or comprises a set specification (e.g., acceptance criteria). Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Ribonucleic acid (RNA): As used herein, the term “RNA” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments where an RNA is a mRNA. In some embodiments where an RNA is a mRNA, a RNA typically comprises at its 3′ end a poly(A) region. In some embodiments where an RNA is a mRNA, an RNA typically comprises at its 5′ end an art-recognized cap structure, e.g., for recognizing and attachment of a mRNA to a ribosome to initiate translation. In some embodiments, a RNA is a synthetic RNA. Synthetic RNAs include RNAs that are synthesized in vitro (e.g., by enzymatic synthesis methods and/or by chemical synthesis methods).

Selective or specific: The term “selective” or “specific”, when used herein in reference to an agent having an activity, is understood by those skilled in the art to mean that the agent discriminates between potential target entities, states, or cells. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of a target-binding moiety for one or more other potential target entities (e.g., competitors). In some embodiments, specificity is evaluated relative to that of a reference specific binding moiety. In some embodiments, specificity is evaluated relative to that of a reference non-specific binding moiety.

Specific binding: As used herein, the term “specific binding” refers to an ability to discriminate between possible binding partners in the environment in which binding is to occur. An antibody agent that interacts with one particular target when other potential targets are present is said to “bind specifically” to the target with which it interacts. In some embodiments, specific binding is assessed by detecting or determining degree of association between CDRs of an antibody agent and their partners; in some embodiments, specific binding is assessed by detecting or determining degree of dissociation of an antibody agent-partner complex; in some embodiments, specific binding is assessed by detecting or determining ability of an antibody agent to compete an alternative interaction between its partner and another entity. In some embodiments, specific binding is assessed by performing such detections or determinations across a range of concentrations.

Subject: As used herein, the term “subject” refers to an organism to be administered with a composition described herein, e.g., for experimental, diagnostic, prophylactic, and/or therapeutic purposes. Typical subjects include animals (e.g., mammals such as mice, rats, rabbits, non-human primates, domestic pets, etc.) and humans. In some embodiments, a subject is a human subject. In some embodiments, a subject is suffering from a disease, disorder, or condition (e.g., cancer). In some embodiments, a subject is susceptible to a disease, disorder, or condition (e.g., cancer). In some embodiments, a subject displays one or more symptoms or characteristics of a disease, disorder, or condition (e.g., cancer). In some embodiments, a subject displays one or more non-specific symptoms of a disease, disorder, or condition (e.g., cancer). In some embodiments, a subject does not display any symptom or characteristic of a disease, disorder, or condition (e.g., cancer). In some embodiments, a subject is someone with one or more features characteristic of susceptibility to or risk of a disease, disorder, or condition (e.g., cancer). In some embodiments, a subject is a patient. In some embodiments, a subject is an individual to whom diagnosis and/or therapy is and/or has been administered.

Suffering from: An individual who is “suffering from” a disease, disorder, and/or condition has been diagnosed with and/or displays one or more symptoms of a disease, disorder, and/or condition.

Synthetic: As used herein, the term “synthetic” refers to an entity that is artificial, or that is made with human intervention, or that results from synthesis rather than naturally occurring. For example, in some embodiments, a synthetic nucleic acid or polynucleotide refers to a nucleic acid molecule that is chemically synthesized, e.g., in some embodiments by solid-phase synthesis. In some embodiments, the term “synthetic” refers to an entity that is made outside of biological cells. For example, in some embodiments, a synthetic nucleic acid or polynucleotide refers to a nucleic acid molecule (e.g., an RNA) that is produced by in vitro transcription using a template.

Therapeutic agent: As used interchangeably herein, the phrase “therapeutic agent” or “therapy” refers to an agent or intervention that, when administered to a subject or a patient, has a therapeutic effect and/or elicits a desired biological and/or pharmacological effect. In some embodiments, a therapeutic agent or therapy is any substance that can be used to alleviate, ameliorate, relieve, inhibit, prevent, delay onset of, reduce severity of, and/or reduce incidence of one or more symptoms or features of a disease, disorder, and/or condition. In some embodiments, a therapeutic agent or therapy is a medical intervention (e.g., surgery, radiation, phototherapy) that can be performed to alleviate, relieve, inhibit, present, delay onset of, reduce severity of, and/or reduce incidence of one or more symptoms or features of a disease, disorder, and/or condition.

Three prime untranslated region: As used herein, the terms “three prime untranslated region” or “3′ UTR” refer to a sequence of an mRNA molecule that begins following a stop codon of a coding region of an open reading frame sequence. In some embodiments, the 3′ UTR begins immediately after a stop codon of a coding region of an open reading frame sequence, e.g., in its natural context. In other embodiments, the 3′ UTR does not begin immediately after stop codon of the coding region of an open reading frame sequence, e.g., in its natural context.

Threshold level (e.g., acceptance criteria): As used herein, the term “threshold level” refers to a level that are used as a reference to attain information on and/or classify the results of a measurement, for example, the results of a measurement attained in an assay. For example, in some embodiments, a threshold level means a value measured in an assay that defines the dividing line between two subsets of a population (e.g. a batch that satisfy quality control criteria vs. a batch that does not satisfy quality control criteria). Thus, a value that is equal to or higher than the threshold level defines one subset of the population, and a value that is lower than the threshold level defines the other subset of the population. A threshold level can be determined based on one or more control samples or across a population of control samples. A threshold level can be determined prior to, concurrently with, or after the measurement of interest is taken. In some embodiments, a threshold level can be a range of values.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition. In some embodiments, treatment may be administered to a subject at a later-stage of disease, disorder, and/or condition.

Unresectable tumor: As used herein, the term “unresectable tumor” typically refers to a tumor that is unable to be removed by surgery. In some embodiments, an unresectable tumor refers to a tumor that involves and/or grows into an essential organ or tissue (including blood vessels that may not be reconstructable) and/or that is otherwise in a location that cannot readily be accessed without unreasonable risk of damage to one or more other critical or essential organs and/or tissues (including blood vessels). In some embodiments, an unresectable tumor refers to a tumor that cannot be resected by surgery without risk of damage to a patient, which is determined in sound medical judgement to outweigh benefit expected to be received for that patient by resection. In some embodiments, “unresectability” of a tumor refers to the likelihood of achieving a margin-negative (R0) resection. In the context of pancreatic cancer, encasement of major vessels by a tumor such as superior mesenteric artery (SMA) or celiac axis, portal vein occlusion, and the presence of celiac or para-aortic lymphadenopathy are generally acknowledged as findings that preclude R0 surgery. Those skilled in the art will understand parameters that determine whether a tumor is unresectable or not.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

Outcomes of Standard of Care (SOC) remain poor for patients with relapsed or refractory advanced solid tumors. Treatment options include further palliative chemotherapy, which might be less tolerated after previous repeated exposure to cytotoxic compounds, or best supportive care, and investigational treatments without proven benefit. Therapy in this population is not curative, with an expected overall survival of a few months. Vaccines have emerged as an effective treatment option in some cancers with high unmet medical need. However, vaccine trials treating patients who have treatment refractory tumors have been largely unsuccessful. Therefore, the medical need is still high to develop vaccines to treat various cancer types, including treatment-refractory cancers.

The present disclosure, among other things, provides insights and technologies for treating cancer (e.g., melanoma (e.g., advanced melanoma)) with a pharmaceutical composition (e.g., an immunogenic composition, e.g., a vaccine) comprising RNA encoding tumor-associated antigens (TAA). The present disclosure, among other things, provides an insight that pharmaceutical compositions described herein may be particularly useful and/or effective when administered to patients with no evidence of disease at time of first administration thereby showing that the pharmaceutical composition induced T cell immunity even the absence of a detectable tumor.

In some embodiments, the present disclosure, among other things, provides methods of administering to a patient at least one dose of a pharmaceutical compositions (e.g., an immunogenic composition, e.g., a vaccine) described herein, which includes RNA molecule(s) and lipid particles (e.g., lipoplexes or lipid nanoparticles). In some embodiments, one or more RNA molecules encode one more tumor-associated antigens (TAA) that when administered to a patient combine to induce a strong adaptive immune response (e.g., a CD4⁺ and/or CD8⁺ T cell immune response) against one or more of the TAAs encoded by the one or more RNA molecules. Without wishing to be bound by any particular theory, the present disclosure proposes that such pharmaceutical compositions may achieve antigen-specific T cell immunity and durable objective responses in cancer patients (e.g., patients with unresectable cancer (e.g., melanoma), patients who have or are receiving checkpoint inhibitors, or patients with both). In particular, the present disclosure also teaches that by administering the pharmaceutical composition (e.g., an immunogenic composition, e.g., a vaccine) as described herein to a patient that was diagnosed with cancer prior to the time of administration but where the patient is classified as having no evidence of disease at the time of administration.

No evidence of disease can be a classification according to a RECIST standard. In some embodiments, no evidence of disease does not mean that a patient does not have any disease, but rather that there is no evidence that disease is present, particularly as determined according to a RECIST standard.

In some embodiments, the present disclosure, among other things, provides insights that mRNA(s) encoding an amino acid sequence comprising a tumor associated antigen (TAA), an immunogenic variant thereof, or an immunogenic fragment of the TAA or the immunogenic variant thereof. Thus, the mRNA(s) encode a peptide or protein comprising at least an epitope of a TAA or an immunogenic variant thereof for inducing an immune response against the TAA. In some embodiments, the present disclosure, among other things, provides RNA technologies to deliver one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof to a patient. In some embodiments, a single RNA molecule encodes all of (i) a New York oesophageal squamous cell carcinoma (NY-ESO-1) antigen, (ii) a melanoma-associated antigen A3 (MAGE-A3) antigen, (iii) a tyrosinase antigen, and (iv) a transmembrane phosphatase with tensin homology (TPTE) antigen. In some embodiments, sequences encoding (i) a New York oesophageal squamous cell carcinoma (NY-ESO-1) antigen, (ii) a melanoma-associated antigen A3 (MAGE-A3) antigen, (iii) a tyrosinase antigen, and (iv) a transmembrane phosphatase with tensin homology (TPTE) antigen are not present on a single RNA molecule. For example, a first RNA molecule could encode two of (i) a New York oesophageal squamous cell carcinoma (NY-ESO-1) antigen, (ii) a melanoma-associated antigen A3 (MAGE-A3) antigen, (iii) a tyrosinase antigen, and (iv) a transmembrane phosphatase with tensin homology (TPTE) antigen, and a second RNA molecule could encode the remaining two. As another example, sequence encoding (i) a New York oesophageal squamous cell carcinoma (NY-ESO-1) antigen, (ii) a melanoma-associated antigen A3 (MAGE-A3) antigen, (iii) a tyrosinase antigen, and (iv) a transmembrane phosphatase with tensin homology (TPTE) antigen could each be present on a different RNA molecule, such that each RNA molecule only encodes one antigen.

In some embodiments, the present disclosure, among other things, provides insights that a pharmaceutical composition (e.g., an immunogenic composition, e.g., a vaccine) is formulated with lipid particles (e.g., lipoplexes or lipid nanoparticles) for administration to a patient (e.g., intravenous (IV), intramuscular, or subcutaneous administration). In particular, a pharmaceutical composition comprising one or more RNA (e.g., mRNA) molecules encoding at least one TAAs (e.g., NY-ESO-1 antigen, MAGE-A3 antigen, tyrosinase antigen, and/or TPTE antigen) or immunogenic fragment thereof is formulated with lipid particles (e.g., lipoplexes or lipid nanoparticles) for administration to a patient (e.g., IV, intramuscular, or subcutaneous administration). Without wishing to be bound by any particular theory, the pharmaceutical composition (e.g., the immunogenic composition, e.g., the vaccine) as described herein can be taken up by immature dendritic cells and RNA molecules translated for augmented antigen presentation on HLA Class I and II molecule. In some embodiments, TAA e.g., NY-ESO-1 antigen, MAGE-A3 antigen, tyrosinase antigen, and/or TPTE antigen) are expressed from RNA (e.g., mRNA), e.g., engineered for minimal immunogenicity, and/or formulated in lipid nanoparticles (e.g., LNPs). In some embodiments, RNA (e.g., mRNA) that encodes at least one TAA (e.g., NY-ESO-1 antigen, MAGE-A3 antigen, tyrosinase antigen, and/or TPTE antigen) may comprise modified nucleotides (e.g., but not limited to pseudouridine).

In some embodiments, the present disclosure, among other things, provides methods of administering to a patient at least one dose of a pharmaceutical composition comprising: (a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and (b) lipid particles (e.g., lipoplexes or lipid nanoparticles); wherein the patient was diagnosed with cancer prior to the time of administration, but the patient is classified as having no evidence of disease at the time of administration (e.g., no evidence of disease is determined by applying a response Evaluation Criteria In Solid Tumors (RECIST) standard, e.g., an RECIST1.1 standard or an irRECIST standard).

In some embodiments, the present disclosure, among other things, provides methods of administering at least one dose of a pharmaceutical composition to a patient suffering from cancer, wherein the pharmaceutical composition comprises: (a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and (b) lipid particles (e.g., lipoplexes or lipid nanoparticles).

In some embodiments, the present disclosure, among other things, provides pharmaceutical compositions for use in inducing an immune response against cancer in a patient, wherein the pharmaceutical composition comprises: (a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and (b) lipid particles (e.g., lipoplexes or lipid nanoparticles); and wherein the patient is classified as having no evidence of disease, but has previously been diagnosed with cancer (e.g., melanoma).

In some embodiments, the present disclosure, among other things, provides pharmaceutical compositions for use in treating cancer, wherein the pharmaceutical composition comprises: (a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and (b) lipid particles (e.g., lipoplexes or lipid nanoparticles); and wherein the patient is classified as having no evidence of disease, but has previously been diagnosed with cancer (e.g., melanoma). No evidence of disease can be determined according to a RECIST standard, e.g., a RECIST1.1 standard or an irRECIST standard.

In some embodiments, the present disclosure, among other things, provides pharmaceutical compositions for use in inducing an immune response against cancer in a patient, wherein the pharmaceutical composition comprises: (a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and (b) lipid particles (e.g., lipoplexes or lipid nanoparticles).

In some embodiments, the present disclosure, among other things, provides pharmaceutical compositions for use in treating cancer, wherein the pharmaceutical composition comprises: (a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and (b) lipid particles (e.g., lipoplexes or lipid nanoparticles).

### I. Prior Approaches

The present disclosure provides technologies for treating cancer. An exemplary cancer that can be treated by technologies described herein is melanoma. Health risks associated with melanoma can be significant, and advanced or metastatic melanoma (e.g., unresectable Stage III, Stage IV) remain lethal disease. For systemic treatment of unresectable Stage III/IV and recurrent melanoma, for example, there are currently two approaches that have demonstrated improvement in progression-free survival (PFS) and overall survival (OS) in randomized trials. Those two approaches are (1) checkpoint inhibition (PD-1/PD-L1 inhibition, CTLA-4 inhibition), and (2) targeting the mitogen-activated protein kinase (MAPK) pathway. While these approaches have experienced some level of success, both experience challenges and may benefit from combination with or replacement by technologies described herein. An overview of the current approaches are described below.

### A. Systemic Therapies

### 1. Checkpoint Inhibitors

Immune checkpoint inhibitors (CPIs) targeting cytotoxic T-lymphocyte-associated antigen 4 (CTLA-4; e.g., ipilimumab) and programmed death 1 (PD-1; e.g., nivolumab and pembrolizumab) have been approved for the treatment of advanced or metastatic melanoma alone or in combination (YERVOY® USPI; OPDIVO® USPI; KEYTRUDA® USPI, each of which is incorporated herein by reference in its entirety). In first-line therapy, nivolumab and ipilimumab combination therapy has been associated with an improved overall response rate (ORR; 57% versus 19% versus 44%) and median PFS (11.5 months versus 2.9 months versus 6.9 months) compared with single-agent ipilimumab or nivolumab, respectively. However, the combination is associated with substantial toxicity and the impact of combination therapy on overall survival has not yet been fully established (Wolchok et al. 2017, which is incorporated herein by reference in its entirety). Monotherapy treatment with anti-PD-1 therapy (e.g., pembrolizumab or nivolumab) or CTLA-4 inhibitors (e.g., ipilimumab) is also an option for patients who are not candidates for combination therapy.

### 2. Signal Transduction Inhibitors

Approximately half of patients with metastatic cutaneous melanoma harbor an activating mutation of proto-oncogene B-Raf (BRAF), an intracellular signaling kinase in the MAPK pathway. BRAF inhibitors (e.g., vemurafenib and dabrafenib) have shown clinical activity in melanomas with BRAF V600 mutations. BRAF inhibitors have monotherapy efficacy in patients with BRAF-mutated melanoma, but half of patients relapse within approximately 6 months due to development of drug resistance. Combination therapy with BRAF and MEK inhibitors circumvents resistance and has better efficacy (e.g., improved ORR, duration of response, PFS, and OS) than BRAF inhibitor monotherapy for patients with previously untreated unresectable or metastatic disease. Nevertheless, 50% of patients who respond to combination therapy still progress within the first 12 months (Mackiewicz et al. 2018; Gellrich et al. 2020, each of which are incorporated herein by reference in its entirety). Pembrolizumab and nivolumab are also approved for first line treatment of patients with BRAF mutations. For patients with BRAF V600-mutated tumors that do not progress very quickly, the currently recommended therapeutic sequence is immunotherapy (e.g., anti-PD-1 therapy) followed by targeted therapy with BRAF/MEK inhibitors (Michielin et al. 2019, which is incorporated herein by reference in its entirety).

### 3. Intralesional Therapy

Talimogene laherparepvec (T-vec, tradename Imlygic®) is a genetically modified oncolytic viral therapy indicated for the local treatment of unresectable cutaneous, subcutaneous, and nodal lesions in patients with melanoma recurrent after initial surgery. T-vec is a modified herpes simplex virus, type 1 (HSV-1) that has undergone genetic modifications (insertion of 2 copies of the human cytokine granulocyte macrophage-colony stimulating factor [GM-CSF]gene) to promote selective viral replication in tumor cells, while reducing viral pathogenicity and promoting immunogenicity. In a randomized Phase III trial, intra-tumoral T-vec compared to subcutaneous GM-CSF showed an objective response rate of 26% versus 5.7%. However, the difference seen in overall survival did not reach statistical significance and the response rate in visceral lesions was poor (Rehman et al. 2016, which is incorporated herein by reference in its entirety). Thus, this treatment option may be appropriate for selected patients.

### 4. Other Therapies

Treatment options for patients with advanced or metastatic melanoma who have progressed on targeted therapy or immunotherapy may include high-dose interleukin (IL)-2 or other cytotoxic therapies (e.g., dacarbazine, carboplatin/paclitaxel, albumin-bound paclitaxel). These agents have modest response rates of less than 20% in the first-line and second-line settings, but no data exist in post PD-1 settings. Furthermore, little consensus exists regarding optimal standard chemotherapy (Swetter et al. 2021, which is incorporated herein by reference in its entirety). Initial promising results were reported for a c-kit-inhibitor with response rates of 23.3% (Guo et al. 2011, which is incorporated herein by reference in its entirety), whereas the multi-kinase-inhibitor sorafenib targeting both the MAPK-cascade as well as the VEGF and PDGF-cascade did not improve median PFS over placebo in a Phase III, randomized, double-blind, placebo-controlled trial in combination with carboplatin and paclitaxel (Hauschild et al. 2009, which is incorporated herein by reference in its entirety).

### 5. Adjuvant Therapy

For treatment of patients with fully resected cutaneous melanoma in stage III and also in completely resected stage IV (no evidence of disease), an adjuvant treatment has been recommended (Swetter et al. 2021, which is incorporated herein by reference in its entirety).

For these patient groups, the adjuvant treatment has been based on a number of prospective clinical trials with immune checkpoint inhibitors and BRAF-targeted therapy. Clinical trials in the adjuvant setting have shown that immune checkpoint inhibitors and BRAF targeting therapies improve the relapse-free survival (RFS) or disease-free survival rates when compared to conventional therapy, as well as provide a higher overall survival (OS) rate at 3 or 5 years. Yet, there are concerns with regards to toxicity of adjuvant treatments, e.g., Grade 3 to 4 adverse events (AEs) in 25 to 41% of patients and a low proportion of patients with life-long AEs (mostly immune-related) after adjuvant immune checkpoint inhibition (Gershenwald et al. 2017, which is incorporated herein by reference in its entirety).

### 6. Overview of Exemplary Features of Described Technologies

Based on the landscape of treatment options described above, significant progress has been made for the use of approved therapies for the treatment of Stage III and IV melanoma. However, approximately 40 to 45% of patients have been reported to experience no response to initial therapy, showing primary resistance, and an additional 30 to 40% have been reported to experience an initial response, but eventually progress, having secondary resistance (Mooradian and Sullivan 2019, which is incorporated herein by reference in its entirety). These subsets of patients with primary refractory disease or secondary relapse represent populations with an unmet medical need, justifying the development of novel therapies for patients with unresectable Stage III and IV melanoma in order to induce higher initial response rates reducing primary resistance, and for patients with recurrent melanoma (Testori et al. 2020, which is incorporated herein by reference in its entirety). Moreover, the addition of novel therapies to anti-PD-1 treatments may increase the response versus anti-PDI therapies alone.

The tolerability of available treatment options currently precludes the use of adjuvant therapy in patients with stage IIB or IIC high-risk disease and partially also for patients with stage III disease. New systemic therapies with better tolerability profiles may allow treatment of this subset of patients and improve available adjuvant therapeutic options for patients with completely resected disease.

Exemplary compositions described herein comprise TAAs: NY-ESO-1, tyrosinase, MAGE-A3, and TPTE. Among other reasons, these cancer vaccine targets were selected based on the following criteria:
Low or lack of expression in toxicity-relevant organs.
Expression in a substantial fraction of melanoma cells.
The ability to induce antigen-specific immune responses.
Tumor biological role as per literature.

Furthermore, these TAAs were selected, at least in part, due to a tissue expression analyses in the Phase I Lipo-MERIT trial. In this trial, approximately 8% of screened patients did not express detectable levels of any of these four antigens in tumors or metastases. Considering the clonal heterogeneity of cancer and limitation of clinically available samples (only one location), the present disclosure provides the recognition that it is likely that more than the observed rate of 92% of patients actually express at least one of the selected TAAs. In addition, in a substantial percentage of patients, several of these TAAs were found to be co-expressed. Therefore, the present disclosure provides the insight that a significant population of melanoma patients would be expected to develop poly-epitopic, vaccine-induced immune responses and to benefit from treatment with compositions described herein. As used herein, the term “BNT111” refers to a pharmaceutical composition comprising a NY-ESO-1 antigen, a tyrosinase antigen, a MAGE-A3 antigen, and a TPTE antigen, preferentially formulated as shown in Table 3.

In some embodiments, compositions described herein (e.g., BNT111) can prime, activate and/or expand CD4⁺ and CD8⁺ T cell specificities, and thus, generate a complementary pool of T cell specificities directed against non-mutant TAAs that are frequently expressed in human melanoma irrespective of the mutational burden of the tumor.

The liposome formulation of compositions described herein (e.g., BNT111) is designed to deliver the antigens into secondary lymphatic tissues and exploits antiviral innate and adaptive immune mechanisms for induction of highly potent antigen-specific T cell responses. Intravenously administered compositions described herein (e.g., BNT111) can be delivered to secondary lymphatic tissues (e.g., spleen, lymph nodes, and bone marrow) and are rapidly taken up by antigen-presenting cells (APCs). The proteins translated from the RNA components of compositions described herein (e.g., BNT111) can be processed and presented on the patients' individual set of both HLA-class I and HLA-class II molecules (Kranz et al. 2016, which is incorporated herein by reference in its entirety). The close proximity of APCs to T cells in lymphatic tissues represents an ideal microenvironment for efficient priming and amplification of CD8⁺ and CD4⁺ T-cell responses (Zinkernagel et al. 1997, which is incorporated herein by reference in its entirety). Components of compositions described herein activate APCs via toll-like receptor signaling, which results in a pulsatile release of pro-inflammatory cytokines, such as IFN-α, IL-6, IFN-γ, and IP-10. Also, secretion of Type-I interferons concomitant to efficient antigen presentation stimulates immune cells and directly inhibits regulatory T cells (Srivastava et al. 2014, which is incorporated herein by reference in its entirety), which in combination with cognate CD4⁺ T cell help, is necessary for overcoming tolerance to self-antigens. Based on this dual mechanism of action, repeated administration compositions described herein (e.g., BNT111) allows potent priming and rapid amplification of antigen specific CD8⁺ T-cell responses.

Together with TAA expression data and the observed dual mechanism of action, the present disclosure provides the expectation that the majority of melanoma patients will develop de novo or intensified poly-epitopic, vaccine-induced, antigen-specific immune responses and derive benefit from treatment with compositions described herein.

Activation, expansion, and differentiation of naïve T cells is physiologically associated with induction of the immune-regulatory checkpoint molecule PD-1 (Sharpe and Pauken 2018, which is incorporated herein by reference in its entirety). Therefore, as discussed further herein, anti-PD-i/anti-PD-L1 blockade will augment the activity of T cell responses induced by compositions herein (e.g., BNT111), as supported by non-clinical data in mouse tumor models. One reason for treatment failure in patients treated with PD-1/PD-L1 blockade has been the lack of pre-formed antigen-specific T lymphocytes recognizing relevant tumor antigens. In some embodiments, such T lymphocytes are elicited by compositions described herein (e.g., BNT111), which induce potent antigen-specific CD4⁺ and CD8⁺ T cell responses. These T cells not only execute direct anti-tumor activity by their cytotoxicity upon recognition of their target antigens on tumor cells, but also induce inflammation (e.g., IFN-γ secretion) in the tumor microenvironment thereby sensitizing tumor cells to the therapeutic effects of checkpoint inhibitors.

In some embodiments, for patients that are refractory to or relapsing after anti-PD-i/anti-PD-L1 therapy (meaning that activation of pre-existing memory T cells only is not sufficient to mediate clinical activity), adding a PD-1 inhibitor (which would rescue newly primed T cell specificities from exhaustion) will amplify the effect of compositions described herein (e.g., BNT111).

In some embodiments, the objective response rate of 25% and disease control rate of 22% for the combination of compositions described herein (e.g., BNT111) and a PD-1 inhibitor—in patients with a median of 5 prior treatments—may be higher if the treatment is used in patient populations that are less pretreated.

### I. Tumor Associated Antigens

In some embodiments, the present disclosure, among other things, provides one or more RNA molecules that encode antigens. In some embodiments, antigens are tumor associated antigens (TAA). The present disclosure provides the insight that a significant percentage of melanoma patients cumulatively express at least one of the four TAAs, irrespective of the mutational burden of the tumor. In some embodiments, one or more RNA molecules collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof. These antigens have been observed at a high prevalence in melanoma patients. These antigens have also been reported to have selective expression in cancer cells. The present disclosure provides the insight that selective expression of a NY-ESO-1 antigen, a MAGE-A3 antigen, a tyrosinase antigen, and/or a TPTE antigen can provide a low risk of on-target/off-tumor toxicity. In some embodiments, one or more RNA molecules that encode antigens (e.g., TAA, e.g., a NY-ESO-1 antigen, a MAGE-A3 antigen, a tyrosinase antigen, and/or a TPTE antigen) can be expected to induce poly-epitopic CD8⁺ and CD4⁺ T cell responses that lead to the killing of tumor cells, which express at least one of the targeted antigens.

In some embodiments, at least one of a NY-ESO-1 antigen, a MAGE-A3 antigen, a tyrosinase antigen, and a TPTE antigen are full-length, non-mutated antigens. In some embodiments, all of a NY-ESO-1 antigen, a MAGE-A3 antigen, a tyrosinase antigen, and a TPTE antigen are full-length, non-mutated antigens. In some embodiments, a NY-ESO-1 antigen, a MAGE-A3 antigen, and a TPTE antigen are full-length, non-mutated antigens. In some embodiments, a NY-ESO-1 antigen and a MAGE-A3 antigen are full-length, non-mutated antigens. In some embodiments, at least one of a NY-ESO-1 antigen, a MAGE-A3 antigen, a tyrosinase antigen, and a TPTE antigen is not a full-length antigen. For example, in some embodiments, a tyrosinase antigen is not full-length, but only comprises a portion of tyrosinase. In some embodiments, a tyrosinase antigen comprises a signal peptide, a EGF-like domain, a CμA domain, a CμB domain, or a combination thereof. In some embodiments, a TPTE antigen is not full-length, but only comprises a portion of a TPTE antigen.

In some embodiments, after administration of one or more RNA molecules (e.g., one or more RNA molecules that collectively encode a (i) NY-ESO-1 antigen, (ii) a MAGE-A3 antigen, (iii) a tyrosinase antigen, (iv) a TPTE antigen, or (v) a combination thereof) at least one of the NY-ESO-1 antigen, the MAGE-A3 antigen, the tyrosinase antigen, and the TPTE antigen are expressed from dendritic cells in lymphoid tissues of the patient.

In some embodiments, at least one of a NY-ESO-1 antigen, a MAGE-A3 antigen, a tyrosinase antigen, and a TPTE antigen are present in the cancer (e.g., melanoma). In some embodiments, the methods described herein include determining the presence and/or abundance (e.g., a level or amount) of at least one of a NY-ESO-1 antigen, a MAGE-A3 antigen, a tyrosinase antigen, and a TPTE antigens in a cancer of a patient. For example, in some embodiments, a sample (e.g., a blood or blood component (e.g., serum or plasma) sample or tumor biopsy) is isolated from a patient and is assessed for a presence and/or abundance (e.g., a level or amount) of one of a NY-ESO-1 antigen, a MAGE-A3 antigen, a tyrosinase antigen, and a TPTE antigens.

A New York oesophageal squamous cell carcinoma (NY-ESO-1) antigen: A NY-ESO-1 antigen is a member of the cancer testis antigen (CTA) gene family. Approximately 50% of all CTA genes form multigene families on the X chromosome and are referred to as CT-X genes. These CTAs are located in specific clusters along the chromosome with the highest density in the Xq24-q28 region (see Thomas et al., Front. Immunol. 9:947 (2018), which is incorporated herein by reference in its entirety). Without wishing to be bound by theory, it is commonly believed that NY-ESO-1 expression is largely restricted to testicular germ cells and placenta trophoblasts with no or low expression at the transcript or protein level in normal healthy adult somatic cells. NY-ESO-1 is expressed in various human cancers including melanoma) (Giavina-Bianchi et al. J. Immunol. Res. 2015, which is incorporated herein by reference in its entirety). According to at least one report). NY-ESO-1 protein was detected in about 20% of invasive melanomas (Giavina-Bianchi).

In some embodiments, an RNA molecule of one or more RNA molecules as described herein encodes a New York oesophageal squamous cell carcinoma (NY-ESO-1) antigen, or an immunogenic fragment thereof. In some embodiments, the single RNA molecule that encodes a NY-ESO-1 antigen is a full-length, non-mutated antigen. In some embodiments, an RNA molecule of the one or more RNA molecules described herein encodes a NY-ESO-1 antigen that does not comprises an amino acid substitution associated with melanoma cancer progression (e.g., a wild type amino acid sequence of the NY-ESO-1 antigen).

In some embodiments, a NY-ESO-1 antigen comprises an amino acid sequence that is at least 80%, 85%, 90%, 95%, 96%, 97%, 98%, or 99% identical to that of SEQ ID NO: 1. In some embodiments, the NY-ESO-1 antigen comprises or consists of an amino acid sequence of SEQ ID NO: 1.

In some embodiments, a NY-ESO-1 antigen is encoded by a nucleic acid sequence that is at least 80%, 85%, 90%, 95%, 96%, 97%, 98%, or 99% identical to that of SEQ ID NO: 2.

A melanoma-associated antigen A3 (MAGE-A3) antigen: A MAGE-A3 antigen is a member of the MAGEA gene family. The MAGEA genes are clustered at chromosomal location Xq28. They have been implicated in some hereditary disorders, such as dyskeratosis congenita. MAGE-A3 is proposed to enhance ubiquitin ligase activity of RING-type zinc finger-containing E3 ubiquitin-protein ligases and may enhance ubiquitin ligase activity of TRIM28 and stimulate p53/TP53 ubiquitination by TRIM28. MAGE-A3 is also proposed to act through recruitment and/or stabilization of the Ubl-conjugating enzyme (E2) at the E3:substrate complex. MAGE-A3 is recognized to play a role in embryonal development and is re-expressed in tumor transformation or aspects of tumor progression. In some embodiment, in vitro expression promotes cell viability in melanoma cell lines. MAGE-A3 antigen is known to be recognized by T cell when expressed on a melanoma.

In some embodiments, an RNA molecule of one or more RNA molecules as described herein encodes a melanoma-associated antigen A3 (MAGE-A3) antigen, or an immunogenic fragment thereof. In some embodiments, the single RNA molecule encodes a full length, non-mutated MAGE-A3 antigen. In some embodiments, an RNA molecule of one or more RNA molecules as described herein encodes a MAGE-A3 antigen that does not comprises an amino acid substitution associated with melanoma cancer progression (e.g., a wild type amino acid sequence of the MAGE-A3 antigen).

In some embodiments, a MAGE-A3 antigen comprises an amino acid sequence that is at least 80%, 85%, 90%, 95%, 96%, 97%, 98%, or 99% identical to that of SEQ ID NO: 3. In some embodiments, a MAGE-A3 antigen comprises or consists of an amino acid sequence of SEQ ID NO: 3.

In some embodiments, a MAGE-A3 antigen is encoded by a nucleic acid sequence that is at least 80%, 85%, 90%, 95%, 96%, 97%, 98%, or 99% identical to that of SEQ ID NO: 4.

A tyrosinase antigen: A tyrosinase antigen is encoded by the TYR gene and is a member of the of the tyrosinase family or proteins, which are widely distributed among animals. This gene encodes a melanosomal enzyme that belongs to the tyrosinase family and plays an important role in the melanin biosynthetic pathway. Tyrosinase is known to be expressed in numerous cancers including melanoma (see Osella-Abate et al., Br. J. Cancer 89(8): 1457-62 (2003), which is incorporated herein by reference in its entirety).

In some embodiments, a single RNA molecule of one or more RNA molecules as described herein encodes a tyrosinase antigen, or an immunogenic fragment thereof. In some embodiments, an RNA molecule encodes a full length, non-mutated tyrosinase antigen. In some embodiments, an RNA molecule of one or more RNA molecules as described herein encodes a tyrosinase antigen that does not comprises an amino acid substitution associated with melanoma cancer progression (e.g., a wild type amino acid sequence of the tyrosinase antigen). In some embodiments, a tyrosinase antigen is not full-length, but only comprises a portion of tyrosinase. In some embodiments, a tyrosinase antigen comprises a signal peptide, a EGF-like domain, a CμA domain, a CμB domain, or a combination thereof.

In some embodiments, a tyrosinase antigen comprises an amino acid sequence that is at least 80%, 85%, 90%, 95%, 96%, 97%, 98%, or 99% identical to that of SEQ ID NO: 5. In some embodiments, the tyrosinase antigen comprises or consists of an amino acid sequence of SEQ ID NO: 5.

In some embodiments, a tyrosinase antigen is encoded by a nucleic acid sequence that is at least 80%, 85%, 90%, 95%, 96%, 97%, 98%, or 99% identical to that of SEQ ID NO: 6.

A transmembrane phosphatase with tensin homology (TPTE) antigen: A TPTE antigen is a member of the cancer testis antigen (CTA) family. CTA antigen expression is highly tissue-restricted. TPTE is a transmembrane phosphatase with tensin homology which may play a role in the signal transduction pathways of endocrine or spermatogenic function of testis. TPTE mRNA expression in healthy adult tissues is confined to the testis, and transcript levels are below the detection limit of highly sensitive RT-PCR in all other normal tissue specimens. (Simon P, et al. Functional TCR retrieval from single antigen specific human T cells reveals multiple novel epitopes. In Cancer Immunol Res. 2(12): 1230-44 (2014), which is incorporated by reference herein in its entirety.)

In some embodiments, an RNA molecule of one or more RNA molecules as described herein encodes a TPTE antigen, or an immunogenic fragment thereof. In some embodiments, a RNA molecule encodes a full length, non-mutated TPTE antigen. In some embodiments, a RNA molecule encodes a truncated TPTE antigen. In some embodiments, a RNA molecule encodes a truncated, non-mutated TPTE antigen. In some embodiments, an RNA molecule of one or more RNA molecules as described herein encodes a TPTE antigen that does not comprises an amino acid substitution associated with melanoma cancer progression (e.g., a wild type amino acid sequence of the TPTE antigen).

In some embodiments, an RNA molecule of one or more RNA molecules as described herein encodes a TPTE antigen or an immunogenic fragment thereof as described in WO2005/026205, the entire content of which is incorporated herein by reference for the purposes described herein.

In some embodiments, a TPTE antigen comprises an amino acid sequence that is at least 80%, 85%, 90%, 95%, 96%, 97%, 98%, or 99% identical to that of SEQ ID NO: 7. In some embodiments, a TPTE antigen comprises or consists of an amino acid sequence of SEQ ID NO: 7.

In some embodiments, a TPTE antigen is encoded by a nucleic acid sequence that is at least 80%, 85%, 90%, 95%, 96%, 97%, 98%, or 99% identical to that of SEQ ID NO: 8.

In some embodiments, exemplary nucleic acid sequence encoding TAAs as described herein and amino acid sequence of TAAs as described herein are provided in Table 1 below.

TABLE 1
Sequences for TAAs

| SEQ | | |
|---|---|---|
| ID | | |
| NO: | Identifier | Sequence |
| 1 | NY-ESO-1 Full | ATGCAGGCCGAGGGCAGAGGAACAGGCGGCAGCACAGGCG |
| | length nucleic | ACGCAGATGGACCAGGCGGCCCTGGAATCCCTGATGGCCCA |
| | acid | GGCGGCAATGCTGGGGGACCAGGAGAAGCTGGCGCCACAGG |
| | | CGGGAGAGGACCTAGAGGAGCTGGAGCCGCTAGAGCTTCTG |
| | | GACCTGGGGGAGGCGCCCCTAGAGGACCACATGGAGGCGCT |
| | | GCCAGCGGCCTGAATGGCTGCTGCAGATGCGGCGCCAGAGG |
| | | CCCTGAGAGCCGGCTGCTGGAATTCTACCTGGCCATGCCCTT |
| | | CGCCACCCCCATGGAAGCCGAGCTGGCCAGAAGATCCCTGG |
| | | CTCAGGACGCTCCTCCTCTGCCTGTGCCCGGCGTGCTGCTGA |
| | | AAGAATTCACCGTGTCCGGCAACATCCTGACCATCAGACTGA |
| | | CAGCCGCCGATCACAGACAGCTCCAGCTGAGCATCAGCTCTT |
| | | GCCTGCAGCAGCTGAGCCTGCTGATGTGGATCACCCAGTGCT |
| | | TTCTGCCCGTGTTCCTGGCCCAGCCACCCAGCGGACAGAGAA |
| | | GG |
| 2 | NY-ESO-1 Full | MQAEGRGTGGSTGDADGPGGPGTPDGPGGNAGGPGEAGATGG |
| | length amino | RGPRGAGAARASGPGGGAPRGPHGGAASGLNGCCRCGARGPE |
| | acid | SRLLEFYLAMPFATPMEAELARRSLAQDAPPLPVPGVLLKEFTV |
| | | SGNILTIRLTAADHRQLQLSISSCLQQLSLLMWITQCFLPVFLAQ |
| | | PPSGQRR |
| 3 | MAGEA3 full | ATGCCCCTTGAACAGCGCTCACAGCACTGCAAACCTGAGGA |
| | length nucleic | GGGCCTTGAAGCAAGGGGCGAAGCTCTGGGGTTGGTCGGTG |
| | acid | CACAAGCACCCGCCACTGAGGAACAGGAAGCCGCGTCTAGC |
| | | TCATCAACCCTGGTTGAAGTGACACTGGGCGAAGTGCCTGCT |
| | | GCGGAGAGTCCAGACCCTCCCCAGTCCCCTCAAGGCGCTTCT |
| | | AGCCTGCCTACCACGATGAACTACCCACTGTGGTCACAGAGC |
| | | TATGAGGACAGTTCCAATCAAGAAGAAGAAGGCCCGTCTAC |
| | | CTTCCCCGATCTTGAGTCCGAGTTTCAGGCCGCTCTGTCCCG |
| | | GAAGGTGGCAGAGCTCGTGCACTTTCTCCTGTTGAAGTATCG |
| | | AGCCCGGGAGCCTGTCACTAAGGCCGAAATGCTGGGCTCTG |
| | | TAGTGGGGAATTGGCAGTATTTCTTCCCCGTGATCTTCAGCA |
| | | AAGCCTCCAGCAGCCTGCAATTGGTGTTCGGTATTGAACTGA |
| | | TGGAAGTAGATCCGATTGGGCATCTGTACATCTTTGCGACAT |
| | | GTCTGGGACTGTCCTATGACGGACTGCTCGGGGATAACCAG |
| | | ATTATGCCGAAAGCCGGTCTGCTGATCATAGTTCTCGCCATC |
| | | ATTGCCAGAGAGGGAGATTGTGCTCCAGAGGAGAAGATCTG |
| | | GGAGGAATTGTCTGTGCTGGAGGTCTTTGAGGGTAGGGAGG |
| | | ACAGCATTCTCGGCGATCCCAAGAAACTCCTGACCCAGCACT |
| | | TTGTCCAGGAGAACTACCTCGAATACAGACAGGTTCCAGGC |
| | | AGTGACCCTGCTTGCTACGAGTTCCTTTGGGGACCCCGTGCA |
| | | TTGGTAGAGACAAGCTATGTCAAAGTGCTGCACCATATGGTG |
| | | AAGATATCTGGAGGACCACACATCAGTTACCCACCCCTTCAT |
| | | GAGTGGGTTCTGCGCGAAGGGGAGGAG |
| 4 | MAGEA3 full | MPLEQRSQHCKPEEGLEARGEALGLVGAQAPATEEQEAASSSS |
| | length amino | TLVEVTLGEVPAAESPDPPQSPQGASSLPTTMNYPLWSQSYEDS |
| | acid | SNQEEEGPSTFPDLESEFQAALSRKVAELVHFLLLKYRAREPVT |
| | | KAEMLGSVVGNWQYFFPVIFSKASSSLQLVFGTELMEVDPIGHL |
| | | YIFATCLGLSYDGLLGDNQIMPKAGLLIIVLAIIAREGDCAPEEKI |
| | | WEELSVLEVFEGREDSILGDPKKLLTQHFVQENYLEYRQVPGS |
| | | DPACYEFLWGPRALVETSYVKVLHHMVKISGGPHISYPPLHEW |
| | | VLREGEE |
| 5 | Tryosinase | ATGCTGCTGGCCGTGCTGTACTGCCTGCTGTGGAGCTTTCAG |
| | (AA1-477) | ACCAGCGCCGGACACTTCCCTAGAGCCTGCGTGAGCAGCAA |
| | nucleic acid | GAACCTGATGGAAAAAGAGTGCTGCCCCCCTTGGAGCGGCG |
| | | ATAGAAGCCCCTGTGGCCAGCTGAGCGGCAGAGGCTCCTGC |
| | | CAGAACATCCTGCTGAGCAACGCCCCTCTGGGCCCCCAGTTC |
| | | CCCTTTACCGGCGTGGACGACAGAGAAAGCTGGCCCAGCGT |
| | | GTTCTACAACCGGACCTGCCAGTGCAGCGGCAACTTCATGGG |
| | | CTTCAACTGCGGCAACTGCAAGTTCGGCTTCTGGGGACCCAA |
| | | CTGCACCGAGAGAAGGCTGCTGGTGCGGAGAAACATCTTCG |
| | | ACCTGAGCGCCCCTGAGAAGGACAAGTTCTTCGCCTACCTGA |
| | | CCCTGGCCAAGCACACCATCAGCAGCGACTACGTGATCCCC |
| | | ATCGGCACCTACGGCCAGATGAAGAACGGCAGCACCCCCAT |
| | | GTTCAACGACATCAACATCTACGATCTGTTCGTGTGGATGCA |
| | | CTACTACGTGTCCATGGACGCCCTGCTGGGCGGCAGCGAGAT |
| | | CTGGAGAGACATCGACTTTGCCCACGAGGCCCCTGCCTTTCT |
| | | GCCCTGGCACCGGCTGTTTCTGCTGAGATGGGAGCAGGAAA |
| | | TCCAGAAGCTGACCGGCGACGAGAACTTCACCATCCCCTACT |
| | | GGGACTGGCGGGACGCCGAGAAGTGCGACATCTGCACCGAC |
| | | GAGTACATGGGCGGCCAGCACCCCACCAACCCCAATCTGCT |
| | | GAGCCCCGCCAGCTTCTTCAGCAGCTGGCAGATCGTGTGCTC |
| | | CCGGCTGGAGGAGTACAACAGCCACCAGAGCCTGTGCAATG |
| | | GCACCCCCGAGGGCCCTCTGAGAAGAAACCCCGGCAACCAC |
| | | GACAAGAGCCGGACCCCCAGACTGCCTAGCAGCGCCGACGT |
| | | GGAGTTCTGCCTGAGCCTGACCCAGTACGAGAGCGGCAGCA |
| | | TGGACAAGGCCGCCAACTTCAGCTTCCGGAACACCCTGGAA |
| | | GGCTTCGCCAGCCCTCTGACCGGCATTGCCGACGCCAGCCAG |
| | | AGCAGCATGCACAACGCCCTGCACATCTACATGAATGGAAC |
| | | CATGAGCCAGGTGCAGGGCAGCGCCAACGACCCCATCTTCC |
| | | TGCTGCACCACGCCTTCGTGGACAGCATCTTCGAGCAGTGGC |
| | | TGCGGAGACACAGACCCCTGCAGGAAGTGTACCCCGAGGCC |
| | | AACGCCCCTATCGGCCACAACCGGGAGAGCTACATGGTGCC |
| | | CTTCATCCCCCTGTACCGGAACGGCGACTTCTTCATCAGCTC |
| | | CAAGGACCTGGGCTACGACTACAGCTACCTGCAGGACAGCG |
| | | ACCCCGACAGCTTCCAGGACTACATCAAGAGCTACCTGGAA |
| | | CAGGCCAGCAGAATCTGGTCCTGG |
| 6 | Tryosinase | MLLAVLYCLLWSFQTSAGHFPRACVSSKNLMEKECCPPWSGD |
| | (AA1-477) | RSPCGQLSGRGSCQNILLSNAPLGPQFPFTGVDDRESWPSVFYN |
| | amino acid | RTCQCSGNFMGFNCGNCKFGFWGPNCTERRLLVRRNIFDLSAP |
| | | EKDKFFAYLTLAKHTISSDYVIPIGTYGQMKNGSTPMENDINIY |
| | | DLFVWMHYYVSMDALLGGSEIWRDIDFAHEAPAFLPWHRLFL |
| | | LRWEQEIQKLTGDENFTIPYWDWRDAEKCDICTDEYMGGQHP |
| | | TNPNLLSPASFFSSWQIVCSRLEEYNSHQSLCNGTPEGPLRRNPG |
| | | NHDKSRTPRLPSSADVEFCLSLTQYESGSMDKAANFSFRNTLEG |
| | | FASPLTGTADASQSSMHNALHIYMNGTMSQVQGSANDPIFLLH |
| | | HAFVDSIFEQWLRRHRPLQEVYPEANAPIGHNRESYMVPFIPLY |
| | | RNGDFFISSKDLGYDYSYLQDSDPDSFQDYIKSYLEQASRIWSW |
| 7 | TPTE fragment | ATGAACGAGAGCCCCGACCCTACAGATCTGGCCGGCGTGAT |
| | nucleic acid | CATCGAGCTGGGACCCAACGATAGCCCTCAGACCAGCGAGT |
| | | TCAAGGGGGCCACAGAGGAAGCCCCTGCCAAAGAGAGCCCC |
| | | CACACCTCCGAGTTTAAGGGCGCTGCTCGGGTGTCCCCTATC |
| | | AGCGAGAGCGTGCTGGCCCGGCTGAGCAAGTTCGAGGTGGA |
| | | GGACGCCGAGAACGTGGCCAGCTACGACAGCAAGATCAAGA |
| | | AAATCGTGCACAGCATCGTGTCCAGCTTCGCCTTCGGCCTGT |
| | | TCGGCGTGTTCCTGGTGCTGCTGGACGTGACACTGATCCTGG |
| | | CCGACCTGATCTTCACCGACAGCAAGCTGTACATCCCCCTGG |
| | | AATACCGGTCCATCAGCCTGGCCATTGCCCTGTTCTTTCTGAT |
| | | GGACGTGCTGCTGCGGGTGTTCGTGGAGCGGCGGCAGCAGT |
| | | ACTTCAGCGACCTGTTCAACATCCTGGACACCGCCATCATCG |
| | | TGATTCTGCTGCTGGTGGATGTGGTGTACATCTTCTTCGACAT |
| | | CAAGCTGCTGAGAAACATCCCCCGGTGGACCCATCTGCTGCG |
| | | GCTGCTGAGACTGATCATCCTGCTGCGGATCTTCCACCTGTT |
| | | CCACCAGAAGCGGCAGCTGGAAAAGCTGATCAGACGGCGGG |
| | | TGTCCGAGAACAAGCGGCGGTACACCAGGGACGGCTTCGAC |
| | | CTGGACCTGACCTACGTGACCGAGCGGATCATTGCCATGAGC |
| | | TTCCCCAGCAGCGGCAGACAGAGCTTCTACCGGAACCCCATC |
| | | AAAGAAGTGGTGCGGTTCCTGGACAAGAAGCACCGGAACCA |
| | | CTACCGGGTGTACAACCTGTGCAGCGAGCGGGCCTACGACC |
| | | CCAAGCACTTCCACAACCGGGTGGTGCGGATCATGATCGAC |
| | | GACCACAACGTGCCCACCCTGCACCAGATGGTGGTGTTCACC |
| | | AAAGAAGTGAACGAGTGGATGGCCCAGGACCTGGAAAACAT |
| | | CGTGGCCATCCACTGCAAGGGCGGCACCGACAGAACCGGCA |
| | | CCATGGTGTGCGCCTTTCTGATCGCCAGCGAGATCTGTAGCA |
| | | CCGCCAAAGAGTCCCTGTACTACTTCGGCGAGCGGAGAACC |
| | | GACAAGACCCACAGCGAGAAGTTCCAGGGCGTGGAGACACC |
| | | CAGCCAGAAAAGATATGTGGCTTACTTCGCCCAGGTGAAGC |
| | | ACCTGTACAACTGGAACCTGCCCCCCAGACGGATTCTGTTCA |
| | | TCAAGCACTTCATCATCTACAGCATCCCCAGATACGTGCGGG |
| | | ACCTGAAGATCCAGATCGAGATGGAAAAGAAAGTGGTGTTC |
| | | AGCACCATCTCCCTGGGCAAGTGCAGCGTGCTGGACAACAT |
| | | CACCACCGACAAGATCCTGATCGACGTGTTCGACGGCCTGCC |
| | | CCTGTACGACGACGTGAAGGTGCAGTTCTTCTACAGCAACCT |
| | | GCCCACCTACTACGACAATTGCAGCTTCTACTTCTGGCTGCA |
| | | CACCAGCTTCATCGAGAACAACAGGCTGTACCTGCCCAAGA |
| | | ACGAGCTGGACAACCTGCACAAGCAGAAGGCCAGAAGAATC |
| | | TACCCCAGCGACTTCGCCGTGGAGATCCTGTTTGGCGAGAAG |
| | | ATGACCAGCAGCGACGTGGTGGCCGGCAGCGAC |
| 8 | TPTE fragment | MNESPDPTDLAGVIIELGPNDSPQTSEFKGATEEAPAKESPHTSE |
| | amino acid | FKGAARVSPISESVLARLSKFEVEDAENVASYDSKIKKIVHSIVS |
| | | SFAFGLFGVFLVLLDVTLILADLIFTDSKLYIPLEYRSISLAIALFF |
| | | LMDVLLRVFVERRQQYFSDLFNILDTAIIVILLLVDVVYIFFDIKL |
| | | LRNIPRWTHLLRLLRLIILLRIFHLFHQKRQLEKLIRRRVSENKRR |
| | | YTRDGFDLDLTYVTERIIAMSFPSSGRQSFYRNPIKEVVRFLDKK |
| | | HRNHYRVYNLCSERAYDPKHFHNRVVRIMIDDHNVPTLHQMV |
| | | VFTKEVNEWMAQDLENIVAIHCKGGTDRTGTMVCAFLIASEIC |
| | | STAKESLYYFGERRTDKTHSEKFQGVETPSQKRYVAYFAQVKH |
| | | LYNWNLPPRRILFIKHFIIYSIPRYVRDLKIQIEMEKKVVFSTISLG |
| | | KCSVLDNITTDKILIDVFDGLPLYDDVKVQFFYSNLPTYYDNCS |
| | | FYFWLHTSFIENNRLYLPKNELDNLHKQKARRIYPSDFAVEILF |
| | | GEKMTSSDVVAGSD |

T-Cell Epitope: In some embodiments, the present disclosure, among other things, provides a pharmaceutical composition including one or more RNA molecules that collectively encode (i) a NY-ESO−) antigen, (ii) a MAGE-A3 antigen, (iii) a tyrosinase antigen, (iv) a TPTE antigen, or (v) a combination thereof; and a T cell epitope.

As used herein, the term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, binding peptides are typically about 8 to about 10 amino acids long although longer or shorter peptides may be effective. In the case of class II MHC/peptide complexes, binding peptides are typically about 10 to about 25 amino acids long and are in particular about 13 to about 18 amino acids long, whereas longer and shorter peptides may be effective.

In some embodiments, an RNA molecule of the one or more RNA molecules encodes a CD4 epitope, or an immunogenic fragment thereof. In some embodiments, an CD4 epitope comprises an amino acid sequence that is at least 80%, 85%, 90%, 95%, 96%, 97%, 98%, or 99% identical to the amino acid sequence of the CD4 epitope depicted as a “P2P16” domain in SEQ ID NOs: 11, 12, 15, 16, 19, 20, 23, or 24.

In some embodiments, a CD4 epitope comprises a tetanus toxoid P2, tetanus toxid P16, or both. In some embodiments, a tetanus toxoid P2 comprises or consists of an amino acid sequence that is at least 80%, 85%, 90%, 95%, 96%, 97%, 98%, or 99% identical to the amino acid sequence of the CD4 epitope depicted as a “P2” domain in SEQ ID NOs: 11, 12, 15, 16, 19, 20, 23, or 24. In some embodiments, a tetanus toxoid P16 comprises or consists of an amino acid sequence that is at least 80%, 85%, 90%, 95%, 96%, 97%, 98%, or 99% identical to the amino acid sequence of the CD4 epitope depicted as a “P16” domain in SEQ ID NOs: 11, 12, 15, 16, 19, 20, 23, or 24.

### II. Exemplary Embodiments of RNAs Encoding Provided Tumor-Associated Antigens

In some embodiments, the present disclosure, among other things, provides a pharmaceutical composition including one or more RNA molecules that collectively encode (i) a NY-ESO-1 antigen, (ii) a MAGE-A3 antigen, (iii) a tyrosinase antigen, (iv) a TPTE antigen, or (v) a combination thereof. In some embodiments, a single RNA molecule can encode at least two of a NY-ESO-1 antigen, a MAGE-A3 antigen, a tyrosinase antigen, and a TPTE antigen. In some embodiments, a single RNA molecule can encode at least three of a NY-ESO-1 antigen, a MAGE-A3 antigen, a tyrosinase antigen, and a TPTE antigen. In some embodiments, a single RNA molecule can encode each of the NY-ESO-1 antigen, the MAGE-A3 antigen, the tyrosinase antigen, and the TPTE antigen.

In some embodiments, a single RNA molecule can encode a polyepitopic polypeptide. For example, in some embodiments, a single RNA molecule encodes a polyepitopic polypeptide that includes at least two of a NY-ESO-1 antigen, a MAGE-A3 antigen, a tyrosinase antigen, and a TPTE antigen. In another example, in some embodiments, a single RNA molecule encodes a polyepitopic polypeptide that includes at least three of a NY-ESO-1 antigen, a MAGE-A3 antigen, a tyrosinase antigen, and a TPTE antigen. In another example, in some embodiments, a single RNA molecule encodes a polyepitopic polypeptide that includes each of a NY-ESO-1 antigen, a MAGE-A3 antigen, a tyrosinase antigen, and a TPTE antigen.

CD4+ epitope: In some embodiments, the present disclosure, among other things, provides a pharmaceutical composition including one or more RNA molecules that collectively encode (i) a NY-ESO-1 antigen, (ii) a MAGE-A3 antigen, (iii) a tyrosinase antigen, (iv) a TPTE antigen, or (v) a combination thereof; and a CD4⁺ epitope. In some embodiments, a CD4+ epitope is delivered by the same RNA molecule(s) that collectively encode the tumor associated antigens described herein. In some embodiments, a CD4+ epitope is delivered by a separate RNA molecule. In some embodiments, a CD4+ epitope is or comprises a non-specific antigen (e.g., an antigen that is not associated with melanoma). In some embodiments, a CD4+ epitope is or comprises a non-specific antigen that provides an adjuvant effect. For example, in some embodiments, a CD4+ epitope can include, without limitation, a tetanus toxid antigenic polypeptide, for example in some embodiments, a tetanus toxid P2 polypeptide and/or a tetanus toxoid P16 polypeptide.

MHC trafficking domain: In some embodiments, an RNA molecule described herein comprises a sequence encoding an MHC trafficking domain. In some embodiments, an MHC trafficking domain is or comprises a transmembrane region and a cytoplasmic region of a chain of an MHC molecule (e.g., a MHC Class I molecule), for example, in some embodiments as described in the International Patent Publication Number WO 2005/038030, the contents of which are incorporated herein by reference in their entireties for the purposes described herein. In some embodiments, an MHC trafficking domain is or comprises a MHC Class I trafficking domain. In some embodiments, an MHC class I trafficking domain comprises an amino acid sequence that is at least 80%, 85%, 90%, 95%, 96%, 97%, 98%, or 99% identical to the amino acid sequence of the MHC Class I trafficking domain depicted as a “MITD” domain in SEQ ID NOs: 11, 12, 15, 16, 19, 20, 23, or 24. In some embodiments, an MHC class I trafficking domain comprises an amino acid sequence that is identical to the amino acid sequence of the MHC Class I trafficking domain as depicted as a “MITD” domain in SEQ ID NOs: 11, 12, 15, 16, 19, 20, 23, or 24.

Signal peptide-encoding region: In some embodiments, an RNA molecule described herein comprises a sequence encoding a signal peptide. In some embodiments, inclusion of such a signal peptide is useful for increased processing and presentation of antigens. In some embodiments, a signal peptide is or comprises a secretion signal peptide. In some embodiments, a secretion signal peptide may correspond to a sequence encoding a human MHC class I complex alpha chain or a fragment thereof. In some embodiments, a secretion signal peptide may corresponds to a 70-80 bp fragment coding for a secretory signal peptide, which in some embodiments can guide translocation of a nascent polypeptide chain into an endoplasmic reticulum. In some embodiments, a signal peptide comprises an amino acid sequence that is at least 80%, 85%, 90%, 95%, 96%, 97%, 98%, or 99% identical to the amino acid sequence of the signal peptide-encoding region depicted as “Sec” in SEQ ID NOs: 11, 12, 15, 16, 19, 20, 23, or 24. In some embodiments, a signal peptide comprises an amino acid sequence that is identical to the amino acid sequence of the signal peptide depicted as “Sec” in SEQ ID NOs: 11, 12, 15, 16, 19, 20, 23, or 24. In some embodiments, a signal peptide is linked to the N-terminus of an antigen included in an RNA molecule.

In some embodiments, an RNA molecule described herein comprises at least one non-coding sequence element. In some embodiments, such a non-coding sequence element is included in an RNA molecule to enhance RNA stability and/or translation efficiency. Examples of non-coding sequence elements include but are not limited to a 3′ untranslated region (UTR), a 5′ UTR, a cap structure, a poly adenine (polyA) tail, and any combinations thereof.

UTRs (5′ UTRs and/or 3′UTRs): In some embodiments, a provided RNA molecule comprises a nucleotide sequence that encodes a 5′UTR of interest and/or a 3′ UTR of interest. One of skill in the art will appreciate that untranslated regions (e.g., 3′ UTR and/or 5′ UTR) of an mRNA sequence can contribute to mRNA stability, mRNA localization, and/or translational efficiency.

In some embodiments, a provided RNA molecule can comprise a 5′ UTR nucleotide sequence and/or a 3′ UTR nucleotide sequence. In some embodiments, such a 5′ UTR sequence can be operably linked to a 3′ of a coding sequence (e.g., encompassing one or more coding regions). Additionally or alternatively, in some embodiments, a 3′ UTR sequence can be operably linked to 5′ of a coding sequence (e.g., encompassing one or more coding regions).

In some embodiments, 5′ and 3′ UTR sequences included in an RNA molecule described herein can consist of or comprise naturally occurring or endogenous 5′ and 3′ UTR sequences for an open reading frame of a gene of interest. Alternatively, in some embodiments, 5′ and/or 3′ UTR sequences included in an RNA molecule are not endogenous to a coding sequence (e.g., encompassing one or more coding regions); in some such embodiments, such 5′ and/or 3′ UTR sequences can be useful for modifying the stability and/or translation efficiency of an RNA sequence transcribed. For example, a skilled artisan will appreciate that AU-rich elements in 3′ UTR sequences can decrease the stability of mRNA. Therefore, as will be understood by a skilled artisan, 3′ and/or 5′ UTRs can be selected or designed to increase the stability of the transcribed RNA based on properties of UTRs that are well known in the art.

For example, one skilled in the art will appreciate that, in some embodiments, a nucleotide sequence consisting of or comprising a Kozak sequence of an open reading frame sequence of a gene or nucleotide sequence of interest can be selected and used as a nucleotide sequence encoding a 5′ UTR. As will be understood by a skilled artisan, Kozak sequences are known to increase the efficiency of translation of some RNA transcripts, but are not necessarily required for all RNAs to enable efficient translation. In some embodiments, a provided RNA molecule can comprise a nucleotide sequence that encodes a 5′ UTR derived from an RNA virus whose RNA genome is stable in cells. In some embodiments, various modified ribonucleotides (e.g., as described herein) can be used in the 3′ and/or 5′ UTRs, for example, to impede exonuclease degradation of the transcribed RNA sequence.

In some embodiments, a 5′ UTR included in an RNA molecule described herein may be derived from human α-globin mRNA combined with Kozak region.

In some embodiments, an RNA molecule may comprise one or more 3′UTRs. For example, in some embodiments, an RNA molecule may comprise two copies of 3′-UTRs derived from a globin mRNA, such as, e.g., alpha2-globin, alpha1-globin, beta-globin (e.g., a human beta-globin) mRNA. In some embodiments, two copies of 3′UTR derived from a human beta-globin mRNA may be used, e.g., in some embodiments which may be placed between a coding sequence of an RNA molecule and a poly(A)-tail, to improve protein expression levels and/or prolonged persistence of an mRNA. In some embodiments, a 3′UTR derived from a human beta-globin as described in WO 2007/036366, the contents of which are incorporated herein by reference in their entireties for the purposes described herein, may be included in an RNA molecule described herein.

In some embodiments, a 3′ UTR included in an RNA molecule may be or comprise one or more (e.g., 1, 2, 3, or more) of the 3′UTR sequences disclosed in WO 2017/060314, the entire content of which is incorporated herein by reference for the purposes described herein. In some embodiments, a 3′-UTR may be a combination of at least two sequence elements (FI element) derived from the “amino terminal enhancer of split” (AES) mRNA (called F) and the mitochondrial encoded 12S ribosomal RNA (called I). These were identified by an ex vivo selection process for sequences that confer RNA stability and augment total protein expression (see WO 2017/060314, herein incorporated by reference).

PolyA tail: In some embodiments, a provided ssRNA can comprise a nucleotide sequence that encodes a polyA tail. A polyA tail is a nucleotide sequence comprising a series of adenosine nucleotides, which can vary in length (e.g., at least 5 adenine nucleotides) and can be up to several hundred adenosine nucleotides. In some embodiments, a polyA tail is a nucleotide sequence comprising at least 30 adenosine nucleotides or more, including, e.g., at least 35, at least 40, at least 45, at least 50, at least 55, at least 60, at least 65, at least 70, at least 75, at least 80, at least 85, at least 90, at least 95, at least 100, or more adenosine nucleotides. In some embodiments, a polyA tail is a nucleotide sequence comprising at least 120 adenosine nucleotides. In some embodiments, a polyA tail as described in WO 2007/036366, the contents of which are incorporated herein by reference in their entireties for the purposes described herein, may be included in an RNA molecule described herein.

In some embodiments, a polyA tail is or comprises a polyA homopolymeric tail. In some embodiments, a polyA tail may comprise one or more modified adenosine nucleosides, including, but not limited to, cordiocipin and 8-azaadenosine.

In some embodiments, a polyA tail may comprise one or more non-adenosine nucleotides. In some embodiments, a polyA tail may be or comprise a disrupted or modified polyA tail as described in WO 2016/005324, the entire content of which is incorporated herein by reference for the purpose described herein. For example, in some embodiments, a polyA tail included in an RNA molecule described herein may be or comprise a modified polyA sequence comprising: a linker sequence; a first sequence of at least 20 A consecutive nucleotides, which is 5′ of the linker sequence; and a second sequence of at least 20 A consecutive nucleotides, which is 3′ of the linker sequence. In some embodiments, a modified polyA sequence may comprise: a linker sequence comprising at least ten non-A nucleotides (e.g., T, G, and/or C nucleotides); a first sequence of at least 30 A consecutive nucleotides, which is 5′ of the linker sequence; and a second sequence of at least 70 A consecutive nucleotides, which is 3′ of the linker sequence.

5′ cap: In some embodiments, an RNA molecule described herein may comprise a 5′ cap, which may be incorporated into such an RNA molecule during transcription, or joined to such an RNA molecule post-transcription. In some embodiments, an RNA molecule may comprise an anti-reverse cap analog (ARCA). In some embodiments, an RNA molecule may comprise a cap analog beta-S-ARCA(D1) (m₂^{7,2′-O}GppₛpG) as illustrated below:

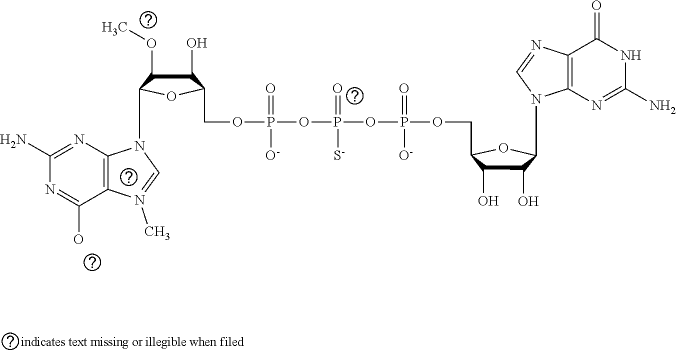

In some embodiments, an RNA molecule may comprise an S-ARCA cap structure as disclosed in WO2011/015347 or in WO2008/157688, the entire contents of each of which are incorporated herein by reference for the purposes described herein.

In some embodiments, an RNA molecule may comprise a 5′ cap structure for co-transcriptional capping of mRNA. Examples of a cap structure for co-transcriptional capping are known in the art, including, e.g., as described in WO 2017/053297, the entire content of which is incorporated herein by reference for the purposes described herein. In some embodiments, a 5′ cap included in an RNA molecule described herein is or comprises m7G(5′)ppp(5′)(2′OMeA)pG. In some embodiments, a 5′ cap included in an RNA molecule described herein is or comprises a Cap1 structure [e.g., but not limited to m₂^{7,3′-O}Gppp(m₁^{2′-O})ApG].

In some embodiments, one or more RNA molecules that collectively encodes a NY-ESO-1 antigen, a MAGE-A3 antigen, a tyrosinase antigen, a TPTE antigen, or a combination thereof comprise natural ribonucleotides. In some embodiments, one or more RNA molecules that collectively encodes a NY-ESO-1 antigen, a MAGE-A3 antigen, a tyrosinase antigen, a TPTE antigen, or a combination thereof comprise at least one modified or synthetic ribonucleotide. In some embodiments, modified or synthetic ribonucleotides are included in an RNA molecule to increase its stability and/or to decrease its cytotoxicity. For example, in some embodiments, at least one of A, U, C, and G ribonucleotide of an RNA molecule described herein may be replaced by a modified ribonucleotide. For example, in some embodiments, some or all of cytidine residues present in an RNA molecule may be replaced by a modified cytidine, which in some embodiments may be, e.g., 5-methylcytidine. Alternatively or additionally, in some embodiments, some or all of uridine residues present in an RNA molecule may be replaced by a modified uridine, which in some embodiments may be, e.g., pseudouridine, such as, e.g., 1-methylpseudouridine. In some embodiments, all uridine residues present in an RNA molecule is replaced by pseudouridine, e.g., 1-methylpseudouridine.

In some embodiments, the present disclosure, among other things, provides a pharmaceutical composition including one or more RNA molecules where an RNA molecule comprises from 5′ to 3′: (i) a 5′ cap or 5′ cap analogue; (ii) at least one 5′ UTR; (iii) a signal peptide; (iv) a coding region that encodes at least one of a NY-ESO-1 antigen, a MAGE-A3 antigen, a tyrosinase antigen, and a TPTE antigen; (v) at least one sequence that encodes a CD4′ epitope; (vi) a sequence encoding an MHC trafficking domain; (vii) at least one 3′UTR; and (viii) a poly-adenine tail. For example, in some embodiments, a cap structure that is included in an RNA molecule described herein can be a cap structure that can increase the resistance of RNA molecules to degradation by extracellular and intracellular RNases and leads to higher protein expression. In some embodiments, an exemplary cap structure is or comprises beta-S-ARCA(D1) (m₂^{7,2′-O}GppₛpG). In some embodiments, an exemplary 5′ UTR sequence element that is included in an RNA molecule described herein is or comprises a characteristic sequence from human α-globin and a Kozak consensus sequence. In some embodiments, an exemplary 3′ UTR sequence element that is included in an RNA molecule described herein may be or comprise two copies of 3′UTR derived from a human beta-globin, or a combination of two sequence elements (FI element) derived from the “amino terminal enhancer of split” (AES) mRNA (called F) and a mitochondrial encoded 12S ribosomal RNA (called I). See, e.g., WO2007/036366 and WO 2017/060314, the entire contents of each of which is incorporated herein by reference for the purposes described herein. In some embodiments, a poly(A)-tail that is included in an RNA molecule described herein can be designed to enhance RNA stability and/or translational efficiency. In some embodiments, an exemplary poly(A)-tail is or comprises a contiguous poly(A) sequence of at least 120 adenosine nucleotides in length. In some embodiments, an exemplary poly(A)-tail is or comprises a modified poly(A) sequence of 110 nucleotides in length including a stretch of 30 adenosine residues, followed by a 10 nucleotide linker sequence and another stretch of 70 adenosine residues (A30L70).

Linker: In some embodiments, at least one sequence that encodes a linker can be present in an RNA molecule to separate individual components present in the RNA molecule. For example, in some embodiments, at least one sequence that encodes a linker can be present between a coding region that encodes one or more tumor associated antigens as described herein and a sequence that encodes a CD4+ epitope. In some embodiments, at least one sequence that encodes a linker can be present between a sequence that encodes a CD4+ epitope and a sequence that encodes an MHC trafficking domain. In some embodiments, a sequence that encodes a linker may encode a peptide linker. In some embodiments, a peptide linker may be enriched in glycine and/or serine. In some embodiments, a peptide linker that is enriched in glycine and/or serine can comprise at least one amino acid that is not glycine or serine. In some embodiments, a peptide linker can have a length of 3 to 20 amino acids or 3 to 15 amino acids, or 3 to 10 amino acids. In some embodiments, a peptide linker can have a length of 10 amino acids.

In some embodiments, one or more RNA molecules described herein is or comprises one or more mRNAs.

In some embodiments, a pharmaceutical composition comprises (i) an RNA molecule encoding a NY-ESO-1 antigen as disclosed in Table 2 below; an RNA molecule encoding a MAGE-A3 antigen as disclosed in Table 2 below; an RNA molecule encoding a Tyrosinase antigen as disclosed in Table 2 below; and an RNA molecule encoding a TPTE antigen as disclosed in Table 2 below. In some such embodiments, a pharmaceutical composition can be prepared by mixing RNA molecules each encoding a tumor associated antigen as described herein in a molar ratio of about 1:1:1:1. In other words, in some embodiments, if total RNA dose is 100 μg, then a pharmaceutical composition can be prepared to include 25 μg NY-ESO-1 antigen encoding RNA, 25 μg MAGE-A3 antigen encoding RNA, 25 μg tyrosinase antigen encoding RNA, 25 μg TPTE antigen encoding RNA. In some embodiments, this can be achieved by forming, e.g., NY-ESO-1 antigen lipid particles (e.g., NY-ESO-1 antigen lipoplexes or lipid nanoparticles), MAGE-A3 antigen lipid particles (e.g., MAGE-A3 antigen lipoplexes or lipid nanoparticles), tyrosinase antigen lipid particles (e.g., tyrosinase antigen lipoplexes or lipid nanoparticles), and TPTE antigen lipid particles (e.g., TPTE antigen lipoplexes or lipid nanoparticles). In this approach, the RNA-lipid particles can then be mixed. In other words, mixing can be after RNA and lipid particles form RNA-lipid particles (e.g., RNA-lipoplexes or RNA-lipid nanoparticles).

TABLE 2
Exemplary constructs of RNA molecules each encoding
a tumor associated antigen described herein

| | New York oesophageal squamous cell |
|---|---|
| Encoded protein | carcinoma (NY-ESO-1) antigen |
| Exemplary RNA | beta-S-ARCA(D1)-hAg-Kozak-sec-GS-NY-ESO- |
| construct 1 (e.g., | 1-GS-P2P16-GS-MITD-2hBg-A120 |
| RBL001.1) | |
| Exemplary RNA | beta-S-ARCA(D1)-hAg-Kozak-sec-GS-NY-ESO- |
| construct 2 (e.g., | 1-GS-P2P16-GS-MITD-FI-A30L70 |
| RBL001.3) | |
| Encoded protein | Melanoma-associated antigen A3 (MAGE-A3) |
| Exemplary RNA | beta-S-ARCA(D1)-hAg-Kozak-sec-GS-MAGEA3- |
| construct 1 (e.g., | GS-P2P16-GS-MITD-2hBg-A120 |
| RBL003.1) | |
| Exemplary RNA | beta-S-ARCA(D1)-hAg-Kozak-sec-GS-MAGEA3- |
| construct 2 (e.g., | GS-P2P16-GS-MITD-FI- A30L70 |
| RBL003,3) | |
| Encoded protein | Tyrosinase antigen |
| Exemplary RNA | beta-S-ARCA(D1)-hAg-Kozak-Tyrosinase(1-477)- |
| construct 1 (e.g., | GS-P2P16-GS-MITD-2hBg-A120 |
| RBL002.2) | |
| Exemplary RNA | beta-S-ARCA(D1)-hAg-Kozak-Tyrosinase(1-477)- |
| construct 2 (e.g., | GS-P2P16-GS-MITD-FI- A30L70 |
| RBL002.4) | |
| Encoded protein | Transmembrane phosphatase with tensin |
| | homology (TPTE) antigen |
| Exemplary RNA | beta-S-ARCA(D1)-hAg-Kozak-sec-GS-TPTE-GS- |
| construct 1 (e.g., | P2P16-GS-MITD-2hBg-A120 |
| RBL004.1) | |
| Exemplary RNA | beta-S-ARCA(D1)-hAg-Kozak-sec-GS-TPTE-GS- |
| construct 2 (e.g., | P2P16-GS-MITD-FI-A30L70 |
| RBL004.3) | |

GS=a glycine/serine linker; MITD=MHC class I trafficking domain; sec=secretory signal peptide; UTR=untranslated region; hAg=human alpha-globin; P2P16=tetanus toxoid-derived P2 and P16 helper epitopes; 2hBg=2 copies of human beta-globin; A120=polyA tail of 120 As in length; A30L70=two contiguous segments of adenine nucleotides (one segment having a length of 30 As in length while another segment having a length of 70 As in length) separated by linker; FI=a combination of at least two sequence elements derived from the “amino terminal enhancer of split” (AES) mRNA (called F) and the mitochondrial encoded 12S ribosomal RNA (called I)

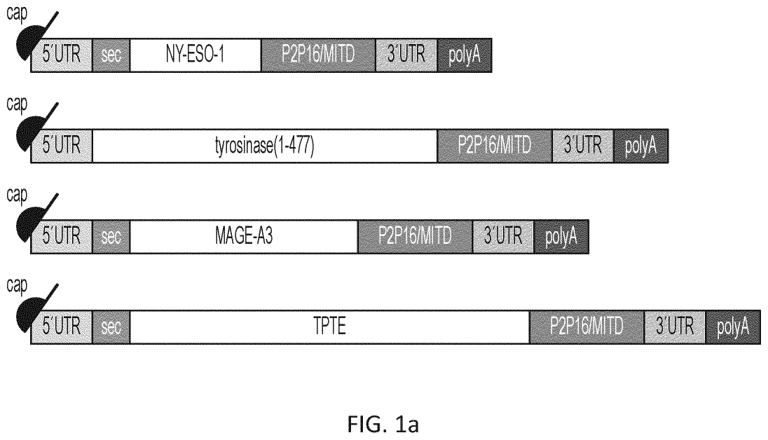

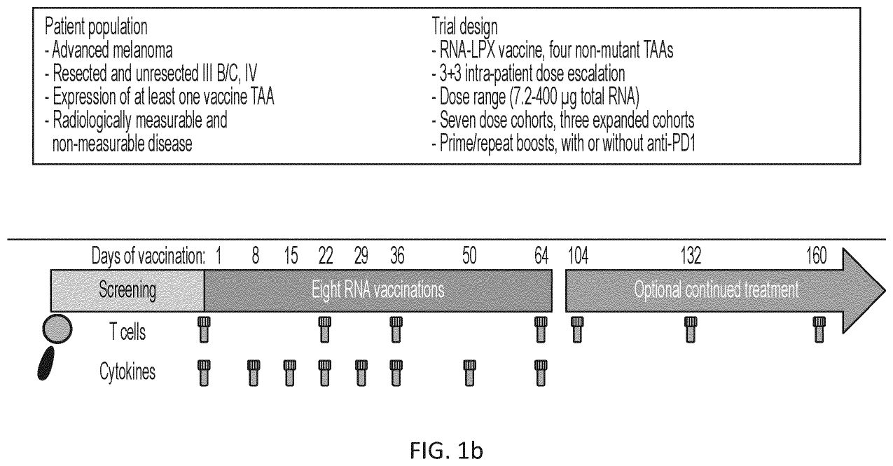

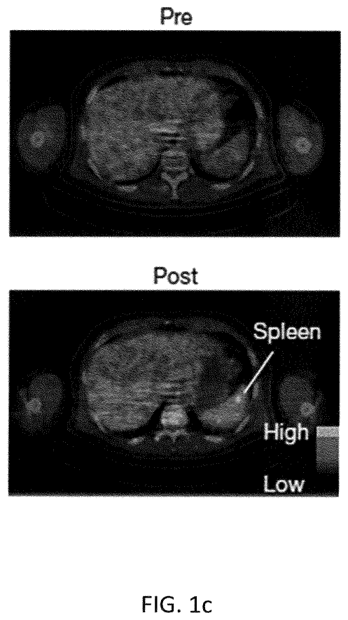

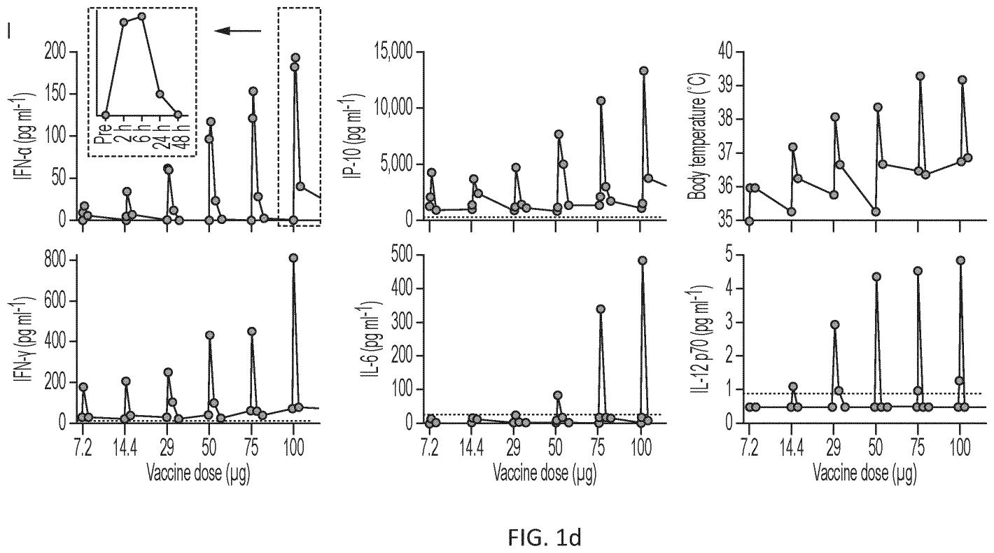

In some embodiments, an RNA molecule encoding a NY-ESO-1 antigen is or comprises the nucleotide sequence of RBL001.1 or RBL001.3. In some embodiments, an RNA molecule encoding a NY-ESO-1 antigen comprises a sequence that encodes a polypeptide having the amino acid sequence of RBL001.1 or RBL001.3. In the following, the sequence alignment of RBL001.1 and RBL003.1 is given for both the nucleotide sequences of the full-length RNAs as well as for the translated proteins (with the amino acid positioned below the third nucleotide of the respective codon triplet). Sequence elements as illustrated in FIG. 1a are displayed above the nucleotide sequences. Differences in the nucleotide and amino acid sequences are indicated by “*”. SEQ ID NO: 9 for RBL001.1 RNA; SEQ ID NO: 10 for RBL001.3 RNA; SEQ ID NO: 11 for RBL001.1 protein; SEQ ID NO: 12 for RBL001.3 protein.

```
                                  [ 5′-UTR ][ sec
RBL001.1 (RNA) GGGCGAACTA GTATTCTTCT GGTCCCCACA GACTCAGAGA GAACCCGCCA CCATGAGAGT 60
RBL001.3 (RNA) GGGCGAACTA GTATTCTTCT GGTCCCCACA GACTCAGAGA GAACCCGCCA CCATGAGAGT 60
RBL001.1 (prot.) M R 2
RBL001.3 (prot.) M R 2
                                                                                                sec
RBL001.1 (RNA) GACCGCCCCC AGAACCCTGA TCCTGCTGCT GTCTGGCGCC CTGGCCCTGA CAGAGACATG 120
RBL001.3 (RNA) GATGGCCCCC AGAACCCTGA TCCTGCTGCT GTCTGGCGCC CTGGCCCTGA CAGAGACATG 120
                                      **
RBL001.1 (prot.) V T A P R T L I L L L S G A L A L T E T 22
RBL001.3 (prot.) V M A R T T L I L L L S G A L A L T E T 22
                                        *
                                      sec ][ GS ][ antigen
RBL001.1 (RNA) GGCCGGAAGC GGAGGATCT ATGCAGGCCG AGGGCAGAGG 159
RBL001.3 (RNA) GGCCGGAAGC GGCGGCTCTG GAGGAGGCGG CTCCGGAGGC ATGCAGGCCG AGGGCAGAGG 180
                                                            * * ********* **********
RBL001.1 (prot.) W A G S G G S M Q A E G R 35
RBL001.3 (prot.) W A G S G G S G G G G S G G M Q A E G R 42
                                                                                * * * * * * *
                                                                                              antigen
RBL001.1 (RNA) AACAGGCGGC AGCACAGGCG ACGCAGATGG ACCAGGCGGC CCTGGAATCC CTGATGGCCC 219
RBL001.3 (RNA) AACAGGCGGC AGCACAGGCG ACGCAGATGG ACCAGGCGGC CCTGGAATCC CTGATGGCCC 240
RBL001.1 (prot.) G T G G S T G D A D G P G G P G I P D G 55
RBL001.3 (prot.) G T G G S T G D A D G P G G P G I P D G 62
                                                                                              antigen
RBL001.1 (RNA) AGGCGGCAAT GCTGGGGGAC CAGGAGAAGC TGGCGCCACA GGCGGGAGAG GACCTAGAGG 279
RBL001.3 (RNA) AGGCGGCAAT GCTGGGGGAC CAGGAGAAGC TGGCGCCACA GGCGGGAGAG GACCTAGAGG 300
RBL001.1 (prot.) P G G N A G G P G E A G A T G G R G P R 75
RBL001.3 (prot.) P G G N A G G P G E A G A T G G R G P R 82
                                                                                              antigen
RBL001.1 (RNA) AGCTGGAGCC GCTAGAGCTT CTGGACCTGG GGGAGGCGCC CCTAGAGGAC CACATGGAGG 339
RBL001.3 (RNA) AGCTGGAGCC GCTAGAGCTT CTGGACCTGG GGGAGGCGCC CCTAGAGGAC CACATGGAGG 360
RBL001.1 (prot.) G A G A A R A S G P G G G A P R G P H G 95
RBL001.3 (prot.) G A G A A R A S G P G G G A P R G P H G 102
                                                                                              antigen
RBL001.1 (RNA) CGCTGCCAGC GGCCTGAATG GCTGCTGCAG ATGCGGCGCC AGAGGCCCTG AGAGCCGGCT 399
RBL001.3 (RNA) CGCTGCCAGC GGCCTGAATG GCTGCTGCAG ATGCGGCGCC AGAGGCCCTG AGAGCCGGCT 420
RBL001.1 (prot.) G A A S G L N G C C R C G A R G P E S R 115
RBL001.3 (prot.) G A A S G L N G C C R C G A R G P E S R 122
                                                                                              antigen
RBL001.1 (RNA) GCTGGAATTC TACCTGGCCA TGCCCTTCGC CACCCCCATG GAAGCCGAGC TGGCCAGAAG 459
RBL001.3 (RNA) GCTGGAATTC TACCTGGCCA TGCCCTTCGC CACCCCCATG GAAGCCGAGC TGGCCAGAAG 480
RBL001.1 (prot.) L L E F Y L A M P F A T P M E A E L A R 135
RBL001.3 (prot.) L L E F Y L A M P F A T P M E A E L A R 142
                                                                                              antigen
RBL001.1 (RNA) ATCCCTGGCT CAGGACGCTC CTCCTCTGCC TGTGCCCGGC GTGCTGCTGA AAGAATTCAC 519
RBL001.3 (RNA) ATCCCTGGCT CAGGACGCTC CTCCTCTGCC TGTGCCCGGC GTGCTGCTGA AAGAATTCAC 540
RBL001.1 (prot.) R S L A Q D A P P L P V P G V L L K E F 155
RBL001.3 (prot.) R S L A Q D A P P L P V P G V L L K E F 162
                                                                                              antigen
RBL001.1 (RNA) CGTGTCCGGC AACATCCTGA CCATCAGACT GACAGCCGCC GATCACAGAC AGCTCCAGCT 579
RBL001.3 (RNA) CGTGTCCGGC AACATCCTGA CCATCAGACT GACAGCCGCC GATCACAGAC AGCTCCAGCT 600
RBL001.1 (prot.) T V S G N I L T I R L T A A D H R Q L Q 175
RBL001.3 (prot.) T V S G N I L T I R L T A A D H R Q L Q 182
                                                                                              antigen
RBL001.1 (RNA) GAGCATCAGC TCTTGCCTGC AGCAGCTGAG CCTGCTGATG TGGATCACCC AGTGCTTTCT 639
RBL001.3 (RNA) GAGCATCAGC TCTTGCCTGC AGCAGCTGAG CCTGCTGATG TGGATCACCC AGTGCTTTCT 660
RBL001.1 (prot.) L S I S S C L Q Q L S L L M W I T Q C F 195
RBL001.3 (prot.) L S I S S C L Q Q L S L L M W I T Q C F 202
                                                                        antigen ][ GS
RBL001.1 (RNA) GCCCGTGTTC CTGGCCCAGC CACCCAGCGG ACAGAGAAGG GGCGGATCCC TGGGAGGCGG 699
RBL001.3 (RNA) GCCCGTGTTC CTGGCCCAGC CACCCAGCGG ACAGAGAAGG GGAGGATCCG GTGGTGGCGG 720
                                                                                                                              * * ** *
RBL001.1 (prot.) L P V F L A Q P P S G Q R R G G S L G G 215
RBL001.3 (prot.) L P V F L A Q P P S G Q R R G G S G G G 222
                                          GS ][ P2P16
RBL001.1 (RNA) GGGAAGCGGC AAGAAGCAGT ACATCAAGGC CAACAGCAAG TTCATCGGCA TCACCGAGCT 759
RBL001.3 (RNA) CAGCGGCGGC AAGAAGCAGT ACATCAAGGC CAACAGCAAG TTCATCGGCA TCACCGAGCT 780
                                  ** **
RBL001.1 (prot.) G G S G K K Q Y I K A N S K F I G I T E 235
RBL001.3 (prot.) G G S G K K Q Y I K A N S K F I G I T E 242
                                        * *
                                                                                              P2P16
RBL001.1 (RNA) GAAGAAGCTG GGAGGGGGCA AACGGGGAGG CGGCAAAAAG ATGACCAACA GCGTGGACGA 819
RBL001.3 (RNA) GAAGAAGCTG GGAGGGGGCA AACGGGGAGG CGGCAAAAAG ATGACCAACA GCGTGGACGA 840
RBL001.1 (prot.) L K K L G G G K R G G G K K M T N S V D 255
RBL001.3 (prot.) L K K L G G G K R G G G K K M T N S V D 262
                                                                                              P2P16
RBL001.1 (RNA) CGCCCTGATC AACAGCACCA AGATCTACAG CTACTTCCCC AGCGTGATCA GCAAAGTGAA 879
RBL001.3 (RNA) CGCCCTGATC AACAGCACCA AGATCTACAG CTACTTCCCC AGCGTGATCA GCAAAGTGAA 900
RBL001.1 (prot.) D A L I N S T K I Y S Y F P S V I S K V 275
RBL001.3 (prot.) D A L I N S T K I Y S Y F P S V I S K V 282
                                                  P2P16 ][ GS
RBL001.1 (RNA) CCAGGGCGCT CAGGGCAAGA AACTGGGCTC TAGCGGAGGG GGAGGCTCTC CTGGGGGGGG 939
RBL001.3 (RNA) CCAGGGCGCT CAGGGCAAGA AACTGGGCTC TAGCGGAGGG GGAGGCTCTC CTGGGGGGGG 960
RBL001.1 (prot.) N Q G A Q G K K L G S S G G G G S P G G 295
RBL001.3 (prot.) N Q G A Q G K K L G S S G G G G S P G G 302
                                    GS ][ MITD
RBL001.1 (RNA) ATCTAGCATC GTGGGAATTG TGGCAGGACT GGCAGTGCTG GCCGTGGTGG TGATCGGAGC 999
RBL001.3 (RNA) ATCTAGCATC GTGGGAATTG TGGCAGGACT GGCAGTGCTG GCCGTGGTGG TGATCGGAGC 1020
RBL001.1 (prot.) G S S I V G I V A G L A V L A V V V I G 315
RBL001.3 (prot.) G S S I V G I V A G L A V L A V V V I G 322
                                                                                                MITD
RBL001.1 (RNA) CGTGGTGGCT ACCGTGATGT GCAGACGGAA GTCCAGCGGA GGCAAGGGCG GCAGCTACAG 1059
RBL001.3 (RNA) CGTGGTGGCT ACCGTGATGT GCAGACGGAA GTCCAGCGGA GGCAAGGGCG GCAGCTACAG 1080
RBL001.1 (prot.) A V V A T V M C R R K S S G G K G G S Y 335
RBL001.3 (prot.) A V V A T V M C R R K S S G G K G G S Y 342
                                                                                        MITD ][3′-UTR
RBL001.1 (RNA) CCAGGCCGCC AGCTCTGATA GCGCCCAGGG CAGCGACGTG TCACTGACAG CC TGACT 1119
RBL001.3 (RNA) CCAGGCCGCC AGCTCTGATA GCGCCCAGGG CAGCGACGTG TCACTGACAG CCTAGTAACT 1140
                                                                                                                                                *** *
RBL001.1 (prot.) S Q A A S S D S A Q G S D V S L T A 353
RBL001.3 (prot.) S Q A A S S D S A Q G S D V S L T A 360
                                                                                              3′-UTR
RBL001.1 (RNA) CGAGAGCTCG CTTTCTTGCT GTCCAATTTC TATTAAA GGTTCCTTT GTTCCCTAAG 1172
RBL001.3 (RNA) CGAGCTGGTA CTGCATGCAC GCAATGCTAG CTGCCCCTTT CCCGTCCTGG GTACCCCGAG 1200
                                          ****** *** **** ****** ** ********** **** ** * **
                                                                                              3′-UTR
RBL001.1 (RNA) TCCAACTACT AAACTGGGGG ATATTATGAA GGGCCTTGAG CATCTGGATT CTGCCTAATA 1232
RBL001.3 (RNA) TCTCCCCCGA CCTCGGGTCC CAGGTATGCT CCCACCTCCA CCTGCCCCAC TCACCACCTC 1260
                                      *** **** *** * *** **** ** **** * *** * ******* *** *** *
                                                                                              3′-UTR
RBL001.1 (RNA) AAAAACATTT ATTTTCATTG CTGCGTCGAG AGCTCGCTTT CTTGCTGTCC AATTTCT 1289
RBL001.3 (RNA) TGCTAGTTCC AGACACCTCC CAAGCACGCA GCAATGCAGC TCAAAACGCT TAGCCTAGCC 1320
                                  **** ** ** **** * ** ***** ** ***** *** ******** * * ********
                                                                                              3′-UTR
RBL001.1 (RNA) A TTAAAGGTTC CTTT GTTCCCTAA GTCCAACTAC TAAACTGGGG 1333
RBL001.3 (RNA) ACACCCCCAC GGGAAACAGC AGTGATTAAC CTTTAGCAAT AAACGAAAGT TTAACTAAGC 1380
                                  ********** *** ***** ** ******* ** ** * * *** * **** * ** *
                                                                                              3′-UTR
RBL001.1 (RNA) GATATTATGA AGGGCCTTGA GCATCTGGAT TCTGCCTAAT AAAAAACATT TATTTTCATT 1393
RBL001.3 (RNA) TATACTAACC CCAGGGTTGG TCAATTTCGT GCCAGCCACA CCGAGACCTG GTCCAGAGTC 1440
                                  * * *** *** ** * * ** *** * *** * ** *** * * * ******** *
                                            3′-UTR ][ poly(A)
RBL001.1 (RNA) GCTGCGTCGA GAGCTAAAAA AAAAAAAAAA AAAAAAAAAA AAAAAAAAAA AAAAAAAAAA 1453
RBL001.3 (RNA) GCTAGCCGCG TCGCTAAAAA AAAAAAAAAA AAAAAAAAAA AAAAAGCATA TGACT 1500
                                        ******* ** ** * ** *******
RBL001.1 (RNA) AAAAAAAAAA AAAAAAAAAA AAAAAAAAAA AAAAAAAAAA AAAAAAAAAA AAAAAAAAAA 1513
RBL001.3 (RNA) AAAAA AAAAAAAAAA AAAAAAAAAA AAAAAAAAAA AAAAAAAAAA AAAAAAAAAA 1550
                                  *****
                                            poly(A) ]
RBL001.1 (RNA) AAAAAAAAAA AAAAA 1528
RBL001.3 (RNA) AAAAAAAAAA AAAAA 1565
```

In some embodiments, an RNA molecule encoding a Tyrosinase antigen is or comprises the nucleotide sequence of RBL002.2 or RBL002.4. In some embodiments, an RNA molecule encoding a Tyrosinase antigen comprises a sequence that encodes a polypeptide having the amino acid sequence of RBL002.2 or RBL002.4. In the following, the sequence alignment of RBL002.2 and RBL002.4 is given for both the nucleotide sequences of the full-length RNAs as well as for the translated proteins (with the amino acid positioned below the third nucleotide of the respective codon triplet). Sequence elements as illustrated in FIG. 1a are displayed above the nucleotide sequences. Differences in the nucleotide and amino acid sequences are indicated by “*”. SEQ ID NO: 13 for RBL002.2 RNA; SEQ ID NO: 14 for RBL002.4 RNA; SEQ ID NO: 15 for RBL002.2 protein; SEQ ID NO: 16 for RBL002.4 protein.

```
                                  [ 5′-UTR ][ antigen
RBL002.2 (RNA) GGGCGAACTA GTATTCTTCT GGTCCCCACA GACTCAGAGA GAACCCGCCA CCATGCTTCT 60
RBL002.4 (RNA) GGGCGAACTA GTATTCTTCT GGTCCCCACA GACTCAGAGA GAACCCGCCA CCATGCTTCT 60
RBL002.2 (prot.) M L 2
RBL002.4 (prot.) M L 2
                                                                                              antigen
RBL002.2 (RNA) GGCTGTGCTC TATTGCTTGC TGTGGTCCTT CCAGACTTCT GCCGGACACT TTCCCAGAGC 120
RBL002.4 (RNA) GGCTGTGCTC TATTGCTTGC TGTGGTCCTT CCAGACTTCT GCCGGACACT TTCCCAGAGC 120
RBL002.2 (prot.) L A V L Y C L L W S F Q T S A G H F P R 22
RBL002.4 (prot.) L A V L Y C L L W S F Q T S A G H F P R 22
                                                                                              antigen
RBL002.2 (RNA) ATGTGTGTCA TCCAAAAACC TCATGGAGAA AGAATGCTGT CCTCCTTGGA GTGGTGATAG 180
RBL002.4 (RNA) ATGTGTGTCA TCCAAAAACC TCATGGAGAA AGAATGCTGT CCTCCTTGGA GTGGTGATAG 180
RBL002.2 (prot.) A C V S S K N L M E K E C C P P W S G D 42
RBL002.4 (prot.) A C V S S K N L M E K E C C P P W S G D 42
                                                                                              antigen
RBL002.2 (RNA) ATCCCCATGT GGACAGCTCT CAGGCAGAGG ATCTTGCCAG AACATTCTGC TGAGCAATGC 240
RBL002.4 (RNA) ATCCCCATGT GGACAGCTCT CAGGCAGAGG ATCTTGCCAG AACATTCTGC TGAGCAATGC 240
RBL002.2 (prot.) R S P C G Q L S G R G S C Q N I L L S N 62
RBL002.4 (prot.) R S P C G Q L S G R G S C Q N I L L S N 62
                                                                                              antigen
RBL002.2 (RNA) ACCTCTTGGC CCACAGTTTC CCTTTACCGG AGTAGATGAC AGAGAAAGTT GGCCCTCCGT 300
RBL002.4 (RNA) ACCTCTTGGC CCACAGTTTC CCTTTACCGG AGTAGATGAC AGAGAAAGTT GGCCCTCCGT 300
RBL002.2 (prot.) A P L G P Q F P F T G V D D R E S W P S 82
RBL002.4 (prot.) A P L G P Q F P F T G V D D R E S W P S 82
                                                                                              antigen
RBL002.2 (RNA) GTTCTACAAC AGAACATGCC AATGCAGCGG CAATTTTATG GGATTTAACT GCGGAAATTG 360
RBL002.4 (RNA) GTTCTACAAC AGAACATGCC AATGCAGCGG CAATTTTATG GGATTTAACT GCGGAAATTG 360
RBL002.2 (prot.) V F Y N R T C Q C S G N F M G F N C G N 102
RBL002.4 (prot.) V F Y N R T C Q C S G N F M G F N C G N 102
                                                                                              antigen
RBL002.2 (RNA) CAAATTTGGA TTTTGGGGCC CAAACTGTAC AGAAAGAAGA CTGCTCGTTA GAAGAAACAT 420
RBL002.4 (RNA) CAAATTTGGA TTTTGGGGCC CAAACTGTAC AGAAAGAAGA CTGCTCGTTA GAAGAAACAT 420
RBL002.2 (prot.) C K F G F W G P N C T E R R L L V R R N 122
RBL002.4 (prot.) C K F G F W G P N C T E R R L L V R R N 122
                                                                                              antigen
RBL002.2 (RNA) CTTTGATCTG AGCGCACCCG AAAAAGACAA ATTCTTCGCT TATCTGACTC TCGCCAAACA 480
RBL002.4 (RNA) CTTTGATCTG AGCGCACCCG AAAAAGACAA ATTCTTCGCT TATCTGACTC TCGCCAAACA 480
RBL002.2 (prot.) I F D L S A P E K D K F F A Y L T L A K 142
RBL002.4 (prot.) I F D L S A P E K D K F F A Y L T L A K 142
                                                                                              antigen
RBL002.2 (RNA) CACAATAAGC AGTGATTATG TCATTCCCAT TGGCACTTAT GGACAGATGA AAAATGGCTC 540
RBL002.4 (RNA) CACAATAAGC AGTGATTATG TCATTCCCAT TGGCACTTAT GGACAGATGA AAAATGGCTC 540
RBL002.2 (prot.) H T I S S D Y V I P I G T Y G Q M K N G 162
RBL002.4 (prot.) H T I S S D Y V I P I G T Y G Q M K N G 162
                                                                                              antigen
RBL002.2 (RNA) AACTCCCATG TTCAACGACA TCAACATATA CGATCTGTTT GTGTGGATGC ATTACTACGT 600
RBL002.4 (RNA) AACTCCCATG TTCAACGACA TCAACATATA CGATCTGTTT GTGTGGATGC ATTACTACGT 600
RBL002.2 (prot.) S T P M F N D I N I Y D L F V W M H Y Y 182
RBL002.4 (prot.) S T P M F N D I N I Y D L F V W M H Y Y 182
                                                                                              antigen
RBL002.2 (RNA) GAGTATGGAT GCTCTGCTGG GTGGCTCCGA AATATGGAGG GATATAGATT TTGCACACGA 660
RBL002.4 (RNA) GAGTATGGAT GCTCTGCTGG GTGGCTCCGA AATATGGAGG GATATAGATT TTGCACACGA 660
RBL002.2 (prot.) V S M A A L L G G S E I W R D I D F A H 202
RBL002.4 (prot.) V S M A A L L G G S E I W R D I D F A H 202
                                                                                              antigen
RBL002.2 (RNA) AGCTCCTGCC TTCCTTCCAT GGCATAGACT GTTCCTGCTG AGATGGGAAC AAGAAATCCA 720
RBL002.4 (RNA) AGCTCCTGCC TTCCTTCCAT GGCATAGACT GTTCCTGCTG AGATGGGAAC AAGAAATCCA 720
RBL002.2 (prot.) E A P A F L P W H R L F L L R W E Q E I 222
RBL002.4 (prot.) E A P A F L P W H R L F L L R W E Q E I 222
                                                                                              antigen
RBL002.2 (RNA) GAAACTTACA GGCGATGAAA ACTTCACTAT CCCTTATTGG GATTGGAGAG ATGCTGAGAA 780
RBL002.4 (RNA) GAAACTTACA GGCGATGAAA ACTTCACTAT CCCTTATTGG GATTGGAGAG ATGCTGAGAA 780
RBL002.2 (prot.) Q K L T G D E N F T I P Y W D W R D A E 242
RBL002.4 (prot.) Q K L T G D E N F T I P Y W D W R D A E 242
                                                                                              antigen
RBL002.2 (RNA) ATGCGATATC TGTACCGATG AATACATGGG TGGTCAACAC CCAACCAACC CCAATCTCCT 840
RBL002.4 (RNA) ATGCGATATC TGTACCGATG AATACATGGG TGGTCAACAC CCAACCAACC CCAATCTCCT 840
RBL002.2 (prot.) K C D I C T D E Y M G G Q H P T N P N L 262
RBL002.4 (prot.) K C D I C T D E Y M G G Q H P T N P N L 262
                                                                                              antigen
RBL002.2 (RNA) ATGCGATATC TGTACCGATG AATACATGGG TGGTCAACAC CCAACCAACC CCAATCTCCT 840
RBL002.4 (RNA) ATGCGATATC TGTACCGATG AATACATGGG TGGTCAACAC CCAACCAACC CCAATCTCCT 840
RBL002.2 (prot.) K C D I C T D E Y M G G Q H P T N P N L 262
RBL002.4 (prot.) K C D I C T D E Y M G G Q H P T N P N L 262
                                                                                              antigen
RBL002.2 (RNA) GAGCCCTGCC TCTTTCTTCA GTTCTTGGCA GATTGTCTGT TCAAGATTGG AAGAATACAA 900
RBL002.4 (RNA) GAGCCCTGCC TCTTTCTTCA GTTCTTGGCA GATTGTCTGT TCAAGATTGG AAGAATACAA 900
RBL002.2 (prot.) L S P A S F F S S W Q I V C S R L E E Y 282
RBL002.4 (prot.) L S P A S F F S S W Q I V C S R L E E Y 282
                                                                                              antigen
RBL002.2 (RNA) TTCCCATCAG TCCCTGTGTA ACGGAACACC AGAAGGACCT CTGAGAAGAA ACCCTGGCAA 960
RBL002.4 (RNA) TTCCCATCAG TCCCTGTGTA ACGGAACACC AGAAGGACCT CTGAGAAGAA ACCCTGGCAA 960
RBL002.2 (prot.) N S H Q S L C N G T P E G P L R R N P G 302
RBL002.4 (prot.) N S H Q S L C N G T P E G P L R R N P G 302
                                                                                              antigen
RBL002.2 (RNA) TCATGACAAA AGCAGGACAC CTAGACTTCC CTCCTCTGCC GACGTTGAAT TTTGCCTCTC 1020
RBL002.4 (RNA) TCATGACAAA AGCAGGACAC CTAGACTTCC CTCCTCTGCC GACGTTGAAT TTTGCCTCTC 1020
RBL002.2 (prot.) N H D K S R T P R L P S S A D V E F C L 322
RBL002.4 (prot.) N H D K S R T P R L P S S A D V E F C L 322
                                                                                              antigen
RBL002.2 (RNA) TCTGACCCAG TACGAAAGCG GTAGCATGGA CAAAGCCGCC AATTTCAGCT TTAGAAATAC 1080
RBL002.4 (RNA) TCTGACCCAG TACGAAAGCG GTAGCATGGA CAAAGCCGCC AATTTCAGCT TTAGAAATAC 1080
RBL002.2 (prot.) S L T Q Y E S G S M D K A A N E S F R N 342
RBL002.4 (prot.) S L T Q Y E S G S M D K A A N E S F R N 342
                                                                                              antigen
RBL002.2 (RNA) CTTGGAAGGA TTTGCCTCAC CTCTGACAGG AATTGCTGAT GCTAGCCAGT CATCCATGCA 1140
RBL002.4 (RNA) CTTGGAAGGA TTTGCCTCAC CTCTGACAGG AATTGCTGAT GCTAGCCAGT CATCCATGCA 1140
RBL002.2 (prot.) T L E G F A S P L T G I A D A S Q S S M 362
RBL002.4 (prot.) T L E G F A S P L T G I A D A S Q S S M 362
                                                                                            antigen
RBL002.2 (RNA) CAACGCTTTG CACATCTATA TGAATGGAAC CATGAGTCAG GTTCAGGGAT CTGCCAATGA 1200
RBL002.4 (RNA) CAACGCTTTG CACATCTATA TGAATGGAAC CATGAGTCAG GTTCAGGGAT CTGCCAATGA 1200
RBL002.2 (prot.) H N A L H I Y M N G T M S Q V Q G S A N 382
RBL002.4 (prot.) H N A L H I Y M N G T M S Q V Q G S A N 382
                                                                                            antigen
RBL002.2 (RNA) TCCTATTTTC CTGCTGCACC ATGCATTCGT GGACTCTATC TTTGAGCAGT GGCTTAGAAG 1260
RBL002.4 (RNA) TCCTATTTTC CTGCTGCACC ATGCATTCGT GGACTCTATC TTTGAGCAGT GGCTTAGAAG 1260
RBL002.2 (prot.) D P I F L L H H A F V D S I F E Q W L R 402
RBL002.4 (prot.) D P I F L L H H A F V D S I F E Q W L R 402
                                                                                            antigen
RBL002.2 (RNA) ACACAGACCA TTGCAGGAAG TCTATCCAGA GGCCAACGCA CCAATCGGCC ATAATAGAGA 1320
RBL002.4 (RNA) ACACAGACCA TTGCAGGAAG TCTATCCAGA GGCCAACGCA CCAATCGGCC ATAATAGAGA 1320
RBL002.2 (prot.) R H R P L Q E V Y P E A N A P I G H N R 422
RBL002.4 (prot.) R H R P L Q E V Y P E A N A P I G H N R 422
                                                                                            antigen
RBL002.2 (RNA) AAGCTACATG GTACCCTTCA TTCCTCTGTA CAGAAATGGA GATTTCTTCA TCAGCTCCAA 1380
RBL002.4 (RNA) AAGCTACATG GTACCCTTCA TTCCTCTGTA CAGAAATGGA GATTTCTTCA TCAGCTCCAA 1380
RBL002.2 (prot.) E S Y M V P F I P L Y R N G D F F I S S 442
RBL002.4 (prot.) E S Y M V P F I P L Y R N G D F F I S S 442
                                                                                            antigen
RBL002.2 (RNA) AGACCTGGGC TACGATTACT CATATCTGCA AGACAGTGAT CCCGATAGCT TCCAAGACTA 1140
RBL002.4 (RNA) AGACCTGGGC TACGATTACT CATATCTGCA AGACAGTGAT CCCGATAGCT TCCAAGACTA 1140
RBL002.2 (prot.) K D L G Y D Y S Y L Q D S D P D S F Q D 462
RBL002.4 (prot.) K D L G Y D Y S Y L Q D S D P D S F Q D 462
                                                                        antigen ][ GS
RBL002.2 (RNA) CATTAAGTCT TATCTCGAAC AGGCGAGCAG AATCTGGTCC TGGGGATCCC TGGGAGGCGG 1500
RBL002.4 (RNA) CATTAAGTCT TATCTCGAAC AGGCGAGCAG AATCTGGTCC TGGGGAGGAT CCGGTGGTGG 1500
                                                                                                                                      **** ** * *
RBL002.2 (prot.) Y I K S Y L E Q A S R I W S W G S L G G 482
RBL002.4 (prot.) Y I K S Y L E Q A S R I W S W G S L G G 482
                                                                                              P2P16
RBL002.2 (RNA) GGGAAGC GGCAAGAAGC AGTACATCAA GGCCAACAGC AAGTTCATCG GCATCACCGA 1557
RBL002.4 (RNA) CGGCAGCGGC GGCAAGAAGC AGTACATCAA GGCCAACAGC AAGTTCATCG GCATCACCGA 1560
                                        ** **
RBL002.2 (prot.) G G S G K K Q Y I K A N S K F I G I T 501
RBL002.4 (prot.) G G S G G K K Q Y I K A N S K F I G I T 502
                                                    *
                                                                                              P2P16
RBL002.2 (RNA) GCTGAAGAAG CTGGGAGGGG GCAAACGGGG AGGCGGCAAA AAGATGACCA ACAGCGTGGA 1617
RBL002.4 (RNA) GCTGAAGAAG CTGGGAGGGG GCAAACGGGG AGGCGGCAAA AAGATGACCA ACAGCGTGGA 1620
RBL002.2 (prot.) E L K K L G G G K R G G G K K M T N S V 521
RBL002.4 (prot.) E L K K L G G G K R G G G K K M T N S V 522
                                                                                              P2P16
RBL002.2 (RNA) CGACGCCCTG ATCAACAGCA CCAAGATCTA CAGCTACTTC CCCAGCGTGA TCAGCAAAGT 1677
RBL002.4 (RNA) CGACGCCCTG ATCAACAGCA CCAAGATCTA CAGCTACTTC CCCAGCGTGA TCAGCAAAGT 1680
RBL002.2 (prot.) D D A L I N S T K I Y S Y F P S V I S K 541
RBL002.4 (prot.) D D A L I N S T K I Y S Y F P S V I S K 542
                                                          P2P16 ][ GS
RBL002.2 (RNA) GAACCAGGGC GCTCAGGGCA AGAAACTGGG CTCTAGCGGA GGGGGAGGCT CTCCTGGCGG 1737
RBL002.4 (RNA) GAACCAGGGC GCTCAGGGCA AGAAACTGGG CTCTAGCGGA GGGGGAGGCT CTCCTGGCGG 1740
RBL002.2 (prot.) V N Q G A Q G K K L G S S G G G G S P G 561
RBL002.4 (prot.) V N Q G A Q G K K L G S S G G G G S P G 562
                                        GS ][ MITD
RBL002.2 (RNA) AGCCGTGGTG GCTACCGTGA TGTGCAGACG GAAGTCCAGC GGAGGCAAGG GCGGCAGCTA 1797
RBL002.4 (RNA) AGCCGTGGTG GCTACCGTGA TGTGCAGACG GAAGTCCAGC GGAGGCAAGG GCGGCAGCTA 1800
RBL002.2 (prot.) G G S S I V G I V A G L A V L A V V V I 581
RBL002.4 (prot.) G G S S I V G I V A G L A V L A V V V I 582
                                                                                              MITF
RBL002.2 (RNA) AGCCGTGGTG GCTACCGTGA TGTGCAGACG GAAGTCCAGC GGAGGCAAGG GCGGCAGCTA 1857
RBL002.4 (RNA) AGCCGTGGTG GCTACCGTGA TGTGCAGACG GAAGTCCAGC GGAGGCAAGG GCGGCAGCTA 1860
RBL002.2 (prot.) G A V V A T V M C R R K S S G G K G G S 601
RBL002.4 (prot.) G A V V A T V M C R R K S S G G K G G S 602
                                                                                              MITD
RBL002.2 (RNA) CAGCCAGGCC GCCAGCTCTG ATAGCGCCCA GGGCAGCGAC GTGTCACTGA CAGCC TG 1914
RBL002.4 (RNA) CAGCCAGGCC GCCAGCTCTG ATAGCGCCCA GGGCAGCGAC GTGTCACTGA CAGCCTAGTA 1920
                                                                                                                                                          *** *
RBL002.2 (prot.) Y S Q A A S S D S A Q G S D V S L T A 620
RBL002.4 (prot.) Y S Q A A S S D S A Q G S D V S L T A 621
                                  ][ 3′-UTR
RBL002.2 (RNA) ACTCGAGAGC TCGCTTTCTT GCTGTCCAAT TTCTATTAAA GGTTCC TTTGTTCCCT 1970
RBL002.4 (RNA) ACTCGAGCTG GTACTGCATG CACGCAATGC TAGCTGCCCC TTTCCCGTCC TGGGTACCCC 1980
                                                *** *** *** * *** ****** ********* ******* ** * *
                                                                                              3′-UTR
RBL002.2 (RNA) AAGTCCAACT ACTAAACTGG GGGATATTAT GAAGGGCCTT GAGCATCTGG ATTCTGCCTA 2030
RBL002.4 (RNA) GAGTCTCCCC CGACCTCGGG TCCCAGGTAT GCTCCCACCT CCACCTGCCC CACTCACCAC 2040
                                  * *** * ****** * ******* ****** * *** * **** ****** **
                                                                                              3′-UTR
RBL002.2 (RNA) ATAAAAAACA TTTATTTTCA TTGCTGCGTC GAGAGCTCGC TTTCTTGCTG TCCAATTTCT 2090
RBL002.4 (RNA) CTCTGCTAGT TCCAGACACC TCCCAAGCAC GCAGCAATGC AGCTCAAAAC GCTTAGCCTA 2100
                                  * ***** ** ** **** * ** ***** ******* ********** * ** *****
                                                                                              3′-UTR
RBL002.4 (RNA) ATTAAAGG TTCCTTT GTTCCC TAAGTCCAAC TACTAAACTG 2131
RBL002.4 (RNA) GCCACACCCC CACGGGAAAC AGCAGTGATT AACCTTTAGC AATAAACGAA AGTTTAACTA 2160
                                  ********** ****** ** ***** **** ***** ** * **** * * *** * *
                                                                                              3′-UTR
RBL002.2 (RNA) GGGGATATTA TGAAGGGCCT TGAGCATCTG GATTCTGCCT AATAAAAAAC ATTTATTTTC 2191
RBL002.4 (RNA) AGCTATACTA ACCCCAGGGT TGGTCAATTT CGTGCCAGCC ACACCGAGAC CTGGTCCAGA 2220
                                  * ** * ****** ** ** ** * ** * *** * ***** * * ********
                                                3′-UTR ][ poly(A)
RBL002.2 (RNA) ATTGCTGCGT CGAGAGCTAA AAAAAAAAAA AAAAAAAAAA AAAAAAAAAA AAAAAAAAAA 2251
RBL002.4 (RNA) GTCGCTAGCC GCGTCGCTAA AAAAAAAAAA AAAAAAAAAA AAAAAAAAGC ATATGACT 2278
                                  * * **** ***** ** * ** ****
                                                                                              poly(A)
RBL002.2 (RNA) AAAAAAAAAA AAAAAAAAAA AAAAAAAAAA AAAAAAAAAA AAAAAAAAAA AAAAAAAAAA 2311
RBL002.4 (RNA) AA AAAAAAAAAA AAAAAAAAAA AAAAAAAAAA AAAAAAAAAA AAAAAAAAAA 2330
                                  ********
                                              poly(A) ]
RBL002.2 (RNA) AAAAAAAAAA AAAAAAAA 2329
RBL002.4 (RNA) AAAAAAAAAA AAAAAAAA 2348
```

In some embodiments, an RNA molecule encoding a MAGE-A3 antigen is or comprises the nucleotide sequence of RBL003.1 or RBL003.3. In some embodiments, an RNA molecule encoding a MAGE-A3 antigen comprises a sequence that encodes a polypeptide having the amino acid sequence of RBL003.1 or RBL003.3. In the following, the sequence alignment of RBL003.1 and RBL003.3 is given for both the nucleotide sequences of the full-length RNAs as well as for the translated proteins (with the amino acid positioned below the third nucleotide of the respective codon triplet). Sequence elements as illustrated in FIG. 1a are displayed above the nucleotide sequences. Differences in the nucleotide and amino acid sequences are indicated by “*”. SEQ ID NO: 17 for RBL003.1 RNA; SEQ ID NO: 18 for RBL003.3 RNA; SEQ ID NO: 19 for RBL003.1 protein; SEQ ID NO: 20 for RBL003.3 protein.

```
                                  [ 5′-UTR ][ sec
RBL003.1 (RNA) GGGCGAACTA GTATTCTTCT GGTCCCCACA GACTCAGAGA GAACCCGCCA CCATGAGAGT 60
RBL003.3 (RNA) GGGCGAACTA GTATTCTTCT GGTCCCCACA GACTCAGAGA GAACCCGCCA CCATGAGAGT 60
RBL003.1 (prot.) M R 2
RBL003.1 (prot.) M R 2
                                                                                                sec
RBL003.1 (RNA) GACCGCCCCC AGAACCCTGA TCCTGCTGCT GTCTGGCGCC CTGGCCCTGA CAGAGACATG 120
RBL003.3 (RNA) GATGGCCCCC AGAACCCTGA TCCTGCTGCT GTCTGGCGCC CTGGCCCTGA CAGAGACATG 120
                                      **
RBL003.1 (prot.) V T A P R T L I L L L S G A L A L T E T 22
RBL003.3 (prot.) V M A P R T L I L L L S G A L A L T E T 22
                                        *
                                        sec ][ GS ][ antigen
RBL003.1 (RNA) GGCCGGAAGC CTGCAG GGAGGATCT ATGCCCCTTG AACAGCGCTC 165
RBL003.3 (RNA) GGCCGGAAGC GGCGGCTCTG GAGGAGGCGG CTCCGGAGGC ATGCCCCTTG AACAGCGCTC 180
                                                        ********** ********** **** ***
RBL003.1 (prot.) W A G S L Q G G S M P L E Q R 37
RBL003.3 (prot.) W A G S G G S G G G G S G G M P L E Q R 42
                                                            * * * * * * * * *
                                                                                              antigen
RBL003.1 (RNA) ACAGCACTGC AAACCTGAGG AGGGCCTTGA AGCAAGGGGC GAAGCTCTGG GGTTGGTCGG 225
RBL003.3 (RNA) ACAGCACTGC AAACCTGAGG AGGGCCTTGA AGCAAGGGGC GAAGCTCTGG GGTTGGTCGG 240
RBL003.1 (prot.) S Q H C K P E E G L E A R G E A L G L V 57
RBL003.3 (prot.) S Q H C K P E E G L E A R G E A L G L V 62
                                                                                              antigen
RBL003.1 (RNA) TGCACAAGCA CCCGCCACTG AGGAACAGGA AGCCGCGTCT AGCTCATCAA CCCTGGTTGA 285
RBL003.3 (RNA) TGCACAAGCA CCCGCCACTG AGGAACAGGA AGCCGCGTCT AGCTCATCAA CCCTGGTTGA 300
RBL003.1 (prot.) G A Q A P A T E E Q E A A S S S S T L V 77
RBL003.3 (prot.) G A Q A P A T E E Q E A A S S S S T L V 82
                                                                                              antigen
RBL003.1 (RNA) AGTGACACTG GGCGAAGTGC CTGCTGCGGA GAGTCCAGAC CCTCCCCAGT CCCCTCAAGG 345
RBL003.3 (RNA) AGTGACACTG GGCGAAGTGC CTGCTGCGGA GAGTCCAGAC CCTCCCCAGT CCCCTCAAGG 360
RBL003.1 (prot.) E V T L G E V P A A E S P D P P Q S P Q 97
RBL003.3 (prot.) E V T L G E V P A A E S P D P P Q S P Q 102
                                                                                              antigen
RBL003.1 (RNA) CGCTTCTAGC CTGCCTACCA CGATGAACTA CCCACTGTGG TCACAGAGCT ATGAGGACAG 405
RBL003.3 (RNA) CGCTTCTAGC CTGCCTACCA CGATGAACTA CCCACTGTGG TCACAGAGCT ATGAGGACAG 420
RBL003.1 (prot.) G A S S L P T T M N Y P L W S Q S Y E D 117
RBL003.3 (prot.) G A S S L P T T M N Y P L W S Q S Y E D 122
                                                                                              antigen
RBL003.1 (RNA) TTCCAATCAA GAAGAAGAAG GCCCGTCTAC CTTCCCCGAT CTTGAGTCCG AGTTTCAGGC 465
RBL003.3 (RNA) TTCCAATCAA GAAGAAGAAG GCCCGTCTAC CTTCCCCGAT CTTGAGTCCG AGTTTCAGGC 480
RBL003.1 (prot.) S S N Q E E E G P S T F P D L E S E F Q 137
RBL003.3 (prot.) S S N Q E E E G P S T F P D L E S E F Q 142
                                                                                              antigen
RBL003.1 (RNA) CGCTCTGTCC CGGAAGGTGG CAGAGCTCGT GCACTTTCTC CTGTTGAAGT ATCGAGCCCG 525
RBL003.3 (RNA) CGCTCTGTCC CGGAAGGTGG CAGAGCTCGT GCACTTTCTC CTGTTGAAGT ATCGAGCCCG 540
RBL003.1 (prot.) A A L S R K V A I L V H F L L L K Y R A 157
RBL003.3 (prot.) A A L S R K V A I L V H F L L L K Y R A 162
                                                                                              antigen
RBL003.1 (RNA) GGAGCCTGTC ACTAAGGCCG AAATGCTGGG CTCTGTAGTG GGGAATTGGC AGTATTTCTT 585
RBL003.3 (RNA) GGAGCCTGTC ACTAAGGCCG AAATGCTGGG CTCTGTAGTG GGGAATTGGC AGTATTTCTT 600
RBL003.1 (prot.) R E P V T K A E M L G S V V G N W Q Y F 177
RBL003.3 (prot.) R E P V T K A E M L G S V V G N W Q Y F 182
                                                                                              antigen
RBL003.1 (RNA) CCCCGTGATC TTCAGCAAAG CCTCCAGCAG CCTGCAATTG GTGTTCGGTA TTGAACTGAT 645
RBL003.3 (RNA) CCCCGTGATC TTCAGCAAAG CCTCCAGCAG CCTGCAATTG GTGTTCGGTA TTGAACTGAT 660
RBL003.1 (prot.) F P V I F S K A S S S L Q L V F G I E L 197
RBL003.3 (prot.) F P V I F S K A S S S L Q L V F G I E L 202
                                                                                              antigen
RBL003.1 (RNA) GGAAGTAGAT CCGATTGGGC ATCTGTACAT CTTTGCGACA TGTCTGGGAC TGTCCTATGA 705
RBL003.3 (RNA) GGAAGTAGAT CCGATTGGGC ATCTGTACAT CTTTGCGACA TGTCTGGGAC TGTCCTATGA 720
RBL003.1 (prot.) M E V D P I G H L Y I F A T C L G L S Y 217
RBL003.3 (prot.) M E V D P I G H L Y I F A T C L G L S Y 222
                                                                                              antigen
RBL003.1 (RNA) CGGACTGCTC GGGGATAACC AGATTATGCC GAAAGCCGGT CTGCTGATCA TAGTTCTCGC 765
RBL003.3 (RNA) CGGACTGCTC GGGGATAACC AGATTATGCC GAAAGCCGGT CTGCTGATCA TAGTTCTCGC 780
RBL003.1 (prot.) D G L L G D N Q I M P K A G L L I I V L 237
RBL003.3 (prot.) D G L L G D N Q I M P K A G L L I I V L 242
                                                                                              antigen
RBL003.1 (RNA) CATCATTGCC AGAGAGGGAG ATTGTGCTCC AGAGGAGAAG ATCTGGGAGG AATTGTCTGT 825
RBL003.3 (RNA) CATCATTGCC AGAGAGGGAG ATTGTGCTCC AGAGGAGAAG ATCTGGGAGG AATTGTCTGT 840
RBL003.1 (prot.) A I I A R E G D C A P E E K I W E E L S 257
RBL003.3 (prot.) A I I A R E G D C A P E E K I W E E L S 262
                                                                                              antigen
RBL003.1 (RNA) GCTGGAGGTC TTTGAGGGTA GGGAGGACAG CATTCTCGGC GATCCCAAGA AACTCCTGAC 885
RBL003.3 (RNA) GCTGGAGGTC TTTGAGGGTA GGGAGGACAG CATTCTCGGC GATCCCAAGA AACTCCTGAC 900
RBL003.1 (prot.) V L E V F E G R E D S I L G D P K K L L 277
RBL003.3 (prot.) V L E V F E G R E D S I L G D P K K L L 282
                                                                                              antigen
RBL003.1 (RNA) CCAGCACTTT GTCCAGGAGA ACTACCTCGA ATACAGACAG GTTCCAGGCA GTGACCCTGC 945
RBL003.3 (RNA) CCAGCACTTT GTCCAGGAGA ACTACCTCGA ATACAGACAG GTTCCAGGCA GTGACCCTGC 960
RBL003.1 (prot.) T Q H F V Q E N Y L E Y R Q V P G S D P 297
RBL003.3 (prot.) T Q H F V Q E N Y L E Y R Q V P G S D P 302
                                                                                              antigen
RBL003.1 (RNA) TTGCTACGAG TTCCTTTGGG GACCCCGTGC ATTGGTAGAG ACAAGCTATG TCAAAGTGCT 1005
RBL003.3 (RNA) TTGCTACGAG TTCCTTTGGG GACCCCGTGC ATTGGTAGAG ACAAGCTATG TCAAAGTGCT 1020
RBL003.1 (prot.) A C Y E F L W G P R A L V E T S Y V K V 317
RBL003.3 (prot.) A C Y E F L W G P R A L V E T S Y V K V
                                                                                              antigen
RBL003.1 (RNA) GCACCATATG GTGAAGATAT CTGGAGGACC ACACATCAGT TACCCACCCC TTCATGAGTG 1065
RBL003.3 (RNA) GCACCATATG GTGAAGATAT CTGGAGGACC ACACATCAGT TACCCACCCC TTCATGAGTG 1080
RBL003.1 (prot.) L H H M V K I S G G P H I S Y P P L H E 337
RBL003.3 (prot.) L H H M V K I S G G P H I S Y P P L H E 342
                                                          antigen ][ GS ][ P2P16
RBL003.1 (RNA) GGTTCTGCGC GAAGGGGAGG AGGGAGGATC CCTGGGAGGC GGGGGAAGCG GCAAGAAGCA 1125
RBL003.3 (RNA) GGTTCTGCGC GAAGGGGAGG AGGGAGGATC CGGTGGTGGC GGCAGCGGCG GCAAGAAGCA 1140
                                                                                                      ** * ** **
RBL003.1 (prot.) W V L R E G E E G G S L G G G G S G K K 357
RBL003.3 (prot.) W V L R E G E E G G S G G G G S G G K K 362
                                                                                                          * * *
                                                                                              P2P16
RBL003.1 (RNA) GTACATCAAG GCCAACAGCA AGTTCATCGG CATCACCGAG CTGAAGAAGC TGGGAGGGGG 1185
RBL003.3 (RNA) GTACATCAAG GCCAACAGCA AGTTCATCGG CATCACCGAG CTGAAGAAGC TGGGAGGGGG 1200
RBL003.1 (prot.) Q Y I K A N S K F I G I T E L K K L G G 377
RBL003.3 (prot.) Q Y I K A N S K F I G I T E L K K L G G 382
                                                                                              P2P16
RBL003.1 (RNA) CAAACGGGGA GGCGGCAAAA AGATGACCAA CAGCGTGGAC GACGCCCTGA TCAACAGCAC 1245
RBL003.3 (RNA) CAAACGGGGA GGCGGCAAAA AGATGACCAA CAGCGTGGAC GACGCCCTGA TCAACAGCAC 1260
RBL003.1 (prot.) G K R G G G K K M T N S V D D A L I N S 397
RBL003.3 (prot.) G K R G G G K K M T N S V D D A L I N S 402
                                                                                              P2P16
RBL003.1 (RNA) CAAGATCTAC AGCTACTTCC CCAGCGTGAT CAGCAAAGTG AACCAGGGCG CTCAGGGCAA 1305
RBL003.3 (RNA) CAAGATCTAC AGCTACTTCC CCAGCGTGAT CAGCAAAGTG AACCAGGGCG CTCAGGGCAA 1320
RBL003.1 (prot.) T K I Y S Y F P S V I S K V N Q G A Q G 417
RBL003.3 (prot.) T K I Y S Y F P S V I S K V N Q G A Q G 422
                                    P2P16 ][ GS ][ MITD
RBL003.1 (RNA) GAAACTGGGC TCTAGCGGAG GGGGAGGCTC TCCTGGCGGG GGATCTAGCA TCGTGGGAAT 1365
RBL003.3 (RNA) GAAACTGGGC TCTAGCGGAG GGGGAGGCTC TCCTGGCGGG GGATCTAGCA TCGTGGGAAT 1380
RBL003.1 (prot.) K K L G S S G G G G S P G G G S S I V G 437
RBL003.3 (prot.) K K L G S S G G G G S P G G G S S I V G 442
                                                                                                MITD
RBL003.1 (RNA) TGTGGCAGGA CTGGCAGTGC TGGCCGTGGT GGTGATCGGA GCCGTGGTGG CTACCGTGAT 1425
RBL003.3 (RNA) TGTGGCAGGA CTGGCAGTGC TGGCCGTGGT GGTGATCGGA GCCGTGGTGG CTACCGTGAT 1440
RBL003.1 (prot.) I V A G L A V L A V V V I G A V V A T V 457
RBL003.3 (prot.) I V A G L A V L A V V V I G A V V A T V 462
                                                                                                MITD
RBL003.1 (RNA) GTGCAGACGG AAGTCCAGCG GAGGCAAGGG CGGCAGCTAC AGCCAGGCCG CCAGCTCTGA 1485
RBL003.3 (RNA) GTGCAGACGG AAGTCCAGCG GAGGCAAGGG CGGCAGCTAC AGCCAGGCCG CCAGCTCTGA 1500
RBL003.1 (prot.) M C R R K S S G G K G G S Y S Q A A S S 477
RBL003.3 (prot.) M C R R K S S G G K G G S Y S Q A A S S 482
                                                                          MITD ][ 3′-UTR
RBL003.1 (RNA) TAGCGCCCAG GGCAGCGACG TGTCACTGAC AGCC TGA CTCGAGAGCT CGCTTTCTTG 1542
RBL003.3 (RNA) TAGCGCCCAG GGCAGCGACG TGTCACTGAC AGCCTAGTAA CTCGAGCTGG TACTGCATGC 1560
                                                                                                            *** * **** ** *** **
RBL003.1 (prot.) D S A Q G S D V S L T A 489
RBL003.3 (prot.) D S A Q G S D V S L T A 494
                                                                                              3′-UTR
RBL003.1 (RNA) CTGTCCAATT TCTATTAAA GGTTCCT TTGTTCCCTA AGTCCAACTA CTAAACTGGG 1598
RBL003.3 (RNA) ACGCAATGCT AGCTGCCCCT TTCCCGTCCT GGGTACCCCG AGTCTCCCCC GACCTCGGGT 1620
                                  ** ****** ********** ****** ** * ** *** ** ***** * *
                                                                                              3′-UTR
RBL003.1 (RNA) GGATATTATG AAGGGCCTTG AGCATCTGGA TTCTGCCTAA TAAAAAACAT TTATTTTCAT 1658
RBL003.3 (RNA) CCCAGGTATG CTCCCACCTC CACCTGCCCC ACTCACCACC TCTGCTAGTT CCAGACACCT 1680
                                  ****** ****** * * ** * ***** ***** *** ***** ** ** **** *
                                                                                              3′-UTR
RBL003.1 (RNA) TGCTGCGTCG AGAGCTCGCT TTCTTGCTGT CCAATTTCT ATTAAAGGT 1706
RBL003.3 (RNA) CCCAAGCACG CAGCAATGCA GCTCAAAACG CTTAGCCTAG CCACACCCCC ACGGGAAACA 1740
                                  ** ***** ******* * ********** ** ****** ********** ***** ***
                                                                                              3′-UTR
RBL003.1 (RNA) TCCTTT GTTCCCT AAGTCCAACT ACTAAACTGG GGGATATTAT GAAGGGCCTT 1759
RBL003.3 (RNA) GCAGTGATTA ACCTTTAGCA ATAAACGAAA GTTTAACTAA GCTATACTAA CCCCAGGGTT 1800
                                  **** ***** **** ** * **** * ** ** * ** ** * * ***** **
                                                                                      3′-UTR ][poly(A)
RBL003.1 (RNA) GAGCATCTGG ATTCTGCCTA ATAAAAAACA TTTATTTTCA TTGCTGCGTC GAGAGCTAAA 1819
RBL003.3 (RNA) GGTCAATTTC GTGCCAGCCA CACCGAGACC TGGTCCAGAG TCGCTAGCCG CGTCGCTAAA 1860
                                    ** ** ** * * *** * ***** * * ********* * ***** ****
                                                                                              poly(A)
RBL003.1 (RNA) AAAAAAAAAA AAAAAAAAAA AAAAAAAAAA AAAAAAAAAA AAAAAAAAAA AAAAAAAAAA 1879
RBL003.3 (RNA) AAAAAAAAAA AAAAAAAAAA AAAAAAAGCA TATGACT AAA AAAAAAAAAA 1910
                                                                                            ** * ** ***** *******
                                                                                              poly(A) ]
RBL003.1 (RNA) AAAAAAAAAA AAAAAAAAAA AAAAAAAAAA AAAAAAAAAA AAAAAAAAAA AAAAAAA 1936
RBL003.3 (RNA) AAAAAAAAAA AAAAAAAAAA AAAAAAAAAA AAAAAAAAAA AAAAAAAAAA AAAAAAA 1967
```

In some embodiments, an RNA molecule encoding a TPTE antigen is or comprises the nucleotide sequence of RBL004.1 or RBL004.3. In some embodiments, an RNA molecule encoding a TPTE antigen comprises a sequence that encodes a polypeptide having the amino acid sequence of RBL004.1 or RBL004.3. In the following, the sequence alignment of RBL004.1 and RBL004.3 is given for both the nucleotide sequences of the full-length RNAs as well as for the translated proteins (with the amino acid positioned below the third nucleotide of the respective codon triplet). Sequence elements as illustrated in FIG. 1a are displayed above the nucleotide sequences. Differences in the nucleotide and amino acid sequences are indicated by “*”. SEQ ID NO: 21 for RBL004.1 RNA; SEQ ID NO: 22 for RBL004.3 RNA; SEQ ID NO: 23 for RBL004.1 protein; SEQ ID NO: 24 for RBL004.3 protein.

```
                                  [ 5′-UTR ][ sec
RBL004.1 (RNA) GGGCGAACTA GTATTCTTCT GGTCCCCACA GACTCAGAGA GAACCCGCCA CCATGAGAGT 60
RBL004.3 (RNA) GGGCGAACTA GTATTCTTCT GGTCCCCACA GACTCAGAGA GAACCCGCCA CCATGAGAGT 60
RBL004.1 (prot.) M R 2
RBL004.3 (prot.) M R 2
                                                                                                sec
RBL004.1 (RNA) GACCGCCCCC AGAACCCTGA TCCTGCTGCT GTCTGGCGCC CTGGCCCTGA CAGAGACATG 120
RBL004.3 (RNA) GATGGCCCCC AGAACCCTGA TCCTGCTGCT GTCTGGCGCC CTGGCCCTGA CAGAGACATG 120
RBL004.1 (prot.) V T A P R T L I L L L S G A L A L I E T 22
RBL004.3 (prot.) V M A P R T L I L L L S G A L A L I E T 22
                                        *
                                      sec ][ GS ][ antigen
RBL004.1 (RNA) GGCCGGAAGC CTG CAG ATGAACGAGA GCCCCGACCC 156
RBL004.3 (RNA) GGCCGGAAGC GGCGGCTCTG GAGGAGGCGG CTCCGGAGGC ATGAACGAGA GCCCCGACCC 180
                                                        ********** ********** **********
RBL004.1 (prot.) W A G S L Q M N E S P D 34
RBL004.3 (prot.) W A G S G G S G G G G S G G M N E S P D 42
                                                                                              antigen
RBL004.1 (RNA) TACAGATCTG GCCGGCGTGA TCATCGAGCT GGGACCCAAC GATAGCCCTC AGACCAGCGA 216
RBL004.3 (RNA) TACAGATCTG GCCGGCGTGA TCATCGAGCT GGGACCCAAC GATAGCCCTC AGACCAGCGA 240
RBL004.1 (prot.) P T D L A G V I I E L G P N D S P Q T S 54
RBL004.3 (prot.) P T D L A G V I I E L G P N D S P Q T S 62
                                                                                              antigen
RBL004.1 (RNA) GTTCAAGGGG GCCACAGAGG AAGCCCCTGC CAAAGAGAGC CCCCACACCT CCGAGTTTAA 276
RBL004.3 (RNA) GTTCAAGGGG GCCACAGAGG AAGCCCCTGC CAAAGAGAGC CCCCACACCT CCGAGTTTAA 300
RBL004.1 (prot.) E F K G A T E E A P A K E S P H T S E F 74
RBL004.3 (prot.) E F K G A T E E A P A K E S P H T S E F 82
                                                                                              antigen
RBL004.1 (RNA) GGGCGCTGCT CGGGTGTCCC CTATCAGCGA GAGCGTGCTG GCCCGGCTGA GCAAGTTCGA 336
RBL004.3 (RNA) GGGCGCTGCT CGGGTGTCCC CTATCAGCGA GAGCGTGCTG GCCCGGCTGA GCAAGTTCGA 360
RBL004.1 (prot.) K G A A R V S P I S E S V L A R L S K F 94
RBL004.3 (prot.) K G A A R V S P I S E S V L A R L S K F 102
                                                                                              antigen
RBL004.1 (RNA) GGTGGAGGAC GCCGAGAACG TGGCCAGCTA CGACAGCAAG ATCAAGAAAA TCGTGCACAG 396
RBL004.3 (RNA) GGTGGAGGAC GCCGAGAACG TGGCCAGCTA CGACAGCAAG ATCAAGAAAA TCGTGCACAG 420
RBL004.1 (prot.) E V E D A E N V A S Y D S K I K K I V H 114
RBL004.3 (prot.) E V E D A E N V A S Y D S K I K K I V H 122
                                                                                              antigen
RBL004.1 (RNA) CATCGTGTCC AGCTTCGCCT TCGGCCTGTT CGGCGTGTTC CTGGTGCTGC TGGACGTGAC 456
RBL004.3 (RNA) CATCGTGTCC AGCTTCGCCT TCGGCCTGTT CGGCGTGTTC CTGGTGCTGC TGGACGTGAC 480
RBL004.1 (prot.) S I V S S F A F G L F G V F L V L L D V 134
RBL004.3 (prot.) S I V S S F A F G L F G V F L V L L D C 142
                                                                                              antigen
RBL004.1 (RNA) ACTGATCCTG GCCGACCTGA TCTTCACCGA CAGCAAGCTG TACATCCCCC TGGAATACCG 516
RBL004.3 (RNA) ACTGATCCTG GCCGACCTGA TCTTCACCGA CAGCAAGCTG TACATCCCCC TGGAATACCG 540
RBL004.1 (prot.) T L I L A D L I F T D S K L Y I P L E Y 154
RBL004.3 (prot.) T L I L A D L I F T D S K L Y I P L E Y 162
                                                                                              antigen
RBL004.1 (RNA) GTCCATCAGC CTGGCCATTG CCCTGTTCTT TCTGATGGAC GTGCTGCTGC GGGTGTTCGT 576
RBL004.3 (RNA) GTCCATCAGC CTGGCCATTG CCCTGTTCTT TCTGATGGAC GTGCTGCTGG GGGTGTTCGT 600
RBL004.1 (prot.) R S I S L A I A L F F L M D V L L R V F 174
RBL004.3 (prot.) R S I S L A I A L F F L M D V L L R V F 182
                                                                                              antigen
RBL004.1 (RNA) GGAGCGGCGG CAGCAGTACT TCAGCGACCT GTTCAACATC CTGGACACCG CCATCATCGT 636
RBL004.3 (RNA) GGAGCGGCGG CAGCAGTACT TCAGCGACCT GTTCAACATC CTGGACACCG CCATCATCGT 660
RBL004.1 (prot.) V E R R Q Q Y F S D L F N I L D T A I I 194
RBL004.3 (prot.) V E R R Q Q Y F S D L F N I L D T A I I 202
                                                                                              antigen
RBL004.1 (RNA) GATTCTGCTG CTGGTGGATG TGGTGTACAT CTTCTTCGAC ATCAAGCTGC TGAGAAACAT 696
RBL004.3 (RNA) GATTCTGCTG CTGGTGGATG TGGTGTACAT CTTCTTCGAC ATCAAGCTGC TGAGAAACAT 720
RBL004.1 (prot.) V I L L L V D V V Y I F F D I K L L R N 214
RBL004.3 (prot.) V I L L L V D V V Y I F F D I K L L R N 222
                                                                                              antigen
RBL004.1 (RNA) CCCCCGGTGG ACCCATCTGC TGCGGCTGCT GAGACTGATC ATCCTGCTGC GGATCTTCCA 756
RBL004.3 (RNA) CCCCCGGTGG ACCCATCTGC TGCGGCTGCT GAGACTGATC ATCCTGCTGC GGATCTTCCA 780
RBL004.1 (prot.) I P R W T H L L R L L R L I I L L R I F 234
RBL004.3 (prot.) I P R W T H L L R L L R L I I L L R I F 242
antigen
RBL004.1 (RNA) CCTGTTCCAC CAGAAGCGGC AGCTGGAAAA GCTGATCAGA CGGCGGGTGT CCGAGAACAA 816
RBL004.3 (RNA) CCTGTTCCAC CAGAAGCGGC AGCTGGAAAA GCTGATCAGA CGGCGGGTGT CCGAGAACAA 840
RBL004.1 (prot.) H L F H Q K R Q L E K L I R R R V S E N 254
RBL004.3 (prot.) H L F H Q K R Q L E K L I R R R V S E N 262
                                                                                              antigen
RBL004.1 (RNA) GCGGCGGTAC ACCAGGGACG GCTTCGACCT GGACCTGACC TACGTGACCG AGCGGATCAT 876
RBL004.3 (RNA) GCGGCGGTAC ACCAGGGACG GCTTCGACCT GGACCTGACC TACGTGACCG AGCGGATCAT 900
RBL004.1 (prot.) K R R Y T R D G F D L D L T Y V T E R I 274
RBL004.3 (prot.) K R R Y T R D G F D L D L T Y V T E R I 282
                                                                                              antigen
RBL004.1 (RNA) TGCCATGAGC TTCCCCAGCA GCGGCAGACA GAGCTTCTAC CGGAACCCCA TCAAAGAAGT 936
RBL004.3 (RNA) TGCCATGAGC TTCCCCAGCA GCGGCAGACA GAGCTTCTAC CGGAACCCCA TCAAAGAAGT 960
RBL004.1 (prot.) I A M S F P S S G R Q S F Y R N P I K E 294
RBL004.3 (prot.) I A M S F P S S G R Q S F Y R N P I K E 302
                                                                                              antigen
RBL004.1 (RNA) GGTGCGGTTC CTGGACAAGA AGCACCGGAA CCACTACCGG GTGTACAACC TGTGCAGCGA 996
RBL004.3 (RNA) GGTGCGGTTC CTGGACAAGA AGCACCGGAA CCACTACCGG GTGTACAACC TGTGCAGCGA 1020
RBL004.1 (prot.) V V R F L D K K H R N H Y R V Y N L C S 314
RBL004.3 (prot.) V V R F L D K K H R N H Y R V Y N L C S 322
                                                                                              antigen
RBL004.1 (RNA) GCGGGCCTAC GACCCCAAGC ACTTCCACAA CCGGGTGGTG CGGATCATGA TCGACGACCA 1056
RBL004.3 (RNA) GCGGGCCTAC GACCCCAAGC ACTTCCACAA CCGGGTGGTG CGGATCATGA TCGACGACCA 1080
RBL004.1 (prot.) E R A Y D P K H F H N R V V R I M I D D 334
RBL004.3 (prot.) E R A Y D P K H F H N R V V R I M I D D 342
                                                                                              antigen
RBL004.1 (RNA) CAACGTGCCC ACCCTGCACC AGATGGTGGT GTTCACCAAA GAAGTGAACG AGTGGATGGC 1116
RBL004.3 (RNA) CAACGTGCCC ACCCTGCACC AGATGGTGGT GTTCACCAAA GAAGTGAACG AGTGGATGGC 1140
RBL004.1 (prot.) H N V P T L H Q M V V F T K E V N E W M 354
RBL004.3 (prot.) H N V P T L H Q M V V F T K E V N E W M 362
                                                                                              antigen
RBL004.1 (RNA) CCAGGACCTG GAAAACATCG TGGCCATCCA CTGCAAGGGC GGCACCGACA GAACCGGCAC 1176
RBL004.3 (RNA) CCAGGACCTG GAAAACATCG TGGCCATCCA CTGCAAGGGC GGCACCGACA GAACCGGCAC 1200
RBL004.1 (prot.) A Q D L E N I V A I H C K G G T D R T G 374
RBL004.3 (prot.) A Q D L E N I V A I H C K G G T D R T G 382
                                                                                              antigen
RBL004.1 (RNA) CATGGTGTGC GCCTTTCTGA TCGCCAGCGA GATCTGTAGC ACCGCCAAAG AGTCCCTGTA 1236
RBL004.3 (RNA) CATGGTGTGC GCCTTTCTGA TCGCCAGCGA GATCTGTAGC ACCGCCAAAG AGTCCCTGTA 1260
RBL004.1 (prot.) T M V C A F L I A S E I C S T A K E S L 394
RBL004.3 (prot.) T M V C A F L I A S E I C S T A K E S L 402
                                                                                              antigen
RBL004.1 (RNA) CTACTTCGGC GAGCGGAGAA CCGACAAGAC CCACAGCGAG AAGTTCCAGG GCGTGGAGAC 1296
RBL004.3 (RNA) CTACTTCGGC GAGCGGAGAA CCGACAAGAC CCACAGCGAG AAGTTCCAGG GCGTGGAGAC 1320
RBL004.1 (prot.) Y Y F G E R R T D K T H S E K F Q G V E 414
RBL004.3 (prot.) Y Y F G E R R T D K T H S E K F Q G V E 422
                                                                                              antigen
RBL004.1 (RNA) ACCCAGCCAG AAAAGATATG TGGCTTACTT CGCCCAGGTG AAGCACCTGT ACAACTGGAA 1356
RBL004.3 (RNA) ACCCAGCCAG AAAAGATATG TGGCTTACTT CGCCCAGGTG AAGCACCTGT ACAACTGGAA 1380
RBL004.1 (prot.) T P S Q K R Y V A Y F A Q V K H L Y N W 434
RBL004.3 (prot.) T P S Q K R Y V A Y F A Q V K H L Y N W 442
                                                                                              antigen
RBL004.1 (RNA) CCTGCCCCCC AGACGGATTC TGTTCATCAA GCACTTCATC ATCTACAGCA TCCCCAGATA 1416
RBL004.3 (RNA) CCTGCCCCCC AGACGGATTC TGTTCATCAA GCACTTCATC ATCTACAGCA TCCCCAGATA 1440
RBL004.1 (prot.) N L P P R R I L F I K H F I I Y S I P R 454
RBL004.3 (prot.) N L P P R R I L F I K H F I I Y S I P R 462
                                                                                              antigen
RBL004.1 (RNA) CGTGCGGGAC CTGAAGATCC AGATCGAGAT GGAAAAGAAA GTGGTGTTCA GCACCATCTC 1476
RBL004.3 (RNA) CGTGCGGGAC CTGAAGATCC AGATCGAGAT GGAAAAGAAA GTGGTGTTCA GCACCATCTC 1500
RBL004.1 (prot.) Y V R D L K I Q I E M E K K V V F S T I 474
RBL004.3 (prot.) Y V R D L K I Q I E M E K K V V F S T I 482
                                                                                              antigen
RBL004.1 (RNA) CCTGGGCAAG TGCAGCGTGC TGGACAACAT CACCACCGAC AAGATCCTGA TCGACGTGTT 1536
RBL004.3 (RNA) CCTGGGCAAG TGCAGCGTGC TGGACAACAT CACCACCGAC AAGATCCTGA TCGACGTGTT 1560
RBL004.1 (prot.) S L G K C S V L D N I T T D K I L I D V 494
RBL004.3 (prot.) S L G K C S V L D N I T T D K I L I D V 502
                                                                                              antigen
RBL004.1 (RNA) CGACGGCCTG CCCCTGTACG ACGACGTGAA GGTGCAGTTC TTCTACAGCA ACCTGCCCAC 1596
RBL004.3 (RNA) CGACGGCCTG CCCCTGTACG ACGACGTGAA GGTGCAGTTC TTCTACAGCA ACCTGCCCAC 1620
RBL004.1 (prot.) F D G L P L Y D D V K V Q F F Y S N L P 514
RBL004.3 (prot.) F D G L P L Y D D V K V Q F F Y S N L P 522
                                                                                              antigen
RBL004.1 (RNA) CTACTACGAC AATTGCAGCT TCTACTTCTG GCTGCACACC AGCTTCATCG AGAACAACAG 1656
RBL004.3 (RNA) CTACTACGAC AATTGCAGCT TCTACTTCTG GCTGCACACC AGCTTCATCG AGAACAACAG 1680
RBL004.1 (prot.) T Y Y D N C S F Y F W L H T S F I E N N 534
RBL004.3 (prot.) T Y Y D N C S F Y F W L H T S F I E N N 542
                                                                                              antigen
RBL004.1 (RNA) GCTGTACCTG CCCAAGAACG AGCTGGACAA CCTGCACAAG CAGAAGGCCA GAAGAATCTA 1716
RBL004.3 (RNA) GCTGTACCTG CCCAAGAACG AGCTGGACAA CCTGCACAAG CAGAAGGCCA GAAGAATCTA 1740
RBL004.1 (prot.) R L Y L P K N E L D N L H K Q K A R R I 554
RBL004.3 (prot.) R L Y L P K N E L D N L H K Q K A R R I 562
                                                                                              antigen
RBL004.1 (RNA) CCCCAGCGAC TTCGCCGTGG AGATCCTGTT TGGCGAGAAG ATGACCAGCA GCGACGTGGT 1776
RBL004.3 (RNA) CCCCAGCGAC TTCGCCGTGG AGATCCTGTT TGGCGAGAAG ATGACCAGCA GCGACGTGGT 1800
RBL004.1 (prot.) Y P S D F A V E I L F G E K M T S S D V 574
RBL004.3 (prot.) Y P S D F A V E I L F G E K M T S S D V 582
                                      antigen ][ GS ][ P2P16
RBL004.1 (RNA) GGCCGGCAGC GACGGATCCC TGGGAGGCGG GGGAAGCGGC AAGAAGCAGT ACATCAAGGC 1836
RBL004.3 (RNA) GGCCGGCAGC GGAGGATCCG GTGGTGGCGG CAGCGGCGGC AAGAAGCAGT ACATCAAGGC 1860
                                  ********** * ****** ** ***** * ***** ********** **********
RBL004.1 (prot.) V A G S D G S L G G G G S G K K Q Y I K 594
RBL004.3 (prot.) V A G S G G S G G G G D G G K K Q Y I K 602
                                                            * * * *
                                                                                              P2P16
RBL004.1 (RNA) CAACAGCAAG TTCATCGGCA TCACCGAGCT GAAGAAGCTG GGAGGGGGCA AACGGGGAGG 1896
RBL004.3 (RNA) CAACAGCAAG TTCATCGGCA TCACCGAGCT GAAGAAGCTG GGAGGGGGCA AACGGGGAGG 1920
RBL004.1 (prot.) A N S K F I G I T E L K K L G G G K R G 614
RBL004.3 (prot.) A N S K F I G I T E L K K L G G G K R G 622
                                                                                              P2P16
RBL004.1 (RNA) CGGCAAAAAG ATGACCAACA GCGTGGACGA CGCCCTGATC AACAGCACCA AGATCTACAG 1956
RBL004.3 (RNA) CGGCAAAAAG ATGACCAACA GCGTGGACGA CGCCCTGATC AACAGCACCA AGATCTACAG 1980
RBL004.1 (prot.) G G K K M T N S V D D A L I N S T K I Y 634
RBL004.3 (prot.) G G K K M T N S V D D A L I N S T K I Y 642
                                                                                    P2P16 ][ GS
RBL004.1 (RNA) CTACTTCCCC AGCGTGATCA GCAAAGTGAA CCAGGGCGCT CAGGGCAAGA AACTGGGCTC 2016
RBL004.3 (RNA) CTACTTCCCC AGCGTGATCA GCAAAGTGAA CCAGGGCGCT CAGGGCAAGA AACTGGGCTC 2040
RBL004.1 (prot.) S Y F P S V I S K V N Q G A Q G K K L G 654
RBL004.3 (prot.) S Y F P S V I S K V N Q G A Q G K K L G 662
                                                                    GS ][ MITD
RBL004.1 (RNA) TAGCGGAGGG GGAGGCTCTC CTGGGGGGGG ATCTAGCATC GTGGGAATTG TGGCAGGACT 2076
RBL004.3 (RNA) TAGCGGAGGG GGAGGCTCTC CTGGCGGGGG ATCTAGCATC GTGGGAATTG TGGCAGGACT 2100
RBL004.1 (prot.) S S G G G G S P G G G S S I V G I V A G 674
RBL004.3 (prot.) S S G G G G S P G G G S S I V G I V A G 682
                                                                                              MITD
RBL004.1 (RNA) GGCAGTGCTG GCCGTGGTGG TGATCGGAGC CGTGGTGGCT ACCGTGATGT GCAGACGGAA 2136
RBL004.3 (RNA) GGCAGTGCTG GCCGTGGTGG TGATCGGAGC CGTGGTGGCT ACCGTGATGT GCAGACGGAA 2160
RBL004.1 (prot.) L A V L A V V V I G A V V A T V M C R R 694
RBL004.3 (prot.) L A V L A V V V I G A V V A T V M C R R 702
                                                                                              MITD
RBL004.1 (RNA) GTCCAGCGGA GGCAAGGGCG GCAGCTACAG CCAGGCCGCC AGCTCTGATA GCGCCCAGGG 2196
RBL004.3 (RNA) GTCCAGCGGA GGCAAGGGCG GCAGCTACAG CCAGGCCGCC AGCTCTGATA GCGCCCAGGG 2220
RBL004.1 (prot.) K S S G G K G G S Y S Q A A S S D S A Q 714
RBL004.3 (prot.) K S S G G K G G S Y S Q A A S S D S A Q 722
                                                          MITD ][ 3′-UTR
RBL004.1 (RNA) CAGCGACGTG TCACTGACAG CC TGACT CGAGAGCTCG CTTTCTTGCT GTCCAATTTC 2253
RBL004.3 (RNA) CAGCGACGTG TCACTGACAG CCTAGTAACT CGAGCTGGTA CTGCATGCAC GCAATGCTAG 2280
                                                                                  *** * ****** *** **** ****** **
RBL004.1 (prot.) G S D V S L T A 722
RBL004.3 (prot.) G S D V S L T A 730
                                                                                              3′-UTR
RBL004.1 (RNA) TATTAAA GGTTCCTTT GTTCCCTAAG TCCAACTACT AAACTGGGGG ATATTATGAA 2309
RBL004.3 (RNA) CTGCCCCTTT CCCGTCCTGG GTACCCCGAG TCTCCCCCGA CCTCGGGTCC CAGGTATGCT 2340
                                  ********** **** ** * ** *** **** *** * *** **** **
                                                                                              3′-UTR
RBL004.1 (RNA) GGGCCTTGAG CATCTGGATT CTGCCTAATA AAAAACATTT ATTTTCATTG CTGCGTCGAG 2369
RBL004.3 (RNA) CCCACCTCCA CCTGCCCCAC TCACCACCTC TGCTAGTTCC AGACACCTCC CAAGCACGCA 2400
                                  **** * *** * ******* *** *** * **** ** ** **** * ** ***** **
                                                                                              3′-UTR
RBL004.1 (RNA) AGCTCGCTTT CTTGCTGTCC AATTTCT A TTAAAGGTTC CTTT 2411
RBL004.3 (RNA) GCAATGCAGC TCAAAACGCT TAGCCTAGCC ACACCCCCAC GGGAAACAGC AGTGATTAAC 2460
                                  ***** *** ******** * * ******** ********** *** ***** ** *******
                                                                                              3′-UTR
RBL004.1 (RNA) GTTCCCTAA GTCCAACTAC TAAACTGGGG GATATTATGA AGGGCCTTGA GCATCTGGAT 2470
RBL004.3 (RNA) CTTTAGCAAT AAACGAAAGT TTAACTAAGC TATACTAACC CCAGGGTTGG TCAATTTCGT 2520
                                  ** ** * * *** * **** * ** * * * *** *** ** * * ** ***
                                                                        3′-UTR ][ poly(A)
RBL004.1 (RNA) TCTGCCTAAT AAAAAACATT TATTTTCATT GCTGCGTCGA GAGCTAAAAA AAAAAAAAAA 2530
RBL004.3 (RNA) GCCAGCCACA CCGAGACCTG GTCCAGAGTC GCTAGCCGCG TCGCTAAAAA AAAAAAAAAA 2580
                                  * *** * ** *** * * * ******** * ******* **
RBL004.1 (RNA) AAAAAAAAAA AAAAAAAAAA AAAAAAAAAA AAAAAAAAAA AAAAAAAAAA AAAAAAAAAA 2590
RBL004.3 (RNA) AAAAAAAAAA AAAAAGCATA TGACT AAAAA AAAAAAAAAA AAAAAAAAAA 2630
                                                                  ** * ** ******* *****
                                                                                poly(A) ]
RBL004.1 (RNA) AAAAAAAAAA AAAAAAAAAA AAAAAAAAAA AAAAAAAAAA AAAAA 2635
RBL004.3 (RNA) AAAAAAAAAA AAAAAAAAAA AAAAAAAAAA AAAAAAAAAA AAAAA 2675
```

### B. Exemplary Manufacturing Processes

Individual RNA molecules can be produced by methods known in the art. For example, in some embodiments, single-stranded RNAs can be produced by in vitro transcription, for example, using a DNA template. A plasmid DNA used as a template for in vitro transcription to generate an RNA molecule described herein is also within the scope of the present disclosure.

A DNA template is used for in vitro RNA synthesis in the presence of an appropriate RNA polymerase (e.g., a recombinant RNA-polymerase such as a T7 RNA-polymerase) with ribonucleotide triphosphates (e.g., ATP, CTP, GTP, UTP). In some embodiments, RNA molecules (e.g., ones described herein) can be synthesized in the presence of modified ribonucleotide triphosphates. By way of example only, in some embodiments, N1-methylpseudouridine triphosphate (^{m1}ΨTP) can be used to replace uridine triphosphate (UTP). As will be clear to those skilled in the art, during in vitro transcription, an RNA polymerase (e.g., as described and/or utilized herein) typically traverses at least a portion of a single-stranded DNA template in the 3′→5′ direction to produce a single-stranded complementary RNA in the 5′→3′ direction.

In some embodiments where an RNA molecule comprises a polyA tail, one of those skill in the art will appreciate that such a polyA tail may be encoded in a DNA template, e.g., by using an appropriately tailed PCR primer, or it can be added to an RNA molecule after in vitro transcription, e.g., by enzymatic treatment (e.g., using a poly(A) polymerase such as an E. coli Poly(A) polymerase).

In some embodiments, those skilled in the art will appreciate that addition of a 5′ cap to an RNA (e.g., mRNA) can facilitate recognition and attachment of the RNA to a ribosome to initiate translation and enhances translation efficiency. Those skilled in the art will also appreciate that a 5′ cap can also protect an RNA product from 5′ exonuclease mediated degradation and thus increases half-life. Methods for capping are known in the art; one of ordinary skill in the art will appreciate that in some embodiments, capping may be performed after in vitro transcription in the presence of a capping system (e.g., an enzyme-based capping system such as, e.g., capping enzymes of vaccinia virus). In some embodiments, a cap may be introduced during in vitro transcription, along with a plurality of ribonucleotide triphosphates such that a cap is incorporated into an RNA molecule ssRNA during transcription (also known as co-transcriptional capping).

Following RNA transcription, a DNA template is digested. In some embodiments, digestion can be achieved with the use of DNase I under appropriate conditions.

In some embodiments, RNA molecules can be purified after in vitro transcription reaction, for example, to remove components utilized or formed in the course of the production, like, e.g., proteins, DNA fragments, and/or or nucleotides. Various nucleic acid purifications that are known in the art can be used in accordance with the present disclosure. In some embodiments, RNA molecules may be purified using magnetic bead-based purification, which in some embodiments may be or comprise magnetic bead-based chromatography. In some embodiments, RNA molecules may be purified using hydrophobic interaction chromatography (HIC) followed by diafiltration.

In some embodiments, dsRNA may be obtained as side product during in vitro transcription. In some such embodiments, a second purification step may be performed to remove dsRNA contamination. For example, in some embodiments, cellulose materials (e.g., microcrystalline cellulose) may be used to remove dsRNA contamination, for examples in some embodiments in a chromatographic format. In some embodiments, cellulose materials (e.g., microcrystalline cellulose) can be pretreated to inactivate potential RNase contamination, for example in some embodiments by autoclaving followed by incubation with aqueous basic solution, e.g., NaOH. In some embodiments, cellulose materials may be used to purify RNA molecules according to methods described in WO 2017/182524, the entire content of which is incorporated herein by reference.

In some embodiments, a batch of ssRNAs may be further processed by one or more steps of filtration and/or concentration. For example, in some embodiments, RNA molecules, for example, after removal of dsRNA contamination, may be further subject to diafiltration, for example, to adjust the concentration of ssRNAs to a desirable RNA concentration and/or to exchange buffer to a drug substance buffer.

In some embodiments, RNA molecules may be processed through 0.2 μm filtration before they are filled into appropriate containers.

In some embodiments, RNA quality control may be performed and/or monitored at any time during production process of RNA molecules and/or compositions comprising the same. For example, in some embodiments, RNA quality control parameters may be assessed and/or monitored after each or certain steps of RNA molecules manufacturing process, e.g., after in vitro transcription, and/or each purification step.

In some embodiments, one or more assessments may be utilized during manufacture, or other preparation or use of RNA molecules (e.g., as a release test).

In some embodiments, one or more quality control parameters may be assessed to determine whether RNA molecules described herein meet or exceed pre-determined acceptance criteria (e.g., for subsequent formulation and/or release for distribution). In some embodiments, such quality control parameters may include, but are not limited to RNA integrity, RNA concentration, residual DNA template and/or residual dsRNA. Methods for assessing RNA quality are known in the art.

In some embodiments, a batch of RNA molecules may be assessed for one or more features to determine next action step(s). For example, a batch of single stranded RNAs can be designated for one or more further steps of manufacturing and/or formulation and/or distribution if RNA quality assessment indicates that such a batch of single stranded RNAs meet or exceed the acceptance criteria. Otherwise, an alternative action can be taken (e.g., discarding the batch) if such a batch of single stranded RNAs does not meet or exceed the acceptance criteria.

In some embodiments, a batch of RNA molecules with exemplary assessment results can be utilized for one or more further steps of manufacturing and/or formulation and/or distribution.

### III. RNA Delivery Technologies

Provided pharmaceutical compositions (e.g., one or more molecules of RNA encoding one or more TAAs) may be delivered for therapeutic applications described herein using any appropriate methods known in the art, including, e.g., delivery as naked RNAs, or delivery mediated by viral and/or non-viral vectors, polymer-based vectors, lipid-based vectors, nanoparticles (e.g., lipid nanoparticles, polymeric nanoparticles, lipid-polymer hybrid nanoparticles, etc.), and/or peptide-based vectors. See, e.g., Wadhwa et al. “Opportunities and Challenges in the Delivery of mRNA-Based Vaccines” Pharmaceutics (2020) 102 (27 pages), the content of which is incorporated herein by reference, for information on various approaches that may be useful for delivery RNA molecules described herein.

In some embodiments, one or more RNA molecules can be formulated with lipid particles for delivery (e.g., in some embodiments by intravenous injection).

In some embodiments, lipid particles can be designed to protect RNA molecules (e.g., mRNA) from extracellular RNases and/or engineered for systemic delivery of the RNA to target cells (e.g., dendritic cells). In some embodiments, such lipid particles may be particularly useful to deliver RNA molecules (e.g., mRNA) when RNA molecules are intravenously administered to a subject in need thereof.

In some embodiments, lipid particles comprise liposomes. In some embodiments, lipid particles comprise cationic liposomes

In some embodiments, lipid particles comprise lipid nanoparticles.

In some embodiments, lipid particles comprise lipoplexes.

In some embodiments, lipid particles comprise N,N,N trimethyl-2-3-dioleyloxy-1-propanaminium chloride (DOTMA), 1,2-dioleoyl-sn-glycero-3-phosphoethanolamine phospholipid (DOPE), or both. In some embodiments, lipid particles comprise at least one ionizable aminolipid. In some embodiments, lipid particles comprise at least one ionizable aminolipid and a helper lipid. In some embodiments, a helper lipid is or comprises a phospholipid. In some embodiments, a helper lipid is or comprises a sterol. In some embodiments, lipid particles comprises at least one polymer-conjugated lipid.

RNA lipoplex particles: In some embodiments, RNA molecules described herein may be delivered by liposomal formulations. In some embodiments, negatively charged RNA molecules described herein are complexed with cationic liposomes to form RNA lipoplex particles. In some embodiments, RNA molecules described herein are embedded in a (phospho)lipid bilayer structure within an RNA lipoplex particle. In some embodiments, cationic liposomes can comprise a cationic lipid or an ionizable aminolipid (e.g., ones as described herein) and optionally an additional or helper lipid (e.g., at least one neutral lipid as described herein) to form injectable particle formulations.

In some embodiments, RNA lipoplex particles may be prepared by mixing liposomes with RNA molecules described herein. In some embodiments, liposomes may be obtained by injecting a solution of lipids in ethanol into water or a suitable aqueous phase. In some embodiments, cationic liposomes are stabilized in an aqueous formulation, e.g., as described in WO 2016/046060, the entire content of which is incorporated herein by reference for the purposes described herein. In some embodiments, cationic liposomes may be produced by a method, e.g., as described in WO 2019/077053, the entire content of which is incorporated herein by reference for the purposes described herein.

In some embodiments, spleen targeting RNA lipoplex particles that are useful for delivering RNA molecules described herein are described in WO 2013/143683, the entire content of which is incorporated herein by reference for the purposes described herein. In some embodiments, RNA molecules and positively charged liposomes are mixed such that cationic lipids and RNA are present at a charge ratio of 1.3:2. Such charge ratio is determined to effectively target RNA to the spleen.

In some embodiments, an RNA lipoplex particle comprises a cationic lipid or an ionizable aminolipid (e.g., ones described herein) and an RNA molecule described herein. In some embodiments, such an RNA lipoplex particle may further comprise an additional or helper lipid (e.g., ones described herein). Without wishing to be bound by theory, electrostatic interactions between positively charged liposomes and negatively charged RNA results in complexation and spontaneous formation of RNA lipoplex particles.

In some embodiments where a cationic lipid or an ionizable aminolipid (e.g., ones described herein) and a helper lipid are used, such a cationic lipid or an ionizable aminolipid and such a helper lipid may be present in a molar ratio of 2:1. In some embodiments, a cationic lipid or an ionizable aminolipid may be or comprise DOTMA. In some embodiments, a helper lipid may be or comprise a neutral lipid. In some embodiments, a neutral lipid may be or comprise DOPE.

In some embodiments, RNA lipoplex particles are nanoparticles. In some embodiments, RNA lipoplex nanoparticles can have a particle size (e.g., Z-average) of about 100 nm to 1000 nm or about 200 nm to 900 nm or about 200 nm to 800 nm, or about 250 nm to about 700 nm.

RNA lipid nanoparticles: In some embodiments, RNA molecules described herein may be delivered by lipid nanoparticle formulations. In some embodiments, RNA lipid nanoparticles may be prepared by mixing lipids with RNA molecules described herein. In some embodiments, at least a portion of RNA molecules are encapsulated by lipid nanoparticles. In some embodiments, at least 90% or higher (including, e.g., at least 95%, 96%, 97%, 98%, 99%, or higher) of RNA molecules are encapsulated by lipid nanoparticles.

In various embodiments, lipid nanoparticles can have an average size (e.g., Z-average) of about 100 nm to 1000 nm, or about 200 nm to 900 nm, or about 200 nm to 800 nm, or about 250 nm to about 700 nm. In some embodiments, lipid nanoparticles can have a particle size (e.g., Z-average) of about 30 nm to about 200 nm, or about 30 nm to about 150 nm, about 40 nm to about 150 nm, about 50 nm to about 150 nm, about 60 nm to about 130 nm, about 70 nm to about 110 nm, about 70 nm to about 100 nm, about 80 nm to about 100 nm, about 90 nm to about 100 nm, about 70 to about 90 nm, about 80 nm to about 90 nm, or about 70 nm to about 80 nm. In some embodiments, an average size of lipid nanoparticles is determined by measuring the particle diameter.

In certain embodiments, RNA molecules (e.g., mRNAs), when present in provided lipid nanoparticles, are resistant in aqueous solution to degradation with a nuclease.

In some embodiments, lipid nanoparticles are cationic lipid nanoparticles comprising one or more cationic lipids (e.g., ones described herein). In some embodiments, cationic lipid nanoparticles may comprise at least one cationic lipid, at least one polymer-conjugated lipid, and at least one helper lipid (e.g., at least one neutral lipid).

### 1. Helper Lipids

In some embodiments, a lipid particle for delivery of RNA molecules described herein comprises at least one helper lipid, which may be a neutral lipid, a positively charged lipid, or a negatively charged lipid. In some embodiments, a helper lipid is a lipid that are useful for increasing the effectiveness of delivery of lipid-based particles such as cationic lipid-based particles to a target cell. In some embodiments, a helper lipid may be or comprise a structural lipid with its concentration chosen to optimize particle size, stability, and/or encapsulation.

In some embodiments, a lipid particle for delivery of RNA molecules described herein comprises a neutral helper lipid. Examples of such neutral helper lipids include, but are not limited to phosphotidylcholines such as 1,2-distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC), phophatidylethanolamines such as 1,2-dioleoyl-sn-glycero-3-phosphoethanolamine (DOPE), sphingomyelins (SM), ceramides, cholesterol, steroids such as sterols and their derivatives. Neutral lipids may be synthetic or naturally derived. Other neutral helper lipids that are known in the art, e.g., as described in WO 2017/075531 and WO 2018/081480, the entire contents of each of which are incorporated herein by reference for the purposes described herein, can also be used in lipid particles described herein. In some embodiments, a lipid particle for delivery of RNA molecules described herein comprises DSPC and/or cholesterol.

In some embodiments, a lipid particle for delivery of RNA molecules described herein comprises at least one helper lipids (e.g., ones described herein). In some such embodiments, a lipid particle may comprise DOPE.

### 2. Cationic Lipids

In some embodiments, a lipid particle for delivery of RNA molecules described herein comprises a cationic lipid. A cationic lipid is typically a lipid having a net positive charge, for example in some embodiments at a certain pH. In some embodiments, a cationic lipid may comprise one or more amine group(s) which bear a positive charge. In some embodiments, a cationic lipid may comprise a cationic, meaning positively charged, headgroup. In some embodiments, a cationic lipid may have a hydrophobic domain (e.g., one or more domains of a neutral lipid or an anionic lipid) provided that the cationic lipid has a net positive charge. In some embodiments, a cationic lipid comprises a polar headgroup, which in some embodiments may comprise one or more amine derivatives such as primary, secondary, and/or tertiary amines, quaternary ammonium, various combinations of amines, amidinium salts, or guanidine and/or imidazole groups as well as pyridinium, piperizine and amino acid headgroups such as lysine, arginine, ornithine and/or tryptophan. In some embodiments, a polar headgroup of a cationic lipid comprises one or more amine derivatives. In some embodiments, a polar headgroup of a cationic lipid comprises a quaternary ammonium. In some embodiments, a headgroup of a cationic lipid may comprise multiple cationic charges. In some embodiments, a headgroup of a cationic lipid comprises one cationic charge. Examples of monocationic lipids include, but are not limited to 1,2-dimyristoyl-sn-glycero-3-ethylphosphocholine (DMEPC), 1,2-di-O-octadecenyl-3-trimethylammonium propane (DOTMA) and/or 1,2-dioleoyl-3-trimethylammonium propane (DOTAP), 1,2-dimyristoyl-3-trimethylammonium propane (DMTAP), 2,3-di(tetradecoxy)propyl-(2-hydroxyethyl)-dimethylazanium bromide (DMRIE), didodecyl(dimethyl)azanium bromide (DDAB), 1,2-dioleyloxypropyl-3-dimethyl-hydroxyethyl ammonium bromide (DORIE), 3P—[N—(N\N′-dimethylamino-ethane)carbamoyl]cholesterol (DC-Choi) and/or dioleyl ether phosphatidylcholine (DOEPC).

In some embodiments, a positively charged lipid structure described herein may also include one or more other components that may be typically used in the formation of vesicles (e.g. for stabilization). Examples of such other components includes, without being limited thereto, fatty alcohols, fatty acids, and/or cholesterol esters or any other pharmaceutically acceptable excipients which may affect the surface charge, the membrane fluidity and assist in the incorporation of the lipid into the lipid assembly. Examples of sterols include cholesterol, cholesteryl hemisuccinate, cholesteryl sulfate, or any other derivatives of cholesterol. In some embodiments, a one cationic lipid comprises DMEPC and/or DOTMA. In some embodiments, a cationic lipid comprises DOTMA.

In some embodiments, a cationic lipid is ionizable such that it can exist in a positively charged form or neutral form depending on pH. For example, in some embodiments, a cationic lipid is an ionizable aminolipid. Such ionization of a cationic lipid can affect the surface charge of the lipid particle under different pH conditions, which in some embodiments may influence plasma protein absorption, blood clearance, and/or tissue distribution as well as the ability to form endosomolytic non-bilayer structures. Accordingly, in some embodiments, a cationic lipid may be or comprise a pH responsive lipid. In some embodiments a pH responsive lipid is a fatty acid derivative or other amphiphilic compound which is capable of forming a lyotropic lipid phase, and which has a pKa value between pH 5 and pH 7.5. This means that the lipid is uncharged at a pH above the pKa value and positively charged below the pKa value. In some embodiments, a pH responsive lipid may be used in addition to or instead of a cationic lipid for example by binding one or more RNA molecules to a lipid or lipid mixture at low pH. pH responsive lipids include, but are not limited to, 1,2-dioleyloxy-3-dimethylamino-propane (DODMA).

In some embodiments, a lipid particle may comprise one or more cationic lipids as described in WO 2017/075531 (e.g., as presented in Tables 1 and 3 therein) and WO 2018/081480 (e.g., as presented in Tables 1-4 therein), the entire contents of each of which are incorporated herein by reference for the purposes described herein.

In some embodiments, a cationic lipid that may be useful in accordance with the present disclosure is an amino lipid comprising a titratable tertiary amino head group linked via ester bonds to at least two saturated alkyl chains, which ester bonds can be hydrolyzed easily to facilitate fast degradation and/or excretion via renal pathways. In some embodiments, such an amino lipid has an apparent pKₐ of about 6.0-6.5 (e.g., in one embodiment with an apparent pKₐ of approximately 6.25), resulting in an essentially fully positively charged molecule at an acidic pH (e.g., pH 5). In some embodiments, such an amino lipid, when incorporated in a lipid particle, can confer distinct physicochemical properties that regulate particle formation, cellular uptake, fusogenicity and/or endosomal release of RNA molecules. In some embodiments, introduction of an aqueous RNA solution to a lipid mixture comprising such an amino lipid at pH 4.0 can lead to an electrostatic interaction between the negatively charged RNA backbone and the positively charged cationic lipid. Without wishing to be bound by any particular theory, such electrostatic interaction leads to particle formation coincident with efficient encapsulation of RNA drug substance. After RNA encapsulation, adjustment of the pH of the medium surrounding the resulting lipid nanoparticles to a more neutral pH (e.g., pH 7.4) results in neutralization of the surface charge of the lipid nanoparticles. When all other variables are held constant, such charge-neutral particles display longer in vivo circulation lifetimes and better delivery to hepatocytes compared to charged particles, which are rapidly cleared by the reticuloendothelial system. Upon endosomal uptake, the low pH of the endosome renders lipid nanoparticle comprising such an amino lipid fusogenic and allows the release of the RNA into the cytosol of the target cell.

Cationic lipids may be used alone or in combination with neutral lipids, e.g., cholesterol and/or neutral phospholipids, or in combination with other known lipid assembly components.

### 3. Polymer-Conjugated Lipids

In some embodiments, a lipid nanoparticle for use in delivery of RNA molecules described herein may comprise at least one polymer-conjugated lipid. A polymer-conjugated lipid is typically a molecule comprising a lipid portion and a polymer portion conjugated thereto.

In some embodiments, a polymer-conjugated lipid is a PEG-conjugated lipid. In some embodiments, a PEG-conjugated lipid is designed to sterically stabilize a lipid particle by forming a protective hydrophilic layer that shields the hydrophobic lipid layer. In some embodiments, a PEG-conjugated lipid can reduce its association with serum proteins and/or the resulting uptake by the reticuloendothelial system when such lipid particles are administered in vivo.

Various PEG-conjugated lipids are known in the art and include, but are not limited to pegylated diacylglycerol (PEG-DAG) such as 1-(monomethoxy-polyethyleneglycol)-2,3-dimyristoylglycerol (PEG-DMG), a pegylated phosphatidylethanolamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω methoxy(polyethoxy)ethyl)carbamate, and the like.

Certain PEG-conjugated lipids (also known as PEGylated lipids) were clinically approved with safety demonstrated in clinical trials. PEG-conjugated lipids are known to affect cellular uptake, a prerequisite to endosomal localization and payload delivery. The pharmacology of encapsulated nucleic acid can be controlled in a predictable manner by modulating the alkyl chain length of a PEG-lipid anchor. In some embodiments, PEG-conjugated lipids may be designed and/or selected based on reasonable solubility characteristics and/or its molecular weight to effectively perform the function of a steric barrier. For example, in some embodiments, a PEGylated lipid does not show appreciable surfactant or permeability enhancing or disturbing effects on biological membranes. In some embodiments, PEG in such a PEG-conjugated lipid can be linked to diacyl lipid anchors with a biodegradable amide bond, thereby facilitating fast degradation and/or excretion. In some embodiments, a LNP comprising a PEG-conjugated lipid retain a full complement of a PEGylated lipid. In the blood compartment, such a PEGylated lipid dissociates from the particle over time, revealing a more fusogenic particle that is more readily taken up by cells, ultimately leading to release of the RNA payload.

In some embodiments, a lipid particle (e.g., a lipid nanoparticle) may comprise one or more PEG-conjugated lipids or pegylated lipids as described in WO 2017/075531 and WO 2018/081480, the entire contents of each of which are incorporated herein by reference for the purposes described herein. For example, in some embodiments, a PEG-conjugated lipid that may be useful in accordance with the present disclosure can have a structure

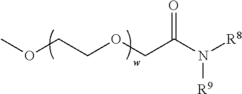

as described in WO 2017/075531, or a pharmaceutically acceptable salt, tautomer or stereoisomer thereof, wherein: R₈ and R₉ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some embodiments, R8 and R9 are each independently straight, saturated alkyl chains containing from 12 to 16 carbon atoms. In some embodiments, w has a mean value ranging from 43 to 53. In other embodiments, the average w is about 45.

In some embodiments, lipids that form lipid nanoparticles described herein comprise: a polymer-conjugated lipid; a cationic lipid; and a helper neutral lipid. In some such embodiments, total polymer-conjugated lipid may be present in about 0.5-5 mol %, about 0.7-3.5 mol %, about 1-2.5 mol %, about 1.5-2 mol %, or about 1.5-1.8 mol % of the total lipids. In some embodiments, total polymer-conjugated lipid may be present in about 1-2.5 mol % of the total lipids. In some embodiments, the molar ratio of total cationic lipid to total polymer-conjugated lipid (e.g., PEG-conjugated lipid) may be about 100:1 to about 20:1, or about 50:1 to about 20:1, or about 40:1 to about 20:1, or about 35:1 to about 25:1.

In some embodiments involving a polymer-conjugated lipid, a cationic lipid, and a helper neutral lipid in lipid nanoparticles described herein, total cationic lipid is present in about 35-65 mol %, about 40-60 mol %, about 41-49 mol %, about 41-48 mol %, about 42-48 mol %, about 43-48 mol %, about 44-48 mol %, about 45-48 mol %, about 46-48 mol %, or about 47.2-47.8 mol % of the total lipids.

In some embodiments involving a polymer-conjugated lipid, a cationic lipid, and a helper neutral lipid in lipid nanoparticles described herein, total neutral lipid is present in about 35-65 mol %, about 40-60 mol %, about 45-55 mol %, or about 47-52 mol % of the total lipids. In some embodiments, total neutral lipid is present in 35-65 mol % of the total lipids. In some embodiments, total non-steroid neutral lipid (e.g., DPSC) is present in about 5-15 mol %, about 7-13 mol %, or 9-11 mol % of the total lipids. In some embodiments, total non-steroid neutral lipid is present in about 9.5, 10 or 10.5 mol % of the total lipids. In some embodiments, the molar ratio of the total cationic lipid to the non-steroid neutral lipid ranges from about 4.1:1.0 to about 4.9:1.0, from about 4.5:1.0 to about 4.8:1.0, or from about 4.7:1.0 to 4.8:1.0. In some embodiments, total steroid neutral lipid (e.g., cholesterol) is present in about 35-50 mol %, about 39-49 mol %, about 40-46 mol %, about 40-44 mol %, or about 40-42 mol % of the total lipids. In certain embodiments, total steroid neutral lipid (e.g., cholesterol) is present in about 39, 40, 41, 42, 43, 44, 45, or 46 mol % of the total lipids. In certain embodiments, the molar ratio of total cationic lipid to total steroid neutral lipid is about 1.5:1 to 1:1.2, or about 1.2:1 to 1:1.2.

In some embodiments, a lipid composition comprising a cationic lipid, a polymer-conjugated lipid, and a neutral lipid can have individual lipids present in certain molar percents of the total lipids, or in certain molar ratios (relative to each other) as described in WO 2018/081480, the entire contents of each of which are incorporated herein by reference for the purposes described herein.

### IV. Provided Pharmaceutical Compositions

The present disclosure provides, among other things, pharmaceutical compositions for delivering antigens (e.g., TAA) to a patient. In some embodiments, a pharmaceutical composition comprises one or more RNA molecules encoding a NY-ESO-1 antigen, a MAGE-A3 antigen, a tyrosinase antigen, a TPTE antigen, or a combination thereof; and lipid particles (e.g., lipoplexes or lipid nanoparticles). In some embodiments, a pharmaceutical composition comprises one or more RNA molecules collectively encoding a NY-ESO-1 antigen, a MAGE-A3 antigen, a tyrosinase antigen, and a TPTE antigen; and lipid particles (e.g., lipoplexes or lipid nanoparticles). In some embodiments, a pharmaceutical composition comprises at least four populations of RNA-lipid particles (e.g., lipoplexes or lipid nanoparticles), wherein each RNA-lipid particle comprises an RNA molecule and a lipid particle, and wherein the RNA molecules of each of the four RNA lipid particles is different, e.g. each RNA encodes a distinct TAA as described herein.

In some embodiments, one or more RNA molecules may be formulated with lipid nanoparticles (e.g., ones described herein) for administration to a patient. Accordingly, in some embodiments, a pharmaceutical composition comprises one or more RNA molecules encoding a NY-ESO-1 antigen, a MAGE-A3 antigen, a tyrosinase antigen, a TPTE antigen, or a combination thereof; and lipid particles (e.g., lipoplexes or lipid nanoparticles), wherein the one or more RNA molecules are encapsulated with the lipid particles (e.g., form an RNA-lipid particle). In some embodiments, an RNA-lipid particle is an RNA-lipoplex particle. In some embodiments, an RNA-lipid particle is an RNA-lipid nanoparticles.

In some embodiments, a pharmaceutical composition is administered as a monotherapy. In some embodiments, a pharmaceutical composition is administered as part of a combination therapy.

In some embodiments, a pharmaceutical composition comprises a first RNA molecule encoding a NY-ESO-1 antigen, a second RNA molecule encoding a MAGE-A3, a third RNA molecule encoding a tyrosinase antigen, and a fourth RNA molecule encoding a TPTE antigen, a first RNA molecule, a second RNA molecule, a third RNA molecule, and a fourth RNA molecule may be present in the pharmaceutical composition in about equimolar amounts (e.g., a molar ratio of about 1:1:1:1).

In some embodiments, a concentration of total RNA (e.g., a total concentration of all of the one or more RNA molecules) in a pharmaceutical composition described herein is of about 0.01 mg/mL to about 0.5 mg/mL, or about 0.05 mg/mL to about 0.1 mg/mL.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety).

In some embodiments, pharmaceutical compositions provided herein may be formulated with one or more pharmaceutically acceptable carriers or diluents as well as any other known adjuvants and excipients in accordance with conventional techniques such as those disclosed in Remington: The Science and Practice of Pharmacy 21st ed., Lippincott Williams & Wilkins, 2005 (incorporated herein by reference in its entirety).

Pharmaceutical compositions described herein can be administered by appropriate methods known in the art. As will be appreciated by a skilled artisan, the route and/or mode of administration may depend on a number of factors, including, e.g., but not limited to stability and/or pharmacokinetics and/or pharmacodynamics of pharmaceutical compositions described herein.

In some embodiments, pharmaceutical compositions described herein are formulated for parenteral administration, which includes modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In some embodiments, pharmaceutical compositions described herein are formulated for intravenous administration. In some embodiments, pharmaceutically acceptable carriers that may be useful for intravenous administration include sterile aqueous solutions or dispersions and sterile powders for preparation of sterile injectable solutions or dispersions.

In some particular embodiments, pharmaceutical compositions described herein are formulated for subcutaneous administration. In some particular embodiments, pharmaceutical compositions described herein are formulated for intramuscular administration.

Therapeutic compositions typically must be sterile and stable under the conditions of manufacture and storage. The composition can be formulated as a solution, dispersion, powder (e.g., lyophilized powder), microemulsion, lipid nanoparticles, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. In some embodiments, prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration.

In some embodiments, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Examples of suitable aqueous and nonaqueous carriers which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into pharmaceutical compositions described herein. In addition, prolonged absorption of the injectable pharmaceutical form may be brought about by the inclusion of agents which delay absorption such as aluminum monostearate and gelatin.

Formulations of pharmaceutical compositions described herein may be prepared by any method known or hereafter developed in the art of pharmacology. In general, such preparatory methods include the step of bringing active ingredient(s) into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of at least one RNA product produced using a system and/or method described herein.

Relative amounts of one or more RNA molecules encapsulated in LNPs, a pharmaceutically acceptable excipient, and/or any additional ingredients in a pharmaceutical composition can vary, depending upon the subject to be treated, target cells, diseases or disorders, and may also further depend upon the route by which the composition is to be administered.

In some embodiments, pharmaceutical compositions described herein are formulated into pharmaceutically acceptable dosage forms by conventional methods known to those of skill in the art. Actual dosage levels of the active ingredients (e.g., one or more RNA molecules encapsulated in lipid nanoparticles) in the pharmaceutical compositions described herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, a physician or veterinarian could start doses of active ingredients (e.g., one or more RNA molecules encapsulated in lipid nanoparticles) employed in the pharmaceutical composition at levels lower than that required in order to achieve the desired therapeutic effect and gradually increase the dosage until the desired effect is achieved. For example, exemplary doses as described Example 7 may be used in preparing pharmaceutically acceptable dosage forms.

In some embodiments, a pharmaceutical composition is formulated (e.g., for intravenous administration) to deliver a dose of about 7.2 μg to about 400 μg (or any of the subranges included therein) of total RNA, e.g., as described in Example 7.

In some embodiments, a pharmaceutical composition described herein may further comprise one or more additives, for example, in some embodiments that may enhance stability of such a composition under certain conditions. Examples of additives may include but are not limited to salts, buffer substances. preservatives. and carriers. For example, in some embodiments, a pharmaceutical composition may further comprise a cryoprotectant (e.g., sucrose) and/or an aqueous buffered solution, which may in some embodiments include one or more salts, including, e.g., alkali metal salts or alkaline earth metal salts such as, e.g., sodium salts, potassium salts, and/or calcium salts.

Exemplary formulations include, but are not limited to those listed in Table 3.

TABLE 3
Exemplary pharmaceutical composition formulations

| | Exemplary Formula 1 | Exemplary Formula 2 |
|---|---|---|
| Ingredient | Concentration (mg/mL) | Concentration (mg/mL) |
| RNA^{[1]} | 0.10 | 0.05 |
| DOTMA | 0.132 | 0.066 |
| DOPE | 0.074 | 0.037 |
| HEPES | 0.48 | 1.80 |
| EDTA | 0.007 | 0.90 |
| NaCl | 6.50 | 1.20 |
| Sucrose | — | 220.0 |
| Water For Injection | q.s. | q.s. |

^{[1]}RNA comprises a first RNA molecule encoding the NY-ESO-1 antigen, a second RNA molecule encoding a MAGE-A3 antigen, a third RNA molecule encoding a tyrosinase antigen, and a fourth RNA molecule encoding a TPTE antigen.

In some embodiments, a pharmaceutical composition described herein may further comprises one or more active agents in addition to RNA (e.g., one or more RNA molecules, e.g., one or more mRNA molecules. For example, in some embodiments, a pharmaceutical composition comprises an immune checkpoint inhibitor (also referred to as a “checkpoint inhibitor”). In some embodiments, an exemplary immune checkpoint inhibitor may be or comprise an immune checkpoint inhibitor indicated for treatment of cancer (e.g., melanoma), including, for example, but not limited to a PD-i inhibitor, a PDL-1 inhibitor, a CTLA4 inhibitor, LAG-3, or a combination thereof. In some embodiments, an immune checkpoint inhibitor is an antibody. Checkpoint inhibitors can include, for example, without limitation, those listed in Table 4.

TABLE 4
Exemplary immune checkpoint molecules and
inhibitors of those checkpoint molecules

| Checkpoint Molecule | Inhibitor |
|---|---|
| CTLA-4 | Ipilimumab |
| PD-1 | Cemiplimab |
| | Nivolumab |
| | Pembrolizumab |
| PD-L1 | Atezolizumab |
| | Avelumab |
| | Durvalumab |
| LAG-3 (CD223) | LAG525 (IMP701), REGN3767 (R3767), |
| | BI 754, 091, tebotelimab (MGD013), |
| | eftilagimod alpha (IMP321), FS118 |
| TIM-3 | MBG453, Sym023, TSR-022 |
| B7-H3, B7-H4 | MGC018, FPA150 |
| A2aR | EOS100850, AB928 |
| CD73 | CPI-006 |
| NKG2A | Monalizumab |
| PVRIG/PVRL2 | COM701 |
| CEACAM1 | CM24 |
| CEACAM 5/6 | NEO-201 |
| FAK | Defactinib |
| CCL2/CCR2 | PF-04136309 |
| LIF | MSC-1 |
| CD47/SIRPα | Hu5F9-G4 (5F9), ALX148, TTI-662, |
| | RRx-001 |
| CSF-1 (M-CSF)/CSF-1R | Lacnotuzumab (MCS110), LY3022855, |
| | SNDX-6352, emactuzumab (RG7155), |
| | pexidartinib (PLX3397) |
| IL-1 and IL-1R3 (IL-1RAP) | CAN04, Canakinumab (ACZ885) |
| IL-8 | BMS-986253 |
| SEMA4D | Pepinemab (VX15/2503) |
| Ang-2 | Trebananib |
| CLEVER-1 | FP-1305 |
| Axl | Enapotamab vedotin (EnaV) |
| Phosphatidylserine | Bavituximab |

In some embodiments, an active agent that may be included in a pharmaceutical composition described herein is or comprises a therapeutic agent administered in a combination therapy described herein. Pharmaceutical compositions described herein can be administered in combination therapy, i.e., combined with other agents. In some embodiments, such therapeutic agents may include agents leading to depletion or functional inactivation of regulatory T cells. For example, in some embodiments, a combination therapy can include a provided pharmaceutical composition with at least one immune checkpoint inhibitor.

In some embodiments, a pharmaceutical composition described herein may be administered in conjunction with radiotherapy and/or autologous peripheral stem cell or bone marrow transplantation.

In some embodiments, a pharmaceutical composition described herein may be combined with a checkpoint inhibitor (e.g., an inhibitor of PD-1, PD-L1, CTLA4, and/or their associated pathways). In some embodiments, a checkpoint inhibitor can include ipilimumab, nivolumab, pembrolizumab, or a combination thereof.

In some embodiments, a pharmaceutical composition described herein may be combined with a signal transduction inhibitor. In some embodiments, a signal transduction inhibitor can include a BRAF inhibitor (e.g., vemurafenib or dabrafenib). In some embodiments, a signal transduction inhibitor can include a MEK inhibitor.

In some embodiments, a pharmaceutical composition described herein may be combined with a intralesional therapy (e.g., talimogene laherparepvec).

In some embodiments, a pharmaceutical composition described herein may be combined with a cytotoxic therapy (e.g., IL-2, dacarbazine, carboplatin/paclitaxel, albumin-bound paclitaxel).

In some embodiments, a pharmaceutical composition described herein can be frozen to allow long-term storage.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

To ensure appropriate quality of useful components (e.g., one or more RNA molecules(s) collectively encoding (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof) in pharmaceutical compositions described herein, one or more quality assessments and/or criteria (e.g., RNA quality assessments) may be performed and/or monitored.

Among other things, the present disclosure provides methods of characterizing one or more features of one or more RNA molecules or composition thereof, which one or more RNA molecules encodes part or all of an antibody agent.

In some embodiments, RNA integrity assessment of one or more RNA molecules (e.g., in some embodiments a pharmaceutical composition comprising one or more RNA molecules collectively encoding a NY-ESO-1 antigen, a MAGE-A3 antigen, a tyrosinase antigen, a TPTE antigen, or a combination thereof can be performed by adaptation of a capillary gel electrophoresis assay.

Additionally or alternatively, in some embodiments, RNA ratio of a pharmaceutical composition comprising one or more one or more RNA molecules each encoding (a NY-ESO-1 antigen, a MAGE-A3 antigen, a tyrosinase antigen, a TPTE antigen, or a combination thereof can be measured by droplet digital PCR.

Additionally or alternatively, in some embodiments, residual DNA template and residual dsRNA are measured as in-process controls with acceptance criteria on the level of the drug substance intermediates to ensure individual RNA quality before mixing to the drug substance, for example, before mixing two or more one or more RNA molecules each encoding different TAA or combinations of TAA (e.g., a NY-ESO-1 antigen, a MAGE-A3 antigen, a tyrosinase antigen, a TPTE antigen, or a combination thereof).

Additionally or alternatively, in some embodiments, residual host cell DNA and/or host cell protein may be measured in compositions comprising RNA molecules.

### V. Patient Populations

Technologies provided herein can be useful for treatment of diseases or conditions associated with cancer. In some embodiments, technologies provided herein can be useful for treatment of diseases and conditions associated with an epithelial cancer.

One type of cancer for which technologies described herein can be useful in treating is melanoma. Melanoma is a malignant tumor of melanocytes. Melanomas can arise in the skin, but they can also arise from mucosal surfaces or at other sites to which neural crest cells migrate, including the uveal tract. (Kuk et al. 2016, which is incorporated herein by reference in its entirety). Mucosal and uveal melanomas differ significantly from cutaneous melanoma in incidence, prognostic factors, molecular characteristics, and treatment (van der Kooij et al. 2019, which is incorporated herein by reference in its entirety).

In the United States, it is estimated that in 2021 approximately 106,110 patients will be diagnosed with melanoma of the skin and there will be approximately 7,180 deaths (Siegel et al. 2021, which is incorporated herein by reference in its entirety). Although the age-standardized incidence rate of melanoma is lower when compared to non-melanoma skin cancer (3.4 vs. 11.0 per 100,000 in 2020, respectively), it has a high mortality rate (Globocan 2020; Coricovac et al. 2018, each of which is incorporated herein by reference in its entirety). Invasive melanoma represents about 1% of skin cancers, but results in the most deaths caused by skin cancers (ACS 2021, which is incorporated herein by reference in its entirety).

The outcome of melanoma depends on the stage at presentation. The 5-year survival for patients with early stage disease (e.g., localized) is approximately 99% of patients and for patients with regional stage (e.g., with spread to lymph nodes) 66% of patients. However, the 5-year survival for patients with distant disease is only approximately 27% (SEER CRS 2021; Swetter et al. 2021, each of which is incorporated herein by reference in its entirety).

In some embodiments, technologies provided herein can be useful for treatment of melanoma. In some embodiments, technologies provided herein can be useful for treatment of cutaneous melanoma. In some embodiments, technologies provided herein can be useful for treatment of advanced stage cancer (e.g., melanoma). Examples of advanced stage cancer include, without limitation, Stage II, Stage III or Stage IV. In some embodiments, technologies provided herein can be useful for treatment of diseases or conditions associated Stage IIIB, Stage IIIC, or Stage IV melanoma. In some embodiments, a cancer is fully resected. In some embodiments, there is no evidence of disease (e.g., cancer). In some embodiments, a cancer is fully resected and there is no evidence of disease.

In some embodiments, technologies provided herein can be useful for treatment of patients (e.g., adult patients) with melanoma that is metastatic. In some embodiments, technologies provided herein can be useful for treatment of patients (e.g., adult patients) with melanoma that is unresectable, e.g., in some embodiments where surgical resection is likely to result in severe morbidity. In some embodiments, technologies provided herein can be useful for treatment of patients (e.g., adult patients) with melanoma that are locally advanced. Additionally or alternatively, in some embodiments, cancer in such patients may have progressed following treatment or such cancer patients may have no satisfactory alternative therapy. In some embodiments, patients who are receiving a treatment described herein may have received other cancer therapy, e.g., but not limited to chemotherapy.

In some embodiments, technologies provided herein can be useful for treatment of advanced melanoma. In some embodiments, technologies provided herein can be useful for treatment of checkpoint-inhibitor (CPI)-experienced patients with unresectable melanoma.

In some embodiments, technologies provided herein can be useful for treatment of patients diagnosed with cancer prior to the time of administration of the pharmaceutical composition, but where the patient is classified as having No Evidence of Disease (NED) at the time of administration. In some embodiments, patients who are classified as NED at the time of administration are patients whose melanoma has been fully resected (e.g., by surgery). In some embodiments, patients who are classified as NED at the time of administration are patients who have been previously diagnosed with a clinical stage 3 or stage 4 melanoma (or a pathological stage 3 or stage 4 melanoma) and whose melanoma has been fully resected (e.g., by surgery). In some embodiments, patients who are classified as NED at the time of administration are patients whose melanoma has been fully resected and who will go on to receive adjuvant treatment. In some embodiments, patients who are classified as NED at the time of administration are patients previously diagnosed with a clinical stage 3 or stage 4 melanoma (or a pathological stage 3 or stage 4 melanoma) and whose melanoma has been fully resected and who will go on to receive adjuvant treatment. Without wishing to be bound by a particular theory, in some embodiments, “no evidence of disease” is determined by applying a RECIST standard, e.g., a RECIST1.1 standard or an immune related Response Evaluation Criteria in Solid Tumors (irRECIST) standard.

For clarity, patients who are classified as NED at the time of administration are different from patients who are classified as having “non-measurable disease.” Patients who have “non-measurable disease” means that there is evidence of disease but it cannot reliably be measured according to a RECIST standard, e.g., a RECIST1.1 standard as described in Eisenhauer et al. “New response evaluation criteria in solid tumours: Revised RECIST guideline (version 1.1)” European Journal of Cancer (2009) 45:228-247, the entire content of which is incorporated herein by references for the purposes described herein. Examples of lesions that are considered as non-measurable lesions include but are not limited to lesions in bones, ascites of pleural effusions, “complex irregular” lesions in tissues or organs. Stated another way, patients who have “non-measurable disease” means that patients have tumor lesions that are not considered as “measurable” lesions according to a RECIST standard, e.g., a RECIST1.1 standard as discussed above. Therefore, a difference between non-measureable disease and NED is that the former means disease is present but cannot be measured, while the latter (NED) means no disease is present, thereby not evaluable and apparently not measureable.

Administration of a pharmaceutical composition as described herein to NED patients may, therefore, seem counterintuitive. The present disclosure recognizes, however, that a patient may be determined to be free of cancer or in remission, but that cancer may re-emerge. Accordingly, the present disclosure provides the insight that such patients may benefit from receiving a pharmaceutical composition as described herein because it may, e.g., boost the patient's immune response to cancer. Boosting the patient's immune response to cancer can allow the patient's body to attack cancer cells, e.g., that are undetected or are developing.

In some embodiments, technologies provided herein can be useful for treatment of melanoma patients who have measurable disease.

In some embodiments, technologies provided herein can be useful for treatment of melanoma patients who have non-measurable disease.

In some embodiments, technologies provided herein can be useful for treatment of patients that are in remission.

In some embodiments, a subject who is administered a pharmaceutical composition described herein may have received a prior anti-cancer therapy. Examples of prior anti-cancer therapies include but are not limited to chemotherapy, interferons and interleukins, monoclonal antibodies, protein kinase inhibitors, radiotherapy, immune checkpoint inhibitors, or combinations thereof. For example, in some embodiments, a subject who is administered a pharmaceutical composition described herein may have received an immune checkpoint inhibitor but did not experience tumor regression. In another example, in some embodiments, a subject who is administered a pharmaceutical composition described herein may have received an immune checkpoint inhibitor and experienced tumor regression. Examples of such immune checkpoint inhibitors include, but are not limited to a PD-1 inhibitor, a PDL-1 inhibitor, a CTLA-4 inhibitor, or a combination thereof. In some embodiments, an immune checkpoint inhibitor is an antibody (e.g., but not limited to, ipilumumab and nivolumab). Additional examples of checkpoint inhibitors are included in Table 4 above or in Example 8.

In some embodiments, a patient who meets one or more of the disease-specific inclusion criteria as described in Example 12 are amenable to treatment described herein (e.g., receiving a provided pharmaceutical composition as monotherapy or as part of a combination therapy). In some embodiments, such a patient that is administered a treatment described herein may further meets one or more of the other inclusive criteria as described in Example 12.

In some embodiments, a cancer patient who has melanoma but meets one or more of the exclusion criteria as described in Example 13 is not administered a treatment described herein.

VI. Readouts of Patients Having been Administered a Pharmaceutical Composition

In some embodiments, administering a pharmaceutical composition comprising one or more RNA molecules that collectively encode a NY-ESO-1 antigen, a MAGE-A3 antigen, a tyrosinase antigen, a TPTE antigen, or a combination thereof, induces an immune response. In some embodiments, the methods described herein further comprise determining a level of immune response in the patient (e.g., patient is classified as having no evidence of disease at the time of administration) administered the pharmaceutical composition. For example, in some embodiments, determining a level of the immune response in the patient occurs before and after administration of the pharmaceutical composition.

Non-limiting examples of methods used to determine a level of immune response in the patient are as described in Examples 1-3. For example, in some embodiments, exploiting the enhanced glucose consumption of following administration of the pharmaceutical composition can be performed by [18F]-fluoro-2-deoxy-2-d-glucose (FDG)-positron emission tomography (PET)/computerized tomography (CT) scans of the spleen can be carried following administration of the pharmaceutical composition. Without wishing to be bound by theory, the (FDG)-(PET)/(CT) scans are used to indicate targeting and at least transient activation of lymphoid tissue-resident immune cells. In some embodiments, a level of immune response in the patient is determined using an interferon-7 enzyme-linked immune absorbent spot (ELISpot) assay, as described in Example 1. In some embodiments, a level of metabolic activity in the patient's spleen is measured using positron emission tomography (PET), computerized tomography (CT) scans, magnetic resonance imaging (MRI), or a combination thereof. In some embodiments, a level of metabolic activity in the patient's spleen is measured using positron emission tomography (PET) and computerized tomography (CT) scans. In some embodiments, a level of metabolic activity in the patient's spleen is measured using positron emission tomography (PET) and magnetic resonance imaging (MRI).

In some embodiments, determining a level of immune response in the patient (e.g., patient is classified as having no evidence of disease at the time of administration) after receiving the pharmaceutical composition includes comparing the level of the immune response in the patient with a level of the immune response in a second patient to which the pharmaceutical composition has been administered. In some embodiments, the second patient was diagnosed with cancer prior to the time of administration and is classified as having evidence of disease at the time of administration.

In some embodiments, a pharmaceutical composition induces a level of the immune response in the patient (e.g., patient is classified as having no evidence of disease at the time of administration) that is comparable to a level of the immune response in a second patient to which the pharmaceutical composition has been administered. In some embodiments, a second patient has previously been diagnosed with cancer, and is classified as having evidence of disease at the time of administration. In some embodiments, a level of the immune response in the patient is comparable if it differs from the level of the immune response in the second patient if it differs by less 20%, less than 15%, less than 10%, or less than 5%.

In some embodiments, comparing a level of the immune response in a patient (e.g., patient is classified as having no evidence of disease at the time of administration) after administration of a pharmaceutical composition with the level of the immune response in the patient before administration of the pharmaceutical composition. For example, in some embodiments, a level of the immune response in a patient (e.g., patient is classified as having no evidence of disease at the time of administration) after administration of the pharmaceutical composition is increased compared with the level of the immune response in the patient before administration of the pharmaceutical composition. In some embodiments, a level of the immune response in a patient (e.g., patient is classified as having no evidence of disease at the time of administration) after administration of the pharmaceutical composition is maintained compared with the level of the immune response in the patient before administration of the pharmaceutical composition.

In some embodiments, a level of the immune response is a de novo immune response induced by a pharmaceutical composition. In some embodiments, a de novo immune response is an immune response that has developed in response to a pharmaceutical composition. In some embodiments, a de novo immune response does not include a background or pre-existing level of the immune response.

In some embodiments, administering a pharmaceutical composition comprising one or more RNA molecules that collectively encode a NY-ESO-1 antigen, a MAGE-A3 antigen, a tyrosinase antigen, a TPTE antigen, or a combination thereof, to the patient (e.g., patient is classified as having no evidence of disease at the time of administration) induces an adaptive immune response. For example, in some embodiments, an immune response in the patient is a T cell response, where the T cell response includes a CD4⁺ and/or CD8⁺ T cell response. In some embodiments, administering a pharmaceutical composition comprising one or more RNA molecules that collectively encode a NY-ESO-1 antigen, a MAGE-A3 antigen, a tyrosinase antigen, a TPTE antigen, or a combination thereof, to the patient (e.g., patient is classified as having no evidence of disease at the time of administration) induces CD4⁺ and/or CD8⁺ T cell immunity.

In some embodiments, the methods described herein include determining a level of immune response in a patient by measuring an amount of one or more cytokines in the patient's plasma. For example, as described in Example 2, the presence and/or amount of one or more cytokines associated with an immune response (e.g., IFN-α, IFN-γ, interleukin (IL)-6, IFN-inducible protein (IP)-10, IL-12 p70 subunit, or a combination there) can be used to determine the level of the immune response in the patient. In some embodiments, measuring the amount of one or more cytokines in the patient's plasma occurs before and after administration of the pharmaceutical composition.

In some embodiments, the methods described herein include measuring a number of cancer lesions in the patient. For example, in some embodiments, the methods described herein include measuring a number of cancer lesions in the patient before and after administration of the pharmaceutical composition. In some embodiments, administering a pharmaceutical composition comprising one or more RNA molecules that collectively encode a NY-ESO-1 antigen, a MAGE-A3 antigen, a tyrosinase antigen, a TPTE antigen, or a combination thereof, to the patient (e.g., patient is classified as having no evidence of disease at the time of administration) reduces the number of cancer lesions, as compared to number cancer lesions in the patient before administration of the pharmaceutical composition.

In some embodiments, the methods described herein include measuring a number of T cells induced by the pharmaceutical composition in the patient. For example, in some embodiments, the methods described herein include measuring the number of T cells induced by the pharmaceutical composition in the patient at a plurality of time points following administration of the pharmaceutical composition. In another example, the methods described herein include measuring the number of T cells induced by the pharmaceutical composition in the patient following administration of a first dose the pharmaceutical composition and following administration of a second dose the pharmaceutical composition. In some embodiments, the number of T cells induced by the pharmaceutical composition in the patient is greater following administration of the second dose of the pharmaceutical composition than following administration of the first dose of the pharmaceutical composition.

In some embodiments, the methods described herein include determining a phenotype of T cells induced by the pharmaceutical composition in the patient following administration of the pharmaceutical composition. For example, in some embodiments, following administration of the pharmaceutical composition, at least a subset of T cells induced by the pharmaceutical composition in the patient have a T-helper-1 phenotype. In some embodiments, the phenotype of the T cells induced by the pharmaceutical composition in the patient have a PD1⁺ effector memory phenotype. In some embodiments, the phenotype of the T cells induced by the pharmaceutical composition in the patient have a T-helper-1 and PD1⁺ effector memory phenotype.

In some embodiments, the methods described herein include, for a patient classified as having evidence of disease, measuring the size of one or more cancer lesions in the patient. For example, in some embodiments, the methods described herein include measuring the size of one or more cancer lesions in the patient before and after administration of the pharmaceutical composition. In some embodiments, administering a pharmaceutical composition comprising one or more RNA molecules that collectively encode a NY-ESO-1 antigen, a MAGE-A3 antigen, a tyrosinase antigen, a TPTE antigen, or a combination thereof, to the patient (e.g., patient is classified as having no evidence of disease at the time of administration) maintains or reduces the size of one or more cancer lesions, as compared to size of one or more cancer lesions in the patient before administration of the pharmaceutical composition. In other words, the size of one or more cancer lesions does not increase after administration of a pharmaceutical composition described herein.

In some embodiments, the methods described herein include, for a patient classified as having evidence of disease, monitoring a duration of progression-free survival. In some embodiments, the methods described herein include comparing the duration of progression-free survival of the patient with than a reference duration of progression-free survival. In some embodiments, a reference duration of progression-free survival is an average duration of progression-free survival of a plurality of comparable patients who have not received a pharmaceutical composition described herein. In some embodiments, duration of progression-free survival of the patient is longer in time than a reference duration of progression-free survival.

In some embodiments, the methods described herein include, for a patient classified as having evidence of disease, measuring a duration of disease stabilization. In some embodiments, disease stabilization is determined by applying an irRECIST or RECIST 1.1 standard. In some embodiments, a method described herein comprises comparing the duration of disease stabilization of the patient to a reference duration of disease stabilization. In some embodiments, a reference duration of disease stabilization is an average duration of disease stabilization of a plurality of comparable patients who have not received the pharmaceutical composition. In some embodiments, a patient administered a pharmaceutical composition described herein exhibits an increased duration of disease stabilization compared to the reference duration of disease stabilization.

In some embodiments, the methods described herein include, for a patient classified as having evidence of disease, measuring a duration of tumor responsiveness. In some embodiments, tumor responsiveness is determined by applying an irRECIST or RECIST 1.1 standard. In some embodiments, a method described herein comprises comparing the duration of tumor responsiveness of the patient to a reference duration of tumor responsiveness. In some embodiments, a reference duration of tumor responsiveness is an average duration of tumor responsiveness of a plurality of comparable patients who have not received the pharmaceutical composition. In some embodiments, a patient administered a pharmaceutical composition described herein exhibits an increased duration of tumor responsiveness compared to the reference duration of tumor responsiveness.

In some embodiments, the methods described herein include, for a patient classified as having no evidence of disease, monitoring a duration of disease-free survival. In some embodiments, a method described herein comprises comparing the duration disease-free survival of the patient to a reference duration of disease-free survival. In some embodiments, a duration of disease-free survival in a patient administered a pharmaceutical composition described herein exhibits longer in time than a reference duration of disease-free survival. In some embodiments, a reference duration of disease-free survival is an average duration of disease-free survival of a plurality of comparable patients who have not received the pharmaceutical composition. In some embodiments, a patient administered a pharmaceutical composition described herein exhibits an increased duration of disease-free survival compared to the reference duration of disease-free survival.

In some embodiments, the methods described herein include, for a patient classified as having no evidence of disease, measuring a duration to disease relapse. In some embodiments, disease relapse is determined by applying an irRECIST or RECIST 1.1 standard. In some embodiments, methods described herein comprise comparing the duration to disease relapse of the patient to a reference duration to disease relapse. In some embodiments, a reference duration to disease relapse is an average duration to disease relapse of a plurality of comparable patients who have not received the pharmaceutical composition. In some embodiments, a patient administered a pharmaceutical composition described herein exhibits an increased duration to disease relapse compared to the reference duration to disease relapse.

### VII. Treatment

In some embodiments, pharmaceutical compositions described herein can be taken up by target cells (e.g., dendritic cells) for translation of antigen-encoding RNA thereby inducing CD4⁺ and CD8⁺ T cell immunity against the antigens.

Accordingly, another aspect of the present disclosure relates to methods of using pharmaceutical compositions described herein. For example, one aspect provided herein is a method comprising administering a provided pharmaceutical composition to a subject suffering from cancer. In some embodiments, a provided pharmaceutical composition is administered by intravenous injection or infusion. Examples of a cancer include but are not limited to a epithelial cancer, including, but not limited to, melanoma (e.g., cutaneous melanoma, Stage IIIB, Stage IIIC, or Stage IV melanoma).

Dosing schedule: Those skilled in the art are aware that cancer therapeutics are often administered using varying ranges of a pharmaceutical composition that can be administered in dosing cycles.

In some embodiments, pharmaceutical compositions described herein are administered in eight doses within 64 days from the first administration, e.g., using a prime-and-boost protocol.

In some embodiments, pharmaceutical compositions described herein are administered in six doses within 43 days from the first administration, e.g., using a prime-and-boost protocol.

In some embodiments, pharmaceutical compositions described herein are administered monthly following completion of an original dosing cycle, e.g., the prime-and-boost protocol.

In some embodiments, pharmaceutical compositions described herein are administered in one or more dosing cycles.

In some embodiments, one dosing cycle is at least 7 or more days (including, e.g., at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30 days, at least 40 days, at least 50 days, or at least 60 days). In some embodiments, one dosing cycle is at least 28 days. In some embodiments, one dosing cycle is at least 35 days. In some embodiments, one dosing cycle is at least 42 days. In some embodiments, one dosing cycle is at least 49 days. In some embodiments, one dosing cycle is at least 56 days. In some embodiments, one dosing cycle is at least 63 days.

In some embodiments, one dosing cycle may involve multiple doses, e.g., according to a pattern such as, for example, a dose may be administered periodically within a cycle, or a dose may be administered every 6 days, every 7 days, every 8 days, every 9 days, every 10 days, every 12 days, or every 14 days within a cycle. In some embodiments, one dosing cycle may involve at least 2 doses, including, e.g., at least 3 doses, at least 4 doses, at least 5 doses, at least 6 doses, at least 7 doses, at least 8 doses, or higher. In some embodiments, one dosing cycle may involve up to 8 doses, which may be administered weekly, biweekly, or combinations thereof.

In some embodiments, multiple cycles may be administered. For example, in some embodiments, at least 2 cycles (including, e.g., at least 3 cycles, at least 4 cycles, at least 5 cycles, at least 6 cycles, at least 7 cycles, at least 8 cycles, at least 9 cycles, at least 10 cycles, or more) can be administered. In some embodiments, the number of dosing cycles to be administered may vary with types of treatment (e.g., monotherapy vs. combination therapy). In some embodiments, at least 2 dosing cycles may be administered. In some embodiments, a first dosing cycle can be different from a second dosing cycle. In some embodiments, a first dosing cycle may comprise 6-8 weekly and/or biweekly doses, and a second dosing cycle that follows the first dosing cycle may comprise at least one monthly dose.

In some embodiments, there may be a “rest period” between cycles; in some embodiments, there may be no rest period between cycles. In some embodiments, there may be sometimes a rest period and sometimes no rest period between cycles.

In some embodiments, a rest period may have a length within a range of several days to several months. For example, in some embodiments, a rest period may have a length of at least 3 days or more, including, e.g., at least 4 days, at least 5 days, at least 6 days, at least 7 days, at least 8 days, at least 9 days, at least 10 days, at least 11 days, at least 12 days, at least 13 days, at least 14 days or more. In some embodiments, a rest period may have a length of at least 1 week or more, including, e.g., at least 2 weeks, at least 3 weeks, at least 4 weeks, or more.

Dose: Dosage of pharmaceutical compositions described herein may vary with a number of factors including, e.g., but not limited to body weight of a subject to be treated, cancer types and/or cancer stages, and/or monotherapy or combination therapy. In some embodiments, a dosing cycle involves administration of a set number and/or pattern of doses. For example, in some embodiments, a pharmaceutical composition described herein is administered at least one dose per dosing cycle, including, e.g., at least two doses per dosing cycle, at least three doses per dosing cycle, at least four doses per dosing cycle, or more.

In some embodiments, a dosing cycle involves administration of a set cumulative dose, e.g., over a particular period of time, and optionally via multiple doses, which may be administered, for example, at set interval(s) and/or according to a set pattern. In some embodiments, a set cumulative dose may be administered via multiple doses at set intervals such that there is at least some temporal overlap in biological and/or pharmacokinetics effects generated by such multiple doses on a target cell or on a subject being treated. In some embodiments, a set cumulative dose may be administered via multiple doses at set intervals such that biological and/or pharmacokinetics effects generated by such multiple doses on a target cell or on a subject being treated may be additive. By way of example only, in some embodiments, a set cumulative dose of X mg may be administered via two doses with each dose of X/2 mg, wherein such two doses are administered sufficiently close in time such that biological and/or pharmacokinetics effects generated by each X/2-mg dose on a target cell or on a subject being treated may be additive.

In some embodiments, each dose or a cumulative dose (e.g., for intravenous administration) is administered at a level such that the one or more RNA molecules that collectively encode a NY-ESO-1 antigen, a MAGE-A3 antigen, a tyrosinase antigen, a TPTE antigen, or a combination thereof, is expected to achieve level (e.g., plasma level and/or tissue level) that is high enough to for translation and antigen presentation in an antigen-presenting cell (e.g., a dendritic cell or immature dendritic cell) that induces a CD4⁺ and CD8⁺ T cell immunity against the one or more antigens throughout a dosing cycle.

In some embodiments, each dose ranges from about 7.2 μg to about 400 μg (e.g., any of the subranges herein) of total RNA.

In some embodiments, a method provided herein comprises a dose escalation. Exemplary methods comprising dose escalation are described, e.g., in WO2018/0077942.

In some embodiments, the methods provided herein include 7 dose escalation cohorts (3+3 design) and 3 expanded cohorts. For example, Table 5 provides exemplary dosing schedules.

TABLE 5
Exemplary dosing schedules

| | | | | | | | | Patients with | | Patients with + | |
|---|---|---|---|---|---|---|---|---|---|---|---|
| | | | | | | | | Monotherapy | | AntiPD-1 | |
| | | | | | | | | | Non- | | Non- |
| | Dose [μg] at day | | | | | | All | Measurable | measurable | Measurable | measurable |
| Cohort | 1 | 8 | 15 | 22 | 29 | 36, 50, 64 | Patients | disease | disease | disease | disease |
| CI | 7.2 | 14.4 | 29 | 29 | 29 | 29 | 3 | 1 | 2 | 0 | 0 |
| CII | 7.2 | 14.4 | 14.4 | 14.4 | 14.4 | 14.4 | 3 | 0 | 3 | 0 | 0 |
| CIII | 7.2 | 14.4 | 29 | 50 | 50 | 50 | 3 | 1 | 2 | 0 | 0 |
| CIV | 7.2 | 14.4 | 29 | 50 | 75 | 75 | 3 | 3 | 0 | 0 | 0 |
| CV | 7.2 | 14.4 | 29 | 50 | 75 | 100 | 4 | 3 | 1 | 0 | 0 |
| CVI | 50 | 100 | 200 | 200 | 200 | 200 | 3 | 0 | 2 | 1* | 0 |
| CVII | 50 | 100 | 200 | 400 | 400 | 400 | 1 | 0 | 1 | 0 | 0 |
| Exp. A | 7.2 | 14.4 | 14.4 | 14.4 | 14.4 | 14.4 | 23 | 10 | 10 | 3 | 0 |
| Exp. B | 50 | 50 | 50 | 50 | 50 | 50 | 17 | 5 | 7 | 5 | 0 |
| Exp. C | 50 | 100 | 100 | 100 | 100 | 100 | 32 | 8 | 7 | 17 | 0 |

In some embodiments, dosing may be adjusted based on response of a subject receiving the therapy. For example, in some embodiments, dosing may involve administration of a higher dose followed later by administration of a lower dose if one or more parameters for safety pharmacology assessment indicates that the prior dose may not satisfy the medical safety requirement according to a physician. In some embodiments, dose escalation may be performed at one or more of the levels shown in Table 5 of Example 7; in some embodiments, dose escalation may involve administration of at least one lower dose from Table 5 followed later by administration of at least one higher dose from Table 5. Without wishing to be bound by any particular theory, the present disclosure, among other things, provides an insight that a pharmaceutically guided dose escalation (PGDE) method may be applied to determine an appropriate dose of pharmaceutical compositions described herein. An exemplary dose escalation study is provided in Example 7.

Also provided herein is also a method of determining a dosing regimen of a pharmaceutical composition comprising the one or more RNA molecules that collectively encode a NY-ESO-1 antigen, a MAGE-A3 antigen, a tyrosinase antigen, a TPTE antigen, or a combination thereof. For example, in some embodiments, such a method comprises steps of: (A) administering a pharmaceutical composition (e.g., ones described herein) to a subject suffering from a melanoma or a subject who has been classified as no evidence of disease under a pre-determined dosing regimen; (B) monitoring or measuring evidence of disease (e.g., tumor lesion size and/or metastases) of the subject periodically over a period of time; (C) evaluating the dosing regimen based on the monitoring or measuring results and/or outcomes. For example, a dose and/or dosage frequency can be increased if reduction in tumor size after the administration of a pharmaceutical composition (e.g., ones described herein) is not therapeutically relevant; or a dose and/or dosage frequency can be decreased if reduction in tumor size after the administration of a pharmaceutical composition (e.g., ones described herein) is therapeutically relevant, but adverse effect (e.g., toxicity effect) is shown in the subject. If reduction in tumor size after the administration of a pharmaceutical composition (e.g., ones described herein) is therapeutically relevant, and no adverse effect (e.g., toxicity effect) is shown in the subject, no changes is made to a dosage regimen.

In some embodiments, such a method of determining a dosing regimen of a pharmaceutical composition comprising the one or more RNA molecules that collectively encode a NY-ESO-1 antigen, a MAGE-A3 antigen, a tyrosinase antigen, a TPTE antigen, or a combination thereof; may be performed in a group of animal subjects (e.g., mammalian non-human subjects) each a bearing a human melanoma xenograft tumor. In some such embodiments, a dose and/or dosage frequency can be increased if less than 30% of the animal subjects exhibit reduction in tumor size after the administration of a pharmaceutical composition (e.g., ones described herein) and/or extent of reduction in tumor size exhibited by the animal subjects is not therapeutically relevant; or a dose and/or dosage frequency can be decreased if reduction in tumor size after the administration of a pharmaceutical composition (e.g., ones described herein) is therapeutically relevant, but significant adverse effect (e.g., toxicity effect) is shown in at least 30% of the animal subjects. If reduction in tumor size after the administration of a pharmaceutical composition (e.g., ones described herein) is therapeutically relevant, and no significant adverse effect (e.g., toxicity effect) is shown in the animal subjects, no changes is made to a dosage regimen.

Although the dosing regimens (e.g., dosing schedule and/or doses) provided herein are principally suitable for administration to humans, it will be understood by the skilled artisan that dose equivalents can be determined for administration to animals of all sorts. The ordinarily skilled veterinary pharmacologist can design and/or perform such determination with merely ordinary, if any, experimentation.

Monotherapy: In some embodiments, pharmaceutical compositions described herein can be administered to patients as monotherapy.

Combination therapy: The present disclosure, among other things, provides an insight that the capability of pharmaceutical compositions comprising the one or more RNA molecules that collectively encode a NY-ESO-1 antigen, a MAGE-A3 antigen, a tyrosinase antigen, a TPTE antigen, or a combination thereof as described herein to induce a CD4⁺ and CD8⁺ T cell immunity against the antigens encoded by the one or more RNA molecules can augment cytotoxic effect(s) of chemotherapy and/or other anti-cancer therapy (e.g., immune checkpoint inhibitors). In some embodiments, such a combination therapy may prolong progression-free and/or overall survival, e.g., relative to individual therapies administered alone and/or to another appropriate reference. Accordingly, in some embodiments, pharmaceutical compositions described herein can be administered in combination with other anti-cancer agents in patients with cancer (e.g., melanoma).

Without wishing to be bound by a particular theory, the present disclosure observes that certain immune check point inhibitors, for example such as PD-1 inhibition, PDL-1 inhibition, and CTLA4 inhibition, synergize with the pharmaceutical compositions described herein when administered as a combination therapy to patients with CPI-experience tumors.

The present disclosure, among other things, provides an insight that pharmaceutical compositions described herein may be particularly useful and/or effective when administered to patients with no evidence of disease at time of first administration thereby showing that the pharmaceutical composition induced T cell immunity even in the absence of a detectable tumor.

In some embodiments, a provided pharmaceutical composition may be administered as part of combination therapy comprising such a pharmaceutical composition and an immune checkpoint inhibitor. Accordingly, in some embodiments, a provided pharmaceutical composition may be administered to a subject suffering from a cancer (e.g., melanoma) who has received an immune check point inhibitor or a chemotherapeutic agent or a subject who has received an immune check point inhibitor or a chemotherapeutic agent and is classified as having no evidence of disease. In some embodiments, a provided pharmaceutical composition may be co-administered with an immune checkpoint inhibitor to a subject suffering from a cancer (e.g., a melanoma) or a subject who has been classified as having no evidence of disease. In some embodiments, a provided pharmaceutical composition and an immune checkpoint inhibitor may be administered concurrently or sequentially. For example, in some embodiments, a first dose of an immune checkpoint inhibitor may be administered after (e.g., at least 30 minutes after) administration of a provided pharmaceutical composition. In some embodiments, an immune checkpoint inhibitor and a provided pharmaceutical composition are concomitantly administered.

For example, in some embodiments, an immune checkpoint inhibitor comprises one or more inhibitors selected from Table 4 above (see, e.g., Marin-Acevdeo et al., J. Hematology & Oncology, 14: 45 (2021), which is herein incorporated by reference in its entirety) or as described in Example 8.

Combination treatment with an anti-cancer therapy comprising ipilimumab: In some embodiments, an administered therapy comprising a provided pharmaceutical composition may be co-administered or overlap with an immune checkpoint inhibitor comprising ipilimumab. Ipilimumab blocks cytotoxic T-lymphocyte antigen-4 (CTLA-4), a critical negative regulator of the anti-tumor T-cell response. Blocking CTLA-4 inhibits T cell activation thereby allowing expansion of pre-existing antigen specific T cells.

Combination treatment with an anti-cancer therapy comprising nivolumab: In some embodiments, an administered therapy comprising a provided pharmaceutical composition may be co-administered or overlap with immune checkpoint inhibitor comprising nivolumab. Nivolumab is a monoclonal antibody that binds to the PD-1 receptor and blocks interaction with PD-L1 and PD-L2. Blocking this interaction releases PD-1 mediated pathway inhibition of the immune response, including the anti-tumor T-cell response, allowing expansion of pre-existing antigen specific T cells.

Combination treatment with an anti-cancer therapy comprising pembrolizumab: In some embodiments, an administered therapy comprising a provided pharmaceutical composition may be co-administered or overlap with an immune checkpoint inhibitor comprising pembrolizumab. Pembrolizumab is a monoclonal antibody that binds to the PD-1 receptor and blocks interaction with PD-L1 and PD-L2. Blocking this interaction releases PD-1 mediated pathway inhibition of the immune response, including the anti-tumor T-cell response, allowing expansion of pre-existing antigen specific T cells.

Combination treatment with an anti-cancer therapy comprising cemiplimab: In some embodiments, an administered therapy comprising a provided pharmaceutical composition may be co-administered or overlap with an immune checkpoint inhibitor comprising cemiplimab. Cemiplimab is a monoclonal antibody that binds to the PD-1 receptor and blocks interaction with PD-L1 and PD-L2. Blocking this interaction releases PD-1 mediated pathway inhibition of the immune response, including the anti-tumor T-cell response, allowing expansion of pre-existing antigen specific T cells.

Efficacy monitoring: In some embodiments, patients receiving a provided treatment may be monitored periodically over the dosing regimen to assess efficacy of the administered treatment. For example, in some embodiments, efficacy of an administered treatment may be assessed by on-treatment imaging periodically, e.g., every 4 weeks, every 5 weeks, every 6 weeks, every 7 weeks, every 8 weeks, or longer.

### EXEMPLARY EMBODIMENTS

Exemplary embodiments provided below are also within the scope of the present disclosure:

Embodiment 1. A method comprising:
administering to a patient at least one dose of a pharmaceutical composition comprising:
(a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and
(b) lipid particles;
wherein the patient was diagnosed with cancer prior to the time of administration, but the patient is classified as having no evidence of disease at the time of administration.

Embodiment 2. The method of embodiment 1, wherein no evidence of disease is or was determined by applying an immune-related Response Evaluation Criteria In Solid Tumors (irRECIST) standard or RECIST 1.1 standard.

Embodiment 3. A method comprising:
administering at least one dose of a pharmaceutical composition to a patient suffering from cancer, wherein the pharmaceutical composition comprises:
(a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and
(b) lipid particles.

Embodiment 4. The method of embodiment 3, wherein the patient is classified as having no evidence of disease at the time of administration.

Embodiment 5. The method of embodiment 3, wherein the patient is classified as having evidence of disease at the time of administration.

Embodiment 6. The method of embodiment 4 or 5, wherein evidence of disease or no evidence of disease is or was determined by applying an immune-related Response Evaluation Criteria In Solid Tumors (irRECIST) standard or RECIST 1.1 standard.

Embodiment 7. The method of any one of embodiments 1-6, wherein the one or more RNA molecules comprise:
(i) a first RNA molecule encoding the NY-ESO-1 antigen,
(ii) a second RNA molecule encoding a MAGE-A3 antigen,
(iii) a third RNA molecule encoding a tyrosinase antigen, and
(iv) a fourth RNA molecule encoding a TPTE antigen.

Embodiment 8. The method of any one of embodiments 1-7, wherein a single RNA molecule of the one or more RNA molecules encodes at least two of the NY-ESO-1 antigen, the MAGE-A3 antigen, the tyrosinase antigen, and the TPTE antigen.

Embodiment 9. The method of any one of embodiments 1-8, wherein a single RNA molecule of the one or more RNA molecules encodes a polyepitopic polypeptide, wherein the polyepitopic polypeptide comprises at least two of the NY-ESO-1 antigen, the MAGE-A3 antigen, the tyrosinase antigen, and the TPTE antigen.

Embodiment 10. The method of any one of embodiments 1-9, wherein the one or more RNA molecules further comprise at least one sequence that encodes a CD4+ epitope.

Embodiment 11. The method of any one of embodiments 1-9, wherein the one or more RNA molecules further comprise at least one sequence that encodes tetanus toxoid P2, a sequence that encodes tetanus toxoid P16, or both.

Embodiment 12. The method of any one of embodiments 1-11, wherein the one or more RNA molecules comprise a sequence encoding an MHC class I trafficking domain.

Embodiment 13. The method of any one of embodiments 1-12, wherein the one or more RNA molecules comprises a 5′ cap or 5′ cap analogue.

Embodiment 14. The method of any one of embodiments 1-13, wherein the one or more RNA molecules comprises a sequence encoding a signal peptide.

Embodiment 15. The method of any one of embodiments 1-14, wherein the one or more RNA molecules comprise at least one non-coding regulatory element.

Embodiment 16. The method of any one of embodiments 1-15, wherein the one or more RNA molecules comprises a poly-adenine tail.

Embodiment 17. The method of embodiment 16, wherein the poly-adenine tail is or comprises a modified adenine sequence.

Embodiment 18. The method of any one of embodiments 1-17, wherein the one or more RNA molecules comprises at least one 5′ untranslated region (UTR) and/or at least one 3′ UTR.

Embodiment 19. The method of embodiment 18, wherein the one or more RNA molecules comprises in 5′ to 3′ order:
(i) a 5′ cap or 5′ cap analogue;
(ii) at least one 5′ UTR;
(iii) a signal peptide;
(iv) a coding region that encodes at least one of the NY-ESO-1 antigen, the MAGE-A3 antigen, the tyrosinase antigen, and the TPTE antigen;
(v) at least one sequence that encodes a tetanus toxoid P2, tetanus toxoid P16, or both;
(vi) a sequence encoding an MHC class I trafficking domain;
(vii) at least one 3′UTR; and
(viii) a poly-adenine tail.

Embodiment 20. The method of any one of embodiments 1-19, wherein the one or more RNA molecules comprise natural ribonucleotides.

Embodiment 21. The method of any one of embodiments 1-20, wherein the one or more RNA molecules comprise modified or synthetic ribonucleotides.

Embodiment 22. The method of any one of embodiments 1-21, wherein at least one of the NY-ESO-1 antigen, the MAGE-A3 antigen, the tyrosinase antigen, and the TPTE antigen are full-length, non-mutated antigens.

Embodiment 23. The method of any one of embodiments 1-22, wherein all of the NY-ESO-1 antigen, the MAGE-A3 antigen, the tyrosinase antigen, and the TPTE antigen are full-length, non-mutated antigens.

Embodiment 24. The method of any one of embodiments 1-23, wherein at least one of the NY-ESO-1 antigen, the MAGE-A3 antigen, the tyrosinase antigen, and the TPTE antigen are expressed from dendritic cells in lymphoid tissues of the patient.

Embodiment 25. The method of any one of embodiments 1-24, wherein at least one of the NY-ESO-1 antigen, the MAGE-A3 antigen, the tyrosinase antigen, and the TPTE antigen are present in the cancer.

Embodiment 26. The method of any one of embodiments 1-25, wherein the lipid particles comprise liposomes.

Embodiment 27. The method of any one of embodiments 1-26, wherein the lipid particles comprise cationic liposomes.

Embodiment 28. The method of any one of embodiments 1-25, wherein the lipid particles comprise lipid nanoparticles.

Embodiment 29. The method of any one of embodiments 1-28, wherein the lipid particles comprise N,N,N trimethyl-2-3-dioleyloxy-1-propanaminium chloride (DOTMA), 1,2-dioleoyl-sn-glycero-3-phosphoethanolamine phospholipid (DOPE), or both.

Embodiment 30. The method of any one of embodiments 1-29, wherein the lipid particles comprise at least one ionizable aminolipid.

Embodiment 31. The method of any one of embodiments 1-30, wherein the lipid particles comprise at least one ionizable aminolipid and a helper lipid.

Embodiment 32. The method of any one of embodiment 31, wherein the helper lipid is or comprises a phospholipid.

Embodiment 33. The method of any one of embodiment 31 or 32, wherein the helper lipid is or comprises a sterol.

Embodiment 34. The method of any one of embodiments 1-33, wherein the lipid particles comprises at least one polymer-conjugated lipid.

Embodiment 35. The method of any one of embodiments 1-34, wherein the patient is a human.

Embodiment 36. The method of any one of embodiments 1-35, wherein the cancer is an epithelial cancer.

Embodiment 37. The method of any one of embodiments 1-36, wherein the cancer is a melanoma.

Embodiment 38. The method of embodiment 37, wherein the melanoma is a cutaneous melanoma.

Embodiment 39. The method of any one of embodiments 1-38, wherein the cancer is advanced stage.

Embodiment 40. The method of any one of embodiments 1-39, wherein the cancer is Stage II, Stage III or Stage IV.

Embodiment 41. The method of any one of embodiments 1-40, wherein the cancer is Stage IIIB, Stage IIIC, or Stage IV melanoma.

Embodiment 42. The method of any one of embodiments 1-41, wherein the cancer is fully resected, there is no evidence of disease, or both.

Embodiment 43. The method of any one of embodiments 1-42, further comprising administering a second dose of the pharmaceutical composition to the patient.

Embodiment 44. The method of any one of embodiments 1-43, further comprising administering at least two doses of the pharmaceutical composition to the patient.

Embodiment 45. The method of any one of embodiments 1-44, further comprising administering at least three doses of the pharmaceutical composition to the patient.

Embodiment 46. The method of embodiment 45, wherein at least one dose of the at least three doses is administered to the patient within 8 days of the patient having received another dose of the at least three doses.

Embodiment 47. The method of embodiment 45 or 46, wherein at least one dose of the at least three doses is administered to the patient within 15 days of the patient having received another dose of the at least three doses.

Embodiment 48. The method of any one of embodiments 1-47, comprising administering at least 8 doses of the pharmaceutical composition to the patient within 10 weeks.

Embodiment 49. The method of embodiment 48, comprising administering a dose of the pharmaceutical composition to the patient weekly for a period of 6 weeks, and then administering a dose of the pharmaceutical composition every two weeks for a period of 4 weeks.

Embodiment 50. The method of embodiment 48 or 49, further comprising administering a dose of the pharmaceutical composition to the patient monthly following the at least 8 doses.

Embodiment 51. The method of any one of embodiments 1-47, comprising administering a dose of the pharmaceutical composition to the patient on a weekly basis for a period of 7 weeks.

Embodiment 52. The method of embodiment 51, further comprising administering a dose of the pharmaceutical composition to the patient every three weeks.

Embodiment 53. The method of any one of embodiments 1-52, wherein the first dose and/or the second dose is 5 μg to 500 μg total RNA.

Embodiment 54. The method of any one of embodiments 1-53, wherein the first dose and/or the second dose is 7.2 μg to 400 μg total RNA.

Embodiment 55. The method of any one of embodiments 1-54, wherein the first dose and/or the second dose is 10 μg to 20 μg total RNA.

Embodiment 56. The method of any one of embodiments 1-55, wherein the first dose and/or the second dose is about 14.4 μg total RNA.

Embodiment 57. The method of any one of embodiments 1-56, wherein the first dose and/or the second dose is about 25 μg total RNA.

Embodiment 58. The method of any one of embodiments 1-54, wherein the first dose and/or the second dose is about 50 μg total RNA.

Embodiment 59. The method of any one of embodiments 1-54, wherein the first dose and/or the second dose is about 100 μg total RNA.

Embodiment 60. The method of any one of embodiments 1-59, wherein the first dose and/or the second dose are administered systemically.

Embodiment 61. The method of any one of embodiments 1-60, wherein the first dose and/or the second dose are administered intravenously.

Embodiment 62. The method of any one of embodiments 1-60, wherein the first dose and/or the second dose are administered intramuscularly.

Embodiment 63. The method of any one of embodiments 1-60, wherein the first dose and/or the second dose are administered subcutaneously.

Embodiment 64. The method of any one of embodiments 1-63, wherein the pharmaceutical composition is administered as monotherapy.

Embodiment 65. The method of any one of embodiments 1-63, wherein the pharmaceutical composition is administered as part of combination therapy.

Embodiment 66. The method of embodiment 65, wherein the combination therapy comprises the pharmaceutical composition and an immune checkpoint inhibitor.

Embodiment 67. The method of any one of embodiments 1-66, wherein the patient has previously received an immune checkpoint inhibitor.

Embodiment 68. The method of any one of embodiments 1-63 and 65-67, further comprising administering to the patient an immune checkpoint inhibitor.

Embodiment 69. The method of any one of embodiments 66-68, wherein the checkpoint inhibitor is or comprises a PD-1 inhibitor, a PDL-1 inhibitor, a CTLA4 inhibitor, a Lag-3 inhibitor, or a combination thereof.

Embodiment 70. The method of any one of embodiments 66-69, wherein the checkpoint inhibitor is or comprises an antibody.

Embodiment 71. The method of any one of embodiments 66-70, wherein the checkpoint inhibitor is or comprises an inhibitor listed in Table 4 herein.

Embodiment 72. The method of any one of embodiments 66-71, wherein the checkpoint inhibitor is or comprises ipilimumab, nivolumab pembrolizumab, avelumab, cemiplimab, atezolizumab, duralumab, or a combination thereof.

Embodiment 73. The method of any one of embodiments 66-72, wherein the checkpoint inhibitor is or comprises ipilimumab.

Embodiment 74. The method of any one of embodiments 66-72, wherein the checkpoint inhibitor is or comprises ipilimumab and nivolumab.

Embodiment 75. The method of any one of embodiments 1-74, wherein the pharmaceutical composition induces an immune response in the patient.

Embodiment 76. The method of any one of embodiments 1-76, further comprising determining a level of the immune response in the patient.

Embodiment 77. The method of embodiment 76, comparing the level of the immune response in the patient with a level of the immune response in a second patient to which the pharmaceutical composition has been administered, wherein the second patient was diagnosed with cancer prior to the time of administration and is classified as having evidence of disease at the time of administration.

Embodiment 78. The method of embodiment 77, wherein the pharmaceutical composition induces a level of the immune response in the patient that is comparable to a level of the immune response in a second patient to which the pharmaceutical composition has been administered, has previously been diagnosed with cancer, and is classified as having evidence of disease at the time of administration.

Embodiment 79. The method of any one of embodiments 75-78, wherein the level of the immune response is a de novo immune response induced by the pharmaceutical composition.

Embodiment 80. The method of any one of embodiments 1-79, further comprising determining a level of the immune response in the patient before and after administration of the pharmaceutical composition.

Embodiment 81. The method of embodiment 80, comparing the level of the immune response in the patient after administration of the pharmaceutical composition with the level of the immune response in the patient before administration of the pharmaceutical composition.

Embodiment 82. The method of embodiment 81, wherein the level of the immune response in the patient after administration of the pharmaceutical composition is increased compared with the level of the immune response in the patient before administration of the pharmaceutical composition.

Embodiment 83. The method of embodiment 81, wherein the level of the immune response in the patient after administration of the pharmaceutical composition is maintained compared with the level of the immune response in the patient before administration of the pharmaceutical composition.

Embodiment 84. The method of any one of embodiments 75-83, wherein the immune response in the patient is an adaptive immune response.

Embodiment 85. The method of any one of embodiments 75-84, wherein the immune response in the patient is a T-cell response.

Embodiment 86. The method of embodiment 85, wherein the T-cell response is or comprises a CD4+ response.

Embodiment 87. The method of embodiment 85 or 86, wherein the T-cell response is or comprises a CD8+ response.

Embodiment 88. The method of any one of embodiments 75-87, wherein the level of the immune response in the patient was determined using an interferon-7 enzyme-linked immune absorbent spot (ELISpot) assay.

Embodiment 89. The method of any one of embodiments 1-88, further comprising measuring a level of one or more of the NY-ESO-1 antigen, the MAGE-A3 antigen, the tyrosinase antigen, and the TPTE antigen in lymphoid tissue of the patient.

Embodiment 90. The method of any one of embodiments 1-89, further comprising measuring a level of one or more of the NY-ESO-1 antigen, the MAGE-A3 antigen, the tyrosinase antigen, and the TPTE antigen in the cancer.

Embodiment 91. The method of any one of embodiments 1-90, further comprising measuring a level of metabolic activity in the patient's spleen.

Embodiment 92. The method of any one of embodiments 1-91, further comprising measuring a level of metabolic activity in the patient's spleen before and after administration of the pharmaceutical composition.

Embodiment 93. The method of embodiment 91 or 92, wherein the level of metabolic activity in the patient's spleen is measured using positron emission tomography (PET), computerized tomography (CT) scans, magnetic resonance imaging (MRI), or a combination thereof.

Embodiment 94. The method of any one of embodiments 1-93, further comprising measuring an amount of one or more cytokines in the patient's plasma.

Embodiment 95. The method of any one of embodiments 1-94, further comprising measuring an amount of one or more cytokines in the patient's plasma before and after administration of the pharmaceutical composition.

Embodiment 96. The method of embodiment 94 or 95, wherein the one or more cytokines comprise interferon (IFN)-α, IFN-γ, interleukin (IL)-6, IFN-inducible protein (IP)-10, IL-12 p70 subunit, or a combination thereof.

Embodiment 97. The method of any one of embodiments 1-96, further comprising measuring a number of cancer lesions in the patient.

Embodiment 98. The method of any one of embodiments 1-97, further comprising measuring a number of cancer lesions in the patient before and after administration of the pharmaceutical composition.

Embodiment 99. The method of embodiment 98, wherein there are fewer cancer lesions in the patient after administration of the pharmaceutical composition than before administration of the pharmaceutical composition.

Embodiment 100. The method of any one of embodiments 1-99, further comprising measuring a number of T cells induced by the pharmaceutical composition in the patient.

Embodiment 101. The method of any one of embodiments 1-100, further comprising measuring a number of T cells induced by the pharmaceutical composition in the patient at a plurality of time points following administration of the pharmaceutical composition.

Embodiment 102. The method of any one of embodiments 1-101, further comprising measuring a number of T cells induced by the pharmaceutical composition in the patient following administration of the first dose the pharmaceutical composition and following administration of the second dose the pharmaceutical composition.

Embodiment 103. The method of embodiment 102, wherein the number of T cells induced by the pharmaceutical composition in the patient is greater following administration of the second dose of the pharmaceutical composition than following administration of the first dose of the pharmaceutical composition.

Embodiment 104. The method of any one of embodiments 1-103, further comprising determining a phenotype of T cells induced by the pharmaceutical composition in the patient following administration of the pharmaceutical composition.

Embodiment 105. The method of embodiment 104, wherein at least a subset of T cells induced by the pharmaceutical composition in the patient have a T-helper-1 phenotype.

Embodiment 106. The method of embodiment 104 or 105, wherein T cells induced by the pharmaceutical composition in the patient comprise T cells having a PD1+ effector memory phenotype.

Embodiment 107. The method of any one of embodiments 3-106, further comprising, for a patient classified as having evidence of disease, measuring a size of one or more cancer lesions.

Embodiment 108. The method of any one of embodiments 3-107, further, for a patient classified as having evidence of disease, comprising measuring a size of one or more cancer lesions in the patient before and after administration of the pharmaceutical composition.

Embodiment 109. The method of embodiment 108, further comprising comparing the size of one or more cancer lesions in the patient before and after administration of the pharmaceutical composition.

Embodiment 110. The method of embodiment 109, wherein the size of at least one cancer lesion in the patient after administration of the pharmaceutical composition is equal to or smaller than the size of the at least one cancer lesion before administration of the pharmaceutical composition.

Embodiment 111. The method of any one of embodiments 3-110, further comprising, for a patient classified as having evidence of disease, monitoring a duration of progression-free survival.

Embodiment 112. The method of embodiment 111, comparing the duration of progression-free survival of the patient with than a reference duration of progression-free survival.

Embodiment 113. The method of embodiment 112, wherein the reference duration of progression-free survival is an average duration of progression-free survival of a plurality of comparable patients who have not received the pharmaceutical composition.

Embodiment 114. The method of embodiment 112 or 113, wherein the duration of progression-free survival of the patient is longer in time than a reference duration of progression-free survival.

Embodiment 115. The method of any one of embodiments 3-114, further comprising, for a patient classified as having evidence of disease, measuring a duration of disease stabilization.

Embodiment 116. The method of 115, wherein disease stabilization is determined by applying an irRECIST or RECIST 1.1 standard.

Embodiment 117. The method of embodiment 115 or 116, further comprising comparing the duration of disease stabilization of the patient to a reference duration of disease stabilization.

Embodiment 118. The method of embodiment 117, wherein the reference duration of disease stabilization is an average duration of disease stabilization of a plurality of comparable patients who have not received the pharmaceutical composition.

Embodiment 119. The method of embodiment 118, wherein the patient exhibits an increased duration of disease stabilization compared to the reference duration of disease stabilization.

Embodiment 120. The method of any one of embodiments 3-119, further comprising, for a patient classified as having evidence of disease, measuring a duration of tumor responsiveness.

Embodiment 121. The method of 120, wherein tumor responsiveness is determined by applying an irRECIST or RECIST 1.1 standard.

Embodiment 122. The method of embodiment 120 or 121, further comprising comparing the duration of tumor responsiveness of the patient to a reference duration of tumor responsiveness.

Embodiment 123. The method of embodiment 122, wherein the reference duration of tumor responsiveness is an average duration of tumor responsiveness of a plurality of comparable patients who have not received the pharmaceutical composition.

Embodiment 124. The method of embodiment 123, wherein the patient exhibits an increased duration of tumor responsiveness compared to the reference duration of tumor responsiveness.

Embodiment 125. The method of any one of embodiments 1-106, further comprising, for a patient classified as having no evidence of disease, monitoring a duration of disease-free survival.

Embodiment 126. The method of embodiment 125, further comprising comparing the duration disease-free survival of the patient to a reference duration of disease-free survival.

Embodiment 127. The method of embodiment 126, wherein the reference duration of disease-free survival is an average duration of disease-free survival of a plurality of comparable patients who have not received the pharmaceutical composition.

Embodiment 128. The method of embodiment 127, wherein the patient exhibits an increased duration of disease-free survival compared to the reference duration of disease-free survival.

Embodiment 129. The method of any one of embodiments 1-106 and 125-128, further comprising, for a patient classified as having no evidence of disease, measuring a duration to disease relapse.

Embodiment 130. The method of 129, wherein disease relapse is determined by applying an irRECIST or RECIST 1.1 standard.

Embodiment 131. The method of embodiment 129 or 130, further comprising comparing the duration to disease relapse of the patient to a reference duration to disease relapse.

Embodiment 132. The method of embodiment 131, wherein the reference duration to disease relapse is an average duration to disease relapse of a plurality of comparable patients who have not received the pharmaceutical composition.

Embodiment 133. The method of embodiment 132, wherein the patient exhibits an increased duration to disease relapse compared to the reference duration to disease relapse.

Embodiment 134. A pharmaceutical composition for use in inducing an immune response against cancer in a patient, wherein the pharmaceutical composition comprises:
(a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and
(b) lipid particles;
and wherein the patient is classified as having no evidence of disease, but has previously been diagnosed with cancer.

Embodiment 135. A pharmaceutical composition for use in treating cancer, wherein the pharmaceutical composition comprises:
(a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and
(b) lipid particles;
and wherein the patient is classified as having no evidence of disease, but has previously been diagnosed with cancer.

Embodiment 136. A pharmaceutical composition for use in inducing an immune response against cancer in a patient, wherein the pharmaceutical composition comprises:
(a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and
(b) lipid particles.

Embodiment 137. A pharmaceutical composition for use in treating cancer, wherein the pharmaceutical composition comprises:
(a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and
(b) lipid particles.

Embodiment 138. The pharmaceutical composition of embodiment 136 or 137, wherein the patient is classified as having no evidence of disease at the time of administration.

Embodiment 139. The pharmaceutical composition of embodiment 136 or 137, wherein the patient is classified as having evidence of disease at the time of administration.

Embodiment 140. The pharmaceutical composition of any one of embodiments 134-139, wherein evidence of disease or no evidence of disease is or was determined by applying an immune-related Response Evaluation Criteria In Solid Tumors (irRECIST) standard or RECIST 1.1 standard.

Embodiment 141. The pharmaceutical composition of any one of embodiments 134-140, wherein the cancer is melanoma.

Embodiment 142. The pharmaceutical composition of any one of embodiments 134-141, wherein the one or more RNA molecules comprise:
(i) a first RNA molecule encoding the NY-ESO-1 antigen,
(ii) a second RNA molecule encoding a MAGE-3 antigen,
(iii) a third RNA molecule encoding a tyrosinase antigen, and
(iv) a fourth RNA molecule encoding a TPTE antigen.

Embodiment 143. The pharmaceutical composition of any one of embodiments 134-142, wherein a single RNA molecule of the one or more RNA molecules encodes at least two of the NY-ESO-1 antigen, the MAGE-3 antigen, the tyrosinase antigen, and the TPTE antigen.

Embodiment 144. The pharmaceutical composition of any one of embodiments 134-143, wherein a single RNA molecule of the one or more RNA molecules encodes a polyepitopic polypeptide, wherein the polyepitopic polypeptide comprises at least two of the NY-ESO-1 antigen, the MAGE-3 antigen, the tyrosinase antigen, and the TPTE antigen.

Embodiment 145. The pharmaceutical composition of any one of embodiments 134-144, wherein the one or more RNA molecules further comprise at least one sequence that encodes a CD4+ epitope.

Embodiment 146. The pharmaceutical composition of any one of embodiments 134-145, wherein the one or more RNA molecules comprise at least one sequence that encodes tetanus toxoid P2, a sequence that encodes tetanus toxoid P16, or both.

Embodiment 147. The pharmaceutical composition of any one of embodiments 134-146, wherein the one or more RNA molecules comprise a sequence encoding an MHC class I trafficking domain.

Embodiment 148. The pharmaceutical composition of any one of embodiments 134-147, wherein the one or more RNA molecules comprises a 5′ cap or 5′ cap analogue.

Embodiment 149. The pharmaceutical composition of any one of embodiments 134-148, wherein the one or more RNA molecules comprises a sequence encoding a signal peptide.

Embodiment 150. The pharmaceutical composition of any one of embodiments 134-149, wherein the one or more RNA molecules comprise at least one non-coding regulatory element.

Embodiment 151. The pharmaceutical composition of any one of embodiments 134-150, wherein the one or more RNA molecules comprises a poly-adenine tail.

Embodiment 152. The pharmaceutical composition of embodiment 151, wherein the poly-adenine tail is or comprises a modified adenine sequence.

Embodiment 153. The pharmaceutical composition of any one of embodiments 134-152, wherein the one or more RNA molecules comprises at least one 5′ untranslated region (UTR) and/or at least one 3′ UTR.

Embodiment 154. The pharmaceutical composition of embodiment 153, wherein the one or more RNA molecules comprises in 5′ to 3′ order:
(i) a 5′ cap or 5′ cap analogue;
(ii) at least one 5′ UTR;
(iii) a signal peptide;
(iv) a coding region that encodes at least one of the NY-ESO-1 antigen, the MAGE-3 antigen, the tyrosinase antigen, and the TPTE antigen;
(v) at least one sequence that encodes a tetanus toxoid P2, tetanus toxoid P16, or both;
(vi) a sequence encoding an MHC class I trafficking domain;
(vii) at least one 3′UTR; and
(viii) a poly-adenine tail.

Embodiment 155. The pharmaceutical composition of any one of embodiments 134-154, wherein the one or more RNA molecules comprise natural ribonucleotides.

Embodiment 156. The pharmaceutical composition of any one of embodiments 134-155, wherein the one or more RNA molecules comprise modified or synthetic ribonucleotides.

Embodiment 157. The pharmaceutical composition of any one of embodiments 134-156, wherein at least one of the NY-ESO-1 antigen, the MAGE-3 antigen, the tyrosinase antigen, and the TPTE antigen are full-length, non-mutated antigens.

Embodiment 158. The pharmaceutical composition of any one of embodiments 134-157, wherein all of the NY-ESO-1 antigen, the MAGE-3 antigen, the tyrosinase antigen, and the TPTE antigen are full-length, non-mutated antigens.

Embodiment 159. The pharmaceutical composition of any one of embodiments 134-158, wherein at least one of the NY-ESO-1 antigen, the MAGE-3 antigen, the tyrosinase antigen, and the TPTE antigen are expressed from dendritic cells in lymphoid tissues of the patient.

Embodiment 160. The pharmaceutical composition of any one of embodiments 134-159, wherein at least one of the NY-ESO-1 antigen, the MAGE-3 antigen, the tyrosinase antigen, and the TPTE antigen are present in the cancer.

Embodiment 161. The pharmaceutical composition of any one of embodiments 134-160, wherein the lipid particles comprise liposomes.

Embodiment 162. The pharmaceutical composition of any one of embodiments 134-160, wherein the lipid particles comprise cationic liposomes.

Embodiment 163. The pharmaceutical composition of any one of embodiments 134-162, wherein the lipid particles comprise lipid nanoparticles.

Embodiment 164. The pharmaceutical composition of any one of embodiments 134-163, wherein the lipid particles comprise N,N,N trimethyl-2-3-dioleyloxy-1-propanaminium chloride (DOTMA), 1,2-dioleoyl-sn-glycero-3-phosphoethanolamine phospholipid (DOPE), or both.

Embodiment 165. The pharmaceutical composition of any one of embodiments 134-164, wherein the lipid particles comprise at least one ionizable aminolipid.

Embodiment 166. The pharmaceutical composition of any one of embodiments 134-165, wherein the lipid particles comprise at least one ionizable aminolipid and a helper lipid.

Embodiment 167. The pharmaceutical composition of any one of embodiment 166, wherein the helper lipid is or comprises a phospholipid.

Embodiment 168. The pharmaceutical composition of any one of embodiment 166 or 167, wherein the helper lipid is or comprises a sterol.

Embodiment 169. The pharmaceutical composition of any one of embodiments 134-168, wherein the lipid particles comprises at least one polymer-conjugated lipid.

Embodiment 170. The pharmaceutical composition of any one of embodiments 134-169, wherein the patient is a human.

Embodiment 171. The pharmaceutical composition of any one of embodiments 134-170, wherein the cancer is an epithelial cancer.

Embodiment 172. The pharmaceutical composition of any one of embodiments 134-171, wherein the cancer is a melanoma.

Embodiment 173. The pharmaceutical composition of embodiment 172, wherein the melanoma is a cutaneous melanoma.

Embodiment 174. The pharmaceutical composition of any one of embodiments 134-173, wherein the cancer is advanced stage.

Embodiment 175. The pharmaceutical composition of any one of embodiments 134-174, wherein the cancer is Stage II, Stage III or Stage IV.

Embodiment 176. The pharmaceutical composition of any one of embodiments 134-175, wherein the cancer is Stage IIIB, Stage IIIC, or Stage IV melanoma.

Embodiment 177. The pharmaceutical composition of any one of embodiments 134-176, wherein the cancer is fully resected, there is no evidence of disease, or both.

Embodiment 178. Use of a pharmaceutical composition for inducing an immune response against cancer in a patient, wherein the pharmaceutical composition comprises:
(a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and
(b) lipid particles;
and wherein the patient is classified as having no evidence of disease, but has previously been diagnosed with cancer.

Embodiment 179. Use of a pharmaceutical composition for treating cancer, wherein the pharmaceutical composition comprises:
(a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and
(b) lipid particles;
and wherein the patient is classified as having no evidence of disease, but has previously been diagnosed with cancer.

Embodiment 180. The use of embodiment 178 or 179, wherein the cancer is melanoma.

Embodiment 181. Use of a pharmaceutical composition for inducing an immune response against cancer in a patient, wherein the pharmaceutical composition comprises:
(a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and
(b) lipid particles.

Embodiment 182. Use of a pharmaceutical composition for treating cancer, wherein the pharmaceutical composition comprises:
(a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and
(b) lipid particles.

Embodiment 183. The use of embodiment 181 or 182, wherein the patient is classified as having no evidence of disease at the time of administration.

Embodiment 184. The use of embodiment 181 or 182, wherein the patient is classified as having evidence of disease at the time of administration.

Embodiment 185. The use of any one of embodiments 178-184, wherein evidence of disease or no evidence of disease is or was determined by applying an immune-related Response Evaluation Criteria In Solid Tumors (irRECIST) standard or RECIST 1.1 standard.

Embodiment 186. The use of any one of embodiments 178-185, wherein the cancer is melanoma.

Embodiment 187. The use of any one of embodiments 178-186, wherein the one or more RNA molecules comprise:
(i) a first RNA molecule encoding the NY-ESO-1 antigen,
(ii) a second RNA molecule encoding a MAGE-3 antigen,
(iii) a third RNA molecule encoding a tyrosinase antigen, and
(iv) a fourth RNA molecule encoding a TPTE antigen.

Embodiment 188. The use of any one of embodiments 178-187, wherein a single RNA molecule of the one or more RNA molecules encodes at least two of the NY-ESO-1 antigen, the MAGE-3 antigen, the tyrosinase antigen, and the TPTE antigen.

Embodiment 189. The use of any one of embodiments 178-188, wherein a single RNA molecule of the one or more RNA molecules encodes a polyepitopic polypeptide, wherein the polyepitopic polypeptide comprises at least two of the NY-ESO-1 antigen, the MAGE-3 antigen, the tyrosinase antigen, and the TPTE antigen.

Embodiment 190. The use of any one of embodiments 178-189, wherein the one or more RNA molecules further comprise at least one sequence that encodes a CD4+ epitope.

Embodiment 191. The use of embodiment 190, wherein the one or more RNA molecules comprise at least one sequence that encodes tetanus toxoid P2, a sequence that encodes tetanus toxoid P16, or both.

Embodiment 192. The use of any one of embodiments 178-191, wherein the one or more RNA molecules comprise a sequence encoding an MHC class I trafficking domain.

Embodiment 193. The use of any one of embodiments 178-192, wherein the one or more RNA molecules comprises a 5′ cap or 5′ cap analogue.

Embodiment 194. The use of any one of embodiments 178-193, wherein the one or more RNA molecules comprises a sequence encoding a signal peptide.

Embodiment 195. The use of any one of embodiments 178-194, wherein the one or more RNA molecules comprise at least one non-coding regulatory element.

Embodiment 196. The use of any one of embodiments 178-195, wherein the one or more RNA molecules comprises a poly-adenine tail.

Embodiment 197. The use of embodiment 196, wherein the poly-adenine tail is or comprises a modified adenine sequence.

Embodiment 198. The use of any one of embodiments 178-197, wherein the one or more RNA molecules comprises at least one 5′ untranslated region (UTR) and/or at least one 3′ UTR.

Embodiment 199. The use of embodiment 198, wherein the one or more RNA molecules comprises in 5′ to 3′ order:
(i) a 5′ cap or 5′ cap analogue;
(ii) at least one 5′ UTR;
(iii) a signal peptide;
(iv) a coding region that encodes at least one of the NY-ESO-1 antigen, the MAGE-3 antigen, the tyrosinase antigen, and the TPTE antigen;
(v) at least one sequence that encodes a tetanus toxoid P2, tetanus toxoid P16, or both;
(vi) a sequence encoding an MHC class I trafficking domain;
(vii) at least one 3′UTR; and
(viii) a poly-adenine tail.

Embodiment 200. The use of any one of embodiments 178-199, wherein the one or more RNA molecules comprise natural ribonucleotides.

Embodiment 201. The use of any one of embodiments 178-200, wherein the one or more RNA molecules comprise modified or synthetic ribonucleotides.

Embodiment 202. The use of any one of embodiments 178-201, wherein at least one of the NY-ESO-1 antigen, the MAGE-3 antigen, the tyrosinase antigen, and the TPTE antigen are full-length, non-mutated antigens.

Embodiment 203. The use of any one of embodiments 178-202, wherein all of the NY-ESO-1 antigen, the MAGE-3 antigen, the tyrosinase antigen, and the TPTE antigen are full-length, non-mutated antigens.

Embodiment 204. The use of any one of embodiments 178-203, wherein at least one of the NY-ESO-1 antigen, the MAGE-3 antigen, the tyrosinase antigen, and the TPTE antigen are expressed from dendritic cells in lymphoid tissues of the patient.

Embodiment 205. The use of any one of embodiments 178-204, wherein at least one of the NY-ESO-1 antigen, the MAGE-3 antigen, the tyrosinase antigen, and the TPTE antigen are present in the cancer.

Embodiment 206. The use of any one of embodiments 178-205, wherein the lipid particles comprise liposomes.

Embodiment 207. The use of any one of embodiments 178-205, wherein the lipid particles comprise cationic liposomes.

Embodiment 208. The use of any one of embodiments 178-207, wherein the lipid particles comprise lipid nanoparticles.

Embodiment 209. The use of any one of embodiments 178-208, wherein the lipid particles comprise N,N,N trimethyl-2-3-dioleyloxy-1-propanaminium chloride (DOTMA), 1,2-dioleoyl-sn-glycero-3-phosphoethanolamine phospholipid (DOPE), or both.

Embodiment 210. The use of any one of embodiments 178-209, wherein the lipid particles comprise at least one ionizable aminolipid.

Embodiment 211. The use of any one of embodiments 178-210, wherein the lipid particles comprise at least one ionizable aminolipid and a helper lipid.

Embodiment 212. The use of any one of embodiment 211, wherein the helper lipid is or comprises a phospholipid.

Embodiment 213. The use of any one of embodiment 211 or 212, wherein the helper lipid is or comprises a sterol.

Embodiment 214. The use of any one of embodiments 178-213, wherein the lipid particles comprises at least one polymer-conjugated lipid.

Embodiment 215. The use of any one of embodiments 178-214, wherein the patient is a human.

Embodiment 216. The use of any one of embodiments 178-215, wherein the cancer is an epithelial cancer.

Embodiment 217. The use of any one of embodiments 178-216, wherein the cancer is a melanoma.

Embodiment 218. The use of embodiment 217, wherein the melanoma is a cutaneous melanoma.

Embodiment 219. The use of any one of embodiments 178-218, wherein the cancer is advanced stage.

Embodiment 220. The use of any one of embodiments 178-219, wherein the cancer is Stage II, Stage III or Stage IV.

Embodiment 221. The use of any one of embodiments 178-220, wherein the cancer is Stage IIIB, Stage IIIC, or Stage IV melanoma.

Embodiment 222. The use of any one of embodiments 178-221, wherein the cancer is fully resected, there is no evidence of disease, or both.

### EXEMPLIFICATION

### Example 1: Trial Design and Material and Methods

Design of Lipo-MERIT clinical trial. The main objectives of this trial (NCT02410733) are to assess the safety and tolerability of melanoma FixVac, its preliminary efficacy and progression-free survival; to investigate vaccine-induced antigen-specific immune responses; and to determine a phase II dose. As used herein, the term “FixVac” refers to a pharmaceutical composition comprising one or more RNA molecules as depicted in FIG. 1 and lipid particles (e.g., lipoplexes or lipid nanoparticles). BNT111 is an embodiment of FixVac. As this is a first-in-human phase I trial, and in line with the objectives, no statistical methods were used to predetermine sample size. The investigators have not been blinded to allocation during experiments and outcome assessments.

The trial is being carried out in Germany in accordance with the Declaration of Helsinki and Good Clinical Practice Guidelines, and with approval by the independent ethics committee (Ethik-Kommission of the Landesärztekammer Rheinland Pfalz, Mainz, Germany) and the competent regulatory authority (Paul-Ehrlich Institute, Langen, Germany). All patients provided written informed consent.

Eligible patients have malignant melanoma stage III B-C or IV (American Joint Committee on Cancer (AJCC) 2009 melanoma classification), both resected and unresected, and thus with measurable and non-measurable disease at baseline, with expression of at least one of the four vaccine TAAs. Patients are also at least 18 years of age and have adequate haematological and end-organ function. Inclusion criteria required that subjects are not eligible for, or have declined, any other available approved therapy, after all available treatment options have been transparently disclosed. Key exclusion criteria are the presence of clinically relevant autoimmune disease, human immunodeficiency virus (HIV), hepatitis B virus (HBV), hepatitis C virus (HCV) or active brain metastases. Patients receive eight injections of RNA-LPX within 64 days (prime/repeat boost protocol), except of patients from cohort 1, who receive only six injections within 43 days. Optional continued treatment of one monthly vaccine dose is offered to patients with measurable disease who do not exhibit disease progression or drug-related toxicities. Patients are treated in seven dose-escalation cohorts with target doses ranging from 14.4 μg to 400 μg total RNA, and in three expanded cohorts that further explore dose levels of 14.4 μg, 50 μg and 100 μg. RNA-LPX administration is performed by four consecutive intravenous slow bolus injections using a venous catheter.

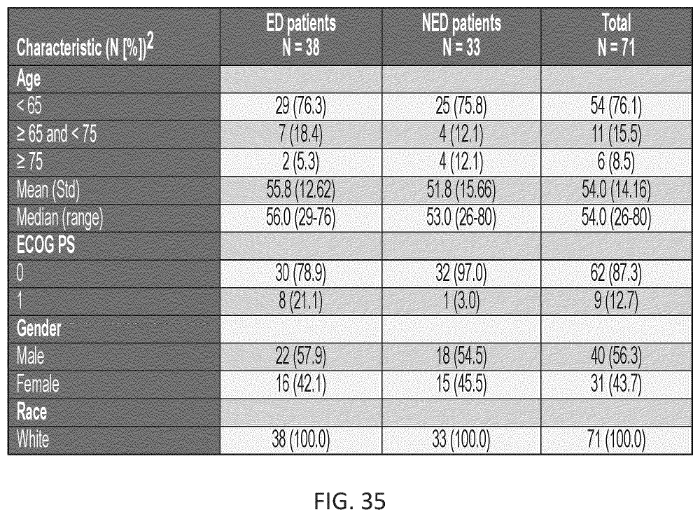

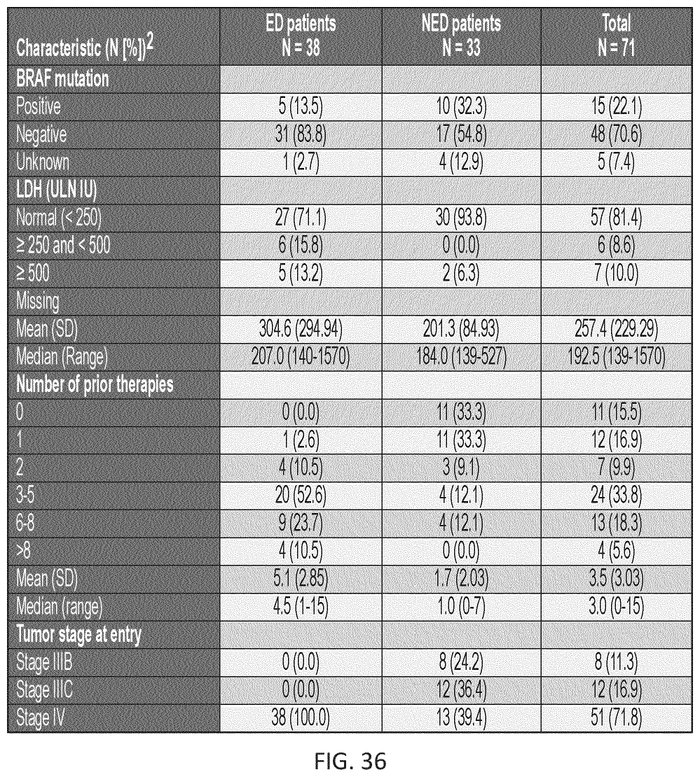

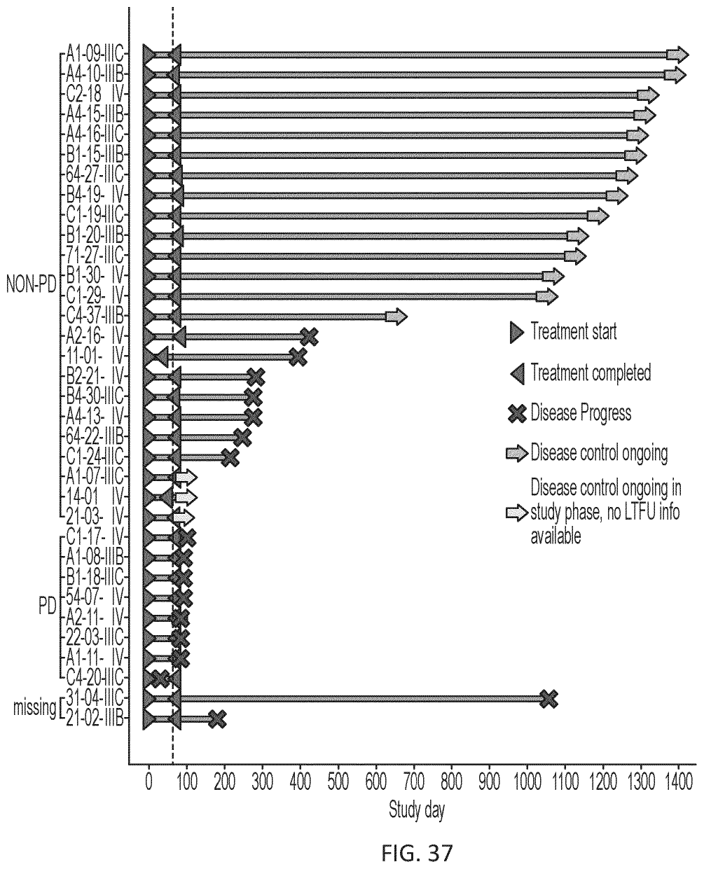

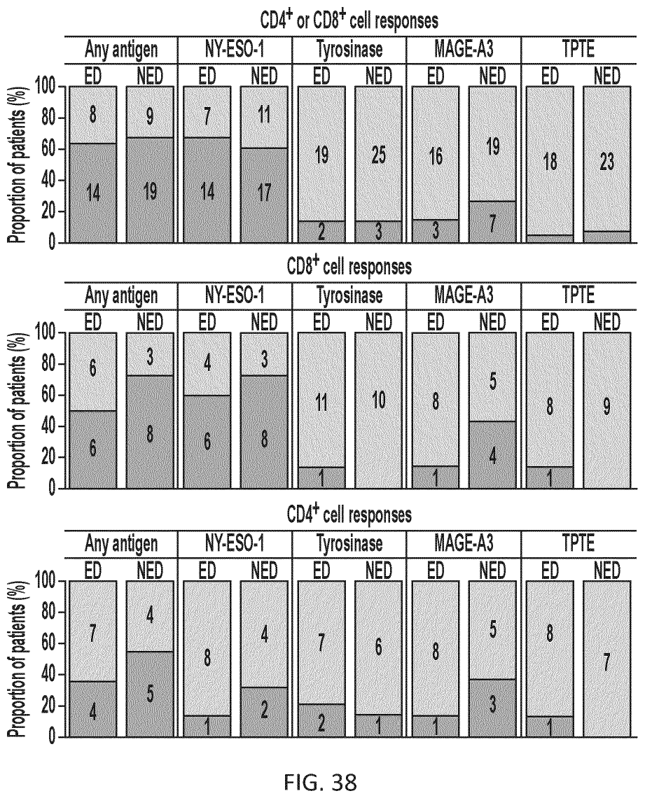

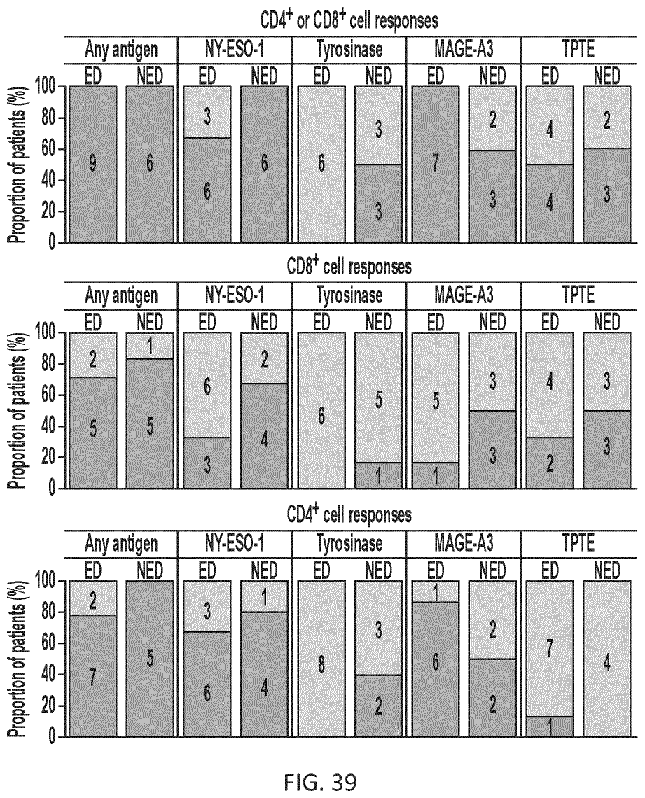

Additional information regarding patients who participated in the study are included in FIGS. 35 and 36.

Key study assessments. Safety and tolerability were evaluated on the basis of changes in physical examination or vital signs, clinical laboratory analysis and reports of any adverse events, including clinically significant laboratory abnormalities. Adverse events were graded according to National Cancer Institute Common Terminology Criteria (NCI CTC version 4.03). Safety was characterized according to these criteria from grades 1 to 5.

Imaging of the thorax, abdomen and brain by CT scans and magnetic resonance imaging (MRI) was performed at baseline, and then every 90 days according to local imaging guidelines and irRECIST version 1.1 (ref. 25).

Vital signs (body temperature, heart rate and blood pressure) were measured before and 4 h after the administration of FixVac and as clinically indicated.

For assessment of vaccine-induced immune responses, blood was sampled at baseline, before the fourth, sixth and eighth vaccine administrations, as well as 7-14 days and 19-33 days after the eighth administration. In cohort 1, blood was sampled at baseline, before the third, fourth, fifth and sixth vaccine administrations, and 7-14 days after the sixth administration. During continued treatment, blood samples were taken before each administration. PBMCs were isolated by Ficoll-Hypaque (Amersham Biosciences) density gradient centrifugation from peripheral blood or leukapheresis samples.

For cytokine analysis, serum was sampled before and 2 h, 6 h, 24 h or 48 h after treatment and shipped at −80° C. Samples were analyzed using a human pan-IFN-α ELISA (PBL Assay Science) and a multisport assay system (Meso Scale Discovery) in duplicate (MLM Medical Labs). Sample sizes per analysis were: IFN-α versus IP-10, n=166; IFN-α versus IFN-γ, n=167; IFN-α versus IL-6, n=167; IFN-α versus IL-12 p70, n=167; from 72 patients and with up to 6 data points per patient.

Delayed-type hypersensitivity (DTH) reactions were assessed in detail in a fraction of patients. After intradermal injection of RNA diluted in concentrated (×2.67) Ringer's solution (manufactured under good manufacturing practice (GMP) guidelines by BAG Health Care), skin-infiltrating lymphocytes (SILs) were recovered from a punch biopsy after two to three weeks of culture in high-dose IL-2 (50,000 U ml-1)-containing medium (RPMI 1640, 7% human AB serum, 1× antimycotic).

Data reporting. This is an ongoing exploratory, open-label, non-randomized first in-human phase I clinical trial. The data presented are based on an exploratory interim analysis with a data extraction date of 29 Jul. 2019. This exploratory analysis was carried out to inform and initiate the design of a randomized phase 2 trial for the FixVac/anti-PD1 combination treatment of CPI-experienced patients. The analysis was triggered by the availability of baseline to three-month comparative immunogenicity data for about half of the study population (n=51) across dose cohorts, and of at least three months of follow-up data for both subsets of patients treated with FixVac monotherapy and the FixVac/anti-PD1 combination. The exploratory interim analysis was focused in particular on vaccine-induced immune responses (a secondary end point). Moreover, preliminary high-level data on the tolerability of the study medication (the primary end point) and the response of patients with measurable disease according to irRECIST 1.1 (a secondary end point) are reported. Clinical data shown are preliminary and not fully source data verified. At the time that this paper was accepted for publication, 109 of 115 (95%) patients had been enrolled.

Exemplary Materials and Methods. The following materials and methods were used in the following examples.

FDG-PET/CT imaging. [18F]FDG uptake in the spleen was assessed by PET/CT imaging after a 4-6 h fasting period (resulting in a blood glucose level of less than 130 mg dl-1) and application of approximately 2 MBq kg-1 FDG after a 60-70 min distribution time. Acquisition was conducted by an EARL-certified Philips Gemini time-of-flight (TOF) PET/CT scanner with 2-2.5 min per bed position according to clinical routine. Mean standardized uptake values (SUVs) were measured in a 2 cm sphere centered within the spleen.

TAA expression profiling. Total RNA from formalin-fixed paraffin-embedded (FFPE) samples of patients was extracted (RNeasy FFPE kit, Qiagen). Complementary DNA was synthesized (Peqstar, VWR International) and analyzed by quantitative polymerase chain reaction (PCR; Applied Biosystems 7300 real-time PCR system, Thermo Fisher Scientific), according to good clinical laboratory practice (GCLP) guidelines, for the expression of the NY-ESO-1, tyrosinase, MAGE-A3 and TPTE RNAs, as well as the reference gene encoding hypoxanthine guanine phosphoribosyltransferase (HPRT1). Median quantification cycle (Cq) values of each TAA were normalized to the median Cq of the reference gene to obtain a relative expression ΔCq value, which was classified as positive or negative on the basis of TAA-specific cut-off points.

Manufacturing of RNA-LPX. RNA, liposomes and RNA-LPX were manufactured under GMP conditions. Manufacturing of the RNA was performed by in vitro transcription of DNA plasmid templates encoding full-length sequences of NY-ESO-1, MAGE-A3, TPTE or amino acids 1-477 of tyrosinase. Manufacturing, analysis and release of the four TAA-encoding RNA drug products was performed as described previously (Ref. 26).

Liposomes with net cationic charges were used to complex the RNAs to form RNA-LPX. The cationic liposomes were manufactured using an adopted proprietary protocol (Ref. 27) based on the ethanol injection technique (Ref. 28) from the cationic synthetic lipid (R)—N,N,N trimethyl-2-3-dioleyloxy-1-propanaminium chloride (R-DOTMA) (Merck and Cie) and the phospholipid 1,2-dioleoyl-sn-glycero-3-phosphoethanolamine phospholipid (DOPE) (Corden Pharma). Release analysis for the liposomes included determination of appearance, lipid concentration, RNase presence, particle size, osmolality, pH, subvisible particles, pyrogen testing and sterility.

The injectable RNA-LPX drug products were prepared in a dedicated pharmacy by incubating the individual concentrated RNA drug products with isotonic NaCl solution (0.9%) (Fresenius Kabi) and cationic liposomes according to a proprietary (Ref. 27) protocol. The RNA-LPX preparation protocol was derived from protocols for nucleotide lipoplex formation as described (Ref. 8, 29). Before injection, RNA-LPX was further diluted with isotonic NaCl solution (0.9%) (Fresenius Kabi) to the intended concentration. Periodic quality control of RNA-LPX drug products included determination of RNA content, RNA integrity, particle size and polydispersity index.

In vitro stimulation of PBMCs. CD4+ and CD8+ T cells were isolated from cryopreserved PBMCs using microbeads (Miltenyi Biotec). For IVS, TAAs encoding RNA or peptides were used. For IVS with RNA, CD4− or CD8-depleted PBMCs were electroporated after overnight rest with RNAs encoding vaccine antigens, enhanced green fluorescent protein (eGFP), influenza matrix protein 1 (M1) or tetanus p2/p16 sequences (influenza M1 and tetanus p2/p16 being positive controls for CD4+ and CD8+ T cells, respectively). The cells were then left to rest for 3 h at 37° C. and irradiated at 15 Gy. Overnight rested CD4+/CD8+ T cells and electroporated and irradiated antigen-presenting cells were combined at an effector to target (E:T) ratio of 2:1. For peptide IVS, CD4+ T cells were expanded in the presence of fast dendritic cells (E:T=10:1) pulsed with PepMixes encoding MAGE-A3, tyrosinase, TPTE or NY-ESO-1. For the expansion of CD8+ T cells, CD4-depleted PBMCs were co-cultured with purified CD8+ T cells (E:T=1:10) in the presence of IL-4 and granulocyte macrophage colony-stimulating factor (GM-CSF) (each 1,000 U ml-1) and the respective peptides. One day after starting the IVS, fresh culture medium containing 10 U ml-1 IL-2 (Proleukin S, Novartis) and 5 ng ml-1 IL-15 (Peprotech) was added. CD8 IVS cultures stimulated with peptides additionally received IL-4 and GM-CSF (each 1,000 U ml-1). For tumor cell lysis experiments, peptide-pulsed bulk PBMCs were used for IVS and harvested after 6-8 days of culture. For longer cultures, IL-2 was replenished 7 days after setting up the IVS cultures. After 11 days of stimulation, cells were analyzed via flow cytometry and used in ELISpot assays.

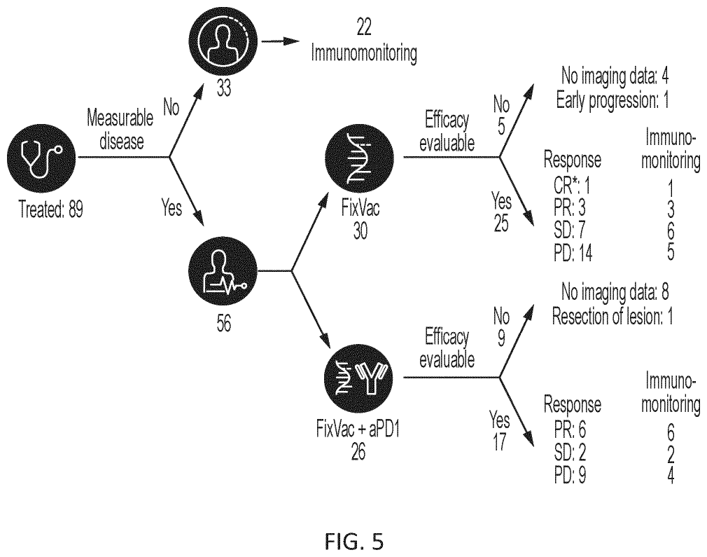

IFN-γ ELISpot. ELISpot analysis was performed for 51 patients (50 patients ex vivo, 20 patients after IVS). In addition to the 49 patients shown in FIG. 5, two patients who also received BRAF/MEK inhibitors were tested in IFN-γ ELISPOT. Multiscreen filter plates (Merck Millipore), precoated with antibodies specific for IFN-γ (Mabtech), were washed with phosphate-buffered saline (PBS) and blocked with X-VIVO 15 (Lonza) containing 2% human serum albumin (CSL-Behring) for 1-5 hours. Next, 0.5×10⁵ to 3×10⁵ effector cells per well were stimulated for 16-20 hours either with peptides (ex vivo setting), with autologous dendritic cells electroporated with RNA or loaded with peptides (after IVS), or with peptide-loaded HLA class I or II transfected K562 cells (for TCR validation). For analysis of ex vivo T-cell responses, cryopreserved PBMCs were subjected to ELISpot after a resting period of 2-5 hours at 37° C. Alternatively, CD4⁻ or CD8⁻ depleted PBMCs were used as CD8 or CD4 effectors. All tests were performed in duplicate or triplicate and included positive controls (Staphylococcus enterotoxin B (Sigma Aldrich), anti-CD3 (Mabtech)) as well as cells from a reference donor with known reactivity. Spots were visualized with a biotin-conjugated anti-IFNγ antibody (Mabtech) followed by incubation with ExtrAvidin-alkaline phosphatase (Sigma-Aldrich) and 5-bromo-4-chloro-3-indolyl phosphate (BCIP)/nitro blue tetrazolium (NBT) (Sigma-Aldrich). Alternatively, a secondary antibody directly conjugated with alkaline phosphatase was used (ELISpot-Pro kit, Mabtech). Plates were scanned using an ImmunoSpot series S5 Versa ELISpot analyzer (CTL, S5Versa-02-9038) or a classic robot ELISPOT reader (AID) and analyzed by ImmunoCapture version 6.3 or AID ELISPOT 7.0 software. Spot counts were summarized as median values for each triplicate or duplicate. T-cell responses stimulated by vaccine antigen encoding RNA or peptides were compared with responses elicited by target cells electroporated with control RNA (luciferase) or by unloaded cells. A response was defined as positive with a minimum of five spots per 1×10⁵ cells in the ex vivo setting or 25 spots per 5×10⁴ cells in the post-IVS setting, as well as a spot count that was more than twice as high as the respective control.

Flow cytometry. Antigen-specific CD8⁺ T cells were identified using fluorophore-coupled HLA multimers (Immudex). Cells were stained first for multimers and then for cell surface markers, as follows (antibody clones in parentheses): CD28 (CD28.8), CD197 (150503), CD45RA (HI100), CD3 (UCHT1 or SK7), CD16 (3G8), CD14 (MφP9), CD19 (SJ25C1), CD27 (L128), CD279 (EH12), CD134 (ACT35) and CD8 (RPA-T8 or SK1), all purchased from BD Biosciences; CD19 (HIB19) and CD4 (OKT4), from Biolegend. Live-dead staining was also carried out using 4′,6-diamidino-2-phenylindole (DAPI; BD) or fixable viability dyes eFluor 780 or eFluor 506 (eBioscience). Singlet, live, multimer-positive events were identified within CD3⁺ (or CD8⁺), CD4⁻CD14⁻CD16⁻CD19⁻ or CD3⁺ (or CD8⁺) CD4-events. For detection of antigen-specific T cells after IVS, single, live, CD3⁺, CD8⁺multimer+ lymphocytes were gated.

For staining of intracellular cytokines, autologous dendritic cells electroporated with RNA encoding single neo-epitopes were added at an E:T ratio of 10:1 and cultured for around 16 h at 37° C. in the presence of brefeldin A and monensin. Cells were stained for viability (using fixable viability dyes eFluor 506 or eFluor 780, eBioscience) and for surface markers CD8 (RPA-T8 or SK1i), CD16 (3G8), CD14 (MφP9) (all from BD Biosciences), CD19 (HIB19), or CD4 (OKT4) (from Biolegend). After permeabilization, intracellular cytokine staining was performed using antibodies against IFN-γ (B27, BD Biosciences) and TNF (Mab11, BD or Biolegend). IFN-γ+ and TNF+ events were identified within the CD8⁺ and CD4⁺ cells pre-gated on single, live and CD14⁻CD16⁻CD19⁻ (not used in all experiments) populations.

Cell surface expression of transfected TCR genes was analyzed using anti-TCR antibodies against the appropriate variable-region family or the constant region of the TCR-β chain (Beckman Coulter) and CD8− or CD4-specific antibodies (SK-1, BD; REA623, Miltenyi Biotec). HLA antigens of the antigen-presenting cells used for evaluating the function of TCR-transfected T cells were detected by staining with HLA class II specific antibodies (9-49, Beckman Coulter) and HLA class I specific antibodies (DX17, BD Biosciences). Acquisition was performed on a LSR Fortessa SORP, FACSCelesta or FACSCanto II cell analyzer (BD Biosciences) and analyzed via FlowJo software (Tree Star).

Cloning of HLA antigens. HLA antigens were synthesized by Eurofins Genomics Germany GmbH according to respective high-resolution HLA typing results. HLA DQA sequences were amplified from donor-specific cDNA with 2.5 U Pfu polymerase using DQA1_s (Pho GCC ACC ATG ATC CTA AAC AAA GCT CTG MTG C) and DQA1_as (TAT GCG ATC GCT CAC AAK GGC CCY TGG TGT CTG) primers. HLA antigens were cloned into appropriately digested IVT vectors (Ref. 10).

RNA transfer into cells. RNA was added to cells suspended in X-VIVO 15 medium (Lonza) in a precooled 4-mm-gap sterile electroporation cuvette (Bio-Rad). Electroporation was performed with a BTX ECM 830 square wave electroporation system with conditions established previously for every cell type (T cells, 500 V, 3 ms per pulse, one pulse; immature dendritic cells, 300 V, 12 ms per pulse, one pulse; SK-MEL-29, 250 V, 3 ms per pulse, three pulses; Jurkat cells, 275 V, 10 ms per pulse, one pulse; K562 cells, 200 V per 8 ms per three pulses).

Peptides. Overlapping peptide pools (PepMix) were used encoding full-length NY-ESO-1, tyrosinase, MAGE-A3 and TPTE, or short (8-11-mer) epitopes derived from these antigens, as well as an HIV gag encoding PepMix as a control. All synthetic peptides were purchased from JPT Peptide Technologies GmbH and dissolved in water with 10% dimethylsulfoxide (DMSO) to a final concentration of 3 mM (short peptides) or in 100% DMSO (PepMix).

Cell lines. The K562 and SK-MEL-28 cell lines were obtained from ATCC. The SK-MEL-29 cell line was obtained from the Memorial Sloan Kettering Cancer Center, New York. The SK-MEL-37 cell line was described in ref. 30. The Jurkat T cell line expressing a luciferase reporter driven by a nuclear factor of activated T cells (NFAT)-response element is manufactured by Promega. Reauthentication of cell lines was performed by short tandem repeat (STR) profiling at the American Type Culture Collection (ATCC) and Eurofins. All cell lines used tested negative for Mycoplasma contamination. No commonly misidentified cell lines were used.

Single-cell sorting. Sorting of single antigen-specific T cells was carried out using either ex vivo PBMCs or IVS cultures based on stimulation-induced IFN-γ secretion or multimer binding. For stimulation, PBMCs were pulsed with overlapping peptides encoding the relevant antigen or a control antigen, while expanded T cells after IVS were cultured with autologous peptide-pulsed dendritic cells. After 4 h, cells were harvested and stained with the viability dye eFluor780 (eBioscience) and fluorochrome-conjugated antibodies directed against CD3, CD8 and CD4 (all BD Biosciences) as well as IFN-γ using an IFN-γ secretion assay kit (Miltenyi Biotec). Alternatively, PBMCs were stained with the respective multimer. Sorting of single neoantigen-specific T cells was conducted on a FACSAria or a FACSMelody flow cytometer (both from BD Biosciences) using BD FACSDiva or BD FACSChorus software, respectively. Antigen-specific T cells were identified with respect to a control sample stimulated with a control antigen or stained without multimer. One T cell per well (gated on single, live CD3+ and CD8+IFN-γ+, CD4+IFN-γ+ or CD8+multimer+ lymphocytes) was harvested into 96-well V-bottom-plates (Greiner Bio-One) containing 6 μl of a mild hypotonic cell lysis buffer per well (consisting of 0.2% Triton X-100, 0.2 μl RiboLock RNase inhibitor (Thermo Scientific), 5 ng poly(A) carrier RNA (Qiagen) and 1 μl dNTP mix (10 mM, Biozym) in RNase-free water). Plates were sealed, centrifuged and stored at −65° C. to −85° C. directly after sorting.

Cloning of antigen-specific TCRs. TCR genes were cloned from single T cells as described10 with the following modifications. Plates with sorted cells were thawed, and template-switch cDNA synthesis was performed with RevertAid H reverse transcriptase (Thermo Fisher) using primers specific to TCR-α and -β constant genes (TRAC, 5′-catcacaggaactttctgggctg-3′; TRBC1, 5′-gctggtaggacaccgaggtaaagc-3′; TRBC2 5′-gctggtaagactcggaggtga agc-3′) followed by preamplification using PfuUltra Hotstart DNA polymerase (Agilent). Residual primers were removed after both cDNA synthesis and PCR by treatment with 5 U of exonuclease I (NEB). Aliquots of the cDNAs were used for Vα/Vβ gene specific multiplex PCRs. Products were analyzed on a capillary electrophoresis system (Qiagen). Samples with bands at 430 bp to 470 bp were size fractionated on agarose gels and the bands were excised and purified using a Gel Extraction Kit (Qiagen). Purified fragments were sequenced and the respective V(D)J junctions analyzed using the IMGT/V-Quest tool (Ref. 31). DNAs of novel and productively rearranged corresponding TCR chains were digested using NotI and cloned into pST1 vectors containing the appropriate constant region for in vitro transcription of complete TCR-α/β chains10.

Single-cell TCR sequencing. For selected patients, TCRs from sorted single cells were obtained by a next generation sequencing (NGS)-based single-cell TCR sequencing (scTCR-seq) workflow. Here template-switch cDNA synthesis was performed using primers specific to TCR-α and TCR-β constant genes (TRAC, 5′-catcacaggaactttctgggctg-3′; TRBC, 5′-cacgtggtcggggwagaagc-3′) followed by treatment with 5 U exonuclease I. Each cDNA was PCR amplified and barcoded by row using 2.5 U PfuUltra Hotstart DNA polymerase (Agilent), 1×PCR buffer, 0.2 mM dNTPs, 0.2 μM of one of eight tagged forward primers (Tag130-RBCx-TS 5′-cgatccagactagacgctcaggaagxxxxxaagcagtggtatcaacgcagagt-3′) and 0.1 μM of each tagged nested TCR-α and TCR-β constant gene specific primer (Tag146-TRAC, 5′-caatatgtgaccgccgagtcccaggttagagtctc tcagctggtacacggcag-3′; Tag146-TRBC, 5′-caatatgtgaccgccgagtccc aggggctcaaacacagcgacctcgggtg-3′) (95° C. for 2 min; 5 cycles of 94° C. for 30 s, 61° C. for 30 s, 72° C. for 1 min; 5 cycles of 94° C. for 30 s, 64° C. for 30 s, 72° C. for 1 min; 8 cycles of 94° C. for 30 s, 72° C. for 2 min; 72° C. for 6 min) (RBC, row bar code; TS, template switch primer). Samples of each column were pooled and purified twice using AMPure XP beads (Agencourt) with an exonuclease I treatment in between. For each pool, a third of the purified TCR cDNA was further amplified by PCR using 1 μl PfuUltra II Fusion Hotstart DNA polymerase (Agilent), lx reaction buffer, 0.2 mM dNTPs, forward primer (Tag-130 5′-(n)nnnncgatccagactagacgctcaggaag-3′) and one of 12 Tag-146 reverse oligos containing a different barcode for each column (5′-xxxxxcaatatgtgaccgccgagtcccagg-3′) (95° C. for 1 minute; 24 cycles of 94° C. for 20 seconds, 64° C. for 20 seconds, 72° C. for 30 seconds; 72° C. for 3 minutes). PCR products were pooled and purified with AMPure XP beads and exonuclease I followed by generation of TCR sequencing libraries using the TruSeq DNA Nano kit (Illumina). The scTCR libraries were sequenced on an Illumina MiSeq with a sequencing depth of 10,000 reads per well using paired-end 300-bp sequencing. Sequencing data were demultiplexed to a single-cell level using bcl2fastq software (Illumina) followed by an in-house Python script. TCR sequences were then obtained using MiXCR-2.1.5 (ref. 32). Selected paired α and β V(D)J fragments were synthesized (Eurofins Genomics) and cloned as above for subsequent in vitro transcription.

Bulk TCR sequencing. Total RNA was isolated from 1×106 snap-frozen PBMCs, collected at multiple time points during vaccination, using the RNeasy Mini kit (Qiagen). Libraries were produced with the SMARTer human TCR-α/β profiling kit (Clontech) and were sequenced using the Illumina MiSeq system. The number of total TCR reads per sample ranged from 1×106 to 4×106. Data were analyzed using VDJtools (Ref. 33) and MiXCR.

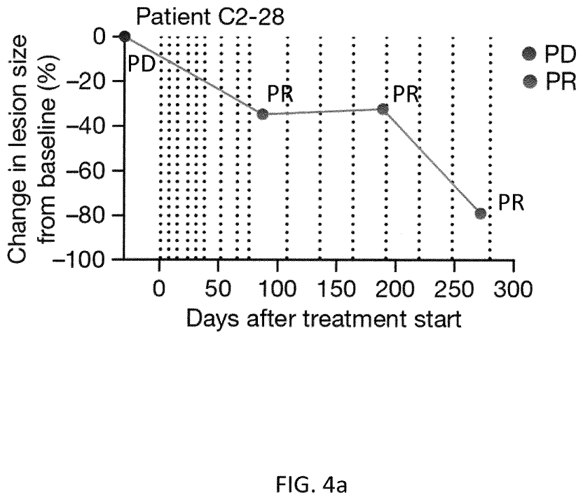

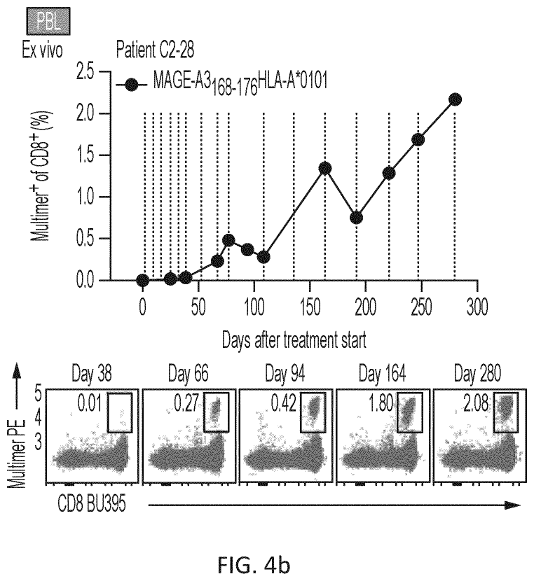

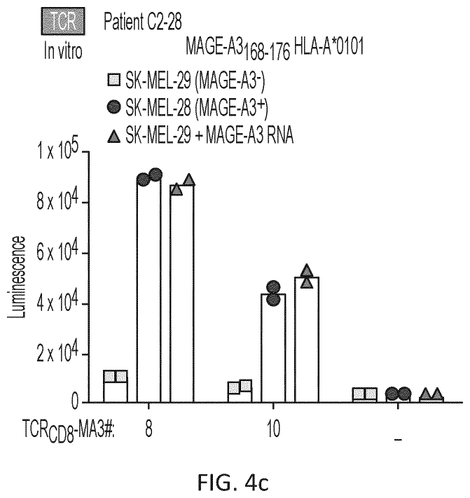

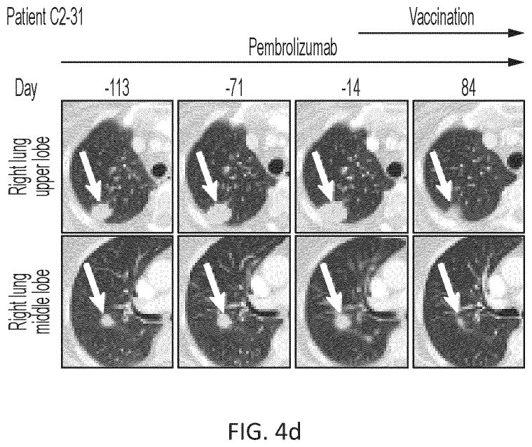

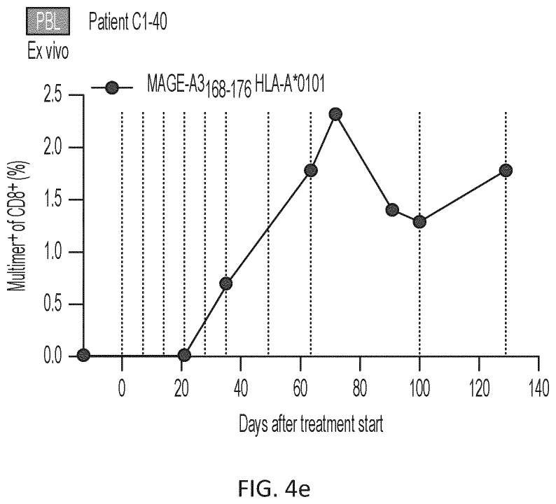

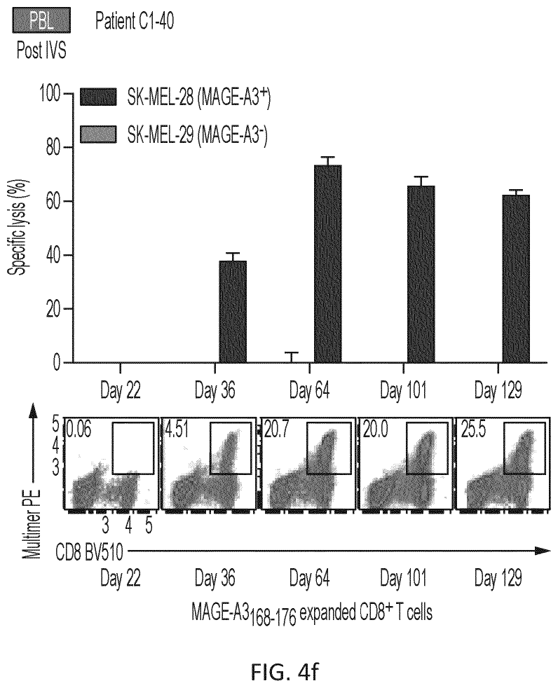

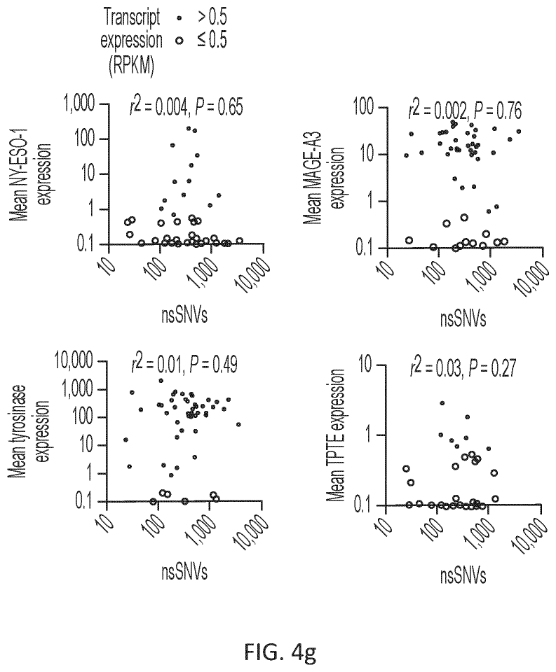

Functional TCR characterization. TCR-transfected CD4+ or CD8+ T cells from healthy donors were co-cultured with peptide-pulsed HLA class I or II transfected K562 cells and tested by IFN-γ ELISpot assay. Alternatively, Jurkat cells of the T-cell activation bioassay (NFAT, Promega) were transfected with RNAs encoding CD8-α and TCR-α/β, and tested against target cells (FIG. 4c). T-cell activation was analyzed after addition of Bio-Glo reagent (Promega) via luminescence measurement (Infinite F200 PRO, Tecan).

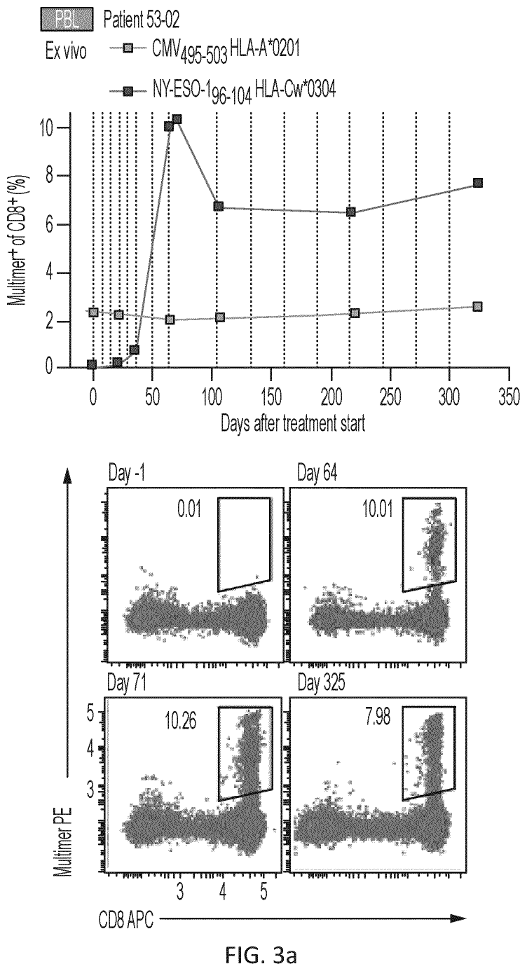

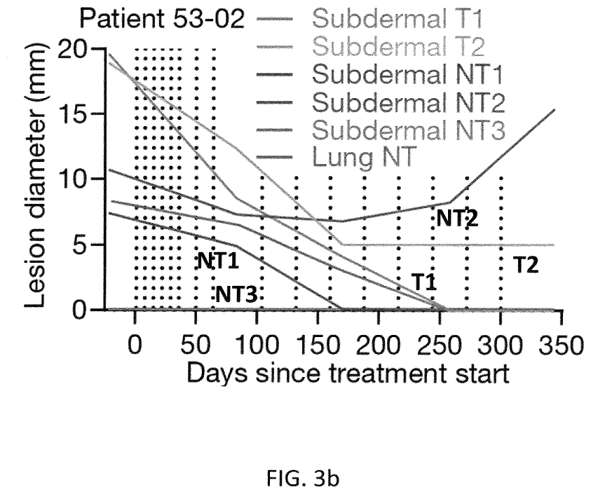

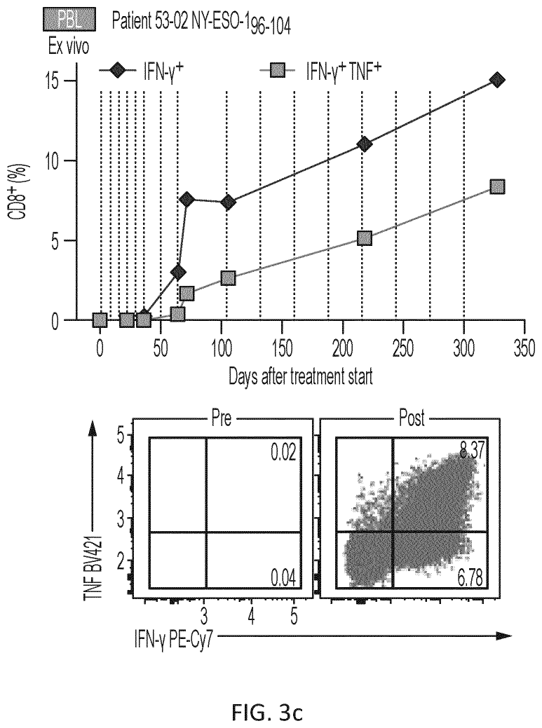

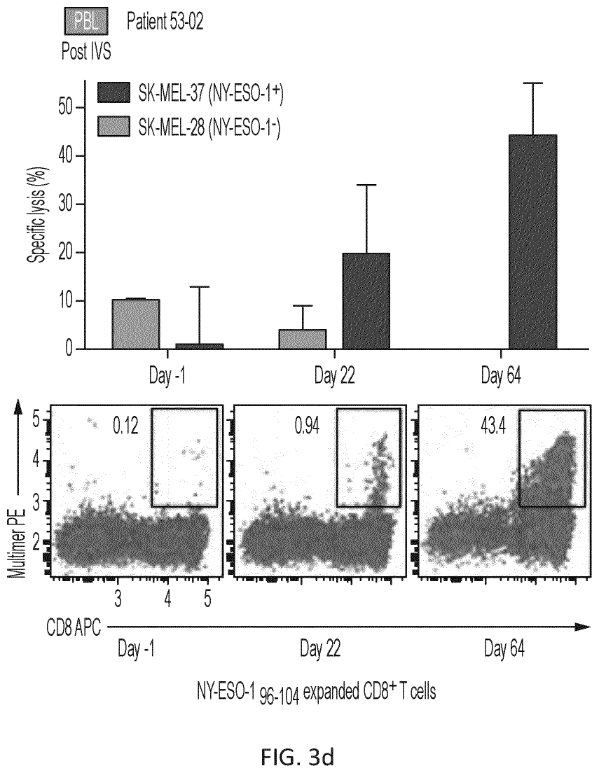

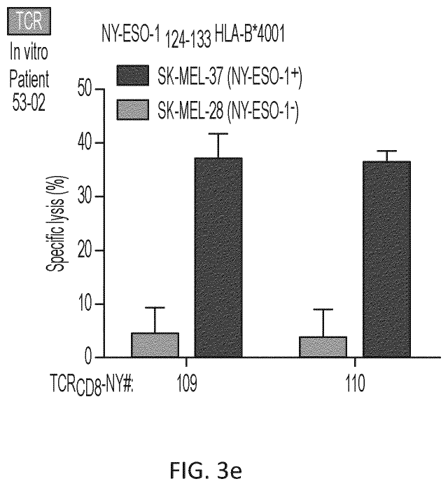

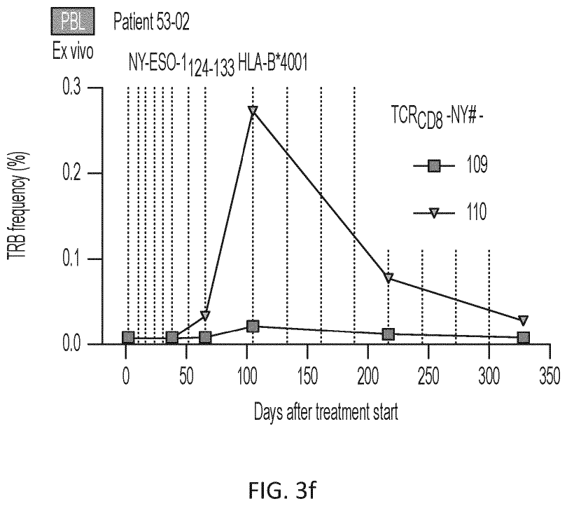

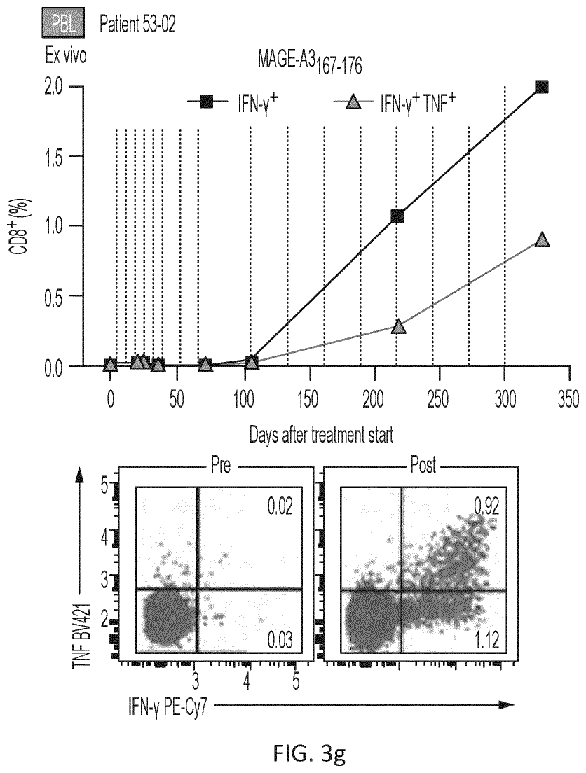

Cytotoxicity assay. T-cell-mediated cytotoxicity was assessed by cell index impedance measurements with the xCELLigence MP system (OMNI Life Science) according to the supplier's instructions. As effector cells, either OKT3-activated TCR-transfected CD8+ T cells from healthy donors or patient-derived CD8+ T cells from IVS cultures were used. As target cells melanoma cell lines transfected with the respective HLA allele were used and seeded at a concentration of 2×104 cells per well in 96-well PET E-plates (ACEA Biosciences). After 24 h, effector T cells were added at different E:T ratios and cell index values were monitored every 30 min for a period of up to 48 h using the xCELLigence system. Specific lysis was calculated after the indicated times of co-culture (FIGS. 21, 3e, 12 hour; FIG. 3d, 63 hour; FIG. 4f, 8 hour) based on a negative control (for TCR, mock-transfected T cells; for IVS cells, pretreatment IVS cultures).

Mutation discovery and gene expression. Mutations were detected as described (Ref. 26). In essence, the genomic sequence reads from each patient were aligned to the human reference genome hg19 using the Burrows-Wheeler Aligner (BWA) software (Ref. 34). Exomes from tumours and matched normal samples were compared to retrieve single nucleotide variants (SNVs). To retain high-confidence SNVs, loci with putative homozygous genotypes were filtered, as were suspected sites from putative heterozygous mutational events, to remove false positives. For the final list of high-confidence mutations, genomic coordinates and University of California at Santa Cruz (UCSC) Genome Browser known genes were incorporated to associate the variants with genes. Non-synonymous mutations were selected for further processing.

Tumor RNA-sequencing data were used to calculate gene-expression values using Sailfish (Ref. 35) and UCSC known gene transcripts as a reference. Transcript counts were normalized to transcripts per million (TPMs).

For comparison of mutational load with gene expression, the mean of transcript expression values was used in those cases where a gene was represented by several transcript isoforms in the UCSC database. Mutational load and expression levels were correlated using patient data from three melanoma cohorts: 13 patients from the NCT02035956 trial (Ref 26), 25 patients from a published melanoma cohort (Ref 0.22), and metastasis data from 12 patients with patients of the MET500 cohort (Ref 36).

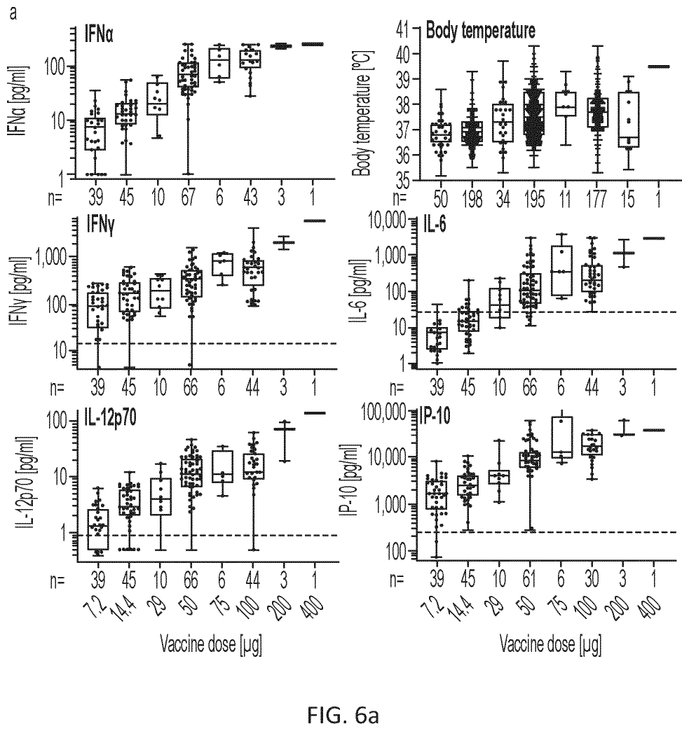

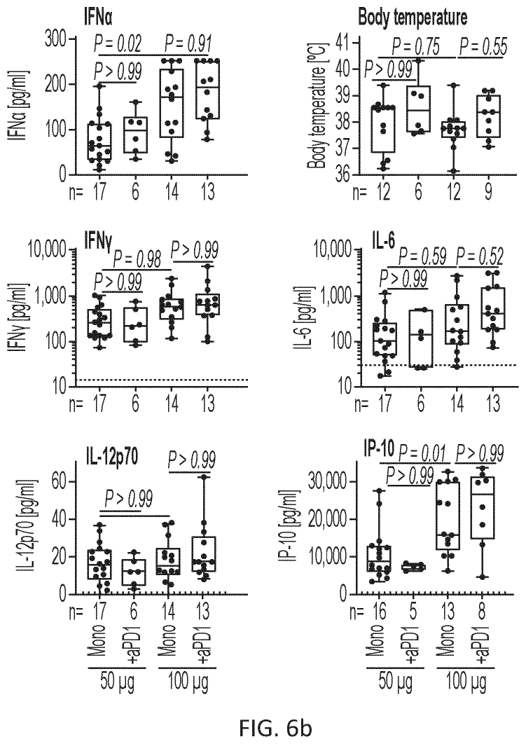

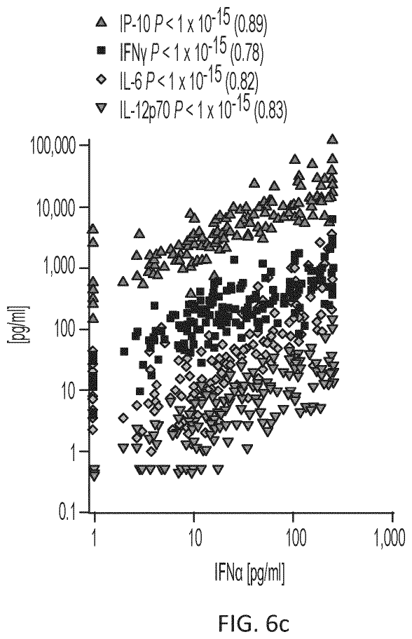

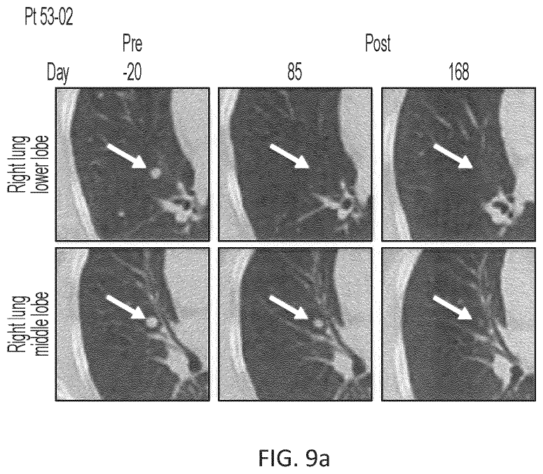

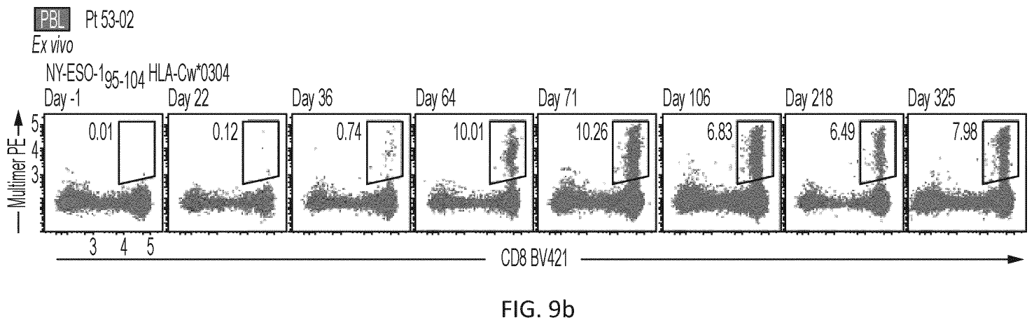

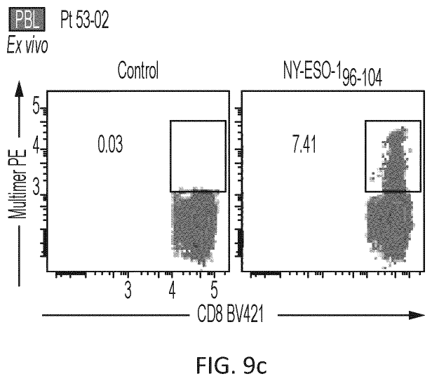

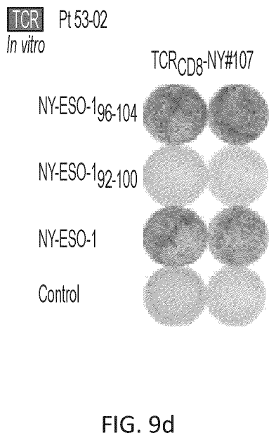

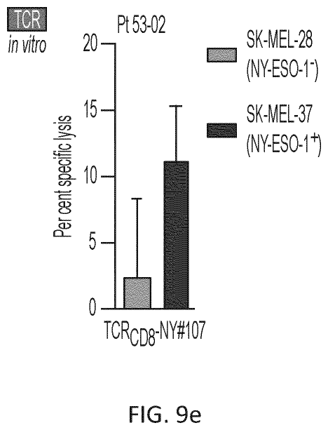

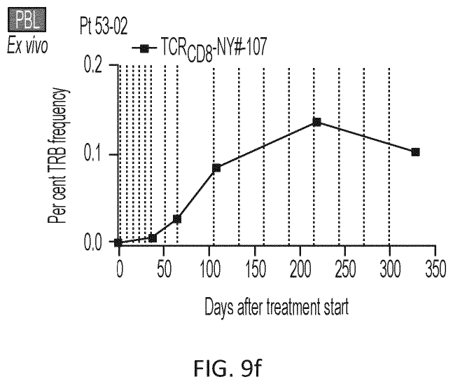

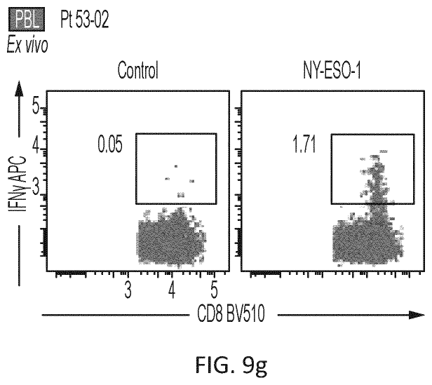

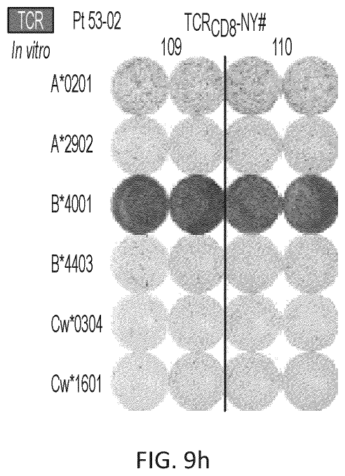

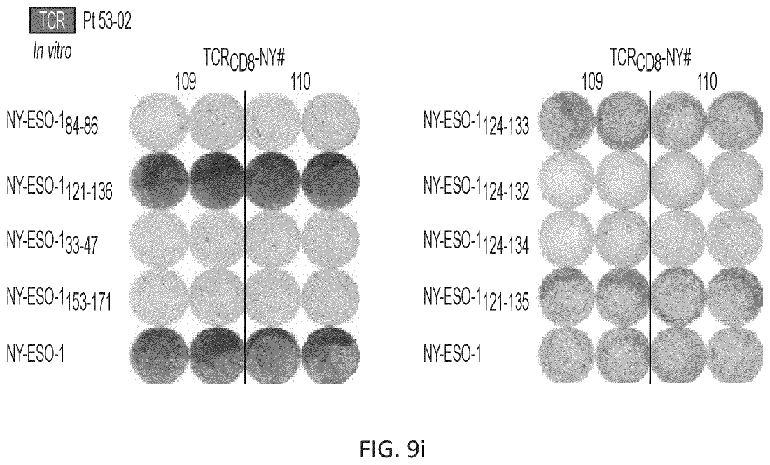

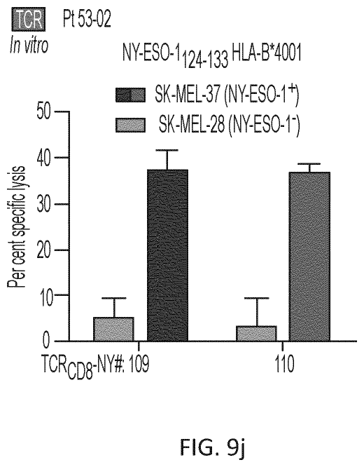

Statistics and reproducibility. Sample sizes (n) represent the number of analyzed patients, except in FIG. 6c, where the sum of multiple measurements (up to six per patient) originating from 72 patients is designated as n. If not otherwise stated, center values represent means, with replicates depicted as symbols. For cytotoxicity experiments in which individual replicate values could not be shown, the dispersion of all technical triplicates used for lysis calculation is indicated as standard deviations. Statistical significance (P) was determined by a Spearman' correlation (FIG. 6c, rs: Spearman's rank correlation coefficient), Pearson correlation, Kruskal-Wallis test followed by Dunn's post hoc test (FIG. 6b) or Brown-Forsythe and Welch analysis of variance (ANOVA) followed by Dunnett T3 multiple comparisons test (FIG. 9d). All analyses were two-tailed and carried out using GraphPad Prism 8.4. All experiments were performed once. The experiments were not randomized.

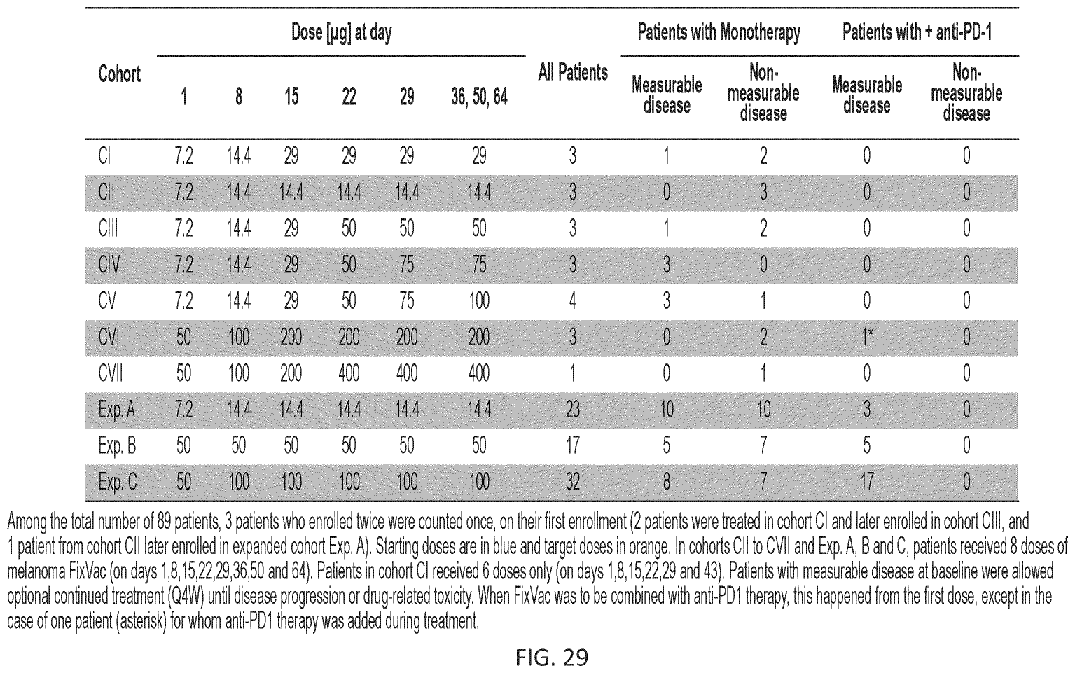

Examples below summarize the results of an exploratory interim analysis (ending 29 Jul. 2019) of 89 patients (FIG. 5) that focused on the immune responses induced by melanoma FixVac. The best objective response to FixVac alone or combined with anti-PD1 antibodies in patients with measurable disease was also assessed (FIG. 29).

### Example 2: In Vivo Characterization of Immune Activation Mediated by an Exemplary RNA Composition Described Herein

The present Example demonstrates in vivo characterization of immune activation following administration of an exemplary pharmaceutical composition comprising one or more RNA molecules that collectively encode a NY-ESO-1 antigen, a MAGE-A3 antigen, a tyrosinase antigen, a TPTE antigen, or a combination thereof; and lipid particles (e.g., lipoplexes or lipid nanoparticles). FIG. 1a shows an exemplary schematic of the one or more RNA molecules that collectively encode a NY-ESO-1 antigen, a MAGE-A3 antigen, a tyrosinase antigen, and a TPTE antigen.

FixVac targeting in the spleen. This Example shows targeting of a FixVac to the spleen by exploiting the enhanced glucose consumption of cells upon TLR ligand stimulation (Ref. 12). In this Example, [18F]-fluoro-2-deoxy-2-d-glucose (FDG)-positron emission tomography (PET)/computerized tomography (CT) scans were carried out shortly after injection of the FixVac. A substantial increase in metabolic activity specifically in the spleen was seen shortly after injection, indicating fast targeting and transient activation of lymphoid-tissue-resident immune cells (FIG. 1c).

Adjuvanticity. To determine adjuvanticity of a FixVac following administration to a patient the amounts of plasma cytokines were measured (Ref. 8). Levels of interferon (IFN)-α, IFN-γ, interleukin (IL)-6, IFN-inducible protein (IP)-10 and IL-12 p70 subunit increased in line with the FixVac dose, accompanied by a transient elevation in body temperature (FIG. 1d; FIG. 6a). Cytokine secretion was pulsatile, transient and self-limiting, peaking 2-6 h after treatment and normalizing within 24 h (FIG. 1d). Combining a FixVac with anti-PD1 antibodies did not affect cytokines (FIG. 6b). Plasma concentrations of IFN-α correlated well with all other measured cytokines (see Spearman Correlation to IFNα (rₛ) as shown in FIG. 6c).

Adverse-event profile. In line with the cytokine patterns, the clinical adverse-event profile was dominated by mild to moderate flu-like symptoms, such as pyrexia and chills. Adverse events were mostly early-onset, transient and manageable with antipyretics, and resolved within 24 hours. The in vivo observations recapitulate findings in mice, in which the mode-of-action of FixVac was driven by translation of the antigen-encoding RNA in dendritic cells resident in lymphoid compartments and by a concomitant inflammatory response induced by TLRs on antigen-presenting cells (Ref. 8, 13, and 20). However, FixVac concentrations that triggered cytokine release in humans were more than 1,000-fold lower than in mice (Kranz et al. 2014, which is incorporated herein by reference in its entirety).

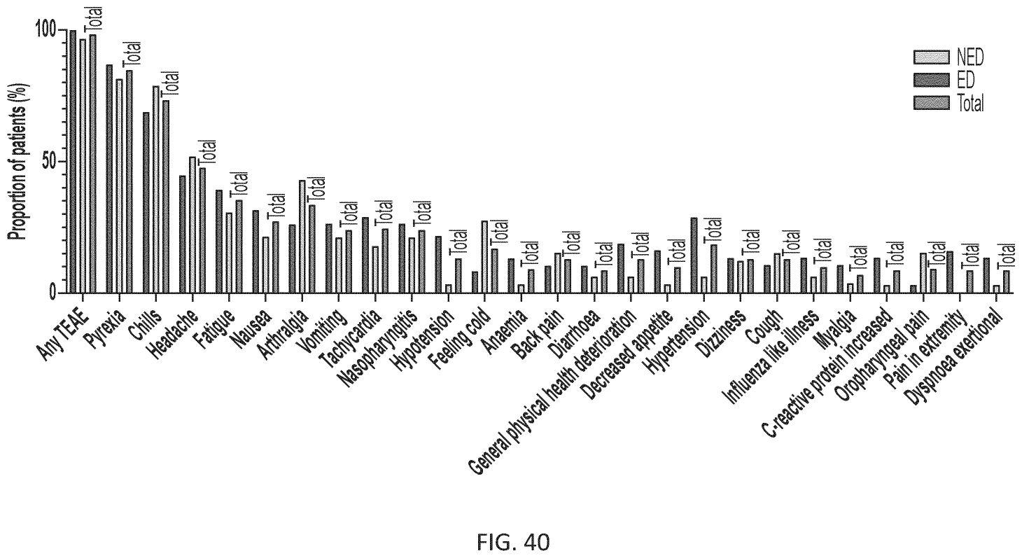

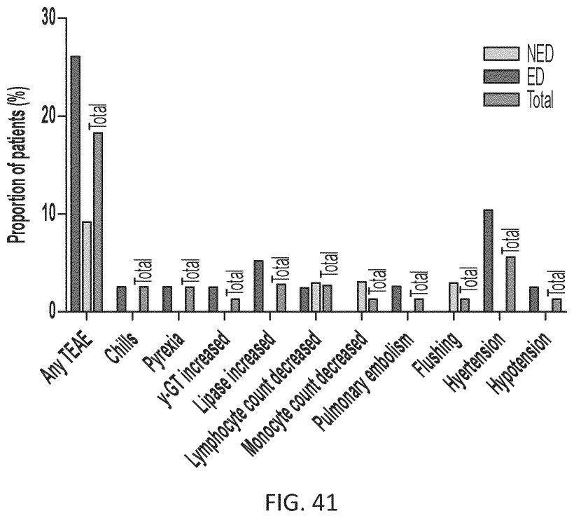

Additional details on adverse events detected during administration are included in FIGS. 40 and 41. As shown, the most frequently occurring related TEAEs were pyrexia, followed by chills, headache, fatigue, nausea, arthralgia, vomiting, and tachycardia. The frequency of these related TEAEs was similar between the ED and NED subgroups. These symptoms were mostly of CTCAE Grade 1 or 2 and are expected reactogenicity due to intrinsic adjuvanticity of RNA-LPX. There was a higher proportion of patients in the ED subgroup that experienced a related TEAE of Grade ≥3 when compared to the NED subgroup (10 patients [26.3%] vs. 3 patients [9.1%], respectively). In the ED and NED subgroups, 4/38 patients (10.5%) and 1/33 patients (3.0%), respectively, experienced a TESAE that was deemed related to the trial treatment (data not shown).

### Example 3: Immunogenicity of Pharmaceutical Compositions

This Example shows immunogenicity after in vitro stimulation (IVS) of samples collected from melanoma patients (e.g., patients having malignant melanoma Stage III B-C or IV (American Joint Committee on Cancer (AJCC) 2009 melanoma classification), both resected and unresected, and thus with measurable and non-measurable disease at baseline, with expression of at least one of the four TAAs included in a FixVac) following administration of a FixVac. In this Example, immunogenicity of a FixVac was measured by IFN-γ ELISpot following IVS.

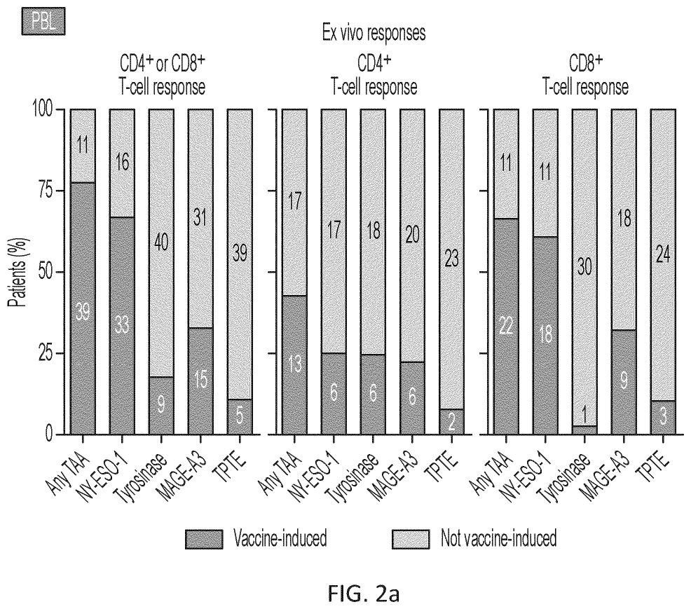

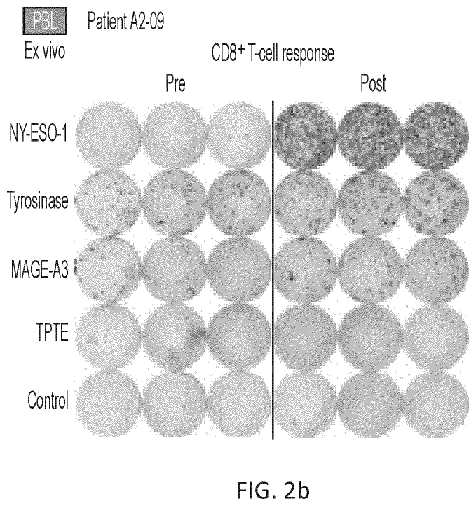

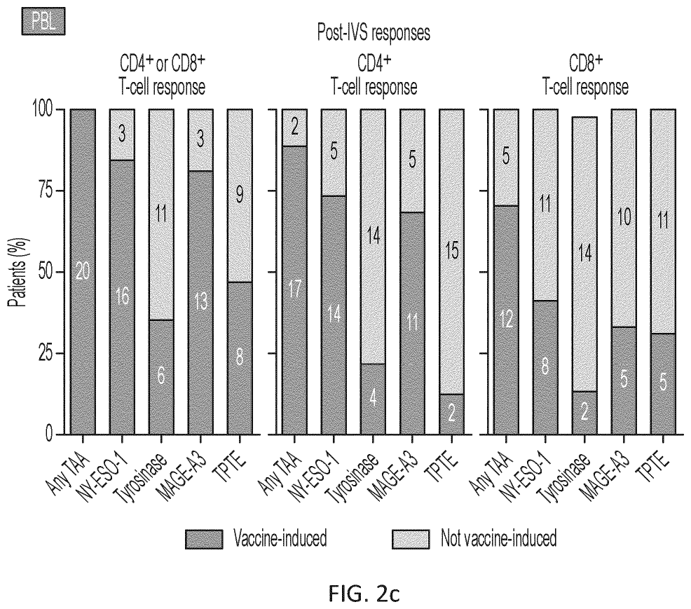

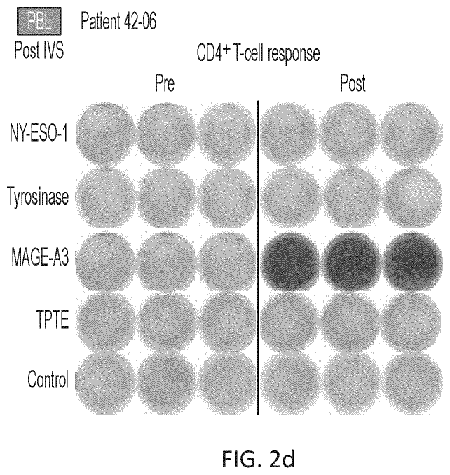

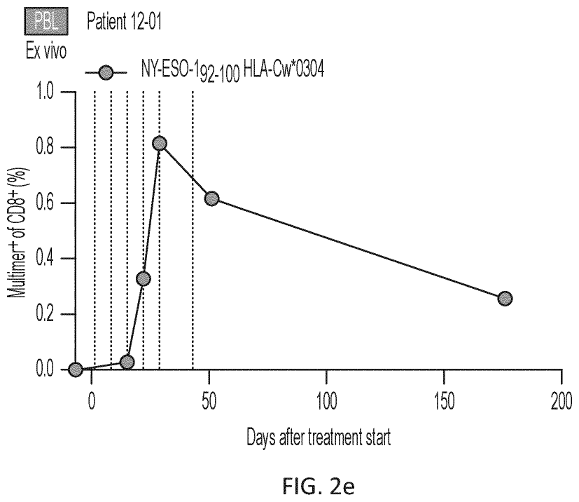

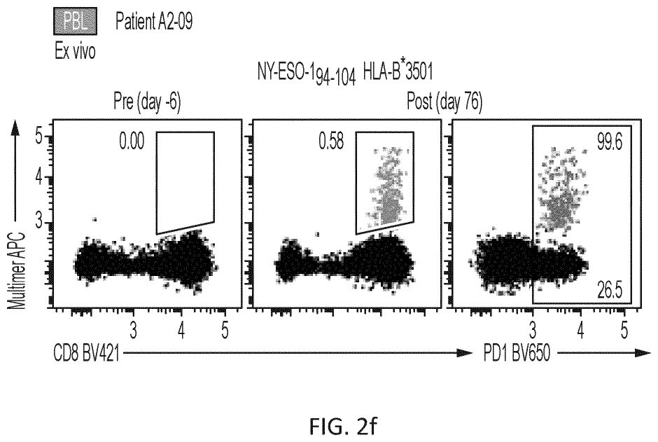

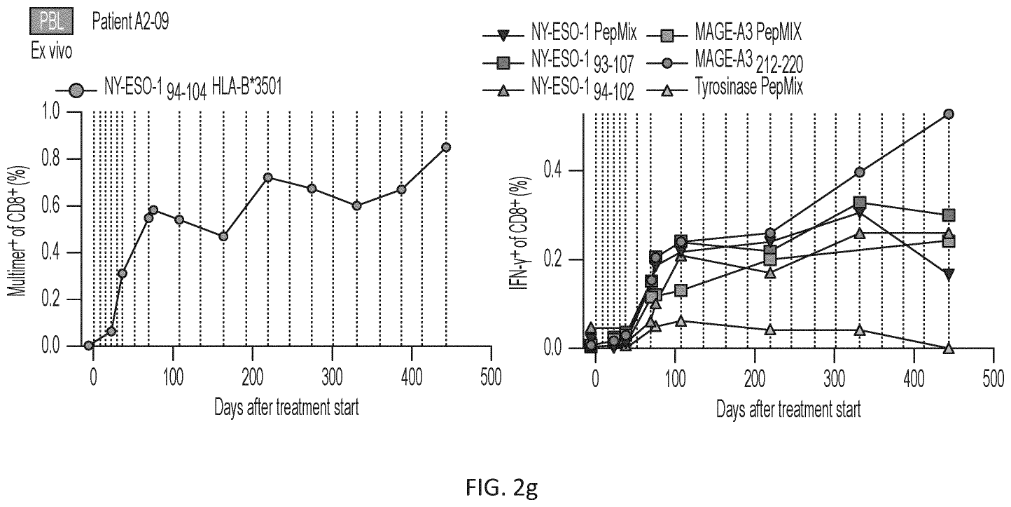

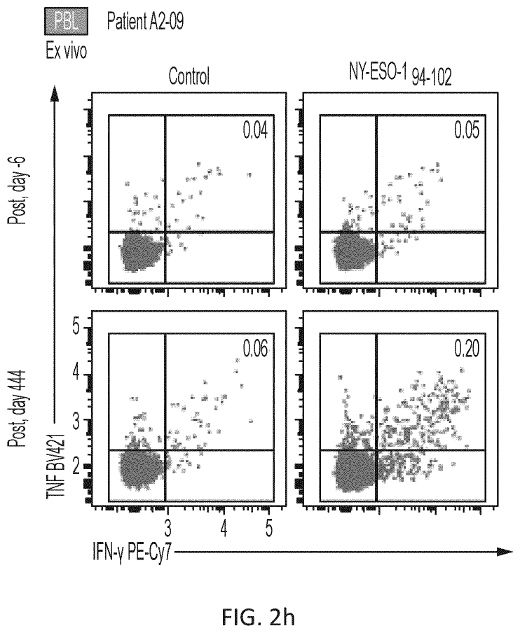

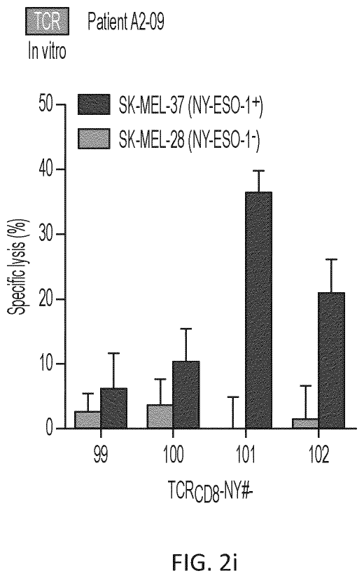

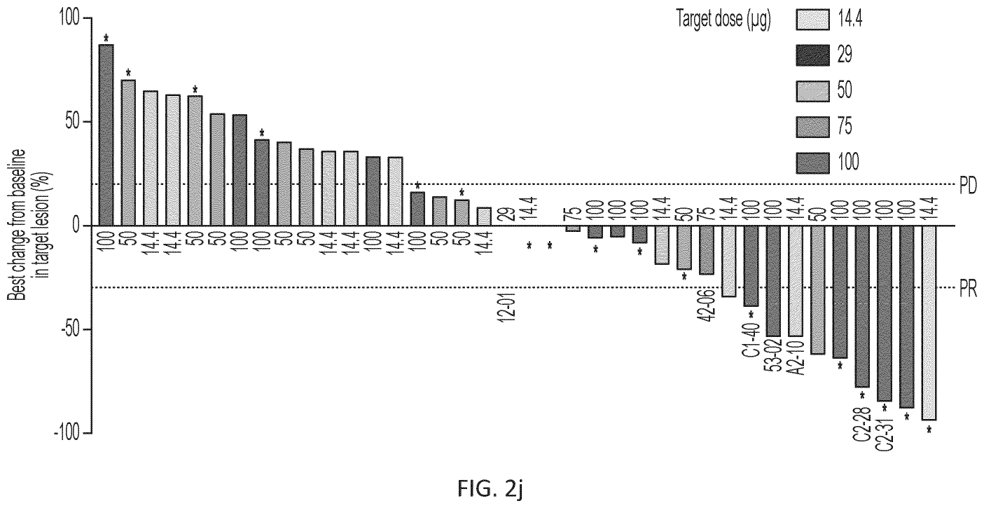

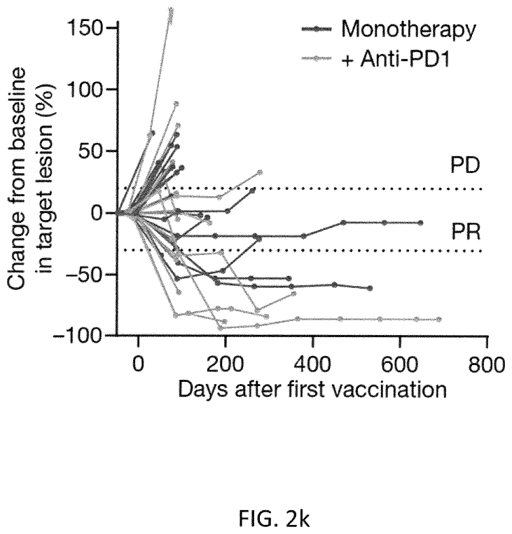

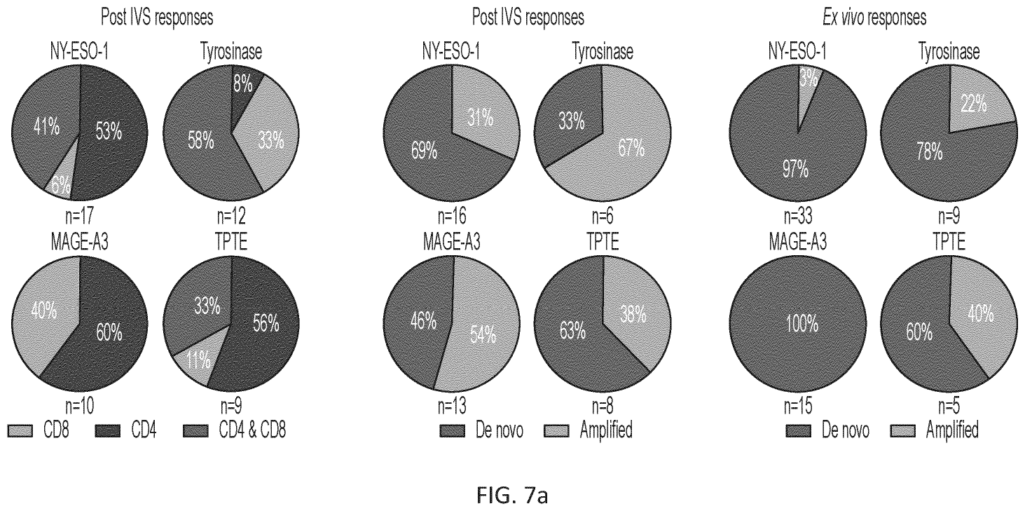

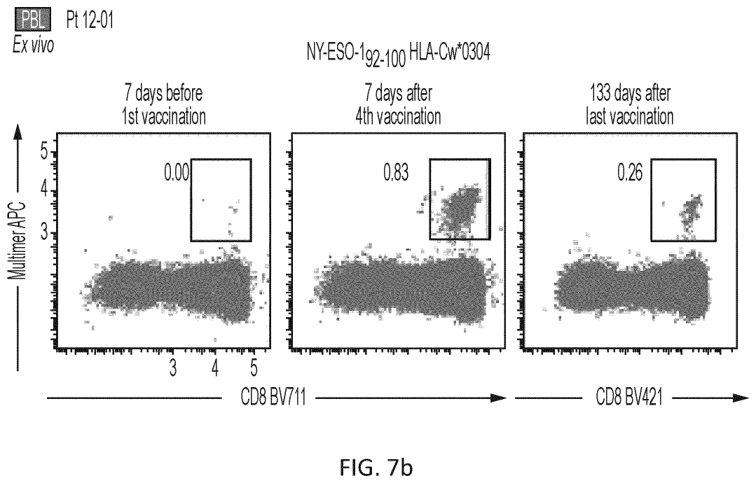

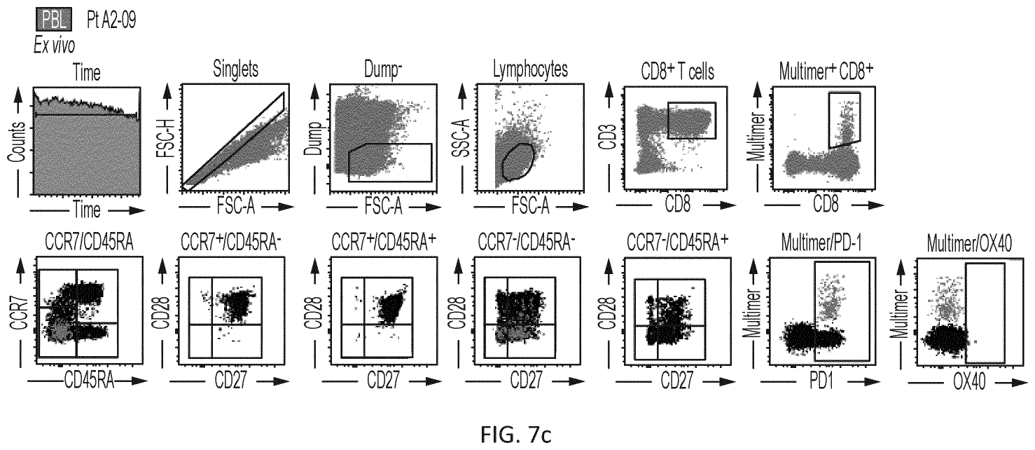

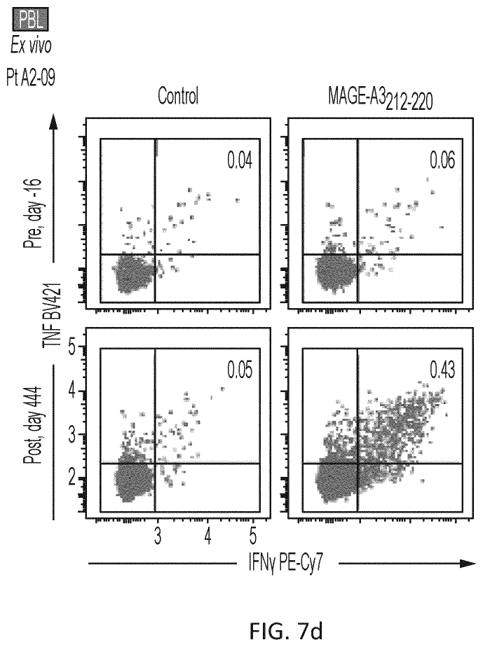

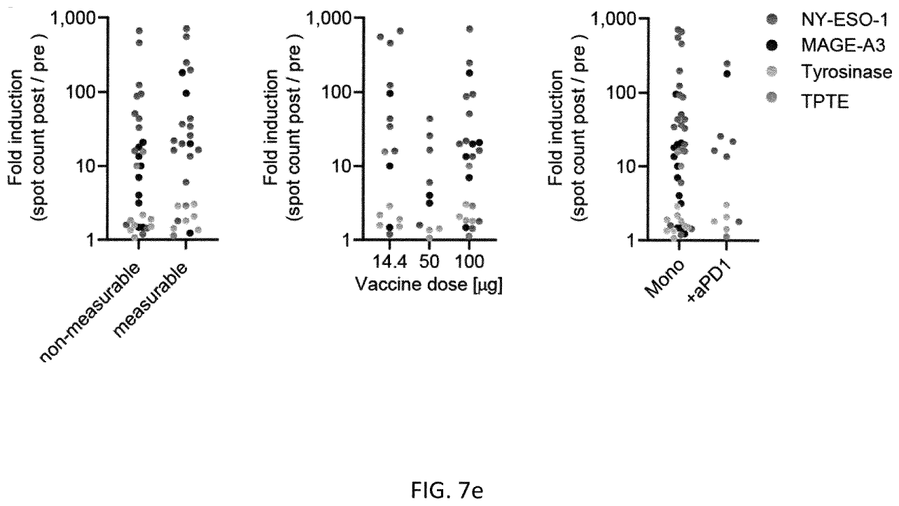

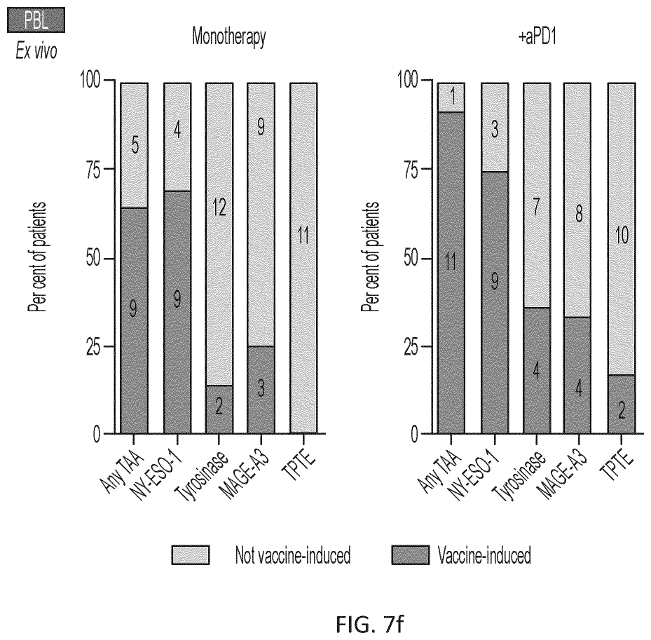

For 50 patients, an ex vivo IFN-γ ELISpot (FIGS. 2a and 2b) was performed before and after vaccination (after eight injections of a FixVac) on bulk or CD4⁻ or CD8⁻ depleted peripheral blood mononuclear cells (PBMCs) incubated with overlapping peptides representing the full-length sequences of TAAs described herein (so-called PepMixes). Samples from 20 patients were also analyzed using post-IVS IFN-γ ELISpot (FIG. 2c) in which autologous dendritic cells loaded with TAA PepMixes were used as targets. Samples from all 20 of these patients showed a T-cell response against at least one TAA (FIG. 2c), mostly a CD4⁺ response alone or both CD8⁺ and CD4⁺ responses (FIG. 7a). Vaccine-induced de novo responses (those not detectable before vaccination) were more frequent than was augmentation of pre-vaccine responses (FIG. 7a). Of the samples from the 50 patients who were analyzed using ex vivo IFN-γ ELISpot, more than 75% showed immune responses against at least one TAA (FIG. 2a). Most of these high-magnitude T-cell responses were CD8⁺ (FIG. 2a).

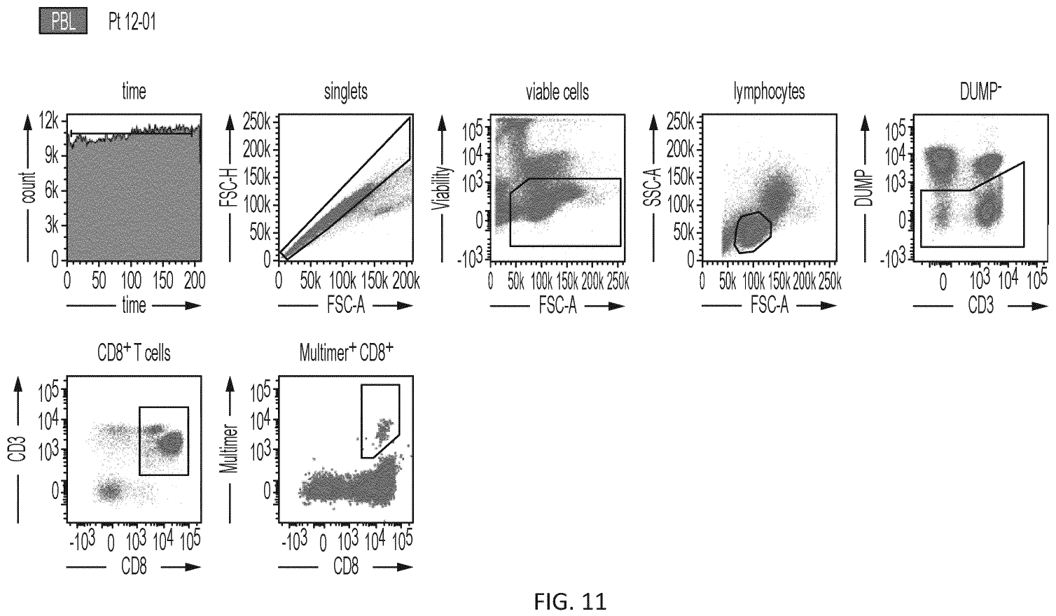

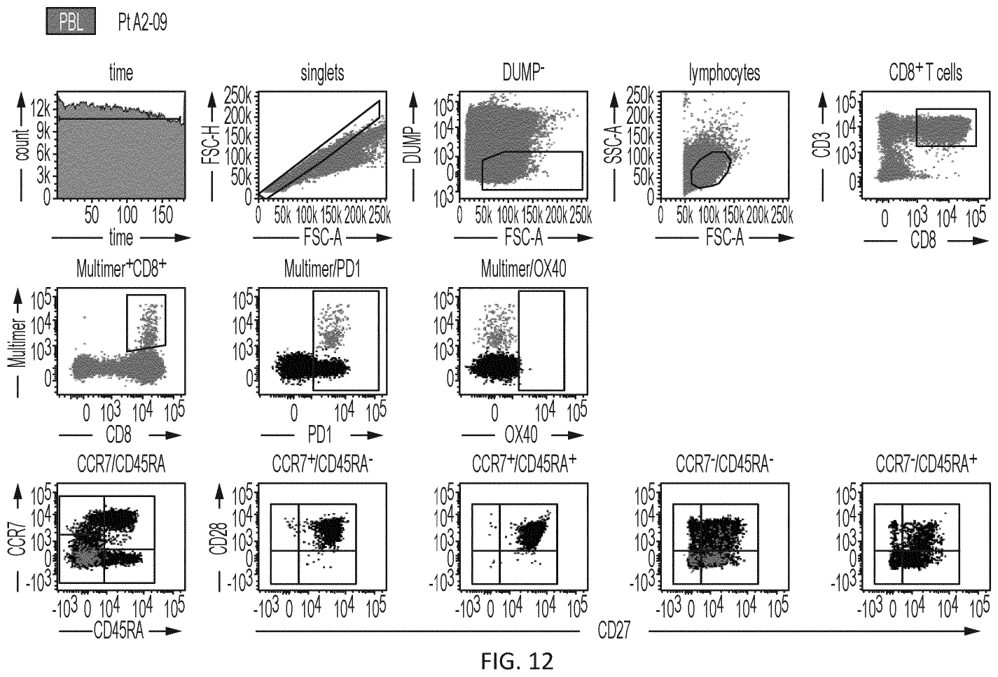

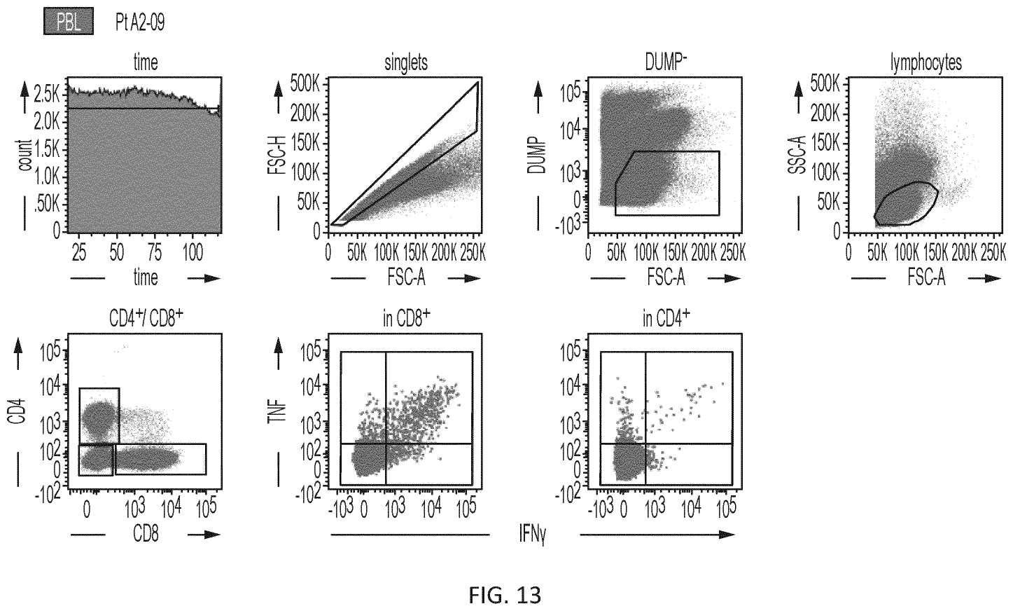

Ex vivo de novo CD8⁺ T cells were measured by HLA multimer analysis and intracellular cytokine staining (ICS). Antigen-specific T cells that ramped up within 4-8 weeks to single-digit or low double-digit percentages of circulating CD8⁺ T cells (FIG. 2e-g; FIG. 3a, FIG. 7b, FIG. 11) were of the PD1⁺CCR7⁻CD27^{+/-}CD45RA⁻ effector memory phenotype (FIG. 2f, FIG. 7c, and FIG. 12, and secreted IFN-γ and tumor necrosis factor (TNF) upon antigen-specific restimulation (FIG. 2h, FIG. 7d, and FIG. 13). Most patients had polyepitopic CD8⁺ immune responses (FIG. 2b, FIG. 2g). In patients undergoing monthly maintenance vaccination after the first 8 vaccinations, TAA-specific T cells continued to increase in frequency or remained stable over more than one year (FIG. 2g). In patients without continuous vaccination, memory T cells remained present over several months with a slow downwards trend (FIG. 2e and FIG. 7b).

### Example 4: Characterization of TAA-Specific T-Cell Receptors (TCRs) from Vaccine Expanded T Cells Isolated from Patients

This Example characterizes T-cell receptors from T cells expanded following administration of a FixVac.

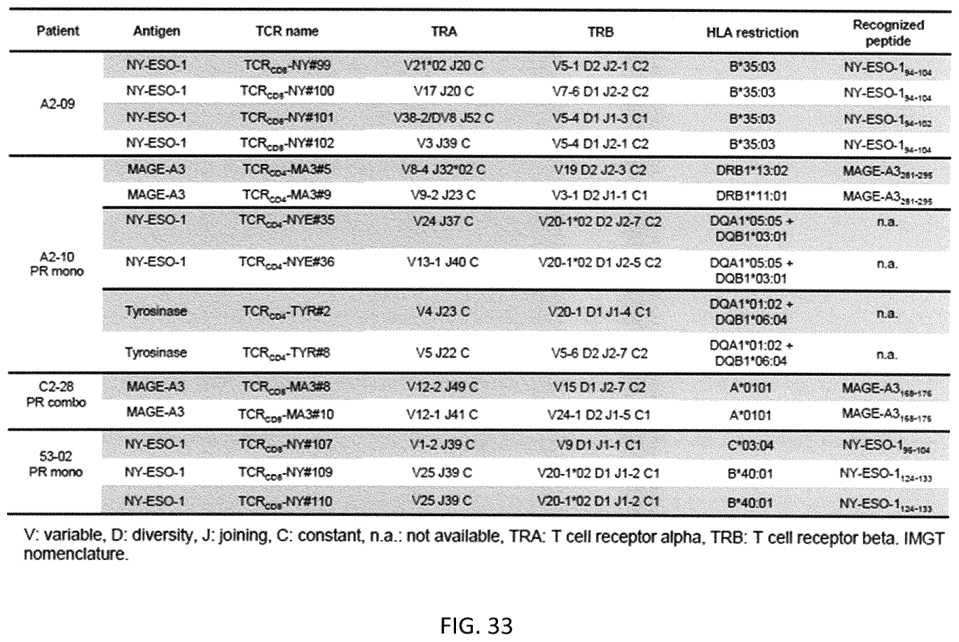

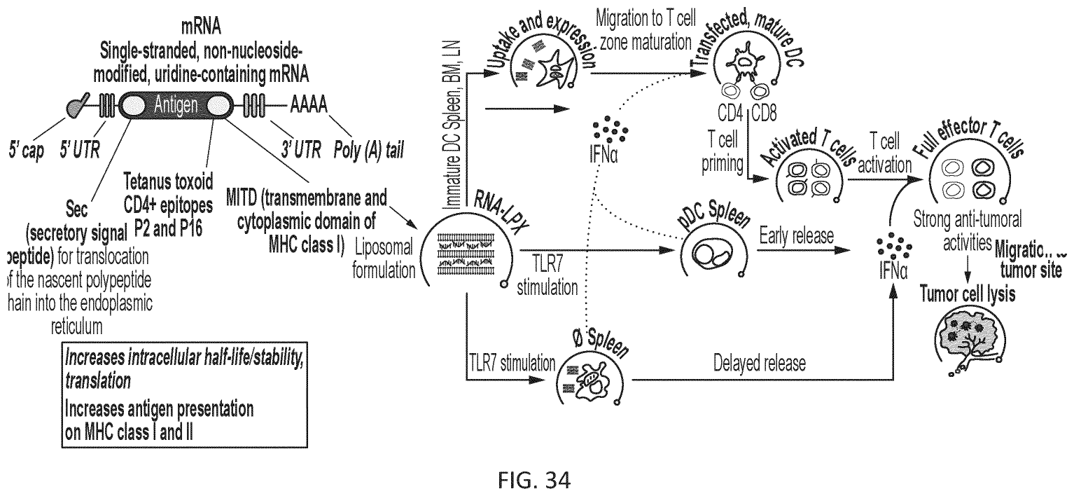

TAA-specific T-cell receptors (TCRs) from vaccine-expanded T cells (FIG. 33) transfected into healthy donor T cells efficiently killed TAA-positive melanoma cells (FIG. 2i). T-cell responses were not affected by the presence or absence of radiologically measurable disease at baseline, by the FixVac treatment dose or by whether FixVac was administered alone or in combination with anti-PD1 antibodies (FIGS. 7e and 7f).

### Example 5: Best Objective Response in 42 Patients with Measurable Metastatic Disease

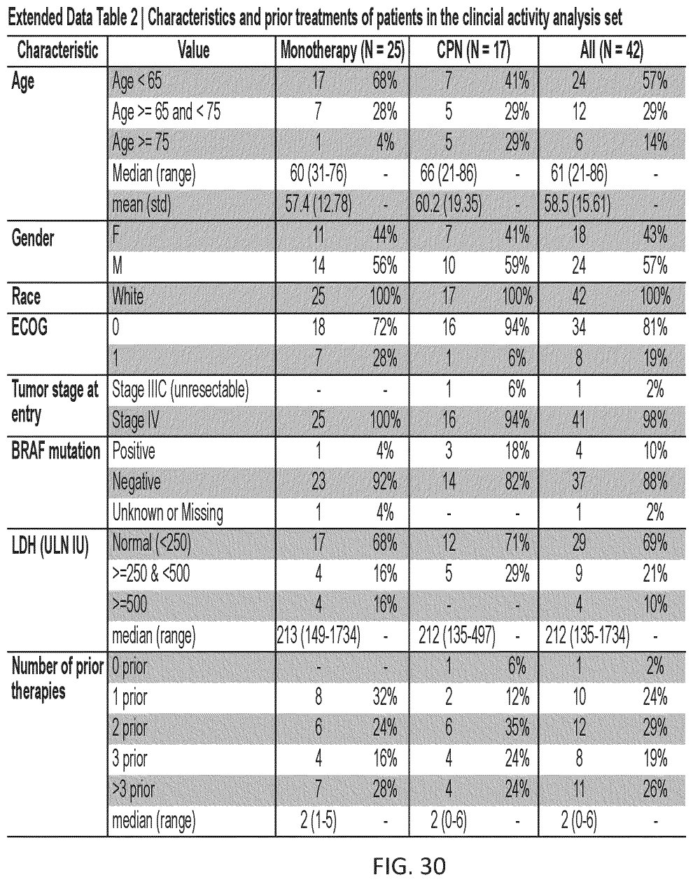

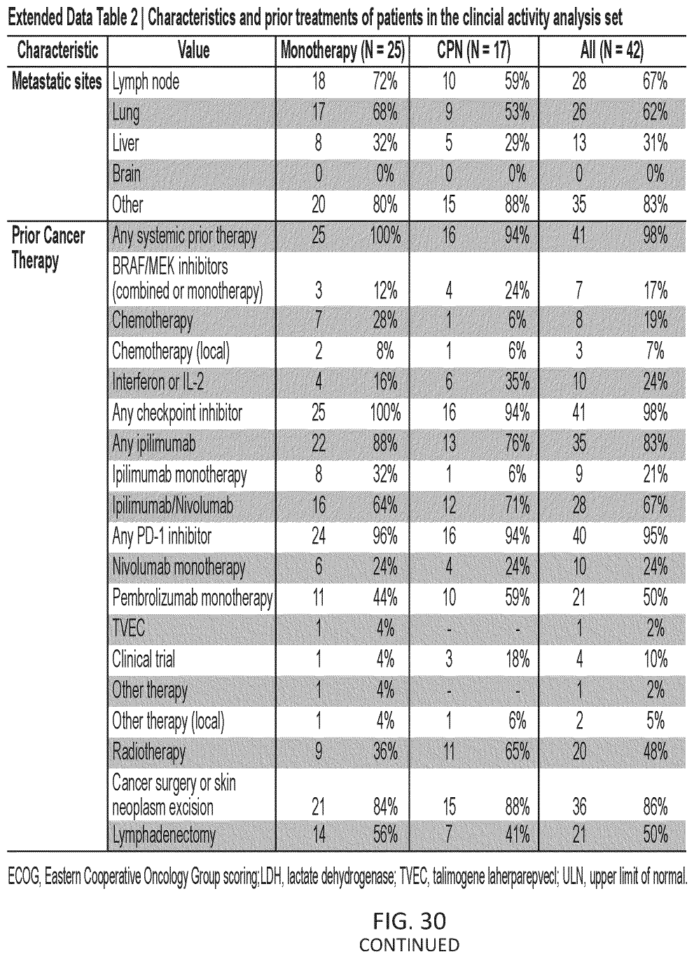

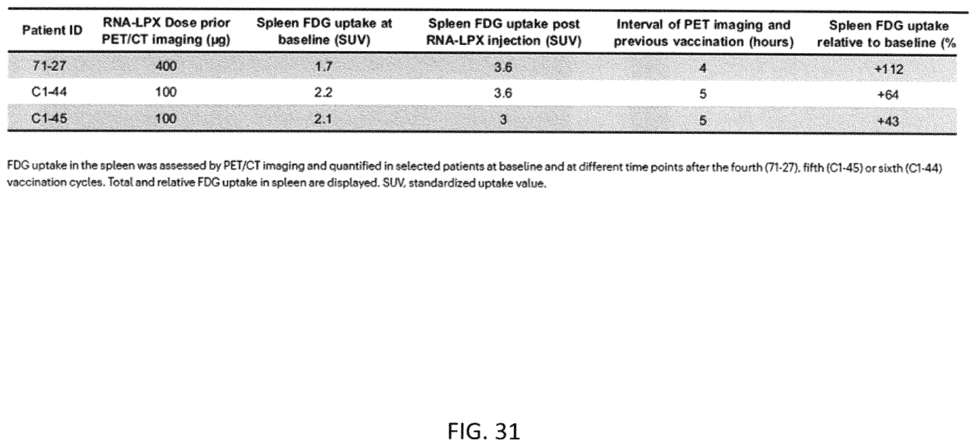

This Examples shows the response for melanoma patients with measurable metastatic disease for whom one scan at baseline and at least one scan following treatment were available. Forty-one patients were at stage IV, had undergone previous lines of systemic treatment and were checkpoint-inhibitor (CPI)-experienced; 35 of these had been exposed to antibodies against both PD1 and cytotoxic T-lymphocyte-associated protein 4 (CTLA4) (FIG. 30).

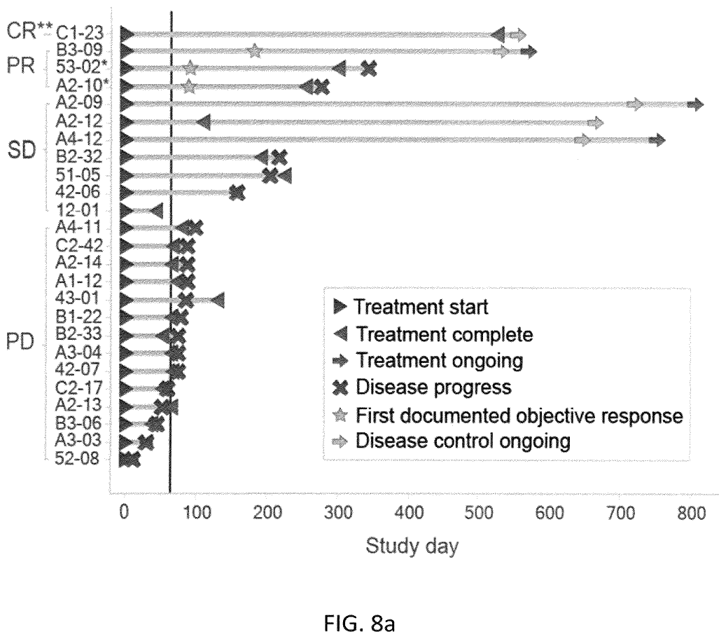

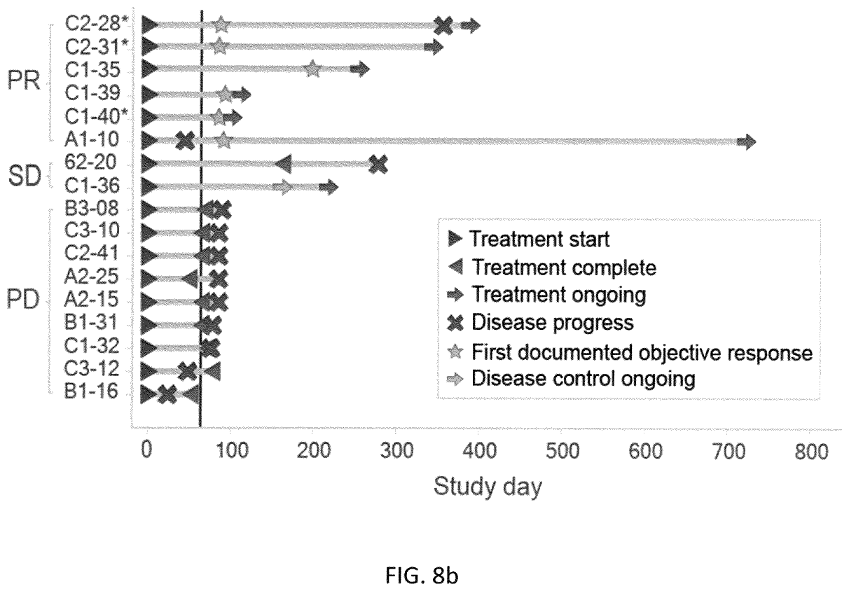

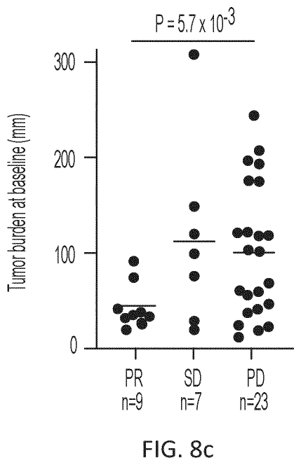

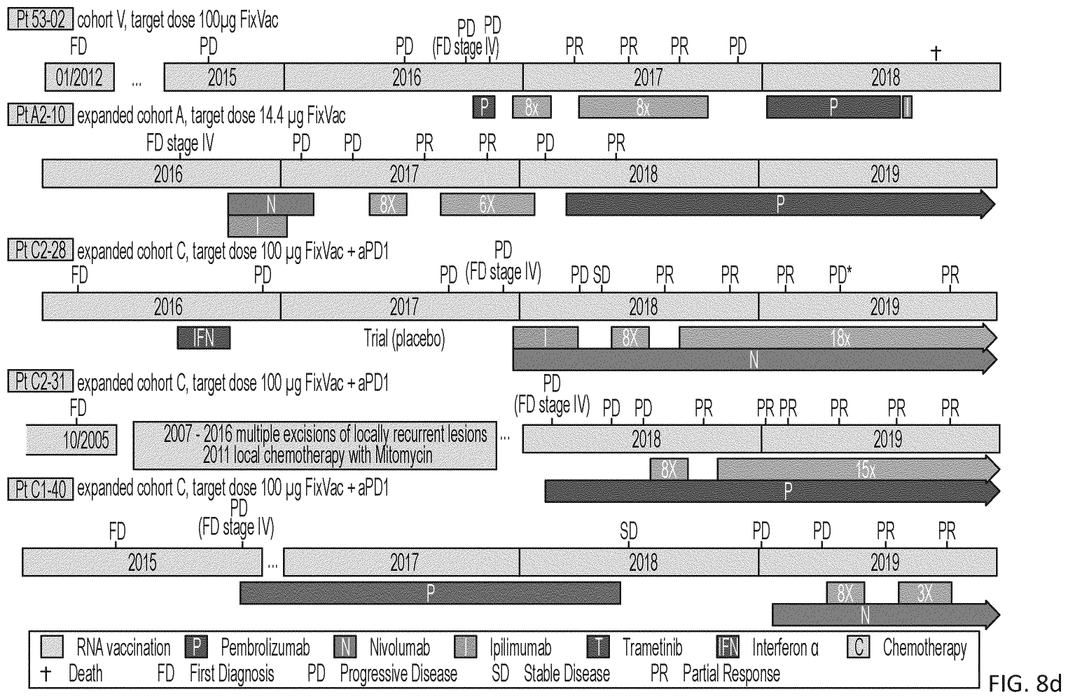

In the FixVac monotherapy group (n=25), three patients experienced a partial response and seven had stable disease (FIG. 2j, FIG. 5). Another patient showed a complete metabolic remission of metastatic lesions in [18F]-FDG-PET/CT imaging. In the FixVac/anti-PD1 combination group, 6 out of 17 patients developed a partial response. Regression of target lesions occurred across all doses, although the rate of partial response was highest in patients treated with 100 μg melanoma FixVac plus anti-PD1 (five out of ten patients; objective response rate 50%) (FIG. 2j. Most patients with a partial response or stable disease showed durable disease control (over an observation period of up to two years) (FIG. 2k; FIG. 8a and FIG. 8b). The objective response correlated with the tumor burden at baseline (FIG. 8c).

### Example 6: Characterization of Immune Responses from Melanoma Patients Who Received FixVac Monotherapy and Who Received a FixVac/Anti-PD1 Combination

This Example shows the response for specific patients following treatment with combination therapy of FixVac and PD-1 inhibition.

Several patients with partial responses (patients 53-02 and A2-10, who received FixVac monotherapy, and patients C2-28, C2-31 and C1-40, who received the FixVac/anti-PD1 combination; FIG. 8d) had sufficient blood samples for detailed characterization of immune responses.

Patient 53-02 entered the trial after progressing under pembrolizumab treatment. On FixVac monotherapy, this patient experienced a partial response that lasted for eight months, with regression of multiple metastases (FIG. 3b and FIG. 9a). Several weeks after vaccination was discontinued at the patient's request, regrowth of metastatic lesions was diagnosed. The patient was rechallenged with pembrolizumab therapy and remained stable for a further seven months (FIG. 8d).

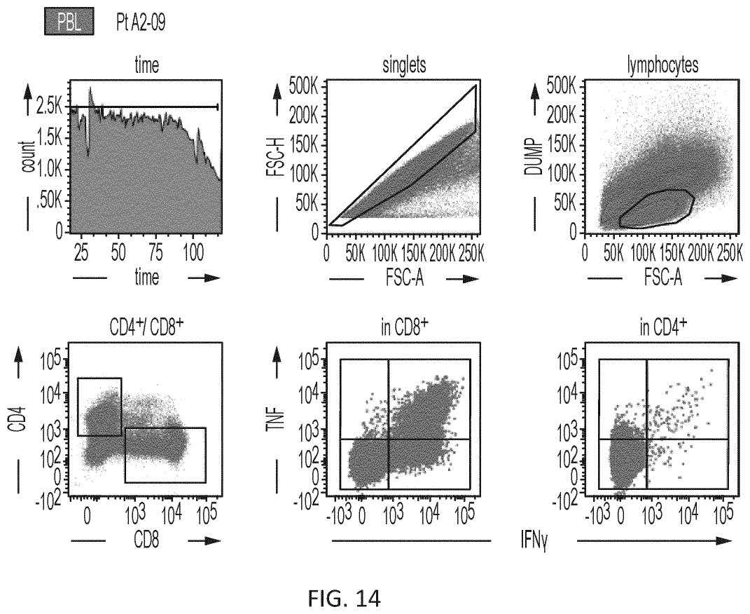

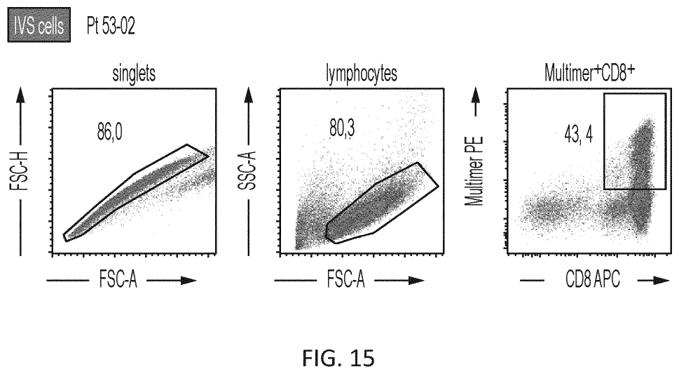

For this patient, strong de novo immune responses against NY-ESO-1 and MAGE-A3 were detected by ex vivo ELISpot. A vaccine-induced HLA-Cw*0304-restricted CD8⁺ T-cell response against the NY-ESO-196-104 epitope15, identified by HLA multimer staining, increased steeply to more than 10% of peripheral blood CD8+ T cells and remained high under continued vaccination (FIG. 3a and FIG. 9b). ICS confirmed that NY-ESO-1-reactive IFN-γ+ T cells expanded to up to 15% of the whole peripheral blood CD8+ T cell population (FIG. 3c and FIG. 14). Short-term IVS cultures of post-vaccination PBMCs expanded against the NY-ESO-196-104 epitope potently killed endogenous NY-ESO-1+ melanoma cells (FIG. 3d and FIG. 15).

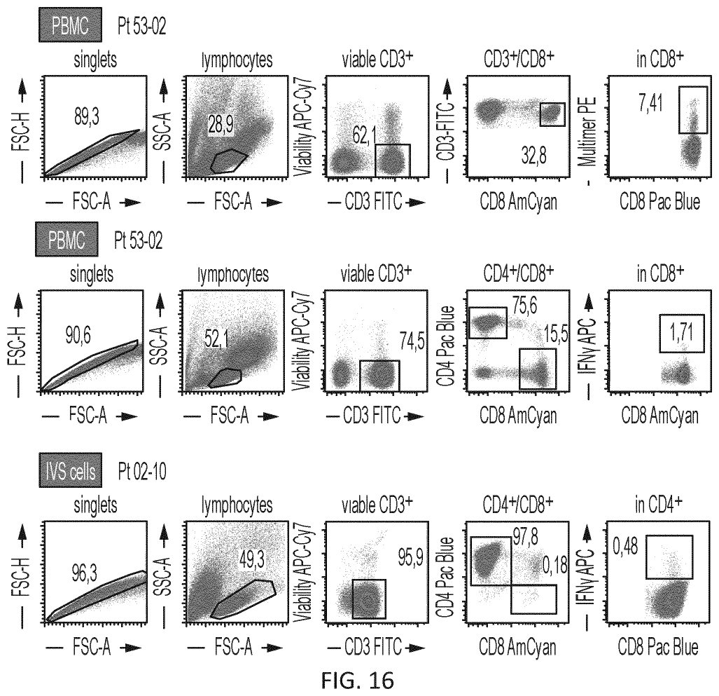

HLA-Cw*0304-restricted (FIG. 9c-9f) and HLA-B*4001-restricted (FIG. 9g-9j) NY-ESO-1-specific TCRs were identified by single-cell cloning from T cells, using HLA multimer binding and antigen-specific cytokine secretion, respectively (FIG. 16). All TCRs mediated killing of NY-ESO-1⁺ melanoma cells (FIG. 3e, FIG. 9e, and FIG. 9j). TCR-β clonotype analyses confirmed that these T cells occurred de novo (FIG. 3f and FIG. 9f). This patient also developed protractedly MAGE-A3₁₆₇₋₁₇₆-specific T cells, comprising about 2% of total CD8⁺ T cells (FIG. 3g).

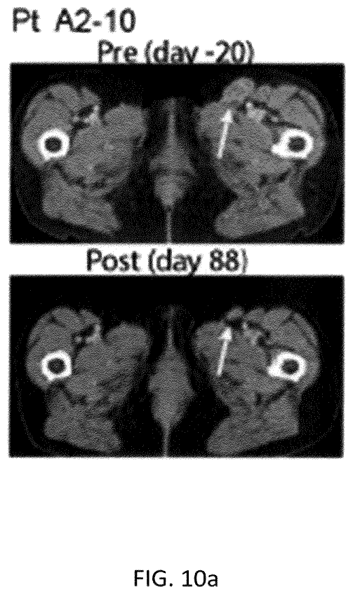

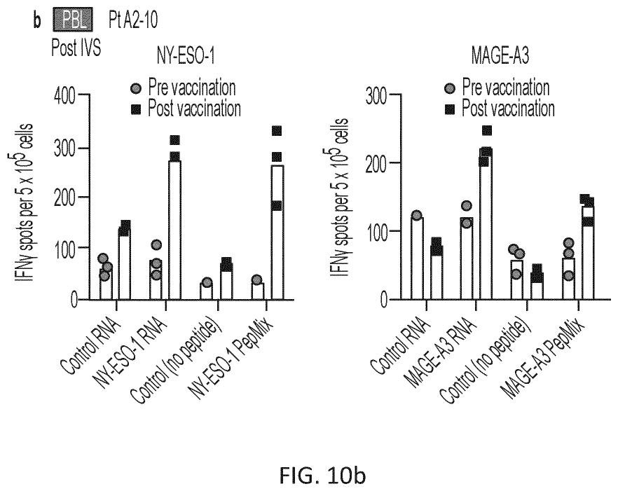

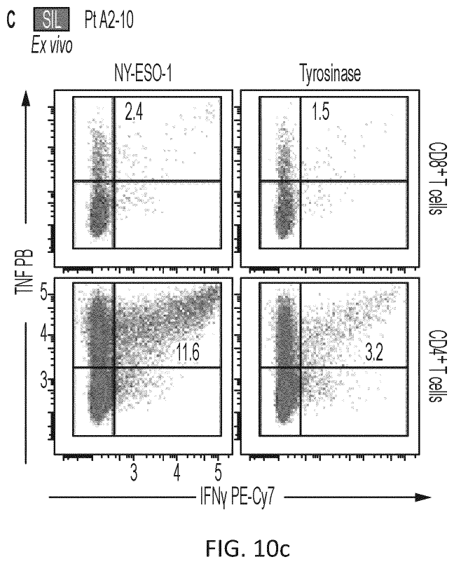

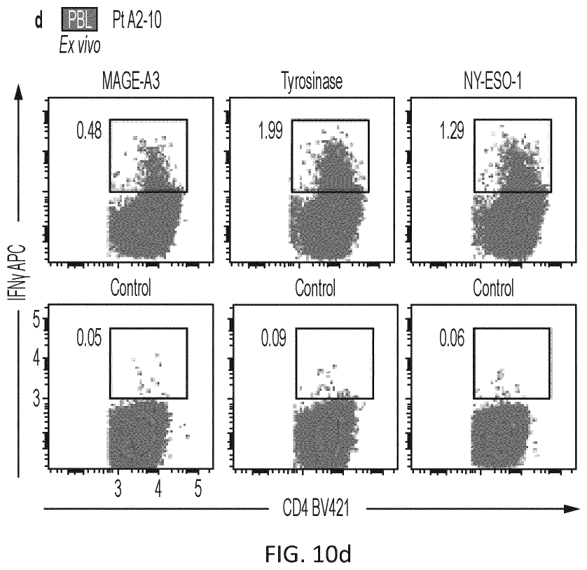

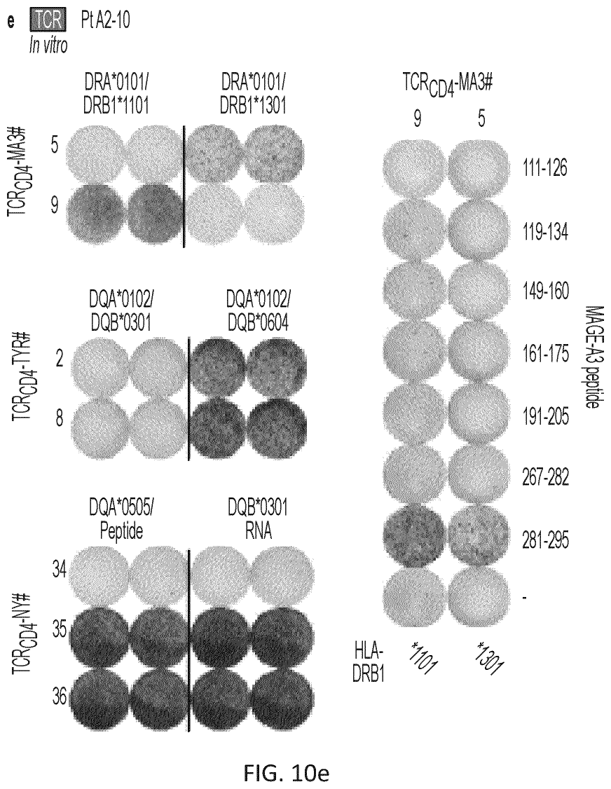

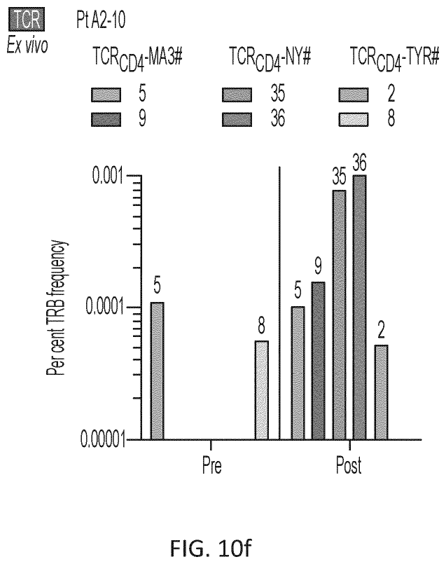

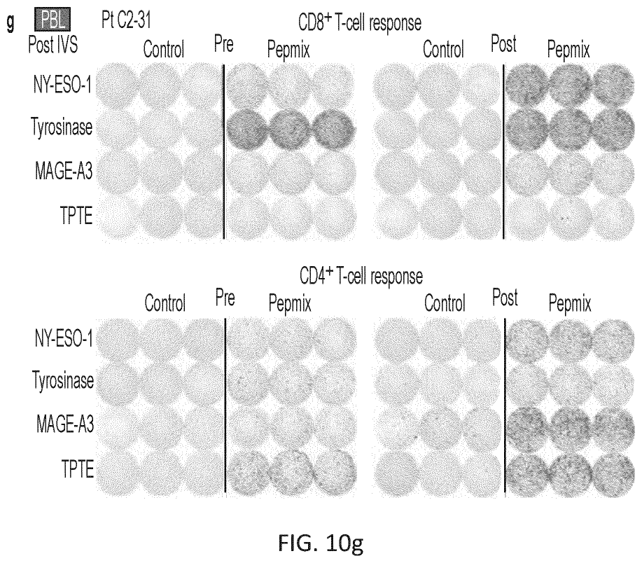

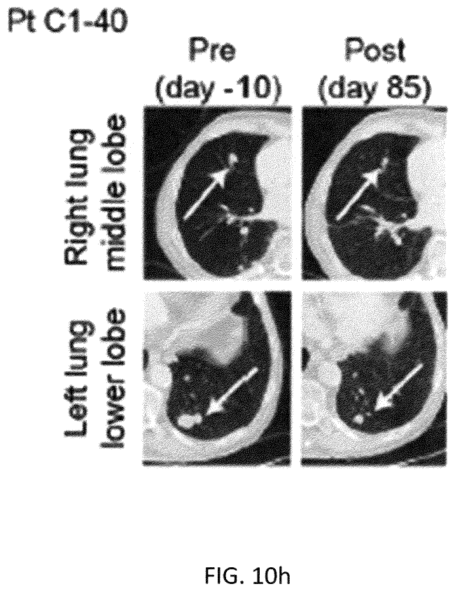

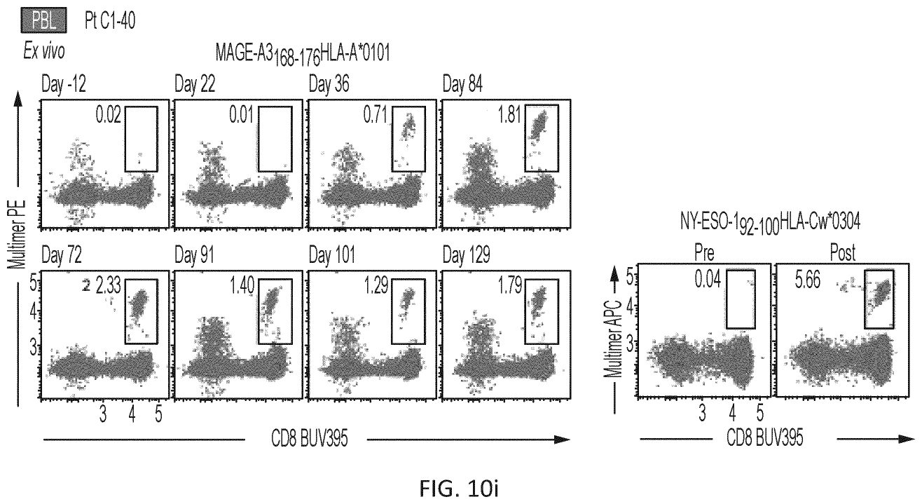

Patient A2-10 showed fast progression of multimetastatic disease under ipilimumab and nivolumab treatment (FIG. 8d). On FixVac monotherapy, this patient experienced a partial response of with a duration of six months with regression of multiple lymph node and lung metastases (FIG. 10a). FixVac was discontinued after eight months owing to progressive disease of an inguinal lymph node. The patient was rechallenged with pembrolizumab monotherapy and experienced a partial response.

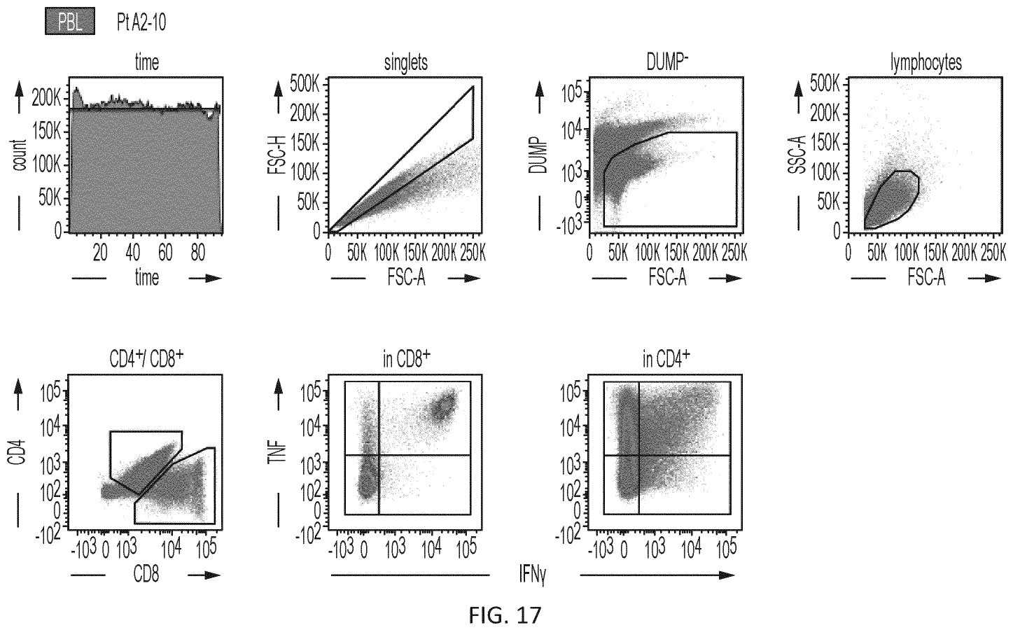

An IFN-γ⁺CD4⁺ T-cell response against MAGE-A3 and NY-ESO-1 was detected in post-treatment PBMCs from this patient (FIG. 10b). Among skin-infiltrating lymphocytes from a delayed-type hypersensitivity (DTH) reaction obtained after eight vaccinations, NY-ESO-1-specific CD4+ T cells were detected (FIG. 10c and FIG. 17). Several NY-ESO-1-, tyrosinase- and MAGE-A3-directed TCR clonotypes from CD4+ T cells of post-treatment PBMCs were cloned (FIG. 10d, FIG. 10e and FIG. 33). These clonotypes included TCRs that recognize the MAGE-A3281-295 epitope, reported as immune dominant and promiscuously presented on various HLA-DRB1 alleles16 (FIG. 10e). TCR frequencies were mostly undetectable by TCR clonotype profiling, and under vaccination increased to easily detectable frequencies (FIG. 10f).

Patient C2-28 had numerous liver and subcutaneous metastases that initially progressed under treatment with the ipilimumab/nivolumab combination, and then stabilized under continued nivolumab monotherapy. The patient was switched to FixVac/nivolumab combination treatment and experienced a partial response (FIG. 4a and FIG. 8d) with reduction of liver and subcutaneous target lesions (the tumour burden reduced from 91 mm to 15 mm). After 11 months of treatment, the patient developed a single bone metastasis, which was irradiated and remained under continued vaccination.

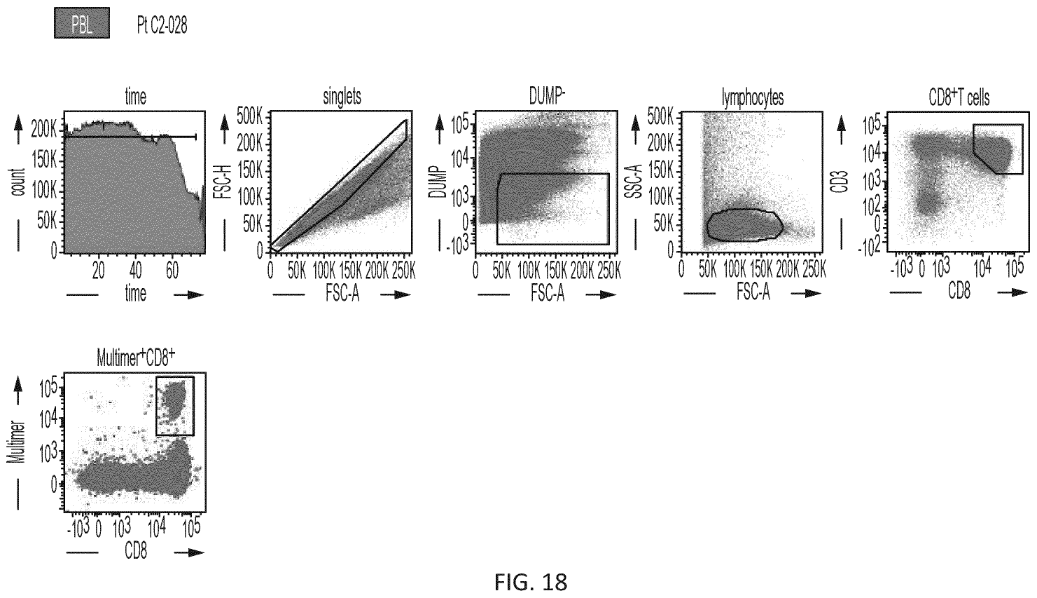

For this patient, NY-ESO-1 and MAGE-A3 T cells were detected by post-IVS ELISpot (data not shown). A de novo HLA-A*0101-restricted T-cell response against the MAGE-A3168-176 epitope17 increased to up to 2% of peripheral blood CD8+ T cells (FIG. 4b and FIG. 18). Two TCRs cloned from MAGE-A3168-176 multimer binding T cells specifically recognized endogenous MAGE-A3+ melanoma cells (FIG. 4c and FIG. 33).

Patient C2-31 had locally recurrent melanoma with recent systemic metastatic dissemination. The patient had progressed under pembrolizumab treatment over seven months, with multiple metastases in lung, liver and lymph nodes. FixVac was added to the ongoing pembrolizumab therapy, and the patient rapidly experienced a partial response (FIG. 4d and FIG. 8d). CD4+ T-cell responses were detected against MAGE-A3, TPTE and NY-ESO-1 and CD8+ T-cell responses against NY-ESO-1 and MAGE-A3, most of which were de novo (FIG. 10g).

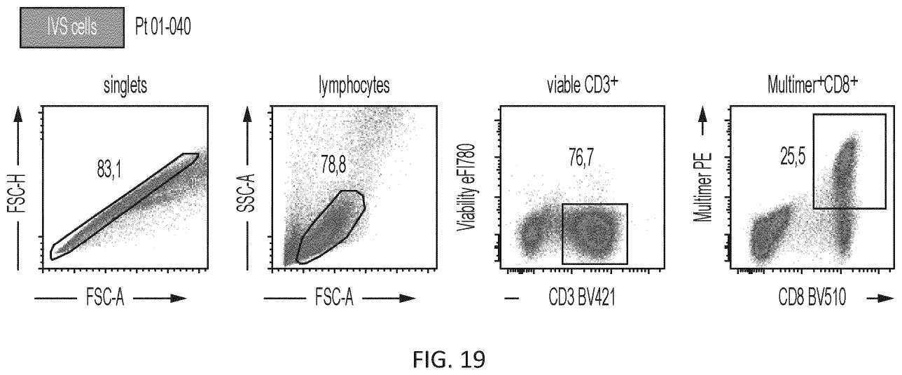

Patient C1-40 had a history of pembrolizumab-responsive metastatic melanoma and, seven months after discontinuation of pembrolizumab, experienced progressive disease with multiple fast-progressing lung lesions. Treatment with nivolumab was initiated, to which melanoma FixVac was added eight weeks later. The patient experienced a partial response with shrinkage of lung metastases (FIG. 8d and FIG. 10h). HLA multimer staining revealed strong vaccine-induced T-cell responses against MAGE-A3₁₆₈₋₁₇₆ and NY-ESO-1₉₂₋₁₀₀ epitopes (FIG. 4e and FIG. 10i). Short-term cultures of post-vaccination lymphocytes efficiently killed MAGE-A3+ melanoma cells, indicating functionality of vaccine-induced T cells (FIG. 4f and FIG. 19).

Summary of findings. Together data provided in Examples 1-6 provide certain key findings. First, the transient cytokine response and the high magnitude and T-helper-1 phenotype of FixVac-induced T cells show that the RNA-LPX vaccine class has the same potent mode of action in humans that was characterized as being pivotal for antitumour effects in mouse models (Ref 8, 18). Several full-length TAAs are delivered together, and patients mount polyclonal CD4⁺ and CD8⁺ T-cell responses. As indicated by the kinetics of HLA multimer positive T cells, over time the prime/repeat boost protocol expands the pool of circulating antigen-specific T cells (in particular those that target NY-ESO-1 and MAGE-A3) by several orders of magnitude.

The patients who experienced partial responses were those with the most prominent and diversified T-cell responses. However, the possibility of bias resulting from the fact that these responders remained in the trial for longer time periods cannot be excluded, allowing us to collect enough blood for epitope identification and multimer analysis—the most informative assay for analyzing T-cell frequencies.

T cells induced by FixVac were fully functional, recognized their target epitopes on melanoma cells and exhibited strong cytotoxic activity. Long-term immune-monitoring data obtained for some patients show that vaccine-induced T cells are maintained by continued vaccination for more than one year.

Second, observations described in Examples 1-6 indicate that although melanoma FixVac is active as a single agent, it also synergizes with anti-PD1 therapy in patients with CPI-experienced tumours. Patients 53-02 and A2-10 started melanoma FixVac treatment after anti-PD1 failure, experienced tumour regression under melanoma FixVac monotherapy, eventually progressed again and then responded to rechallenge with anti-PD1 therapy. T cells induced by melanoma FixVac are of the PD1+ effector memory phenotype, and accordingly are stimulated by anti-PD1 antibodies. In agreement with this notion, PD1 blockade augments the antitumour effect of RNA-LPX vaccines in mouse models with advanced tumours that are insensitive to anti-PD1 monotherapy (Ref. 18). Notably, the tumour-regression rate (of more than 35%) that was observed with the melanoma FixVac/anti-PD1 combination in pretreated, CPI-experienced patients is in the range of the objective response rates that PD1 blockade alone exerts in patients with CPI-naive metastatic melanoma (Ref 19).

Third, findings presented in Examples 1-6 support the usefulness of non-mutated shared TAAs as cancer vaccine targets. Clinical effects in TAA-based cancer vaccine trials over the past two decades have largely been disappointing and often associated with relatively weak vaccine-induced immunity in patients with advanced cancers (Ref 20). The identification of T cells directed against cancer mutations as drivers of CPI-blockade-mediated clinical efficacy—together with the advancement of technologies that enable individualized cancer vaccination—promoted the idea that cancer mutations that are not compromised by central tolerance mechanisms are the more attractive vaccine targets. However, data shown in Examples 1-6 show that T-cell tolerance against non-mutant TAAs can be overcome by a potent vaccine class. PD1 blockade works through expansion of pre-existing antigen-specific T cells, many of which are directed against mutation-derived neoantigens (Ref 21). More than half of patients with metastatic melanoma have a moderate to low mutational burden, associated with a lower probability of pre-formed neoantigen-specific T cells, and are at higher risk of failure of anti-PD1 treatment and hence disease progression (Ref 22). Given that the four TAAs targeted here are highly prevalent in human melanoma (Ref 10,23,24), and that their expression does not correlate with the tumor mutational burden (FIG. 4g), melanoma FixVac primes, activates and expands a complementary pool of CD4⁺ and CD8⁺ T cells. Thus, vaccines based on non-mutant TAAs may be of particular clinical utility in combination with anti-PD1 therapy for tumour control in patients with a lower mutational burden, including those who have already experienced CPI therapy.

### Example 7: Exemplary Dosing (e.g. Dose Escalation)

In some embodiments, pharmaceutical compositions provided herein can be administered to patients with melanoma as monotherapy and/or in combination with other anti-cancer therapies such as, e.g., immune checkpoint inhibitors. In some embodiments, melanoma patients to be treated are patients with anti-PD1 refractory/relapsed, unresectable Stage III or IV melanoma.

In some embodiments, administration involves at least 8 doses within 10 weeks. In some embodiments, administration can further involve a monthly dose following the 10-week dosing schedule.

In some embodiments, administration involves 6 weekly doses of a pharmaceutical composition described herein (e.g., a FixVac), followed by 2 biweekly doses of a pharmaceutical composition described herein (e.g., a FixVac). In some embodiments, administration can further involve a monthly dose following administration of the 2 biweekly doses.

In some embodiments, administration involves 5 weekly doses of a pharmaceutical composition described herein (e.g., a FixVac), followed by 2 biweekly doses of a pharmaceutical composition described herein (e.g., a FixVac). In some embodiments, administration can further involve a monthly dose following administration of the 2 biweekly doses.

In some embodiments where a combination therapy is administered, a pharmaceutical composition described herein (e.g., a FixVac) may be administrated on the same day as an immune checkpoint inhibitor therapy. In some such embodiments, a pharmaceutical composition described herein (e.g., a FixVac) and an immune checkpoint inhibitor therapy may be administered separately.

In some embodiments, a pharmaceutical composition described herein (e.g., a FixVac) is administrated on the same day as an immune checkpoint inhibitor therapy.

In some embodiments, dose escalation may be performed. In some such embodiments, dosing may be performed at one or more of the levels shown in Table 6; in some embodiments, dose escalation may involve administration of at least one lower dose from Table 6 followed later by administration of at least one higher dose from Table 6.

TABLE 6
Exemplary Dosing

| Dose Level | Dose (μg of total RNA) |
|---|---|
| 1 | 7.2 |
| 2 | 14.4 |
| 3 | 29 |
| 4 | 50 |
| 5 | 75 |
| 6 | 100 |
| 7 | 200 |
| 8 | 400 |

In some embodiments, additional or alternative doses levels may be evaluated, for example, including, e.g., dose levels at 7.5, 8, 9, 10, 11, 12, 13, 14 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 225, 250, 275, 300, 325, 350, 375, 400 μg of total RNA. Efficacy of a treatment can be assessed by immune monitoring and/or clinical anti-tumor activity.

### Example 8: Exemplary Immune Checkpoint Inhibitors that can be Used in Combination with Pharmaceutical Compositions Described Herein

Approved immune checkpoint inhibitors are available for treatment of certain cancers including melanoma. Non-limiting examples of FDA-approved immune checkpoint inhibitors include ipilimumab, Cemiplimab, Nivolumab, Pembrolizumab, Atezolizumab, Avelumab, and Durvalumab. Additional examples of immune checkpoint inhibitors that are currently under studies may include Dostarlimab, INCMGA00012, Toripalimab, SHR-1210, INCB086550 (oral PD-1 inhibitor), PDR001, HX008, and CX-072.

In some embodiments, an immune checkpoint inhibitor may be administered according to a regimen indicated as monotherapy for treatment of certain cancer, e.g., in some embodiments every 3 weeks.

### Example 9: Exemplary Adverse Events

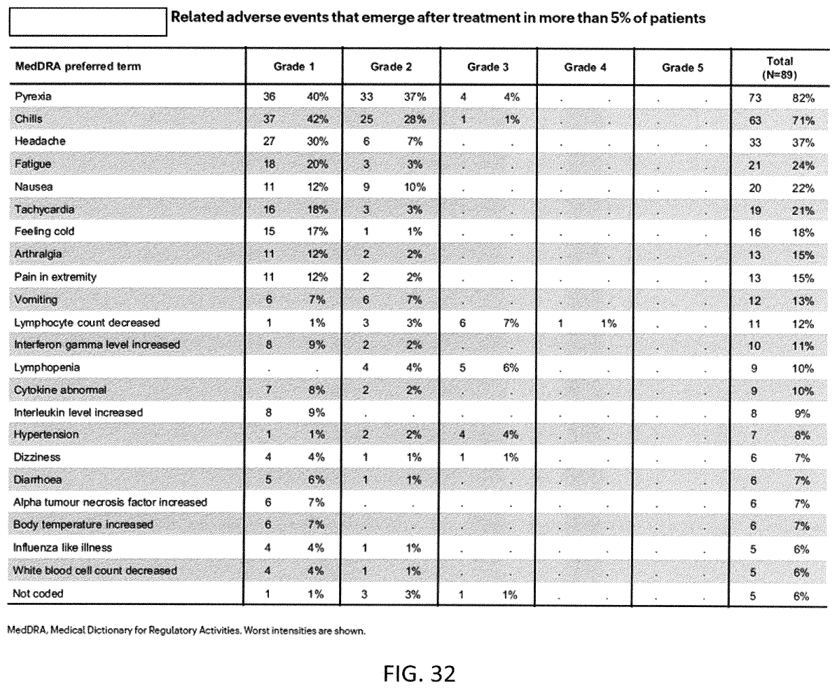

In some embodiments, subjects to whom monotherapy as described herein is administered may be monitored over a period of treatment regimen for one or more indicators of a potential adverse event. The clinical adverse-event profile was dominated by mild to moderate flu-like symptoms, such as pyrexia and chills. Adverse events were mostly early-onset, transient and manageable with antipyretics, and resolved within 24 hours (FIG. 32). In some embodiments, particularly for subjects receiving monotherapy as described herein, subjects may be monitored for one or more pyrexia, chills, headache, fatigue, nausea, tachycardia, feeling cold, arthralgia, pain in extremity, vomiting, lymphocyte count decreased, interferon gamma level increased, hypertension, dizziness, diarrhea, alpha tumor necrosis factor increased, influenza like illness, and white blood cell count decreased.

### Example 10: Exemplary Discontinuation Criteria

In some embodiments, a therapy as described herein may be discontinued if, for example, (i) a patient experiences an adverse event (AE) fulfilling drug limiting toxicities (DLT) criteria; (ii) a patient experiences an AE fulfilling the DLT criteria after a dosing cycle that fails to resolve to Grade ≤1 within a pre-determined time period; (iii) a dose delay of more than a dosing cycle due to toxicity that may be related to the administered therapy; (iv) a drug-related or life-threatening Grade 4 AE that does not fulfill the DLT criteria (excluding asymptomatic Grade 4 elevations in non-hematological laboratory values that resolve to ≤Grade 2 within 14 days [with or without medical intervention]) unless otherwise approved by the medical monitor; (v) second occurrence of an infusion related reaction (IRR) of Grade ≥3 despite premedication prior to second administration; and/or (vi) first occurrence of anaphylaxis or Grade 4 IRR.

### Example 11: Exemplary Assessments and/or Criteria for RNA Molecules Described Herein

In some embodiments, one or more assessments as described herein may be utilized during manufacture, or other preparation or use of RNA molecules (e.g., as a release test).

In some embodiments, one or more quality control parameters may be assessed to determine whether RNA molecules described herein meet or exceed acceptance criteria (e.g., for subsequent formulation and/or release for distribution). In some embodiments, such quality control parameters may include, but are not limited to RNA integrity, RNA concentration, residual DNA template and/or residual dsRNA. Methods for assessing RNA quality are known in the art; for example, one of skill in the art will recognize that in some embodiments, one or more analytical tests such as, e.g., capillary gel electrophoresis for RNA integrity, UV absorption spectrophotometry for RNA content and/or concentration, Quantitative PCR for residual DNA template, immuno-based assay for residual dsRNA, detection of translated antigen, can be used for RNA quality assessment.

In some embodiments, a batch of RNAs may be assessed, e.g., for RNA integrity, RNA content and/or concentration, residual DNA template, residual dsRNA, expression of antigen, or combinations thereof, to determine next action step(s). For example, a batch of RNA molecules can be designated for one or more further steps of manufacturing and/or formulation and/or distribution if RNA quality assessment indicates that such a batch of RNA molecules meet or exceed the pre-determined acceptance criteria. Otherwise, an alternative action can be taken (e.g., discarding the batch) if such a batch of RNA molecules does not meet or exceed the acceptance criteria.

### Example 12: Exemplary Inclusion Criteria

In some embodiments, cancer patients who meets one or more of the following disease-specific inclusion criteria are selected for treatment with compositions and/or methods described herein:
Cohort I: stage IV malignant melanoma (AJCC 2009 melanoma classification)
Cohorts II-VII end expanded cohorts: stage IIIB-C, or stage IV of malignant melanoma (AJCC 2009 melanoma classification) Expanded cohorts C only patients with stage IV melanoma (AJCC 2009 melanoma classification) with measurable disease (at least one target lesion according irRECIST 1.1) [applicable for all patients after approval of protocol version 10.0]
Therapy only for subjects not eligible or declining any other available approved therapy after all available treatment options have been transparently disclosed (to be documented).
Expression of either one of four TAA confirmed by RT-qPCR analysis from FFPE
≥18 years of age
Written informed consent
ECOG performance status (PS) 0-1
Life expectancy >/=6 months
WBC≥3×10E9/L
Hemoglobin ≥9 g/dL
Platelet count ≥100,000/mm³
ALT/AST<3×ULN (except patients with liver metastasis)
Negative pregnancy test (measured by β-HCG) for females with childbearing age

### Example 13: Exemplary Exclusion Criteria

In some embodiments, cancer patients have melanoma that is not amenable to compositions and/or methods described and/or utilized herein.

In some embodiments, cancer patients who (i) have recently received a cancer treatment; (ii) are concurrently receiving systemic steroid therapy; (iii) have recently had a major surgery; (iv) are suffering from active infection and being treated with an anti-infective therapy; and/or (v) are diagnosed with growing brain or leptomeningeal metastases, are not amenable to compositions and/or methods described and/or utilized herein.

In some embodiments, the following cancer patients may not be recommended for treatment with the pharmaceutical composition described herein. Exclusion criteria includes:
Pregnancy or breastfeeding
Primary ocular melanoma
Concurrence of a second malignancy other than squamous or basal cell carcinoma, non-active prostate cancer, or cervical carcinoma in situ or non-active treated urothelial carcinoma
Brain metastases
   Patients with history of treated or inactive brain metastasis are eligible for treatment in expanded cohort C, provided they meet all of the following criteria:
   measurable disease outside of the brain (in addition to inactive brain metastasis);
   no ongoing requirement of corticosteroids as therapy for brain metastases,
   with corticosteroids discontinued ≥1 week prior to visit 2 (day 1) with no ongoing symptoms attributable to brain metastasis;
   the screening brain radiographic imaging is ≥4 weeks since completion of radiotherapy
Post-splenectomy Patients
Known hypersensitivity to the active substance or to any of the excipients
A serious local infection (e.g. cellulitis, abscess) or systemic infection (e.g. pneumonia, septicemia) which requires systemic antibiotic treatment within 2 weeks prior to the first dose of study medication
Positive test for acute or chronic active hepatitis B or C infection
Clinically relevant active autoimmune disease
Systemic immune suppression:
   HIV disease
   Use of chronic oral or systemic steroid medication (topical or inhalational steroids are permitted)
   Other clinical relevant systemic immune suppression
Symptomatic congestive heart failure (NYHA 3 or 4)
Unstable angina pectoris
Radiotherapy and minor surgery within 14 days prior to the first study treatment administration
Myelosuppressive chemotherapy within 14 days and after reconstitution of blood values prior to the first study treatment administration
Ipilimumab within 28 days prior to the first study treatment administration
Treatments with BRAF inhibitors, MEK inhibitors, or the combination of both, and anti-PD-1 antibodies within 14 days prior to the first administration of study treatment (not applicable for patients with parallel treatment in expanded cohorts A, B, or C at the discretion of the investigator)
Interferon, major surgery, vaccination, and other investigational agents within 28 days or 5 half-lives depending on what gives the longer range before the first treatment
Approved BRAF inhibitors vemurafenib or dabrafenib, approved anti-PD-1 inhibitors nivolumab or pembrolizumab as well as approved MEK inhibitor trametinib, or the approved combination of BRAF-MEK inhibitors in patients in dose escalation cohorts. Concomitant treatment with approved BRAF inhibitors, approved anti-PD-1 antibodies or MEK inhibitor as well as the approved combination of BRAF-MEK inhibitors is allowed for patients included in the expanded cohorts, after analysis of safety data collected for the dose escalation cohorts and DSMB approval. Local radiation will be allowed as concurrent treatment for patients in expanded cohort as well.
   After approval of protocol version 10.0 only anti-PD-1 antibodies are allowed for treatment of patients in expanded cohort C.
Fertile males and females who are unwilling to use a highly effective method of birth control (less than 1% per year, e.g. condom with spermicide, diaphragm with spermicide, birth control pills, injections, patches or intrauterine device) during study treatment and for at least 28 days (male patients) and 90 days (female patients of childbearing potential) after the last dose of study treatment
Presence of a severe concurrent illness or other condition (e.g. psychological, family, sociological, or geographical circumstances) that does not permit adequate follow-up and compliance with the protocol

### Example 14: Exemplary Efficacy Assessments and/or Monitoring

In some embodiments, a cancer patient administered with a pharmaceutical composition described herein as a monotherapy or in combination with an additional anti-cancer therapy may be periodically monitored for efficacy of the treatment and/or adjustment of the treatment dosage/schedule.

In some embodiments, efficacy of a treatment may be assessed by computer tomography and/or magnetic resonance imaging scans. In some embodiments, a MRI scan may be performed using a 3 Tesla whole body instrument. In some embodiments, when evaluating lesions for efficacy assessments, one or more of following criteria may be used:
Complete response: disappearance of all target lesions. Any pathological lymph nodes (whether target or non-target) must have reduction in short axis to <10 mm.
Partial response: at least a 30% decrease in the sum of diameters of target lesions, taking as reference the baseline sum diameters.
Progressive disease: at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progression.
Stable disease: neither sufficient shrinkage to qualify for PR nor sufficient increase to qualify for progressive disease, taking as reference the smallest sum diameters while on study.

### Example 15: Immune Response from Patients with Evidence of Disease Versus Patients with No Evidence of Disease

The present Example shows ex vivo characterization of the immune response following administration of an exemplary pharmaceutical composition comprising one or more RNA molecules that collectively encode a NY-ESO-1 antigen, a MAGE-A3 antigen, a tyrosinase antigen, a TPTE antigen, or a combination thereof, and lipid particles to patients with Evidence of Disease (ED) and patients with No Evidence of Disease (NED).

Background: Lipo-MERIT is an ongoing, first-in-human, open-label, dose-escalation Phase I trial investigating the safety, tolerability and immunogenicity of BNT111 in patients with advanced melanoma. BNT111 is a ribonucleic acid lipoplex (RNA-LPX) vaccine targeting the melanoma tumor-associated antigens (TAAs) New York esophageal squamous cell carcinoma 1 (NY-ESO-1), tyrosinase, melanoma-associated antigen 3 (MAGE-A3), and transmembrane phosphatase with tensin homology (TPTE). As demonstrated in Examples 1-6, BNT111, alone or combined with an immune checkpoint inhibitor (CPI), has a favorable adverse event (AE) profile, gives rise to antigen-specific T-cell responses and induces durable objective responses in CPI-experienced patients with unresectable melanoma. This Example shows immunogenicity, efficacy and safety data in patients with no evidence of disease (NED) at trial inclusion in the BNT111 monotherapy subgroup.

Methods: Patients with stage IIIB, IIIC and IV cutaneous melanoma were intravenously administered with BNT111 according to a prime-and-boost protocol. Patients were treated in seven dose escalation cohorts (dose range: 7.2 μg to 400 μg total RNA) and three expanded cohorts to further explore dose levels of 14.4 μg, 50 μg and 100 μg. In this analysis, patients receiving BNT111 monotherapy were grouped as having evidence of disease (ED) or NED, and immunogenicity, efficacy (by immune-related Response Evaluation Criteria In Solid Tumors) and safety were evaluated. Vaccine-induced immune responses were analyzed using an interferon-γ enzyme-linked immune absorbent spot (ELISpot) assay directly ex vivo.

Results: As of 24 May 2021, 115 patients have received BNT111 within the Lipo-MERIT trial. Of 71 patients treated with BNT111 monotherapy, 38 patients had ED and 33 patients had NED after prior therapies. Baseline characteristics were similar between the two groups. ELISpot data revealed comparable BNT111-induced T-cell responses against at least one TAA in ED vs. NED patients (14/22 [64%] and 19/28 [68%] patients with available ELISpot-evaluable samples, respectively), indicating that BNT111 has the ability to induce T-cell immunity, even in the absence of a detectable tumor. In NED patients, clinical efficacy was promising, with a median disease-free survival of 34.8 months (95% confidence interval: 7.0—not reached). The safety profile was similar in ED vs. NED patients, with 38/38 patients (100%) and 32/33 patients (97%) experiencing related treatment-emergent AEs (TEAEs), respectively, of which the majority were mild-to-moderate flu-like symptoms.

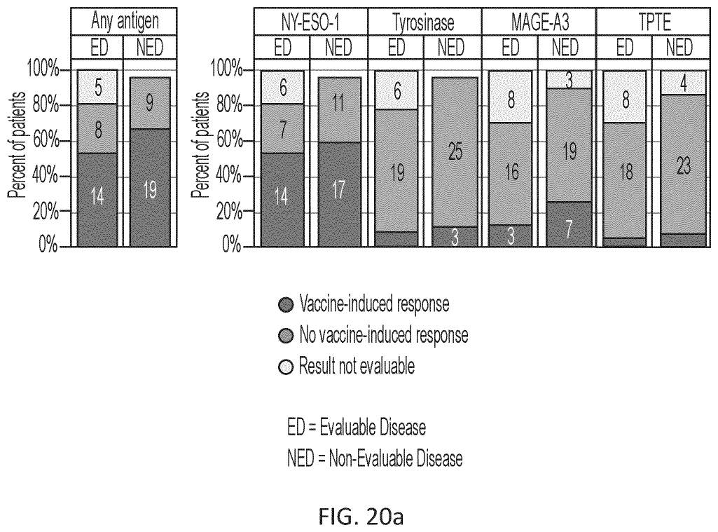

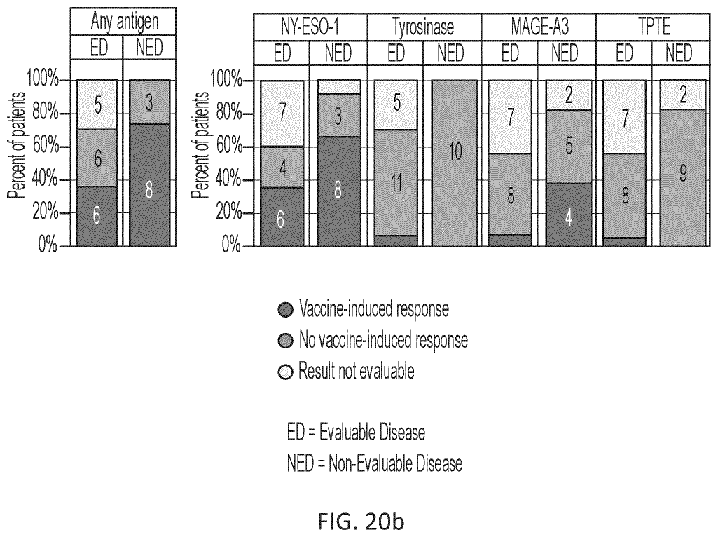

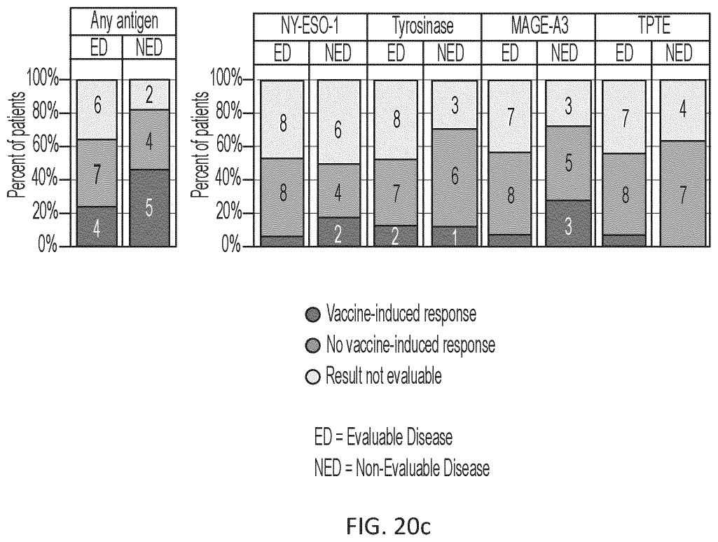

In particular, samples from patients having ED and NED were analyzed using ex vivo ELISpot (FIGS. 20a-c) in which autologous dendritic cells loaded with TAA PepMixes were used as targets. FIGS. 20a-c show the frequency of patients with vaccine-induced (amplified or de novo) response: CD4⁺ or CD8⁺ (FIG. 20a); CD4⁺ (FIG. 20b); or CD8⁺ (FIG. 20c) responses. Numbers in bar segments represent number of evaluated patients per segment. Only patients treated in monotherapy are included. Surprisingly, samples showed greater vaccine-induced response (e.g., CD4⁺ or CD8⁺ (FIG. 20a); CD4⁺ (FIG. 20b); or CD8⁺ (FIG. 20c) in NED patients than ED patients.

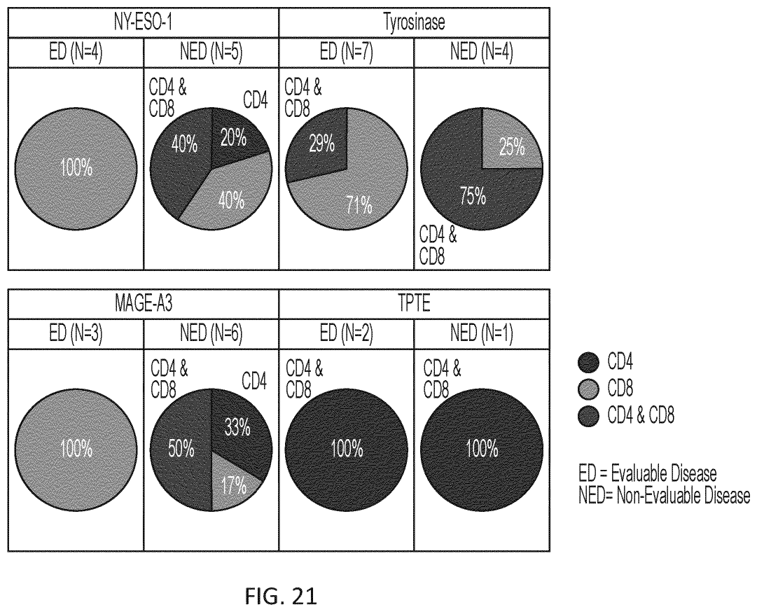

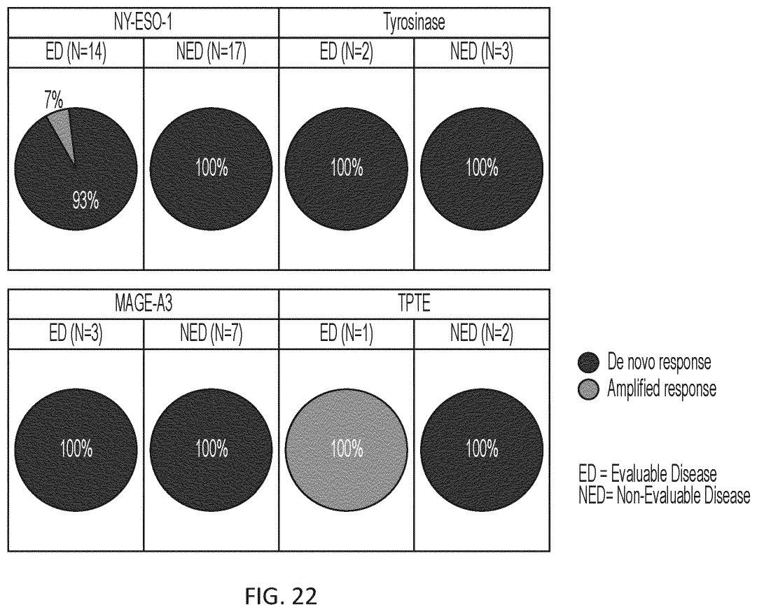

Results of ex vivo ELISPOT were also compared by cell type. As shown in FIGS. 21-22, the TAAs induced more prominent and diversified immune responses in NED patients as compared to ED patients. Comparing de novo response versus amplified responses in an ex vivo ELISPOT assay (assessing CD4⁺ or CD8⁺ response to any cell type) revealed 100% de novo response in each (4/4 antigens) NED patient population compared to half for the ED (2/4 antigens) patient population.

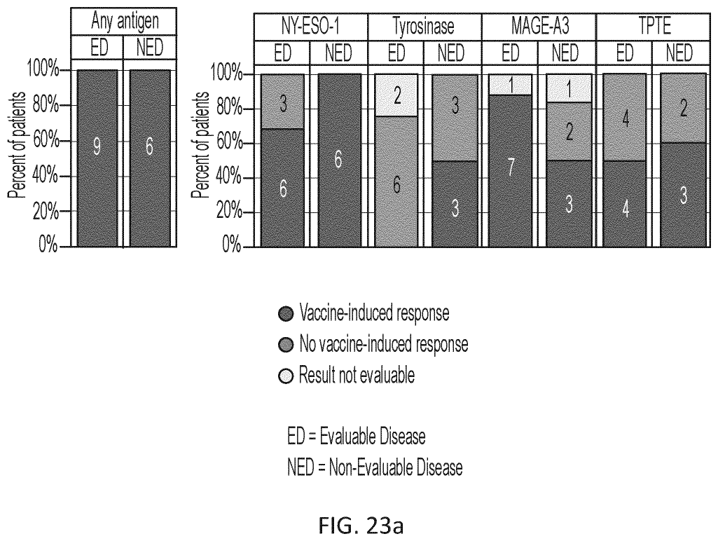

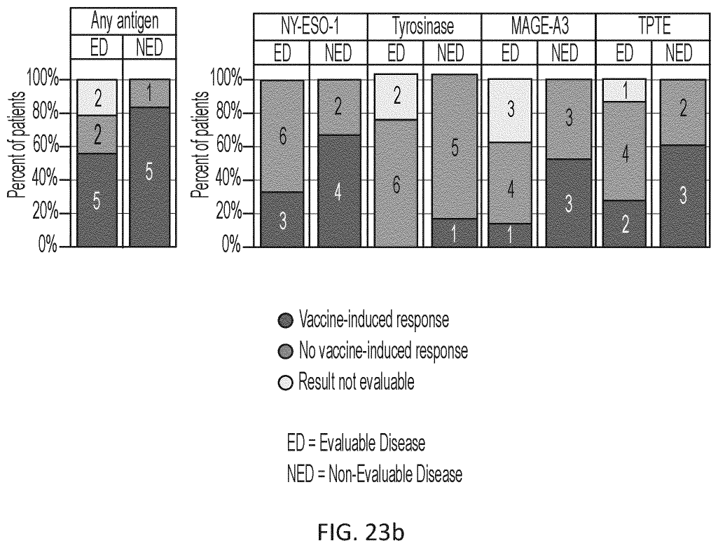

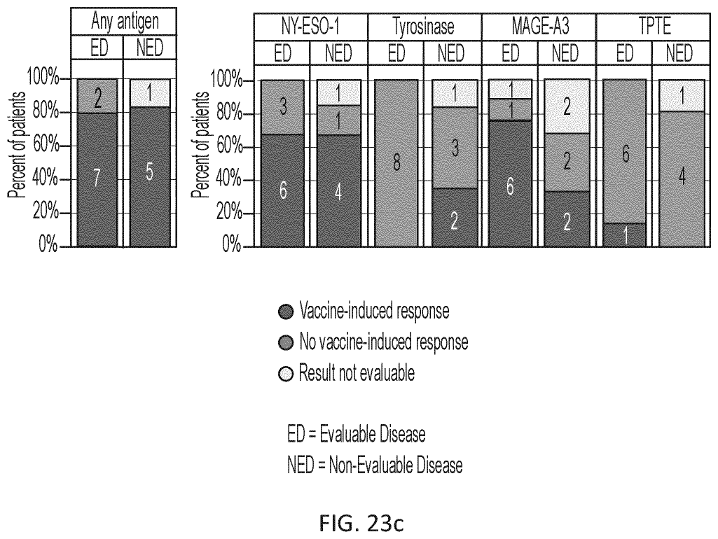

Samples from patients having ED and NED were analyzed using post-IVS ELISpot (FIGS. 23a-c) in which autologous dendritic cells loaded with TAA PepMixes were used as targets. FIGS. 23a-c show the frequency of patients with vaccine-induced (amplified or de novo) response: CD4⁺ or CD8⁺ (FIG. 23a); CD4+ (FIG. 23b); or CD8+ (FIG. 23c) responses. Numbers in bar segments represent number of evaluated patients per segment. Only patients treated in monotherapy are included. Surprisingly, samples showed greater vaccine-induced response (e.g., CD4⁺ or CD8⁺ (FIG. 23a); CD4⁺ (FIG. 23b); or CD8⁺ (FIG. 23c) in NED patients than ED patients.

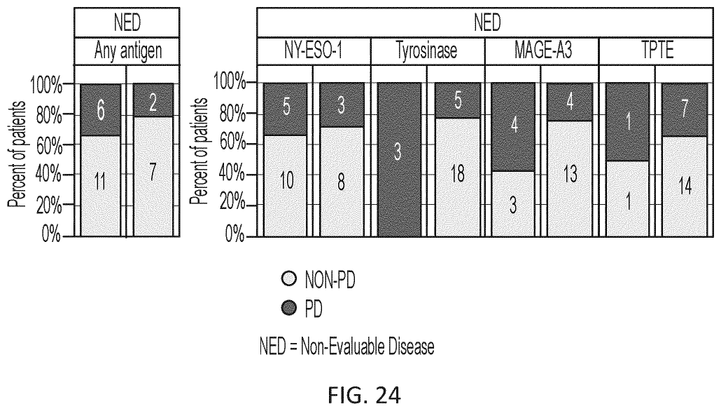

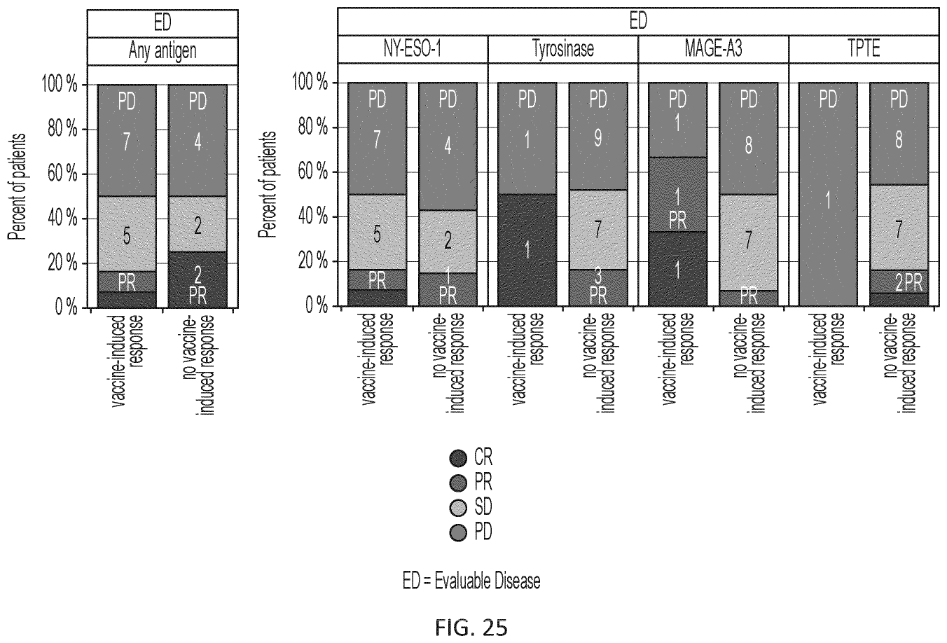

As shown in FIGS. 24 and 25, upper panel show non-evaluable disease patients and the lower panel: show evaluable disease patients. The Numbers in bar segments represent number of patients with evaluated ex-vivo ELISPOT measurements per segment. Only patients treated with monotherapy are included.

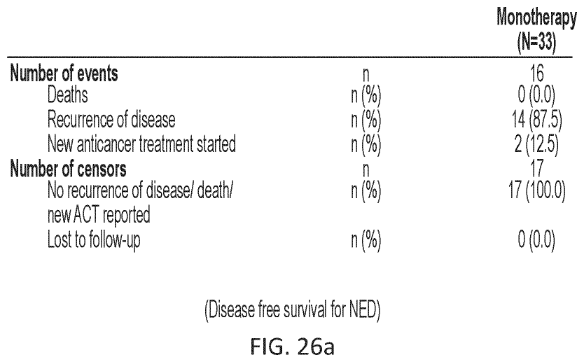

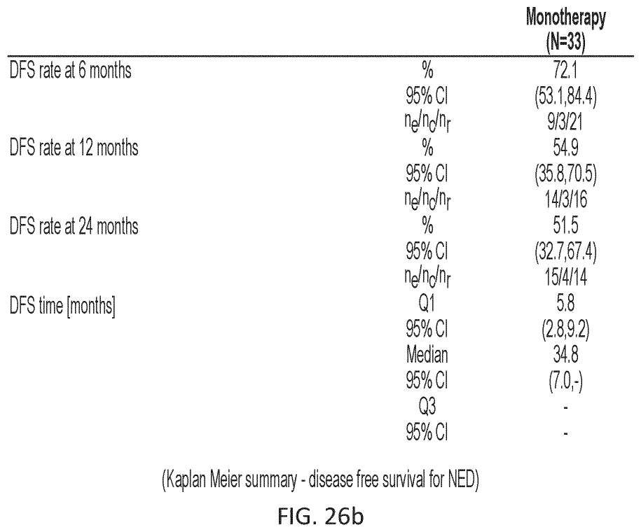

FIG. 26a shows disease free survival data for NED patients based on number of events (e.g., deaths, recurrence, and new treatment started) and numbers of censors. FIG. 26b shows Kaplan-Meier summary of disease free survival data for NED patients.

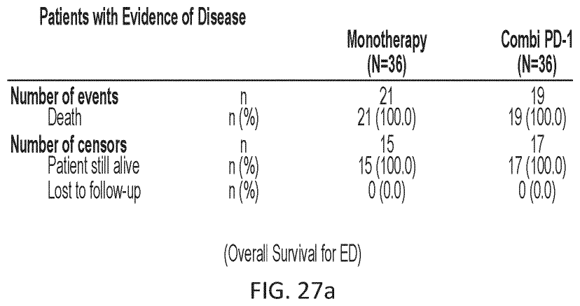

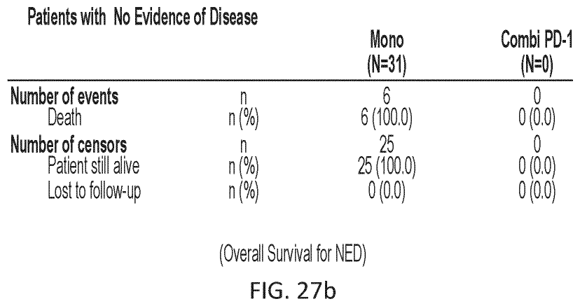

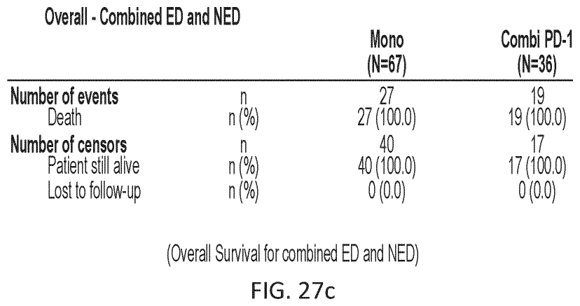

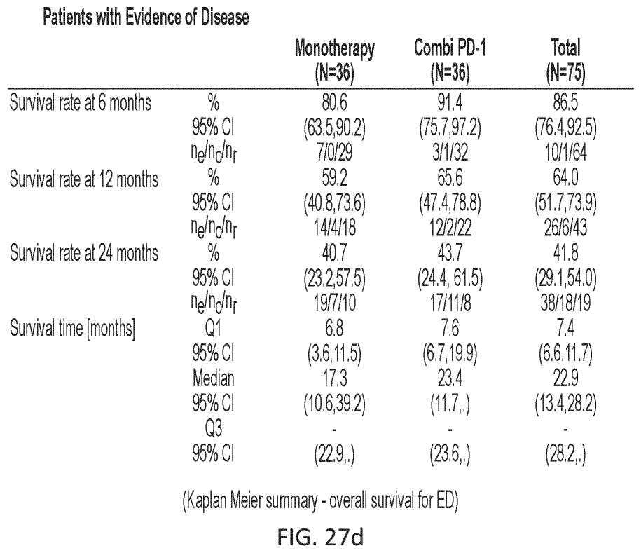

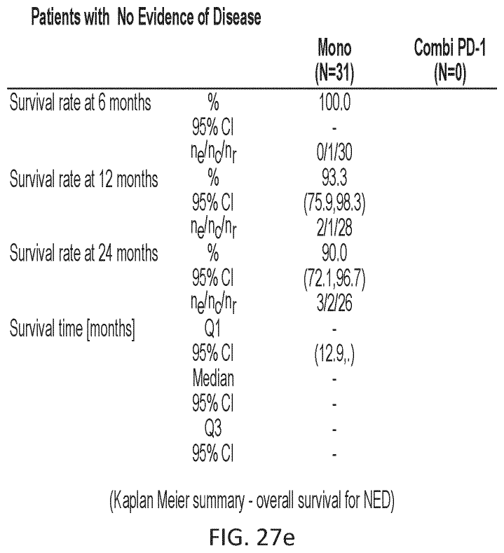

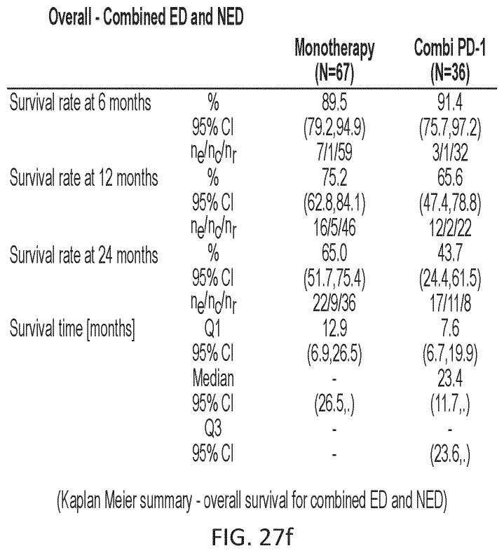

FIGS. 27a-27c show overall survival data for ED patients (FIG. 27a), NED patients (FIG. 27b), and combined ED and NED patients (FIG. 27c) based on number of events (e.g., deaths, recurrence, and new treatment started) and numbers of censors. FIGS. 27d-27f shows Kaplan-Meier summary of overall survival data for ED patients (FIG. 27d), NED patients (FIG. 27e), and combined ED and NED patients (FIG. 27f).

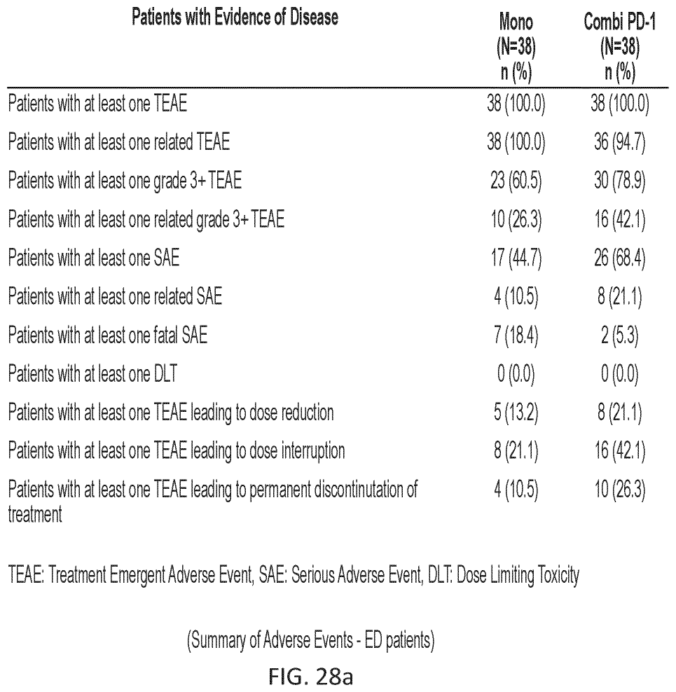

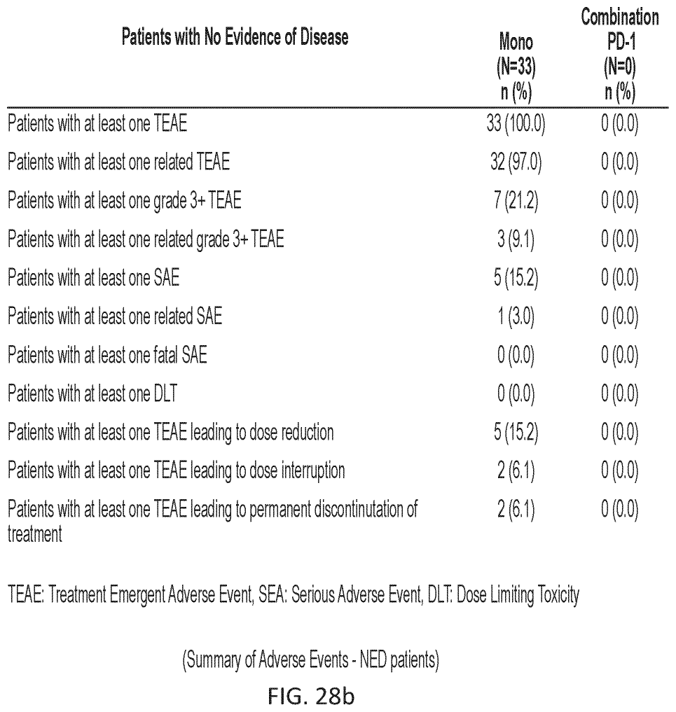

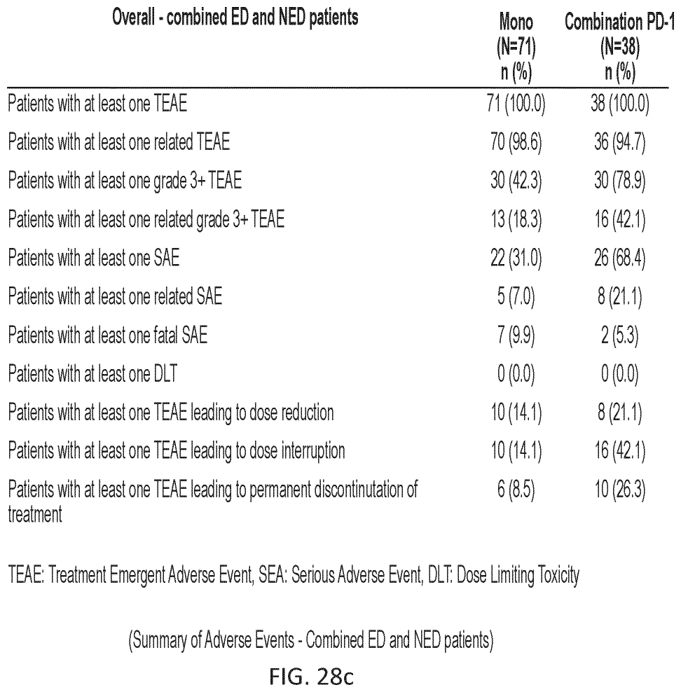

FIGS. 28a-28c show summary of adverse events for ED patients (FIG. 28a), NED patients (FIG. 28b), and combined ED and NED patients (FIG. 28c).

Conclusions: The immunogenicity and safety profiles of BNT111 as monotherapy was comparable in ED and NED patients, and promising signs of clinical activity were observed in NED patients.

### Example 16: Pharmacology and Immune Response Following BNT111 Administration

The present Example shows the immune responses detected following administration of BNT111 to patients.

The cytokines, e.g., IFN-γ, IFN-α, TNF-α, IP-10, IL-2, IL-6, IL-10, and IL-12 (p70), were analyzed at different time points ranging from baseline (i.e., pre-vaccination), up to 36 d post-vaccination, with frequent sampling during the first 48 h post-vaccination. Patients demonstrated a dose-dependent transient increase of plasma levels of a distinct spectrum of cytokines and elevation of body temperature. Cytokine release was pulsatile with peaks at approximately 2 to 6 h after dosing and values returning to baseline by 24 h or earlier. Activation of an IFN-α dominated pattern of cytokines including IFN-γ and consecutively IP-10 and also of IL-12, IL-6, and TNF-α was observed.

In blood samples from 20 patients analyzed with IFN-γ-enzyme-linked immune absorbent spot (ELISpot) after in vitro expansion, T cell responses were observed for at least one TAA in each patient. These included T cell specificities undetectable at baseline and induced de novo by the vaccine, as well as T cell specificities which were present at low levels at baseline and were expanded and amplified by vaccine antigens.

In 80 patients, IFN-γ-ELISpot was conducted ex vivo without prior in vitro stimulation. In 72.5% of these patients, robust immune responses against at least one TAA were induced to a level that was detectable ex vivo.

All four TAAs were immunogenic. The majority of patients exhibited either a CD4⁺ response alone or concurrent CD4⁺ and CD8⁺ T cell responses against the individual TAAs.

T cell responses, including de novo primed ones, were found to be induced rapidly within 4 to 8 wks, reached high magnitudes and were durable over several months. In some patients, antigen-specific CD8⁺ T cell responses representing more than 10% of all peripheral blood CD8⁺ T cells were observed.

In selected cases, the expansion of T cell specificities was observed to parallel reduction of tumor burden.

### Example 17: Efficacy Data Following BNT111 Administration

This Examples provides an overview of the preliminary efficacy data observed following administration of BNT111.

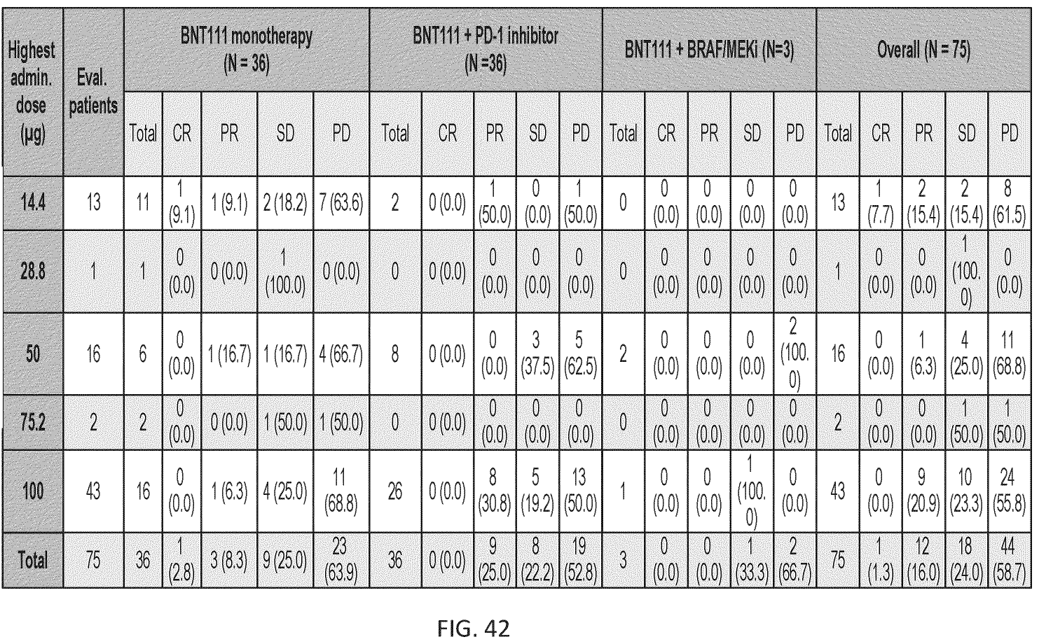

The preliminary efficacy is presented for BNT111 monotherapy, BNT111 with either nivolumab or pembrolizumab and for BNT111 in combination with BRAF/MEK inhibitors. FIG. 42 provides details on the best overall response for each of these treatment groups according to the highest dose administered.

Out of the 115 patients, 75 (68%) patients with unresectable Stage III or IV melanoma presented with evaluable disease at baseline included four patients with non-target lesions only. The subgroup efficacy analysis set included patients with evaluable disease at baseline, who received at least one dose of BNT111 and have a baseline and at least one on/post treatment tumor response assessment (N=75).

The efficacy was presented for 36 patients who received BNT111 monotherapy, 36 patients who received BNT111 with either nivolumab or pembrolizumab and three patients who received BNT111 with BRAF/MEK inhibitors. All 36 BNT111 monotherapy patients had received prior treatment with checkpoint inhibitors, and of those treated with BNT111 in a PD-1 inhibitor combination, 35/36 patients had received prior treatment with checkpoint inhibitors. The majority of patients had progressive disease at the start of treatment.

The best overall response (the best response recorded from the start of the trial treatment until the disease progression/recurrence) in the 36 patients with evaluable disease at baseline treated with the BNT111 monotherapy comprised one patient (3%) with a CR, three patients (8%) with a PR and nine patients (25%) with SD. The overall response rate was 11% and the disease control rate was 36%. Median duration of response was 8.4 months (95% confidence interval [CI]: 6.2 to 33.3 months).

The best overall response in the 36 patients that were evaluable for efficacy analysis treated with the BNT111 cancer vaccine in combination with either nivolumab or pembrolizumab comprised nine (25%) patients who achieved PR, and eight (22%) with SD, resulting in an overall response rate of 25% and a disease control rate of 47%. Median duration of response was 22.9 months (95% CI: 3.0 to 22.9 months).

Of the three patients evaluable for efficacy and treated with the BNT111 cancer vaccine in combination with BRAF/MEK inhibitors, one (33.3%) patient achieved SD.

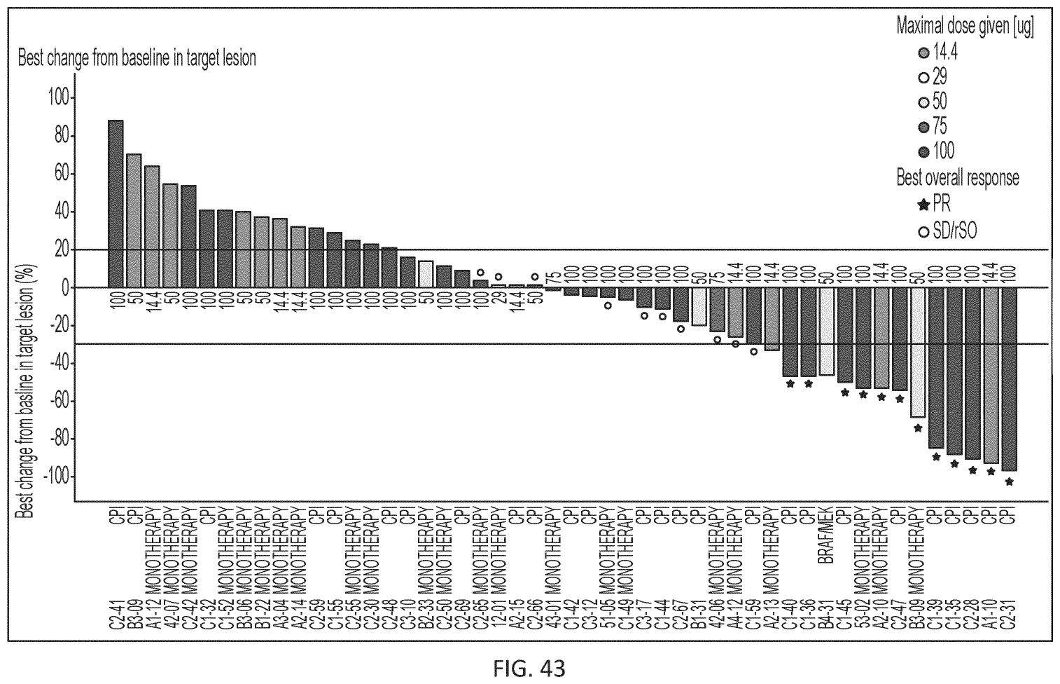

FIG. 43 depicts the best change from baseline in target lesion according to irRECIST in patients with measurable disease treated with monotherapy or combination with either nivolumab or pembrolizumab or BRAF/MEK inhibition.

### Example 18: Safety Analysis

This Examples provides an assessment of the safety of an exemplary composition described herein.

BNT111 was administered to 115 patients with melanoma. BNT111 demonstrated a favorable safety and tolerability profile as monotherapy (n=38). Thirty-eight patients received BNT111 in combination with either pembrolizumab or nivolumab, both dosed according to the respective product labels. Favorable safety and tolerability of the combination was also demonstrated.

Almost all patients in the subgroups had a TEAE related to the study drug. The overall safety profile between treatment subgroups, e.g., BNT111 monotherapy versus BNT111 in combination with a PD-1 inhibitor or BRAF/MEK, was comparable with only a few differences noted. However, the number of patients treated in combination with BRAF/MEK inhibitors was too small to draw any conclusions.

The overall safety profile for the combination therapy with a PD-1 inhibitor versus BNT111 monotherapy was comparable in regard to flu-like symptoms (reactogenicity) such as pyrexia, chills, tachycardia and headache. The most important TEAEs more frequent in the PD-1 combination therapy subgroup compared to BNT111 monotherapy were syncope (13% vs. 0%) and melanocytic naevus (13% vs. 3%).

Differences were noted in the PD-1 inhibitor combination subgroup versus the BNT111 monotherapy subgroup for gastrointestinal AEs such as nausea (55% vs. 17%), vomiting (29% vs. 17%), diarrhea (11% vs. 3%), and decreased appetite (13% vs. 3%).

In addition, a difference was also noted between the PD-i inhibitor combination subgroup versus the BNT111 monotherapy subgroup for hypotension (24% vs. 9%). A higher number of arthralgia (31% vs. 11%;) was reported for the BNT111 monotherapy subgroup. In the Lipo-MERIT Phase I trial, no dose-limiting toxicities (DLTs) were reported during dose escalation (from 7.2 pig up to the highest administered dose of 400 μg total RNA).

There were no drug-related deaths reported. There were 11/115 (8%) patients who died within the main course of the trial, i.e., within 90 days after the last trial treatment. None of the deaths were considered related to BNT111. Most of the patients died due to disease progression and general physical health deterioration.

The TEAEs considered related to study drugs were transient, mostly flu-like symptoms and of Common Terminology Criteria for Adverse Events (CTCAE) Grade 1 and 2.

Thirteen of 115 (11%) patients experienced treatment-related TESAEs; 30/115 (26%) patients experienced treatment-related TEAEs of CTCAE Grade ≥3; 19/115 (17%) patients with treatment-related TEAEs leading to permanent trial treatment discontinuation and 19/115 (17%) patients with treatment-related TEAEs leading to dose reductions. Table 7 provides an overview of the TEAEs by category.

TABLE 7
Lipo-MERIT-Overview of the Number and Percentage of Patients with at
Least One TEAE by Subgroup¹⁻⁴

| | Frequency by Patient Subgroups | | | | | | |
|---|---|---|---|---|---|---|---|
| | Evaluable disease | | | | | | |
| | | BNT111 + | | | Non-evaluable disease | | |
| | BNT111 | PD-1 | BNT111 + | | BNT111 | BNT111 + | |
| | monotherapy | inhibitor | BRAF/MEK | All | monotherapy | BRAF/MEK | All |
| Category | N = 38 | N = 38 | N = 6 | N = 81 | N = 33 | N = 1 | N = 34 |
| TEAE by PT | 38 (100.0) | 38 (100.0) | 6 (100.0) | 81 (100.0) | 33 (100.0) | 1 (100.0) | 34 (100.0) |
| TEAE related to | 38 (100.0) | 36 (94.7) | 6 (100.0) | 79 (97.5) | 32 (97.0) | 1 (100.0) | 33 (97.1) |
| BNT111 | | | | | | | |
| CTCAE | 23 (60.5) | 30 (78.9) | 6 (100.0) | 59 (72.8) | 7 (21.2) | 0 (0.0) | 7 (20.6) |
| Grade 3 to 5 | | | | | | | |
| TEAE | | | | | | | |
| Grade 3 to 5 | 10 (26.3) | 16 (42.1) | 1 (16.7) | 27 (33.3) | 3 (9.1) | 0 (0.0) | 3 (8.8) |
| TEAE³ related | | | | | | | |
| to BNT111 | | | | | | | |
| SAE | 17 (44.7) | 26 (68.4) | 5 (83.3) | 48 (59.3) | 5 (15.2) | 0 (0.0) | 5 (14.7) |
| SAE related to | 4 (10.5) | 8 (21.1) | 0 (0.0) | 12 (14.8) | 1 (3.0) | 0 (0.0) | 1 (2.9) |
| BNT111 | | | | | | | |
| SAE leading to | 7 (18.4) | 2 (5.3) | 2 (33.3) | 11 (13.6) | 0 (0.0) | 0 (0.0) | 0 (0.0) |
| death | | | | | | | |
| DLT | 0 (0.0) | 0 (0.0) | 0 (0.0) | 0 (0.0) | 0 (0.0) | 0 (0.0) | 0 (0.0) |
| TEAE leading | 5 (13.2) | 8 (21.1) | 1 (16.7) | 14 (17.3) | 5 (15.2) | 0 (0.0) | 5 (14.7) |
| to dose | | | | | | | |
| reduction⁵ | | | | | | | |
| TEAE leading | 8 (21.1) | 16 (42.1) | 1 (16.7) | 25 (30.9) | 2 (6.1) | 0 (0.0) | 2 (5.9) |
| to dose | | | | | | | |
| interruption | | | | | | | |
| TEAE leading | 4 (10.5) | 10 (26.3) | 3 (50.0) | 17 (21.0) | 2 (6.1) | 0 (0.0) | 2 (5.9) |
| to permanent | | | | | | | |
| discontinuation | | | | | | | |
| of treatment⁶ | | | | | | | |

¹Data extraction date/eCRF data extract as of 24 May 2021.
²AEs with missing ACTION TAKEN with BNT111 were not conservatively considered as dose reduction. For one event this entry is missing in eCRF: one event of CRP increased (not MedDRA coded yet, CTCAE grade not reported, considered not related) in one patient of Expanded Cohort C (BNT111 + PD-1 inhibitor treatment group).
³AEs with missing causality are not conservatively considered as related to BNT111. This applies for the following event: One event of pain flank right (not MedDRA coded yet in eCRF); no CTCAE grade provided in eCRF in one patient of Expanded Cohort C (BNT111 + PD-1 inhibitor treatment group).
⁴One patient who received BNT111 as monotherapy in the first enrolment and BNT111 + BRAF/MEK in the second enrolment is represented on this table. Therefore, there is a difference between the total number and the sum of individual therapies displayed.
AE = adverse event; CRP = C-reactive protein; CTCAE = Common Terminology Criteria for Adverse Events; DLT = dose-limiting toxicity; eCRF = electronic case report form; MedDRA = Medical Dictionary for Regulatory Activities; MEK = mitogen-activated protein kinase kinase; PD-1 = programmed death 1; PT = preferred term; SAE = serious adverse event; TE = treatment-emergent; TEAE = treatment-emergent adverse event; TESAE = treatment-emergent serious adverse event.

Table 8 provides a summary of the frequency of related treatment-emergent serious adverse events (TESAEs) by worst CTCAE grade and Table 9 provides a summary of the same data by treatment sub-populations.

TABLE 8
Lipo-MERIT - Number of Patients with Related
TESAE¹ of Worst CTCAE Grade by PT (N = 115)²

| Related Treatment- | Worst AE grade | | | |
|---|---|---|---|---|
| Emergent Serious | Grade 2 | Grade 3 | Grade 4 | Total |
| Adverse Event (TESAE) | N (%) | N (%) | N (%) | N (%) |
| Patients with any related TESAE | 4 (3.5) | 8 (7.0) | 1 (0.9) | 13 (11.3) |
| Pyrexia | 2 (1.7) | — | — | 2 (1.7) |
| Dizziness | 1 (0.9) | 1 (0.9) | — | 2 (1.7) |
| Syncope | — | 2 (1.7) | — | 2 (1.7) |
| Hypotension | — | 2 (1.7) | — | 2 (1.7) |
| Lymphadenopathy | 1 (0.9) | | | 1 (0.9) |
| Serous retinopathy | — | 1 (0.9) | — | 1 (0.9) |
| Autoimmune pancreatitis | | 1 (0.9) | — | 1 (0.9) |
| Nausea | 1 (0.9) | — | — | 1 (0.9) |
| Asthenia | 1 (0.9) | — | — | 1 (0.9) |
| General physical health | 1 (0.9) | — | — | 1 (0.9) |
| deterioration | | | | |
| Anaphylactic reaction | | 1 (0.9) | — | 1 (0.9) |
| Cytokine release syndrome | — | — | 1 (0.9) | 1 (0.9) |
| Encephalitis autoimmune | — | 1 (0.9) | — | 1 (0.9) |
| Epilepsy | — | 1 (0.9) | — | 1 (0.9) |
| Posterior reversible | — | 1 (0.9) | — | 1 (0.9) |
| encephalopathy syndrome | | | | |

¹TESAE is defined as occurring after start of study drug administration until 90 d after the last study drug intake. The table includes TESAE from both treatment cohorts for the four double included patients.
²Data extraction date/eCRF data extract as of 24 May 2021.
AE = adverse event; PT = preferred term; TESAE = treatment-emergent serious adverse event.

TABLE 9
Lipo-MERIT - Number of Patients with Related TESAE by BNT111
Monotherapy or PD-1 Inhibitor Combination Therapy (N = 115)¹⁻⁴

| | BNT111 | BNT111 + PD-1 | BNT111 + | |
|---|---|---|---|---|
| | Monotherapy | inhibitor | BRAF/MEK | Total |
| TESAE | N = 71 | N = 38 | (N = 7) | (N = 115) |
| Patients with any related TESAE | 5 (7.0) | 8 (21.1) | 0 (0.0) | 13 (11.3) |
| Pyrexia | 2 (2.8) | 0 (0.0) | 0 (0.0) | 2 (1.7) |
| Dizziness | 1 (1.4) | 1 (2.6) | 0 (0.0) | 2 (1.7) |
| Syncope | 0 (0.0) | 2 (5.3) | 0 (0.0) | 2 (1.7) |
| Hypotension | 1 (1.4) | 1 (2.6) | 0 (0.0) | 2 (1.7) |
| Lymphadenopathy | 1 (1.4) | 0 (0.0) | 0 (0.0) | 1 (0.9) |
| Serous retinopathy | 0 (0.0) | 1 (2.6) | 0 (0.0) | 1 (0.9) |
| Autoimmune pancreatitis | 0 (0.0) | 1 (2.6) | 0 (0.0) | 1 (0.9) |
| Nausea | 0 (0.0) | 1 (2.6) | 0 (0.0) | 1 (0.9) |
| Asthenia | 1 (1.4) | 0 (0.0) | 0 (0.0) | 1 (0.9) |
| General physical health deterioration | 0 (0.0) | 1 (2.6) | 0 (0.0) | 1 (0.9) |
| Anaphylactic reaction | 0 (0.0) | 1 (2.6) | 0 (0.0) | 1 (0.9) |
| Cytokine release syndrome | 0 (0.0) | 1 (2.6) | 0 (0.0) | 1 (0.9) |
| Encephalitis autoimmune | 0 (0.0) | 1 (2.6) | 0 (0.0) | 1 (0.9) |
| Epilepsy | 0 (0.0) | 1 (2.6) | 0 (0.0) | 1 (0.9) |
| Posterior reversible encephalopathy syndrome | 0 (0.0) | 1 (2.6) | 0 (0.0) | 1 (0.9) |

¹Data extraction date/eCRF data extract as of 24 May 2021.
²TESAE is defined as occurring after start of study drug administration until 90 d after the last study drug intake.
³One patient may have suffered from a TESAE that was coded with more than one preferred term.
⁴One patient who received BNT111 as monotherapy in the first enrolment and BNT111 + BRAF/MEK in the second enrolment is represented on this table. Therefore, there is a difference between the total number and the sum of individual therapies displayed.
PD-1 = Programmed death 1; TESAE = treatment-emergent serious adverse event.

Of note, eight patients are still on trial treatment with BNT111 monotherapy (n=2) or the combination of BNT1111 with a PD-i inhibitor (n=6) in the Lipo-MERIT trial. These eight patients, all with multiple prior lines of therapy, were in the so called ‘continued treatment’ with treatment durations between 15 up to 52 months. Initially, ‘continued treatment’ was only offered as long as all IMP components (based on four precursor RNAs RBL001.1, RBL002.2, RBL003.1 and RBL004.1) were in stock. However, since these eight heavily pretreated patients achieved at least stabilization of their disease or a response (partial remission or complete remission according to irRECIST) and thus continue to derive clinical benefit from trial therapy, the trial was not stopped, but instead, further trial treatment was offered onwards with the current BNT111 material (so called ‘extended treatment’). The trial was allowed to continue for the benefit of these patients.

### Example 19: Pharmacology Data Obtain in Mice

Mice can be a relevant species to assess primary and secondary pharmacological as well as potential toxicological effects of the RNA-LPX complex and thus to capture potential substance-specific (i.e., RNA molecule-specific) toxicities of RNA-LPX. Mice exhibit all primary and secondary pharmacological effects from induction of CD4+ and/or CD8+ T cell responses to immunomodulatory effects that enhance the immunological response and lead to subsequent TLR triggering, cellular activation, and cytokine secretion. However, given the species-specificity of the BNT111 TAAs and the unique set of MHC molecules in every patient which can present a large set of antigenic peptides, there are no relevant and conclusive mouse tumor models for the human melanoma TAAs encoded by BNT111, and pharmacodynamic studies in mice for BNT111 as a single-agent or in combination with checkpoint blockade are not feasible. Therefore, most primary pharmacodynamics, mechanism of action, and anti-tumor activity studies were conducted with RNA-LPX vaccines encoding model antigens in mice.

Nonclinical studies conducted have demonstrated that vaccination with RNA LPX induces DC maturation and activation of major lymphocyte subsets in the spleen, and systemic cytokine release including IFNα, TNFα, IP-10 and IL 6 in response to TLR7 triggering by the single-stranded RNA within the first 3 to 6 hours in mice (Kranz et al. 2016, which is incorporated herein by reference in its entirety). Transient leukopenia coincides with IFNα peak levels and can be attributed to IFNα downstream effects.

Vaccination with RNA LPX in mice efficiently de novo primes and expands cytotoxic CD4+ and CD8+ T cells targeting the BNT111-encoded antigens NY ESO 1, tyrosinase, MAGE A3, TPTE and other melanoma-associated or model antigens. BNT111 RNA-loaded human DCs are capable of stimulating IFN-γ production by antigen-specific CD8+ T cells expressing the corresponding TCRs RNA dose-dependently after in vitro co-incubation.

Induced antigen-specific CD8+ T cells were demonstrated to be able to infiltrate mouse tumors, and RNA LPX vaccination was associated with the polarization of the tumor microenvironment towards a pro-inflammatory, cytotoxic, and less immune-suppressive contexture. RNA LPX vaccination appears to trigger antigen release from the tumor which enables vaccine-induced, tumor-specific T cells to expand further, even after treatment discontinuation.

Tumor-infiltrating CD8+ Tcells upregulate the expression of PD 1 in response to RNA LPX vaccination, and PD L1 is significantly expressed by the tumor. T cells with high PD 1 expression are considered to have a high antigen affinity. As hypothesized, the combination of RNA LPX vaccination with PD 1/PD L1 checkpoint blockade synergizes in the inhibition of tumor growth and improvement of survival by rendering PD 1/PD L1 blockade resistant mouse tumors susceptible to this treatment combination. Enhancement of vaccine-induced break of tolerance against B16 melanoma-expressed self-antigens by PD 1/PD L1 blockade further demonstrates the strong anti-tumoral activity of the combination of RNA LPX vaccination with PD 1/PD L1 blockade.

Table 10 summarizes the nonclinical primary pharmacodynamic studies performed with BNT111.

TABLE 10
Summary of BNT111 Nonclinical Primary Pharmacodynamic Studies

| Parameter | Test | | | |
|---|---|---|---|---|
| Evaluated | System | Test Items* | Dosage (μg/dose) | Key Findings |
| In vivo | Mice, | BNT111: | IV, SD | RNA-LPX induces DC and lymphocyte |
| immune | C57BL/6, | NY-ESO-1 | BNT111: 15 μg | activation in the spleen 24 h after injection. |
| stimulation; | C57BL/6 | (RBL001.1), tyrosinase | (3.75 μg per | RNA-LPX induces systemic release of IFNα |
| monotherapy | IFNAR1^{-/-} | (RBL002.2), MAGE- | target) RNA-LPX | and TNFα (max: 6 h, return to baseline: |
| | | A3 (RBL003.1), TPTE | | 24 h). |
| | | (RBL004.1) RNA-LPX | | RNA-LPX induces transient leukopenia |
| | | (ATM) | | (max: 8 h, return to baseline: 48 h). |
| | | | | RNA component drives immune stimulation |
| | | | | rather than the lipid component. |
| | | | | Clinical relevance: RNA molecules have |
| | | | | immunomodulatory effects based on their |
| | | | | ability to induce cellular activation processes |
| | | | | and subsequent cytokine production by |
| | | | | binding to intracellular and endosomal |
| | | | | pattern recognition receptors. Pro- |
| | | | | inflammatory cytokine release, leukocyte |
| | | | | activation and leukopenia, are hallmarks of |
| | | | | systemic inflammatory cytokine secretion. |
| In vivo | Mice, | BNT111: NY-ESO-1 | IV, SD | RNA-LPX induces activation of splenic DC |
| immune | C57BL/6 | (RBL001.1), | BNT111: 40 μg | subsets, macrophages, NK, T and B cells |
| stimulation; | | tyrosinase | RNA-LPX per | 24 h after injection. |
| monotherapy | | (RBL002.2), MAGE- | target and mouse | Clinical relevance: These data confirm the |
| | | A3 (RBL003.1), TPTE | | immunostimulatory effect of RNA-LPX and |
| | | (RBL004.1) RNA-LPX | | suggest that these effects are largely |
| | | (ATM) | | independent of RNA sequence and length. |
| In vitro T cell | Co- | BNT111: NY-ESO-1 | 0.25, 1, 4, | BNT111 RNA-loaded human DCs are |
| immunity; | incubation | (RBL001.1), | 16 μg | capable of stimulating IFN-γ production by |
| monotherapy | of target- | tyrosinase | | antigen-specific CD8⁺ T cells expressing the |
| | expressing | (RBL002.2), MAGE- | | corresponding TCRs RNA dose-dependently |
| | hiDCs with | A3 (RBL003.1), TPTE | | after in vitro co-incubation. |
| | TCR- | (RBL004.1) RNA-LPX | | Clinical relevance: These data confirm the |
| | electropo- | (ATM) | | antigen-specific, immunostimulatory and |
| | rated T cells | | | dose-dependent effect of RNA-LPX. |
| In vivo T cell | Mice, HLA- | BNT111: NY-ESO-1 | IV, RD (Day 1, 4, | RNA-LPX induces antigen-specific CD8⁺ |
| immunity; | A2.1^{+/+} | (RBL001.1), | 8, 11 [also on | T cells in human MHC-tg A2/DR1 mice. |
| monotherapy | HLA-DR1^{+/+} | tyrosinase | Day 18, MAGE-3 | Induced T cells secrete IFN-γ upon ex vivo |
| | double-tg | (RBL002.2), MAGE- | only]) | target recognition and kill target cells in vivo. |
| | | A3 (RBL003.1), TPTE | BNT111: 30 μg | Clinical relevance: Induced T cells secrete |
| | | (RBL004.1) RNA-LPX | per target and | IFN-γ upon ex vivo target recognition and |
| | | (ATM) | mouse | kill target cells in vivo. |
| In vivo T cell | Mice, HLA- | BNT111: NY-ESO-1 | IV, RD (Day 1, 8, | RNA-LPX induces antigen-specific CD8⁺ |
| immunity: | A2.1^{+/+} | (RBL001.1), | 15, 22) | T cells in human MHC-tg A2/DR1 mice. |
| monotherapy | HLA-DR1^{+/+} | tyrosinase | BNT111: 30 μg | Induced T cells secrete IFN-γ upon ex vivo |
| | double-tg | (RBL002.2), MAGE- | per target and | target recognition. |
| | | A3 (RBL003.1), TPTE | mouse | Clinical relevance: Induced T cells secrete |
| | | (RBL004.1) RNA-LPX | | IFN-γ upon ex vivo target recognition. |
| | | (CTM) | | |

DC = dendritic cell; dsRNA = double-stranded RNA; HA = influenza hemagglutinin; hiDC = human immature DC; HPV = human papilloma virus; IFN = interferon; IFNAR1 = interferon α and β receptor subunit 1; IL = interleukin; IP10 = interferon-γ-inducible protein 10; IV = intravenous; MHC = major histocompatibility complex; NK = natural killer; OVA = ovalbumin; PBMC = peripheral blood mononuclear cell; pDC = plasmacytoid DC; PD-1 = programmed death ligand 1; PD-L1 = programmed death protein 1; SD = single-dose; RD = repeated dose; TAM = tumor-associated macrophage; TCR = T cell receptor; tg = transgenic; TIL = tumor-infiltrating leukocytes; TLR = toll-like receptor; TME = tumor microenvironment; TNF = tumor necrosis factor; Treg = CD4⁺ CD25⁺ FoxP3⁺ T regulatory cell; TRP = tyrosinase-related protein; WB = whole blood.
*The initial development (applied in Lipo-MERIT trial) was based on the four precursor Drug Products RBL001.1, RBL002.2, RBL003.1 and RBL004.1, which are encoding the same targets, but have been slightly improved, e.g., for RNA translatability and stability.

To further elucidate the primary pharmacodynamics, mechanism of action and anti-tumor activity of BNT111, and to generate a rationale for the combination of BNT111 with PD-1/PD-L1 checkpoint blockade, RNA-LPX vaccines encoding model antigens (e.g., human papillomavirus 16 oncoprotein E7) or other melanoma-associated antigens (tyrosinase-related protein 1 and 2) were applied.

TABLE 11
Summary of BNT111 Supportive Non-clinical Primary Pharmacodynamic Studies

| Parameter | Test | | Dosage | |
|---|---|---|---|---|
| Evaluated | System | Test Items | (μg/dose) | Key Findings |
| In vivo | Mice, | HA RNA-LPX | HA: 20 or 40 μg | Immune cell activation, cytokine release and |
| immune | C57BL/6, | | RNA-LPX | leukopenia are dependent on TLR7 and |
| stimulation | C57BL/6 | | | IFNAR1 signaling. |
| | IFNAR1^{-/-}, | | | |
| | C57BL/6 | | | |
| | TLR7^{-/-} | | | |
| In vivo | Mice, | HA RNA-LPX | IV, SD | Pseudouridine-modified and dsRNA- |
| immune | C57BL/6 | HA RNA-LPX, | HA: 10 μg | depleted RNA-LPX is immunosilent and |
| stimulation | | pseudouridine-modified | RNA-LPX | neither induces activation of splenic immune |
| | | and dsRNA-purified | | cell subsets or systemic IFNα release. |
| | | | | RNA component drives immune stimulation |
| | | | | rather than the lipid component. |
| In vivo | Mice, | TRP1 RNA-LPX | IV, RD (Day 8, | RNA-LPX induces antigen-specific CD8⁺ |
| T cell | C57BL/6, | | 15, 22) | T cells. |
| immunity | B16-F10 | | 20 μg TRP1 | Induction of antigen-specific T cells by |
| | tumor- | | RNA-LPX | vaccination against self-antigens |
| | bearing | | | demonstrates break of immunological |
| | | | | tolerance. |
| | | | | T_{regs} are not expanded by vaccination, |
| | | | | resulting in beneficial antigen-specific CD8⁺ |
| | | | | T cell to T_{reg} ratio. |
| In vivo | Mice, | HPV16 E7 (research- | IV, SD (Day 13) | RNA-LPX induces antigen-specific CD8⁺ |
| T cell | C57BL/6, | grade RBL016.1) | HPV16 E7: 40 μg | T cells after single vaccination similarly with |
| immunity | TC-1 | RNA-LPX | RNA-LPX | and without PD-L1 blockade. |
| | tumor- | Control RNA-LPX | Control: 40 μg | Induced T cells are PD-1-positive. |
| | bearing | Anti-PD-L1 blocking | RNA-LPX | Induced T cells are provided with the ability |
| | | antibody | IP, RD (Day 18, | to expand independently of vaccination in the |
| | | | 25, 32, 39, 46, | presence of tumor-released antigen, which is |
| | | | 53, 60) | further promoted by PD-L1 blockade. |
| | | | anti-PD-L1: | |
| | | | 200 μg once, then | |
| | | | 100 μg | |
| In vivo | Mice, | gp70 RNA-LPX | IV, RD (Day 0, 3, | RNA-LPX induces antigen-specific CD8⁺ T cells. |
| T cell | BALB/c | | 8, 15 or Day 0, 3, | Induced T cells secrete IFNγ upon ex vivo |
| immunity | | | 7, 14) | target recognition and kill target cells in vivo. |
| | | | 40 μg gp70 | |
| | | | RNA-LPX | |
| In vivo | Mice, | BNT113: HPV16 | IV, RD (Day 17, | E7 RNA-LPX inhibits tumor growth and |
| anti-tumor | C57BL/6 | E6 (RBL015.1), | 19, 25 or Day 13, | increases survival to 90% (treatment start |
| activity | | HPV16 E7 (RBL016.1) | 20, 27). | Day 10), and 30% (treatment start Day 13). |
| TC-1 tumor | | RNA-LPX | HPV16 E6 and | Large tumors initially regress but relapse |
| model (SC) | | (research-grade) | E7: 40 μg | after treatment discontinuation. |
| | | OVA RNA-LPX | RNA-LPX per | Overall therapeutic success correlates with |
| | | | target and mouse | tumor size at treatment start. |
| | | | OVA: 40 μg | E6 RNA-LPX did not have any effect. |
| | | | RNA-LPX | |
| In vivo | Mice, | OVA RNA-LPX, gp70 | IV, RD (Day 10, | RNA-LPX delays tumor growth and prolongs |
| anti-tumor | C57BL/6, | RNA-LPX | 13, 17, 24, 31, 38, | survival. |
| activity | BALB/c | | 45, 53, 63); OVA: | RNA-LPX-induced antigen-specific CD8⁺ |
| B16-OVA | | | 40 μg RNA-LPX | T cells correlate with delayed tumor growth |
| tumor model | | | IV, RD (Day 6, 9, | and survival. |
| (SC), CT26 | | | 13, 21, 28, 35); | |
| tumor | | | gp70: 40 μg | |
| model (SC) | | | RNA-LPX | |
| In vivo TIL | Mice, | HPV16 (research-grade | IV, SD (Day 11) | HPV16 E6/E7⁺ tumors are highly infiltrated |
| analysis | C57BL/6 | RBL016.1) E7 | HPV16 E7: 40 μg | with CD4⁺ and CD8⁺ T cells, NK cells, DCs |
| TC-1 tumor | | RNA-LPX | RNA-LPX | and TAMs, and especially antigen-specific |
| model (SC) | | | | CD8⁺ T cells in response to RNA-LPX. |
| | | | | Antigen-specific CD8⁺ T cells induced by |
| | | | | RNA-LPX express effector cytokines IFNγ |
| | | | | and gzmB- upon ex vivo target recognition. |
| | | | | RNA-LPX polarizes the TME towards a pro- |
| | | | | inflammatory, cytotoxic and less immune- |
| | | | | suppressive contexture. |
| | | | | Antigen-specific CD8⁺ T cells induced by |
| | | | | RNA-LPX are PD-1-positive while tumor |
| | | | | cells upregulate PD-L1. |
| In vivo TIL | Mice, | HPV16 E7 (research- | IV, SD (Day 11) | HPV16 E6/E7⁺ tumors display signature of |
| analysis | C57BL/6 | grade RBL016.1) | HPV16 E7: 40 μg | CD8⁺ T cell function (CD8, IFNγ, PD-1) and |
| TC-1 tumor | | RNA-LPX | RNA-LPX | T cell infiltration-promoting factors (CCL19, |
| model (SC) | | | | CCL21, CXCL9) early on after RNA-LPX (Day 7). |
| | | | | Regressing tumors demonstrate a broad |
| | | | | immune activatory profile (Day 13), |
| | | | | consisting of T cell costimulatory markers |
| | | | | (ICOS, CD28, CD69, CD27), infiltration- |
| | | | | promoting chemokines and their receptors |
| | | | | (CCL5, CCL19, CXCL9, CXCL12; CCR5, |
| | | | | CXCR3), pro-inflammatory cytokines (IL- |
| | | | | 1β, IL-6, IFNγ), Th1 differentiation |
| | | | | (TBX21), DC maturation (CD40, CD86), and |
| | | | | monocyte/macrophage recruitment (F4/80, |
| | | | | CCL2, GM-CSF). |
| | | | | These tumors express PD-L1 and CTLA-4 to |
| | | | | counteract and suppress immune attack. |

CCL = CC chemokine ligand, CCR = CC chemokine receptor, CTLA = cytotoxic T-lymphocyte-associated protein, CXCL = CXC chemokine ligand, HA = influenza hemagglutinin, HPV = Human papillomavirus, ICOS = inducible T cell costimulatory, IFN = interferon, IL = interleukin, gzm = granzyme, PD-1 = programmed death-1, PD-L1 = programmed death ligand 1, SC = subcutaneous, TAM = tumor-associated macrophages, TBX = T-box transcription factor, TCR = T cell receptor, tg = transgenic, TIL = tumor-infiltrating leukocytes, TLR = toll-like receptor, TME = tumor microenvironment, TNF = tumor necrosis factor, Treg = CD4⁺ CD25⁺ FoxP3⁺ T regulatory cell, TRP = tyrosinase-related protein, WB = whole blood.

### REFERENCES

References below may be referred throughout this disclosure. Each is incorporated herein by reference in its entirety.
1. Melero, I. et al. Therapeutic vaccines for cancer: an overview of clinical trials. Nat. Rev. Clin. Oncol. 11, 509-524 (2014).
2. Romero, P. et al. The Human Vaccines Project: a roadmap for cancer vaccine development. Sci. Transl. Med. 8, 334ps9 (2016).
3. Coulie, P. G., Van den Eynde, B. J., van der Bruggen, P. & Boon, T. Tumour antigens recognized by T lymphocytes: at the core of cancer immunotherapy. Nat. Rev. Cancer 14, 135-146 (2014).
4. Kyewski, B. & Derbinski, J. Self-representation in the thymus: an extended view. Nat. Rev. Immunol. 4, 688-698 (2004).
5. Holtkamp, S. et al. Modification of antigen-encoding RNA increases stability, translational efficacy, and T-cell stimulatory capacity of dendritic cells. Blood 108, 4009-4017 (2006).
6. Orlandini von Niessen, A. G. et al. Improving mRNA-based therapeutic gene delivery by expression-augmenting 3′ UTRs identified by cellular library screening. Mol. Ther. 27, 824-836 (2019).
7. Kreiter, S. et al. Increased antigen presentation efficiency by coupling antigens to MHC class I trafficking signals. J. Immunol. 180, 309-318 (2008).
8. Kranz, L. M. et al. Systemic RNA delivery to dendritic cells exploits antiviral defence for cancer immunotherapy. Nature 534, 396-401 (2016).
9. De Vries, J. & Figdor, C. Immunotherapy: cancer vaccine triggers antiviral-type defences. Nature 534, 329-331 (2016).
10. Simon, P. et al. Functional TCR retrieval from single antigen-specific human T cells reveals multiple novel epitopes. Cancer Immunol. Res. 2, 1230-1244 (2014).
11. Cheever, M. A. et al. The prioritization of cancer antigens: a national cancer institute pilot project for the acceleration of translational research. Clin. Cancer Res. 15, 5323-5337 (2009).
12. Pektor, S. et al. Toll like receptor mediated immune stimulation can be visualized in vivo by [18F]FDG-PET. Nucl. Med. Biol. 43, 651-660 (2016).
13. Reinhard, K. et al. An RNA vaccine drives expansion and efficacy of claudin-CAR-T cells against solid tumors. Science 367, 446-453 (2020).
14. Pektor, S. et al. In vivo imaging of the immune response upon systemic RNA cancer vaccination by FDG-PET. EJNMMI Res. 8, 80 (2018).
15. Jackson, H. et al. Striking immunodominance hierarchy of naturally occurring CD8+ and CD4+ T cell responses to tumor antigen NY-ESO-1. J. Immunol. 176, 5908-5917 (2006).
16. Hu, Y. et al. Immunologic hierarchy, class II MHC promiscuity, and epitope spreading of a melanoma helper peptide vaccine. Cancer Immunol. Immunother. 63, 779-786 (2014).
17. Hanagiri, T., van Baren, N., Neyns, B., Boon, T. & Coulie, P. G. Analysis of a rare melanoma patient with a spontaneous CTL response to a MAGE-A3 peptide presented by HLA-A1. Cancer Immunol. Immunother. 55, 178-184 (2006).
18. Grunwitz, C. et al. HPV16 RNA-LPX vaccine mediates complete regression of aggressively growing HPV-positive mouse tumors and establishes protective T cell memory. OncoImmunology 8, e1629259 (2019).
19. Robert, C. et al. Pembrolizumab versus ipilimumab in advanced melanoma. N. Engl. J. Med. 372, 2521-2532 (2015).
20. Rosenberg, S. A., Yang, J. C. & Restifo, N. P. Cancer immunotherapy: moving beyond current vaccines. Nat. Med. 10, 909-915 (2004).
21. Ribas, A. & Wolchok, J. D. Cancer immunotherapy using checkpoint blockade. Science 359, 1350-1355 (2018).
22. Hugo, W. et al. Genomic and transcriptomic features of response to anti-PD-1 therapy in metastatic melanoma. Cell 165, 35-44 (2016).
23. Simpson, A. J. G., Caballero, O. L., Jungbluth, A., Chen, Y.-T. & Old, L. J. Cancer/testis antigens, gametogenesis and cancer. Nat. Rev. Cancer 5, 615-625 (2005).
24. Hofbauer, G. F., Kamarashev, J., Geertsen, R., Böni, R. & Dummer, R. Tyrosinase immunoreactivity in formalin-fixed, paraffin-embedded primary and metastatic melanoma: frequency and distribution. J. Cutan. Pathol. 25, 204-209 (1998).
25. Nishino, M., Gargano, M., Suda, M., Ramaiya, N. H. & Hodi, F. S. Optimizing immunerelated tumor response assessment: does reducing the number of lesions impact response assessment in melanoma patients treated with ipilimumab? J. Immunother. Cancer 2, 17 (2014).
26. Sahin, U. et al. Personalized RNA mutanome vaccines mobilize poly-specific therapeutic immunity against cancer. Nature 547, 222-226 (2017).
27. Grabbe, S. et al. Translating nanoparticulate-personalized cancer vaccines into clinical applications: case study with RNA-lipoplexes for the treatment of melanoma. Nanomedicine 11, 2723-2734(2016).
28. Batzri, S. & Korn, E. D. Single bilayer liposomes prepared without sonication. Biochim. Biophys. Acta 298, 1015-1019 (1973).
29. Barichello, J. M., Ishida, T. & Kiwada, H. Complexation of siRNA and pDNA with cationic liposomes: the important aspects in lipoplex preparation. Methods Mol. Biol. 605, 461-472 (2010).
30. Carey, T. E., Takahashi, T., Resnick, L. A., Oettgen, H. F. & Old, L. J. Cell surface antigens of human malignant melanoma: mixed hemadsorption assays for humoral immunity to cultured autologous melanoma cells. Proc. Natl Acad. Sci. USA 73, 3278-3282 (1976).
31. Brochet, X., Lefranc, M.-P. & Giudicelli, V. IMGTN-QUEST: the highly customized and integrated system for IG and TR standardized V-J and V-D-J sequence analysis. Nucleic Acids Res. 36, W503-W508 (2008).
32. Bolotin, D. A. et al. MiXCR: software for comprehensive adaptive immunity profiling. Nat. Methods 12, 380-381 (2015).
33. Shugay, M. et al. VDJtools: unifying post-analysis of T cell receptor repertoires. PLOS Comput. Biol. 11, e1004503 (2015).
34. Li, H. & Durbin, R. Fast and accurate short read alignment with Burrows-Wheeler transform. Bioinformatics 25, 1754-1760 (2009).
35. Patro, R., Mount, S. M. & Kingsford, C. Sailfish enables alignment-free isoform quantification from RNA-seq reads using lightweight algorithms. Nat. Biotechnol. 32, 462-464(2014).
36. Robinson, D. R. et al. Integrative clinical genomics of metastatic cancer. Nature 548, 297-303 (2017).
37. American Cancer Society: Cancer Facts & Figures 2021. Atlanta, GA: American Cancer Society; 2021. Available from: https://www.cancer.org/content/dam/cancer-org/research/cancer-facts-and-statistics/annual-cancer-facts-and-figures/2021/cancer-facts-and-figures-2021.pdf (accessed on Aug. 26, 2021).
38. Banchereau J, Ueno H, Dhodapkar M, et al. Immune and clinical outcomes in patients with stage IV melanoma vaccinated with peptide-pulsed dendritic cells derived from CD34+ progenitors and activated with type I interferon. J Immunother. 2005; 28(5): 505-16.
39. Brichard V G, Lejeune D. GSK's antigen-specific cancer immunotherapy programme: pilot results leading to Phase III clinical development. Vaccine. 2007; 27; 25 Suppl 2:B61-71.
40. Carrasco J, Van Pel A, Neyns B, et al. Vaccination of a melanoma patient with mature dendritic cells pulsed with MAGE-3 peptides triggers the activity of nonvaccine anti-tumor cells. J Immunol 2008; 180(5): 3585-93.
41. Chen Q, Jackson H, Shackleton M, et al. Characterization of antigen-specific CD8+ T lymphocyte responses in skin and peripheral blood following intradermal peptide vaccination. Cancer Immun. 2005; 5: 5.
42. Coricovac D, Dehelean C, Moaca E A, et al. Cutaneous Melanoma-A Long Road from Experimental Models to Clinical Outcome: A Review. Int J Mol Sci. 2018; 19(6):1566.
43. Demotz S, Lanzavecchia A, Eisel U, et al. Delineation of several DR-restricted tetanus toxin T cell epitopes. J Immunol. 1989; 142(2): 394-402.
44. Dredge K, Marriott J B, Todryk S M, Dalgleish A G. Adjuvants and the promotion of Th1-type cytokines in tumour immunotherapy. Cancer Immunol Immunother. 2002; 51(10): 521-31.
45. Gellrich F F, Schmitz M, Beissert S, Meier F. Anti-PD-1 and novel combinations in the treatment of melanoma—an update. J Clin Med. 2020; 14; 9(1): 223.
46. Gershenwald J E, Scolyer R A, Hess, D R, et al. Melanoma staging: evidence-based changes in the American Joint Committee on Cancer Eighth Edition Cancer Staging Manual. CA Cancer J Clin 2017; 67:472-492.
47. Guo J, Si L, Kong Y, et al. Phase II, open-label, single-arm trial of imatinib mesylate in patients with metastatic melanoma harboring c-Kit mutation or amplification. J Clin Oncol 2011; 29(21): 2904-9.
48. Hauschild A, Kahler K C, Schafer M, Fluck M. Interdisciplinary management recommendations for toxicity associated with interferon-alfa therapy. J Dtsch Dermatol Ges. 2008; 6(10): 829-38.
49. Holtkamp S, Kreiter S, Selmi A, et al. Modification of antigen-encoding RNA increases stability, translational efficacy, and T cell stimulatory capacity of dendritic cells. Blood. 2006; 108(13): 4009-17.
50. International Agency for Research on Cancer. GLOBOCAN 2020: Population factsheets. Available at: https://gco.iarc.fr/today/data/factsheets/populations/908-europe-fact-sheets.pdf. Accessed: 16 Jun. 2021.
51. KEYTRUDA® United States Prescribing Information. Available at: https://www.merck.com/product/usa/pi_circulars/k/keytruda/keytruda_pi.pdf (accessed on Aug. 26, 2021).
52. Kranz L M, Diken M, Haas H, et al. Systemic RNA delivery to dendritic cells exploits antiviral defence for cancer immunotherapy. Nature. 2016; 534(7607): 396-401.
53. Kreiter S, Selmi A, Diken M, et al. Increased antigen presentation efficiency by coupling antigens to MHC class I trafficking signals. J Immunol. 2008; 180(1): 309-18.
54. Kuk D, Shoushtari A N, Barker C A, et al. Prognosis of mucosal, uveal, acral, nonacral cutaneous, and unknow primary melanoma from the time of first metastasis. The Oncologist. 2016; 21:848-854.
55. LIBTAYO® United States Prescribing Information. Available at: https://www.accessdata.fda.gov/drugsatfda_docs/label/2021/61097s007lbl.pdf (accessed on Aug. 26, 2021).
56. Livingston K A, Jiang X, Stephensen C B. CD4 T-helper cell cytokine phenotypes and antibody response following tetanus toxoid booster immunization. J Immunol Methods. 2013; 390(1-2): 18-29.
57. Mackiewicz J, Mackiewicz A. BRAF and MEK inhibitors in the era of immunotherapy in melanoma patients. Contemp Oncol 2018; 22(1A): 68-72.
58. Marchand M, Punt C J, Aamdal S, et al. Immunisation of metastatic cancer patients with MAGE-3 protein combined with adjuvant SBAS-2: a clinical report. Eur J Cancer 2003; 39(1): 70-7.
59. Michielin O, van Akkooi A C J, Ascierto P A, et al. Cutaneous melanoma: ESMO Clinical Practice. Guidelines for diagnosis, treatment and follow-up. Annals of Oncology. 2019; 30:1884-1901.
60. Mooradian M J, Sullivan R J. What to do when anti-PD-1 therapy fails in patients with melanoma. Oncology (Williston Park). 2019; 33(4): 141-48.
61. OPDIVO® United States Prescribing Information. Available at: https://packageinserts.bms.com/pi/pi_opdivo.pdf (accessed on Aug. 26, 2021).
62. Orlandini von Niessen A G; Poleganov M A, Rechner C, et al. Improving mRNA-Based Therapeutic Gene Delivery by Expression-Augmenting 3′ UTRs Identified by Cellular Library Screening. Mol Ther. 2019; 27(4): 824-36.
63. Oshita C, Takikawa M, Kume A, et al. Dendritic cell-based vaccination in metastatic melanoma patients: phase II clinical trial. Oncol Rep 2012; 28(4): 1131-8.
64. Rehman H, Silk A W, Kane M P, Kaufman H L. Into the clinic: Talimogene aherparepvec (TVEC), a first-in-class intratumoral oncolytic viral therapy. J Immunother Cancer 2016; 4:53.
65. Sanderson K, Scotland R, Lee P, et al. (2005): Autoimmunity in a phase I trial of a fully human anti-cytotoxic T-lymphocyte antigen-4 monoclonal antibody with multiple melanoma peptides and Montanide ISA 51 for patients with resected stages III and IV melanoma. J Clin Oncol. 2005; 23(4): 741-50.
66. SEER Cancer Statistics Review (CSR) 1975-2017. National Cancer Institute. 16. Melanoma of the Skin. https://seer.cancer.gov/csr/1975_2017/results_merged/sect_16_melanoma_skin.pdf. (Table 16.8) (accessed on Aug. 26, 2021).
67. Shackleton M, Davis I D, Hopkins W, et al. The impact of imiquimod, a Toll-like receptor-7 ligand (TLR7L), on the immunogenicity of melanoma peptide vaccination with adjuvant Flt3 ligand. Cancer Immun. 2004; 4: 9.
68. Sharpe A H, Pauken K E. The diverse functions of the PD1 inhibitory pathway. In Nature reviews. Immunology. 2018; 18(3): 153-67.
69. Siegal R L, Miller K D, Fuchs H E, et al. Cancer statistics 2021. CA Cancer J Clin. 2021; 71:7-33.
70. Slingluff C L Jr, Petroni G R, Yamshchikov G V, et al. Clinical and immunologic results of a randomized phase II trial of vaccination using four melanoma peptides either administered in granulocyte-macrophage colony-stimulating factor in adjuvant or pulsed on dendritic cells. J Clin Oncol. 2003; 21(21): 4016-26.
71. Srivastava S, Koch M A, Pepper M, Campbell D J. Type I interferons directly inhibit regulatory T cells to allow optimal antiviral T cell responses during acute LCMV infection. J Exp Med. 2014; 211(5): 961-74.
72. Swetter S M, Thompson J A, Albertini M R, et al. (2021). Melanoma: Cutaneous—NCCN Clinical Practice Guidelines in Oncology. Version 2.2021—Feb. 19, 2021.
73. Testori A A E, Chellino S, and van Akkooi A C J. Adjuvant therapy for melanoma: past, current, and future developments. Cancers 2020; 12:1-15.
74. Toungouz M, Libin M, Bulté F, et al. Transient expansion of peptide-specific lymphocytes producing IFN-gamma after vaccination with dendritic cells pulsed with MAGE peptides in patients with mage-A1/A3-positive tumors. J Leukoc Biol 2001; 69(6): 937-43.
75. Tyagi P, Mirakhur B. MAGRIT: the largest-ever phase III lung cancer trial aims to establish a novel tumor-specific approach to therapy. Clin Lung Cancer. 2009; 10(5): 371-74.
76. Van der Kooij M K, Speetjens F M, van der Burg S H, et al. Uveal versus cutaneous melanoma; same origin, very distinct tumor types. Cancers. 2019; 11:3-16.
77. Weide B, Pascolo S, Scheel B, et al. Direct injection of protamine-protected mRNA: results of a phase 1/2 vaccination trial in metastatic melanoma patients. J Immunother. 2009; 32(5): 498-07.
78. Wilgenhof S, Van Nuffel A M, Corthals J, et al. Therapeutic vaccination with an autologous mRNA electroporated dendritic cell vaccine in patients with advanced melanoma. J Immunother 2011; 34(5): 448-56.
79. Wolchok J D, Chiarion-Sileni V, Gonzalez R, et al. Overall survival with combined nivolumab and ipilimumab in advanced melanoma. N Engl J Med. 2017; 377(14):1345-1356.
80. YERVOY® United States Prescribing Information. Available at: https://packageinserts.bms.com/pi/pi_yervoy.pdf (accessed on Aug. 26, 2021).
81. Zinkernagel R M, Ehl S, Aichele P, et al. Antigen localisation regulates immune responses in a dose- and time-dependent fashion: a geographical view of immune reactivity. Immunol Rev. 1997; 156: 199-209.

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

1. A method comprising:
administering to a patient at least one dose of a pharmaceutical composition comprising:
(a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and
(b) lipid particles;
wherein the patient was diagnosed with cancer prior to the time of administration, but the patient is classified as having no evidence of disease at the time of administration.

2. The method of claim 1, wherein no evidence of disease is or was determined by applying an immune-related Response Evaluation Criteria In Solid Tumors (irRECIST) standard or RECIST 1.1 standard.

3. A method of treating a patient suffering from cancer with a combination therapy, wherein the combination therapy comprises:
(i) administering to the patient at least one dose of a pharmaceutical composition comprising:
(a) one or more RNA molecules that collectively encode (i) a New York oesophageal squamous cell carcinoma (NY-ESO-1) antigen, (ii) a melanoma-associated antigen A3 (MAGE-A3) antigen, (iii) a tyrosinase antigen, (iv) a transmembrane phosphatase with tensin homology (TPTE) antigen, or (v) a combination thereof; and
(b) lipid particles; and
(ii) administering to the patient a PD-1 inhibitor.

4. The method of claim 3, wherein the patient is classified as having no evidence of disease at the time of treatment.

5. The method of claim 3, wherein the patient is classified as having evidence of disease at the time of treatment.

6. The method of claim 4, wherein evidence of disease or no evidence of disease is or was determined by applying an immune-related Response Evaluation Criteria In Solid Tumors (irRECIST) standard or RECIST 1.1 standard.

7. The method of claim 1, wherein the one or more RNA molecules comprise:
(i) a first RNA molecule encoding the NY-ESO-1 antigen,
(ii) a second RNA molecule encoding a MAGE-A3 antigen,
(iii) a third RNA molecule encoding a tyrosinase antigen, and
(iv) a fourth RNA molecule encoding a TPTE antigen.

8. (canceled)

9. The method of claim 1, wherein a single RNA molecule of the one or more RNA molecules encodes a polyepitopic polypeptide, wherein the polyepitopic polypeptide comprises at least two of the NY-ESO-1 antigen, the MAGE-A3 antigen, the tyrosinase antigen, and the TPTE antigen.

10. The method of claim 1, wherein the one or more RNA molecules further comprise at least one sequence that encodes a CD4+ epitope.

11. The method of claim 1, wherein the one or more RNA molecules further comprise at least one sequence that encodes tetanus toxoid P2, a sequence that encodes tetanus toxoid P16, or both.

12. The method of claim 1, wherein the one or more RNA molecules comprise a sequence encoding an MHC class I trafficking domain.

13. The method of claim 1, wherein the one or more RNA molecules comprises a 5′ cap or 5′ cap analogue.

14. The method of claim 1, wherein the one or more RNA molecules comprises a sequence encoding a signal peptide.

15. The method of claim 1, wherein the one or more RNA molecules comprise at least one non-coding regulatory element.

16. The method of claim 1, wherein the one or more RNA molecules comprises a poly-adenine tail.

17. The method of claim 16, wherein the poly-adenine tail is or comprises a modified adenine sequence.

18. The method of claim 1, wherein the one or more RNA molecules comprises at least one 5′ untranslated region (UTR) and/or at least one 3′ UTR.

19. The method of claim 18, wherein the one or more RNA molecules comprises in 5′ to 3′ order:
(i) a 5′ cap or 5′ cap analogue;
(ii) at least one 5′ UTR;
(iii) a signal peptide;
(iv) a coding region that encodes at least one of the NY-ESO-1 antigen, the MAGE-A3 antigen, the tyrosinase antigen, and the TPTE antigen;
(v) at least one sequence that encodes tetanus toxoid P2, tetanus toxoid P16, or both;
(vi) a sequence encoding an MHC class I trafficking domain;
(vii) at least one 3′UTR; and
(viii) a poly-adenine tail.

20. The method of claim 1, wherein the one or more RNA molecules comprise natural ribonucleotides.

21. The method of claim 1, wherein the one or more RNA molecules comprise modified or synthetic ribonucleotides.

22. The method of claim 1, wherein at least one of the NY-ESO-1 antigen, the MAGE-A3 antigen, the tyrosinase antigen, and the TPTE antigen are full-length, non-mutated antigens.

23. The method of claim 1, wherein all of the NY-ESO-1 antigen, the MAGE-A3 antigen, the tyrosinase antigen, and the TPTE antigen are full-length, non-mutated antigens.

24. The method of claim 1, wherein at least one of the NY-ESO-1 antigen, the MAGE-A3 antigen, the tyrosinase antigen, and the TPTE antigen are expressed from dendritic cells in lymphoid tissues of the patient.

25. The method of claim 1, wherein at least one of the NY-ESO-1 antigen, the MAGE-A3 antigen, the tyrosinase antigen, and the TPTE antigen are present in the cancer.

26.-34. (canceled)

35. The method of claim 1, wherein the patient is a human.

36. (canceled)

37. The method of claim 1, wherein the cancer is a melanoma.

38.-41. (canceled)

42. The method of claim 1, wherein the cancer is fully resected, there is no evidence of disease, or both.

43.-71. (canceled)

72. The method of claim 3, wherein the PD-1 inhibitor is or comprises nivolumab, pembrolizumab, or cemiplimab.

73. The method of claim 3, wherein the PD-1 inhibitor is or comprises cemiplimab.

74. (canceled)

75. The method of claim 1, wherein the pharmaceutical composition induces an immune response in the patient.

76. The method of claim 1, further comprising determining a level of the immune response in the patient.

77. (canceled)

78. The method of claim 1, wherein the pharmaceutical composition induces a level of the immune response in the patient that is comparable to a level of the immune response in a second patient to which the pharmaceutical composition has been administered, has previously been diagnosed with cancer, and is classified as having evidence of disease at the time of administration.

79.-83. (canceled)

84. The method of claim 75, wherein the immune response in the patient is an adaptive immune response.

85. The method of claim 75, wherein the immune response in the patient is a T-cell response.

86. The method of claim 85, wherein the T-cell response is or comprises a CD4+ response.

87. The method of claim 85, wherein the T-cell response is or comprises a CD8+ response.

88.-145. (canceled)
